(19) United States
(12) Patent Application Publication
(10) Pub. No.: US 2016/0031935 A1
(43) Pub. Date: Feb. 4, 2016

(54) SMALL MOLECULE MODULATORS OF PCSK9 AND METHODS OF USE THEREOF

(71) Applicants: ADAERATA, LIMITED PARTNERSHIP, Montréal (CA); AMORCHEM HOLDINGS INC., Montréal (CA)

(72) Inventors: DANIEL GUAY, LACHINE (CA); SHELDON CRANE, ILE PERROT (CA); NICOLAS LACHANCE, PIERREFONDS (CA); JEAN-FRANCOIS CHIASSON, SAINT-CONSTANT (CA); VOUY LINH TRUONG, PIERREFONDS (CA); PATRICK LACOMBE, MONTREAL (CA); KATHRYN SKOREY, BEACONSFIELD (CA); NABIL G. SEIDAH, ILE-DES-SOEURS (CA)

(21) Appl. No.: 14/776,701

(22) PCT Filed: Mar. 14, 2014
(86) PCT No.: PCT/CA2014/050255

§ 371 Date: Sep. 14, 2015

Related U.S. Application Data
(60) Provisional application No. 61/792,249, filed on Mar. 15, 2013.

(51) Int. Cl. C07K 5/08; C07K 5/087; C07K 5/083; A61K 38/06; A61K 45/06

(52) U.S. Cl. CPC C07K 5/0827; A61K 38/06; A61K 45/06; C07K 5/0808; C07K 5/0812

(57) ABSTRACT

A compound of Formula (I): or a pharmaceutically acceptable salt, hydrate, solvate, or racemic mixture or stereoisomer thereof, and methods for preventing or treating an LDL-cholesterol-related disease or disorder using such compound(s), and kits and compositions comprising such compound(s).

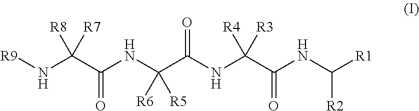

### CROSS REFERENCE TO RELATED APPLICATIONS

This application is a National Entry Application of PCT application Serial No PCT/CA2014/* filed on Mar. 14, 2014 and published in English under PCT Article 21(2), which itself claims benefit of U.S. provisional application Ser. No. 61/792,249, filed on Mar. 15, 2013. All documents above are incorporated herein in their entirety by reference.

### STATEMENT REGARDING FEDERALLY SPONSORED RESEARCH OR DEVELOPMENT

N. A.

### FIELD OF THE INVENTION

The present invention relates to small molecule modulators of proprotein convertase subtilisin-kexin type 9 (PCSK9) and methods of use thereof. More specifically, the present invention is concerned with the use of these molecules in the treatment of low-density lipoproteins (LDL)-cholesterol-related diseases or disorders.

### BACKGROUND OF THE INVENTION

Complications resulting from cardiovascular disorders are the main cause of death worldwide, affecting ˜13 million individuals/year, as compared to ˜6 million/year due to various forms of cancer. One of the most potent cardiovascular risk factors is elevated levels of low-density lipoprotein (LDL) cholesterol (LDL-C). The incidence of cardiovascular pathologies is expected to increase dramatically in the next two decades. Clinical trial data has demonstrated that reductions in LDL cholesterol levels are related to the rate of coronary events (Law et al., 2003 BMJ 326:1423-1427). Moderate lifelong reduction in plasma LDL cholesterol levels has been shown to be substantially correlated with a significant reduction in the incidence of coronary events (Cohen et al., N. Engl. J. Med. 354:1264-1272), even in populations with a high prevalence of non-lipid-related cardiovascular risk factors. Accordingly, there is great benefit to be gained from the managed control of LDL cholesterol levels. Among important cholesterol-lowering drugs are statins (Briel, M., Nordmann, A. J., and Bucher, H. C. Curr. Opin. Lipidol., 16: 601-605, 2005). Though well tolerated by the majority of patients, adverse side effects are being compiled (https://www.statineffects.com/info/). The combination of statins with ezetimibe, an intestinal sterol transporter blocker, further reduces LDL-C by ≦20%.

Thus, there is a need for the development of alternative strategies to decrease levels of circulating LDL-C (Brown, M. S. and Goldstein, J. L. Science, 311: 1721-1723, 2006; Tall, A. R. N. Engl. J Med., 354: 1310-1312, 2006).

The low-density lipoprotein receptor (LDLR) is a key player in the regulation of circulating levels of LDL in the bloodstream. Reduced levels of LDLR are associated with elevated plasma cholesterol (LDL-C) which is strongly correlated with an increased risk of atherosclerosis and coronary heart disease—the leading cause of death in industrialized civilizations. Agents that effect an increase of LDLR protein levels at the cell surface would thereby provide a means to normalize LDL-C in an individual affected by plasma hypercholesterolemia. The proprotein convertase PCSK9 decreases LDLR levels by enhancing its degradation in endosomes/lysosomes. It has been established in a number of clinical trials that inhibition of PCSK9-mediated LDLR degradation with monoclonal antibodies dose-dependently decrease LDL-C in healthy human subjects (Lambert et. al., The PCSK9 Decade’, J. Lipid Res. 2012, 53(12), 2515-24 and references cited therein). From a cost and convenience of dosing standpoint, the development of an orally-available, small molecule agent which decreases the amount of secreted PCSK9 would be a desirable alternative to intravenous or subcutaneous delivery of a biologic agent such as an antibody or an oligonucleotide tailored to disrupt the PCSK9 mediated degradation of LDLR.

### SUMMARY OF THE INVENTION

More specifically, in accordance with the present invention, there is provided a compound of Formula (I):

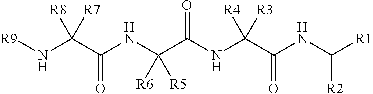

or a pharmaceutically acceptable salt, hydrate, solvate, or racemic mixture or stereoisomer thereof,
wherein:

### R1 is —CH(OH)Rₐ or —B(OR_{b})(OR_{c});

R2 is —H, —CH₂R_{d}, —CHR_{d}(Rₑ), —CH₂(CH₂)ₘC(O)Rⱼ, —CH₂(CH₂)ₘC(O)N(R_{d})Rₑ or —CH₂(CH₂)ₘS(O)ₙN(R_{d})Rₑ;
R3, R4, R5, R6, R7 and R8 are identical or different, and are independently hydrogen or one of the following groups: a C1-6 alkyl, a C1-6 haloalkyl, a C1-6 thioalkyl, an alkenyl, an alkynyl, an aryl, and an heteroaryl group, wherein said group is optionally substituted with one or more C1-6 alkyl, C3-8 cycloalkyl, C1-6 haloalkyl, aryl, aheteroaryl, —CN, —C(O)N(R_{f})R_{g}, —C(O)OR_{f}, —C(R_{f})(R_{g})ORₕ, —OR_{f}, —OC(O)OR_{f}, —OC(O)NR_{f}(R_{g}), —SR_{f}, —S(O)ₙR_{f}, S(O)ₙN(R_{f})R_{g}, —S(O)ₙN(R_{f})C(O)R_{g}, —N(R_{f})R_{g}, —N(R_{f})C(O)R_{g}, —N(R_{f})C(O)OR_{g}, —N(R_{f})C(O)N(R_{g})(Rₕ), —N(R_{f})S(O)ₙR_{g}, and —N(R_{f})S(O)N(R_{g})Rₕ substituents;
when (R3 and R4) or (R5 and R6) or (R7 and R8) are not hydrogen, the bracketed pairs can also be linked with —C(O)—, —CO₂—, —C(O)N(H)—, —C(O)N(Rₓ)— —O—, —NH—, —N(Rₓ)—, —S—, —S(O)—, —S(O)ₙN(H)—, —S(O)ₙN(Rₓ)— radicals to form cyclic structures;
R9 is RᵢC(O)—, RᵢS(O)—, RᵢOC(O)—, RᵢNHC(O)—, RᵢNHS(O)—, Rₖ(Rₗ)NC(O)—, Rₗ(Rₗ)NS(O)—; or one or more amino acids residues;
Rₐ is C1-3 alkyl, C1-2 fluoroalkyl or cyclopropyl;
R_{b} and R_{c} are identical or different, and are independently H or C1-6 alkyl, or can be connected together to form a cyclic 5- or 6-membered ring structure, or fused with additional aliphatic or aromatic ring systems, the cyclic 5- or 6-membered ring structure or aliphatic or aromatic ring systems being optionally substituted with one or more C1-6 alkyl and/or C1-6 haloalkyl substituents;
R_{d} and Rₑ are identical or different, and are independently H or one of the following groups: a C1-3 alkyl, a C1-3 haloalkyl or a C3-4 cycloalkyl group, or can be connected together directly or with —C(O)—, —C(O)O—, —C(O)N(Rₓ)— —O—, —N(Rₓ)—, —S—, —S(O)—, or —S(O)ₙN(Rₓ)— radicals to form cyclic 3-8 membered ring structures;
R_{f}, R_{g}, Rₕ, Rₖ and Rₗ are identical or different, and are independently H or one of the following groups: a C1-6 alkyl, a C1-6 haloalkyl or a C3-4 cycloalkyl group, or can be connected together directly or with —C(O)—, —C(O)O—, —C(O)N(Rₓ)— —O—, —N(Rₓ)—, —S—, —S(O)— or —S(O)ₙN(Rₓ)— radicals to form cyclic 3-8 membered ring structures;
Rᵢ is a C1-10 alkyl, a C3-8 cycloalkyl, a C1-10 haloalkyl, a heterocyclyl, an aryl or a heteroaryl group, wherein the group is optionally substituted with one or more of a halogen, C-1-6 aminoalkyl, C1-6 heteroalkyl, C1-6 alkyl, C3-8 cycloalkyl, C1-6 haloalkyl, aryl, heteroaryl and heterocyclyl groups;

### Rⱼ is OR_{d} or N(R_{d})(Rₑ);

Rₓ is H, C1-6 alkyl, C1-6 haloalkyl, C3-4 cycloalkyl, —C(O)R_{y}, —C(O)OR_{y}, —C(O)NH₂, —C(O)NH(R_{y}) or —C(O)NHS(O)ₙR_{y};
R_{y} is C1-6 alkyl, C1-6 haloalkyl or C3-4 cycloalkyl;
m is an integer of value 0 or 1; and
n is an integer of value 1 or 2,
provided that:
when R1 is —CH(OH)Rₐ, R2 is —CHR_{d}(Rₑ), —CH₂(CH₂)ₘC(O)Rⱼ, —CH₂(CH₂)ₘC(O)N(R_{d})Rₑ or —CH₂(CH₂)ₘS(O)ₙN(R_{d})Rₑ; and
when R1 is —B(OR_{b})(ORₑ), R2 is —H, —CH₃, —CHR_{d}(Rₑ), —CH₂(CH₂)ₘC(O)Rⱼ, —CH₂(CH₂)ₘC(O)N(R_{d})Rₑ or —CH₂(CH₂)ₘS(O)ₙN(R_{d})Rₑ.

More specifically, in accordance with the present invention, there is provided a Compound of Formula (I):

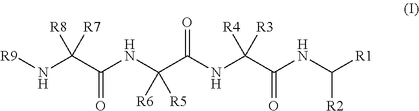

or a pharmaceutically acceptable salt, hydrate, solvate, or racemic mixture or stereoisomer thereof,
wherein:

### R1 is —CH(OH)Rₐ or —B(OR_{b})(OR_{c});

R2 is —H, —CH₂R_{d}, —CHR_{d}(Rₑ), —CH₂(CH₂)ₘC(O)Rⱼ, —CH₂(CH₂)ₘC(O)N(R_{d})Rₑ or —CH₂(CH₂)ₘS(O)ₙN(R_{d})Rₑ;
R3, R4, R5, R6, R7 and R8 are identical or different, and are independently hydrogen or one of the following groups: a C1-6 alkyl, a C1-6 haloalkyl, a C1-6 thioalkyl, a C1-6 aminoalkyl, an alkenyl, an alkynyl, a cycloalkyl, an heterocyclyl, an aryl, and an heteroaryl group, wherein said group is optionally substituted with one or more C1-6 alkyl, C3-8 cycloalkyl, C1-6 haloalkyl, aryl, an heteroaryl, —CN, —C(O)N(R_{f})R_{g}, —C(O)OR_{f}, —C(R_{f})(R_{g})ORₕ, —OR_{f}, —OC(O)OR_{f}, —OC(O)NR_{f}(R_{g}), —SR_{f}, —S(O)ₙR_{f}, —S(O)ₙN(R_{f})R_{g}, —S(O)ₙN(R_{f})C(O)R_{g}, —N(R_{f})R_{g}, —N(R_{f})C(O)R_{g}, —N(R_{f})C(O)OR_{g}, —N(R_{f})C(O)N(R_{g})(Rₕ), —N(R_{f})S(O)ₙR_{g}, and —N(R_{f})S(O)N(R_{g})Rₕ substituents;
when (R3 and R4) or (R5 and R6) or (R7 and R8) are not hydrogen, the bracketed pairs can also be linked with —C(O)—, —CO₂—, —C(O)N(H)—, —C(O)N(Rₓ)—, —O—, —NH—, —N(Rₓ)—, —S—, —S(O)—, —S(O)ₙN(H)—, —S(O)ₙN(Rₓ)— radicals to form cyclic structures;
R9 is RᵢC(O)—, RᵢS(O)—, RᵢOC(O)—, RᵢNHC(O)—, RᵢNHS(O)—, Rₖ(Rₗ)NC(O)—, Rₗ(Rₗ)NS(O)ₙ—; RₘORᵢC(O)—, RₘC(O)RᵢC(O)— or one or more amino acids residues;
Rₐ is C1-3 alkyl, C1-2 fluoroalkyl or cyclopropyl;
R_{b} and R_{c} are identical or different, and are independently H or C1-6 alkyl, or can be connected together to form a cyclic 5- or 6-membered ring structure, or fused with additional aliphatic or aromatic ring systems, the cyclic 5- or 6-membered ring structure or aliphatic or aromatic ring systems being optionally substituted with one or more C1-6 alkyl and/or C1-6 haloalkyl substituents;
R_{d} and Rₑ are identical or different, and are independently H or one of the following groups: a C1-3 alkyl, a C1-3 haloalkyl or a C3-4 cycloalkyl group, or can be connected together directly or with —C(O)—, —C(O)O—, —C(O)N(Rₓ)— —O—, —N(Rₓ)—, —S—, —S(O)—, or —S(O)ₙN(Rₓ)— radicals to form cyclic 3-8 membered ring structures;
R_{f}, R_{g}, Rₕ, Rₖ and Rₗ are identical or different, and are independently H or one of the following groups: a C1-6 alkyl, a C1-6 haloalkyl or a C3-4 cycloalkyl group, or can be connected together directly or with —C(O)—, —C(O)O—, —C(O)N(Rₓ)— —O—, —N(Rₓ)—, —S—, —S(O)— or —S(O)ₙN(Rₓ)— radicals to form cyclic 3-8 membered ring structures;
Rᵢ is a C1-10 alkyl, a C1-10 heteroalkyl, a C3-8 cycloalkyl, a C1-10 haloalkyl, a heterocyclyl, an aryl, a heteroaryl, a C1-10 alkyl-C3-8 cycloalkyl, a C1-10 alkyl-heterocyclyl, a C1-10 alkyl-aryl, a C1-10 alkyl-heteroaryl, a C1-10 heteroalkyl-C3-8 cycloalkyl, a C1-10 heteroalkyl-heterocyclyl, a C1-10 heteroalkyl-aryl or a C1-10 heteroalkyl-heteroaryl group, wherein the group is optionally substituted with one or more of a halogen, C1-6 aminoalkyl, C1-6 heteroalkyl, C1-6 alkyl, C3-8 cycloalkyl, C1-6 haloalkyl, aryl, heteroaryl and heterocyclyl groups;
Rₘ is a C1-10 alkyl, a C1-10 heteroalkyl, a C3-8 cycloalkyl, a C1-10 haloalkyl, a heterocyclyl, an aryl or a heteroaryl group, wherein the group is optionally substituted with one or more of a halogen, C1-6 aminoalkyl, C1-6 heteroalkyl, C1-6 alkyl, C3-8 cycloalkyl, C1-6 haloalkyl, aryl, heteroaryl and heterocyclyl groups;

### Rⱼ is OR_{d} or N(R_{d})(Rₑ);

Rₓ is H, C1-6 alkyl, C1-6 haloalkyl, C3-4 cycloalkyl, —C(O)R_{y}, —C(O)OR_{y}, —C(O)NH₂, —C(O)NH(R_{y}) or —C(O)NHS(O)ₙR_{y};
R_{y} is C1-6 alkyl, C1-6 haloalkyl or C3-4 cycloalkyl;
m is an integer of value 0 or 1; and
n is an integer of value 1 or 2,
provided that:
1) when R1 is —CH(OH)Rₐ, R2 is —CHR_{d}(Rₑ), —CH₂(CH₂)ₘC(O)Rⱼ, —CH₂(CH₂)ₘC(O)N(R_{d})Rₑ or —CH₂(CH₂)ₘS(O)ₙN(R_{d})Rₑ; and
2) when R1 is —B(OR_{b})(ORₑ), R2 is —H, —CH₃, —CHR_{d}(Rₑ), —CH₂(CH₂)ₘC(O)Rⱼ, —CH₂(CH₂)ₘC(O)N(R_{d})Rₑ or —CH₂(CH₂)ₘS(O)ₙN(R_{d})Rₑ.

In a specific embodiment of the compound of formula I, (i) when R1 is —CH(OH)—CF3, R9 is not —C(O)OCH₂-phenyl or —C(O)OCH₃; (ii) when R1 is B(OH)₂, R9 is not —C(O)OCH₃ or —C(O)-phenyl-phenyl; and/or (iii) when R1 is CH(OH)CH₂F, R9 is not —C(O)(CH₂)₈CH₃.

In a specific embodiment of the compound of formula I, R1 is —B(OR_{b})(OR_{c}), or —CH(OH)Rₐ; R2 is H or —CH₂(CH₂)ₘC(O)Rⱼ; R3 is H; R4 is C1-6 alkyl substituted or not, C1-6 thioalkyl substituted or not or aryl substituted or not; R5 is H; R6 is C1-6 alkyl substituted with aryl; R7 is H; R8 is C1-6 alkyl substituted with C1-6 alkyl; and/or R9 is RiOC(O)— or RiC(O)—.

In another specific embodiment of the compound of formula I, R1 is —B(OR_{b})(OR_{c}), or —CH(OH)Rₐ; R2 is H or —CH₂(CH₂)ₘC(O)Rⱼ; R3 is H; R4 is C1-6 alkyl substituted or not, C1-6 thioalkyl substituted or not or aryl substituted or not; R5 is H; R6 is C1-6 alkyl substituted with aryl; R7 is H; R8 is C1-6 alkyl substituted with C1-6 alkyl; and R9 is RiOC(O)— or RiC(O)—.

In another specific embodiment of the compound of formula I, R1 is —B(OR_{b})(OR_{c}), or —CH(OH)Rₐ; R2 is H or —CH₂(CH₂)ₘC(O)Rⱼ; R3 is H or C1-6 alkyl substituted or not; R4 is H, C1-6 alkyl substituted or not, C1-6 thioalkyl substituted or not, aryl substituted or not or cycloalkyl substituted or not; R5 is H; R6 is H, C1-6 alkyl substituted or not, C1-6 thioalkyl substituted or not, aryl substituted or not, or cycloalkane substituted or not; R7 is H; R8 is H, C1-6 alkyl substituted or not, C1-6 thioalkyl substituted or not, aryl substituted or not, or cycloalkyl substituted or not; and/or R9 is RiOC(O)—, RiC(O)—, RₘORᵢC(O)—, RₘC(O)RᵢC(O)—, or RᵢS(O)—.

In another specific embodiment of the compound of formula I, R1 is —B(OR_{b})(OR_{c}), or —CH(OH)Rₐ; R2 is H or —CH₂(CH₂)ₘC(O)Rⱼ; R3 is H or C1-6 alkyl substituted or not; R4 is H, C1-6 alkyl substituted or not, C1-6 thioalkyl substituted or not, aryl substituted or not or cycloalkyl substituted or not; R5 is H; R6 is H, C1-6 alkyl substituted or not, C1-6 thioalkyl substituted or not, aryl substituted or not, or cycloalkane substituted or not; R7 is H; R8 is H, C1-6 alkyl substituted or not, C1-6 thioalkyl substituted or not, aryl substituted or not, or cycloalkyl substituted or not; and R9 is RiOC(O)—, RiC(O)—, RₘORᵢC(O)—, RₘC(O)RᵢC(O)—, or RᵢS(O)—.

In a specific embodiment of the compound of formula I, R2; R3 or R4; R5 or R6; and/or R7 R8 can be identical or different and can be the side chain of any natural amino acid except proline in D or L configuration. Other substituents are as defined herein. In a more specific embodiment, (i) R2 is the side chain of glutamine or glycine; (ii) R3 or R4 is the side chain of alanine, phenylalanine, methionine, leucine, valine, glutamine or glycine; (iii) R5 or R6 is the side chain of phenylalanine, glycine, alanine, methionine, or valine; (iv) R7 or R8 is valine, methionine, phenylalanine, glycine or alanine; or (v) any combination of at least two of (i) to (iv).

In another specific embodiment of the compound of formula I, R6 is phenyl-CH₂—; and/or R8 is (CH₃)₂CH—. In another specific embodiment, R1 is —B(OR_{b})(OR_{c}). In another specific embodiment, Rb and Rc are H. In another specific embodiment, Rb and Rc are connected to form a cyclic 5 membered ring structure or fused with an aliphatic ring system. In another specific embodiment, R1 is 2,9,9-trimethyl-3,5-dioxa-4-boratricyclo[6.1.1.0^{2,6}]decan-4-yl. In another specific embodiment, R1 is —CH(OH)Rₐ. In another specific embodiment, Ra is C1-2 fluoroalkyl. In another specific embodiment, Ra is —CF₃. In another specific embodiment, Ra is —CH₂F. In another specific embodiment, Ra is C1-3 alkyl. In another specific embodiment, Ra is —CH₃. In another specific embodiment, R2 is H. In another specific embodiment, R2 is —CH₂(CH₂)ₘC(O)Rⱼ. In another specific embodiment, Rj is OR_{d}. In another specific embodiment, Rd is CH₃. In another specific embodiment, Rj is NH₂.

In another specific embodiment of the compound of formula I, R3 is H. In another specific embodiment, R3 is C1-6 alkyl substituted or not. In another specific embodiment, R3 is unsubstituted C1-6 alkyl. In another specific embodiment, R3 is CH₃.

In another specific embodiment of the compound of formula I, R4 is H. In another specific embodiment, R4 is C1-6 alkyl substituted or not. In another specific embodiment, R4 is unsubstituted C1-6 alkyl. In another specific embodiment, R4 is aryl substituted C1-6 alkyl. In another specific embodiment, R4 is —CH₃. In another specific embodiment, R4 is CH₃CH₂—. In another specific embodiment, R4 is —CH₂CH(CH₃)₂. In another specific embodiment, R4 is aryl substituted C1-6 alkyl. In another specific embodiment, R4 is phenyl-CH₂—. In another specific embodiment, R4 is —(CH₂)₂C(O)NH₂. In another specific embodiment, R4 is C1-6 thioalkyl substituted or not. In another specific embodiment, R4 is CH₃S(CH₂)₂—. In another specific embodiment, R4 is aryl substituted or not. In another specific embodiment, R4 is phenyl-. In another specific embodiment, R4 is cycloalkyl.

In another specific embodiment, R6 is H. In another specific embodiment, R6 is C1-6 alkyl substituted or not. In another specific embodiment, R6 is substituted C1-6 alkyl. In another specific embodiment, R6 is aryl substituted C1-6 alkyl. In another specific embodiment, R6 is -alkyl-phenyl. In another specific embodiment, R6 is —CH₂-phenyl. In another specific embodiment, R6 is —CH₂-hydroxyphenyl. In another specific embodiment, R6 is —(CH₂)₂-phenyl. In another specific embodiment, R6 is —CH₂-indole. In another specific embodiment, R6 is unsubstituted C1-6 alkyl. In another specific embodiment, R6 is unsubstituted C1-6 alkyl. In another specific embodiment, R6 is CH₃. In another specific embodiment, R6 is —CH(CH₃)₂. In another specific embodiment, R6 is C1-6 thioalkyl substituted or not. In another specific embodiment, R6 is CH₃S(CH₂)₂—. In another specific embodiment, R6 is aryl substituted or not. In another specific embodiment, R6 is phenyl. In another specific embodiment, R6 is cycloalkyl substituted or not. In another specific embodiment, R6 is benzocyclopentyl.

In another specific embodiment, R8 is H. In another specific embodiment, R8 is C1-6 alkyl substituted or not. In another specific embodiment, R8 is unsubstituted C1-6 alkyl. In another specific embodiment, R8 is —CH(CH₃)₂. In another specific embodiment, R8 is —CH₃. In another specific embodiment, R8 is substituted C1-6 alkyl. In another specific embodiment, R8 is aryl substituted C1-6 alkyl. In another specific embodiment, R8 is —CH₂-phenyl. In another specific embodiment, R8 is C1-6 thioalkyl substituted or not. In another specific embodiment, R8 is CH₃S(CH₂)₂—. In another specific embodiment, R8 is aryl substituted or not. In another specific embodiment, R8 is unsubstituted aryl. In another specific embodiment, R8 is phenyl. In another specific embodiment, R8 is cycloalkyl substituted or not. In another specific embodiment, R8 is unsubstituted cycloalkyl. R8 is cyclopentyl. In another specific embodiment, R8 is cyclopropyl.

In another specific embodiment, R9 is RiOC(O)—. In another specific embodiment, Ri is C1-10 alkyl, substituted or not-. In another specific embodiment, Ri is unsubstituted C1-10 alkyl-. In another specific embodiment, Ri is CH₃—. In another specific embodiment, Ri is substituted C1-10 alkyl-. In another specific embodiment, Ri is (CH₃)₃C—. In another specific embodiment, Ri is phenyl-CH₂—.

In another specific embodiment, R9 is RiC(O)—. In another specific embodiment, Ri is aryl substituted or not-. In another specific embodiment, Ri is substituted aryl-. In another specific embodiment, Ri is aryl substituted or not or heteroaryl substituted or not. In another specific embodiment, Ri is aryl substituted or not. In another specific embodiment, Ri is substituted aryl. In another specific embodiment, Ri is aryl-phenyl-. In another specific embodiment, Ri is phenyl-phenyl-. In another specific embodiment, Ri is heteroaryl-phenyl-. In another specific embodiment, Ri is diazirine-phenyl-. In another specific embodiment, Ri is phenyl-phenyl-. In another specific embodiment, Ri is fluorophenyl-. In another specific embodiment, Ri is fluoroalkyl-diazirin-phenyl-. In another specific embodiment, Ri is trifluoromethyl-diazirin-phenyl-. In another specific embodiment, Ri is pyridine-phenyl-. In another specific embodiment, Ri is oxadiazole-phenyl-. In another specific embodiment, Ri is heterocyclyl-phenyl-. In another specific embodiment, Ri is morpholine-phenyl-. In another specific embodiment, Ri is alkyl-phenyl-. In another specific embodiment, Ri is (CH₃)₂CH-phenyl-. In another specific embodiment, Ri is OHCH₂-phenyl-. In another specific embodiment, Ri is fluorophenyl. In another specific embodiment, Ri is unsubstituted aryl. In another specific embodiment, Ri is phenyl. In another specific embodiment, Ri is heteroaryl substituted or not. In another specific embodiment, Ri is substituted heteroaryl. In another specific embodiment, Ri is aryl-heteroaryl-. In another specific embodiment, Ri is phenyl-heteroaryl-. In another specific embodiment, Ri is phenyl-pyrazole-. In another specific embodiment, Ri is phenyl-methylpyrazole-. In another specific embodiment, Ri is phenyl-thiazole-. In another specific embodiment, Ri is phenyl-pyridine-. In another specific embodiment, Ri is phenyl-furazan-. In another specific embodiment, Ri is heteroaryl-heteroaryl-. In another specific embodiment, Ri is pyridine-isothiazole-. In another specific embodiment, Ri is unsubstituted heteroaryl. In another specific embodiment, Ri is pyridine. In another specific embodiment, Ri is pyrazine. In another specific embodiment, Ri is indole. In another specific embodiment, Ri is 4-[3-trifluoromethyl)-3H-diazirin-3-yl]phenyl-. In another specific embodiment, Ri is 3-[3-trifluoromethyl)-3H-diazirin-3-yl]phenyl-. In another specific embodiment, Ri is unsubstituted aryl-. In another specific embodiment, Ri is phenyl-. In another specific embodiment, Ri is C1-10 alkyl, substituted or not-. In another specific embodiment, Ri is substituted C1-10 alkyl-. In another specific embodiment, Ri is aryl-C1-10 alkyl-. In another specific embodiment, Ri is phenyl-C1-10 alkyl-. In another specific embodiment, Ri is phenyl-alkyne-. In another specific embodiment, Ri is phenyl-CH₂—.

In another specific embodiment, Ri is fluoromethoxy-aryl-(C1-6 alkyl)-. In another specific embodiment, Ri is fluoromethoxy-phenyl-CH₂—. In another specific embodiment, Ri is trifluoromethoxy-phenyl-CH₂—. In another specific embodiment, Ri is 4-(trifluoromethoxy)phenyl-CH₂—. In another specific embodiment, Ri is fluoroalkyl-diazirin-phenyl-(C1-10 alkyl). In another specific embodiment, Ri is trifluoromethyl-diazirin-phenyl-CH₂—. In another specific embodiment, Ri is 4-[3-trifluoromethyl)-3H-diazirin-3-yl]phenyl-. In another specific embodiment, Ri is 3-[3-trifluoromethyl)-3H-diazirin-3-yl]phenyl-. In another specific embodiment, Ri is unsubstituted C1-10 alkyl-. In another specific embodiment, Ri is CH₃(CH₂)₈—.

In another specific embodiment, R9 is RₘORᵢC(O)—. In another specific embodiment, Rᵢ is substituted or unsubstituted C1-10 alkyl. In another specific embodiment, Rᵢ is —CH₂—. In another specific embodiment, Rₘ is substituted or unsubstituted C1-10 alkyl. In another specific embodiment, Rₘ is —CH₂—. In another specific embodiment, Rₘ is aryl-CH₂—. In another specific embodiment, Rₘ is phenyl-CH₂—. In another specific embodiment, Rₘ is substituted or unsubstituted aryl. In another specific embodiment, Rₘ is phenyl.

In another specific embodiment, R9 is RₘC(O)RᵢC(O)—. In another specific embodiment, Rₘ is heteroaryl. In another specific embodiment, Rₘ is morpholine. In another specific embodiment, Rᵢ is aryl. In another specific embodiment, Rᵢ is phenyl.

In another specific embodiment, R9 is RᵢS(O)—. In another specific embodiment, Rᵢ is substituted or unsubstituted aryl. In another specific embodiment, Rᵢ is substituted aryl. In another specific embodiment, Rᵢ is substituted phenyl. In another specific embodiment, Rᵢ is phenyl-phenyl.

In another specific embodiment, the compound of formula I is:

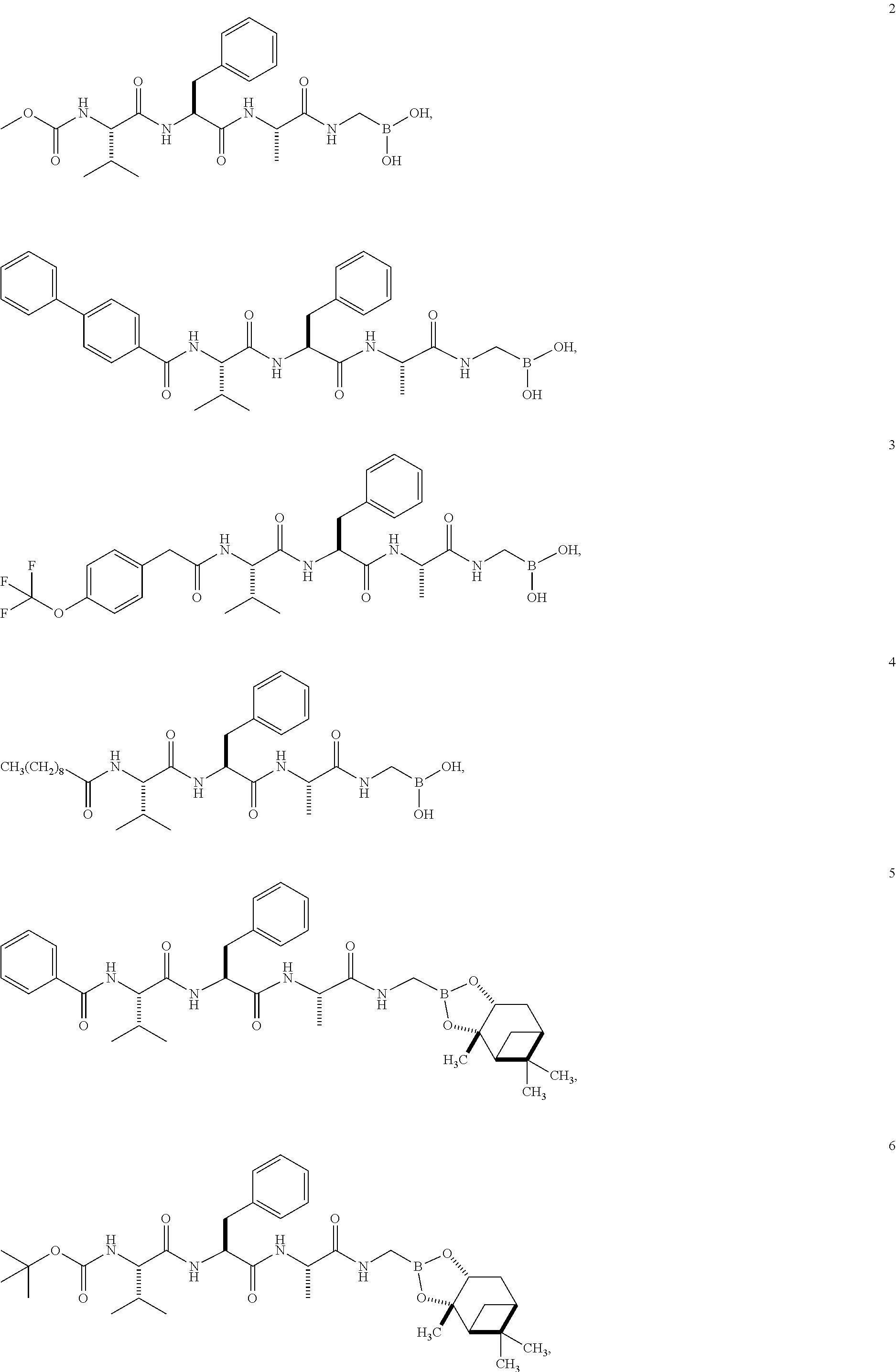

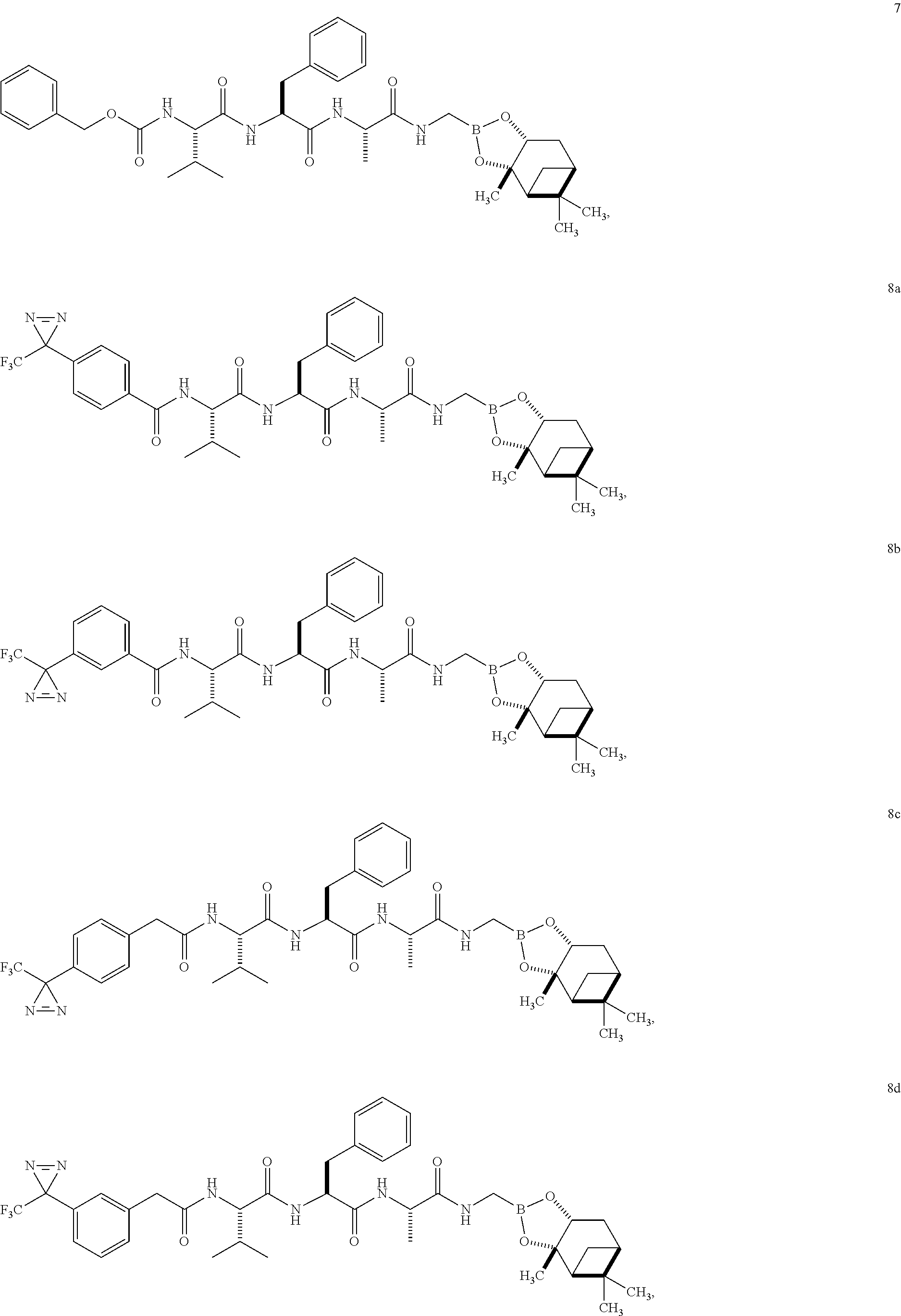

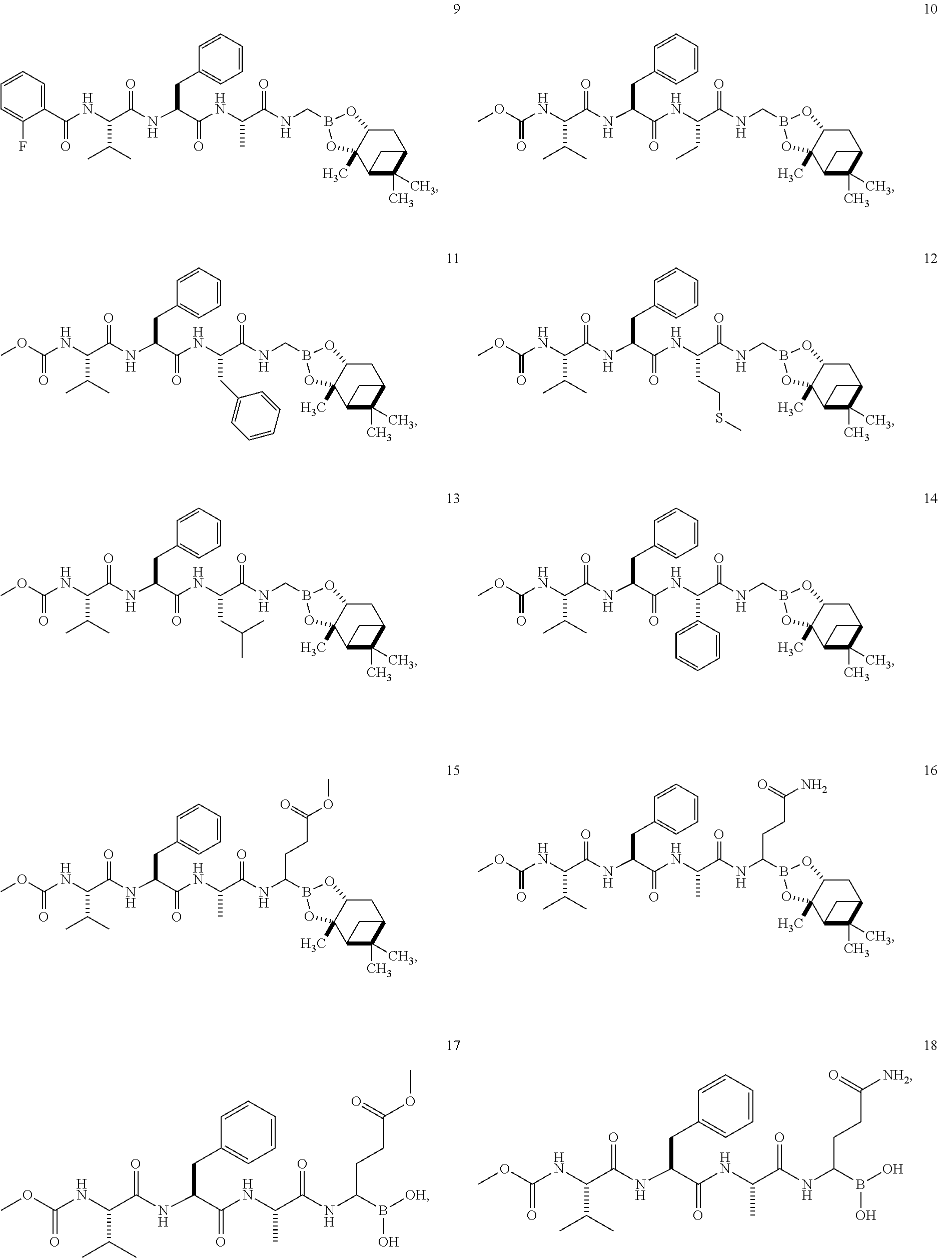

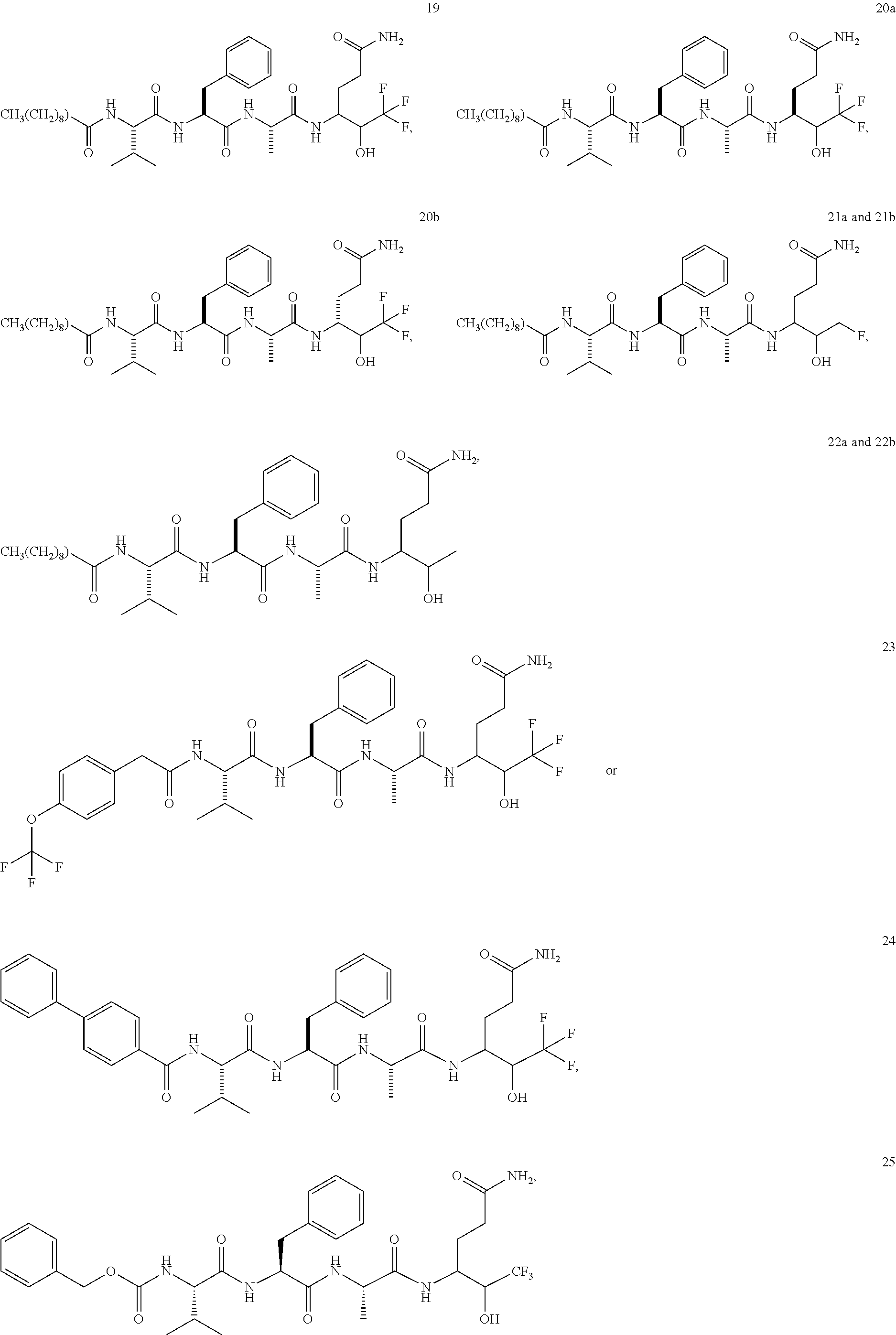

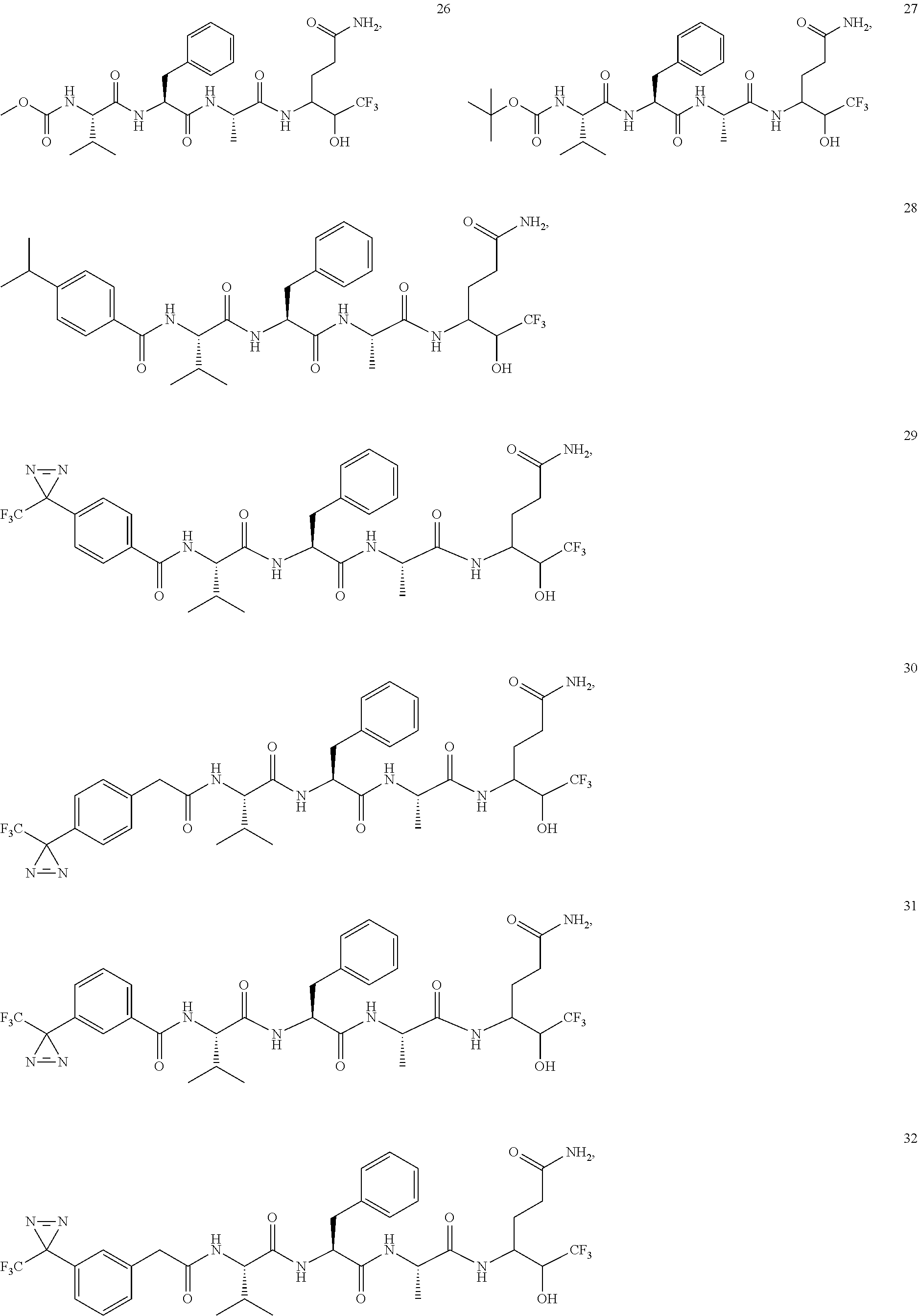

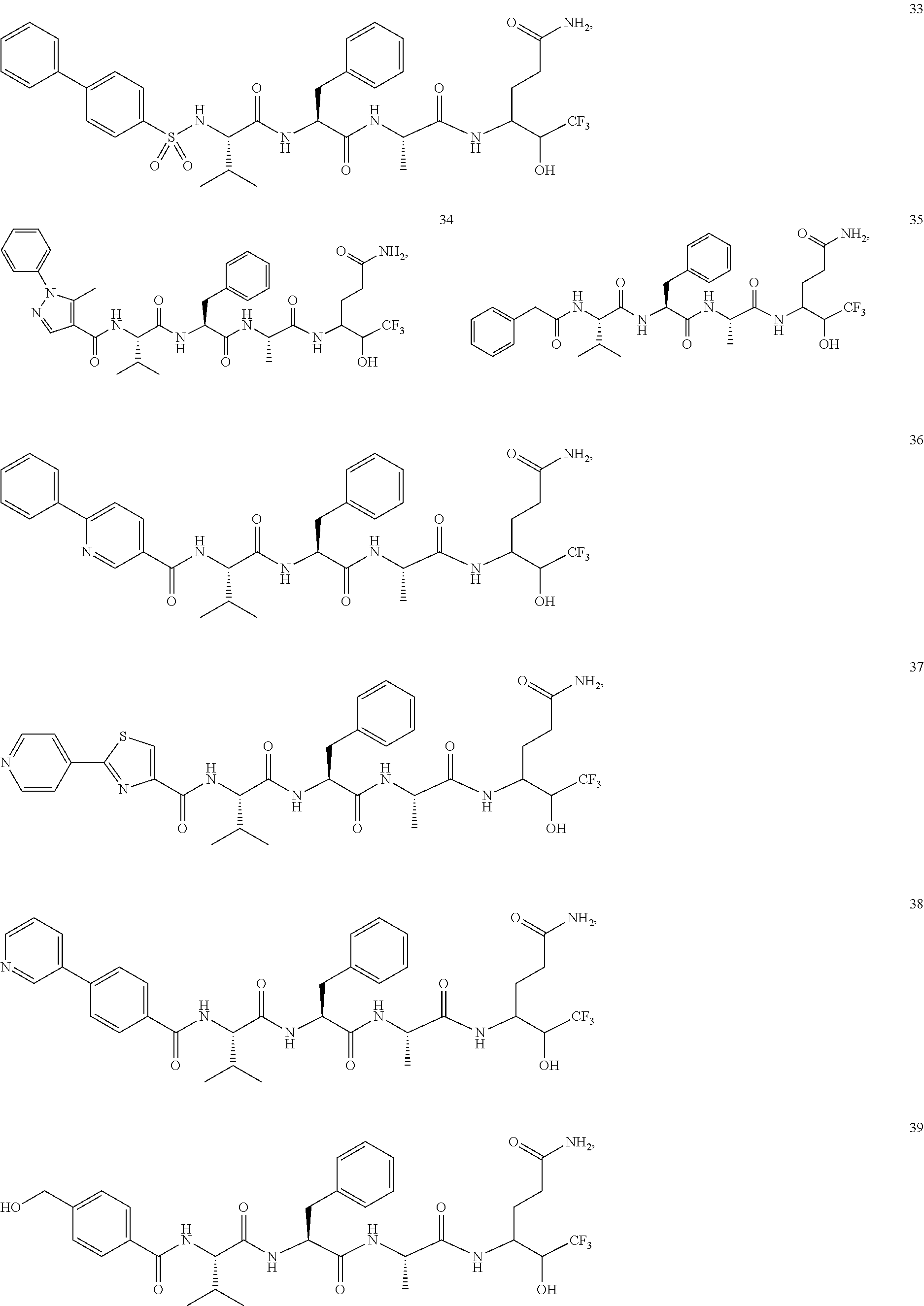

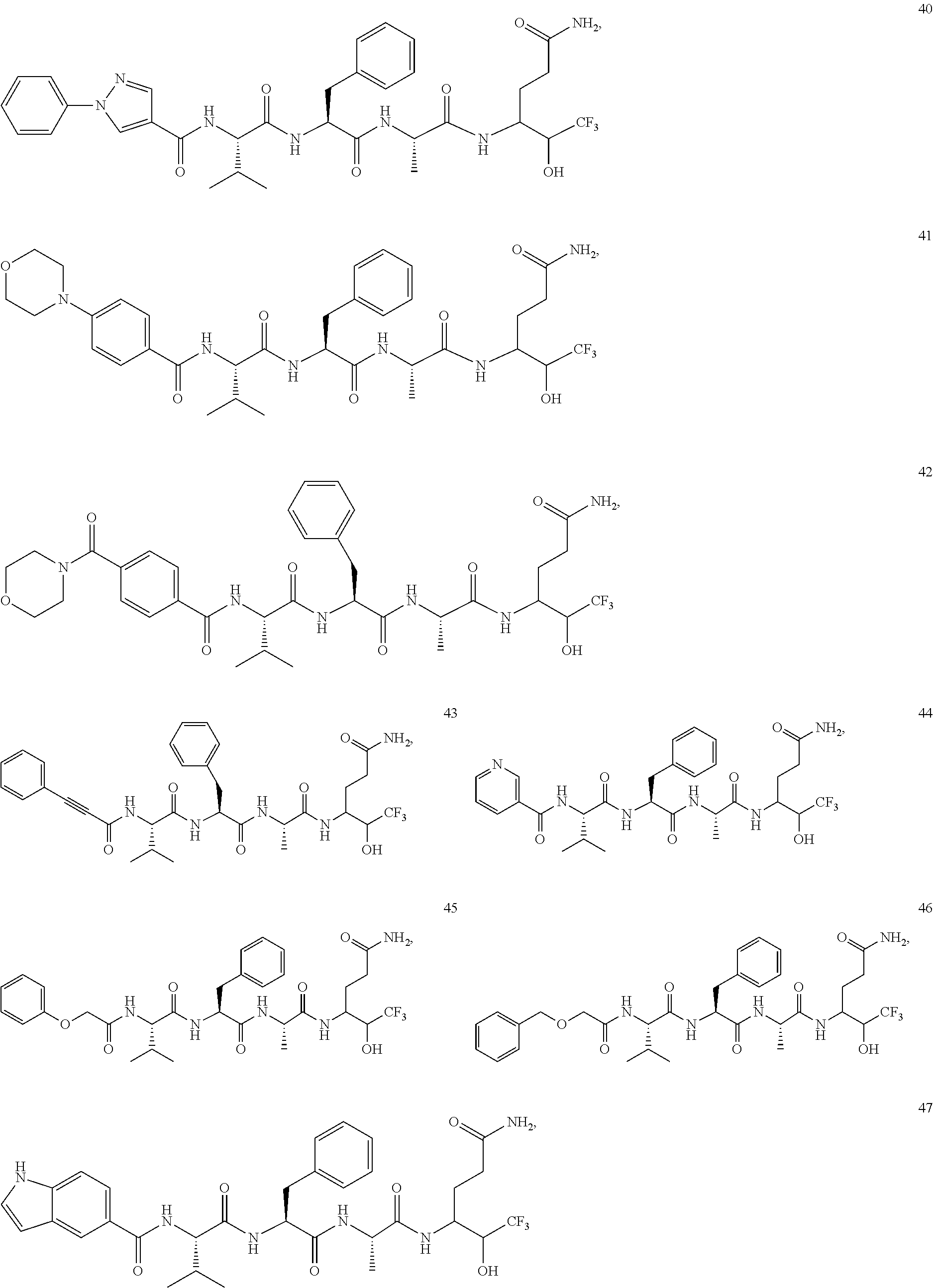

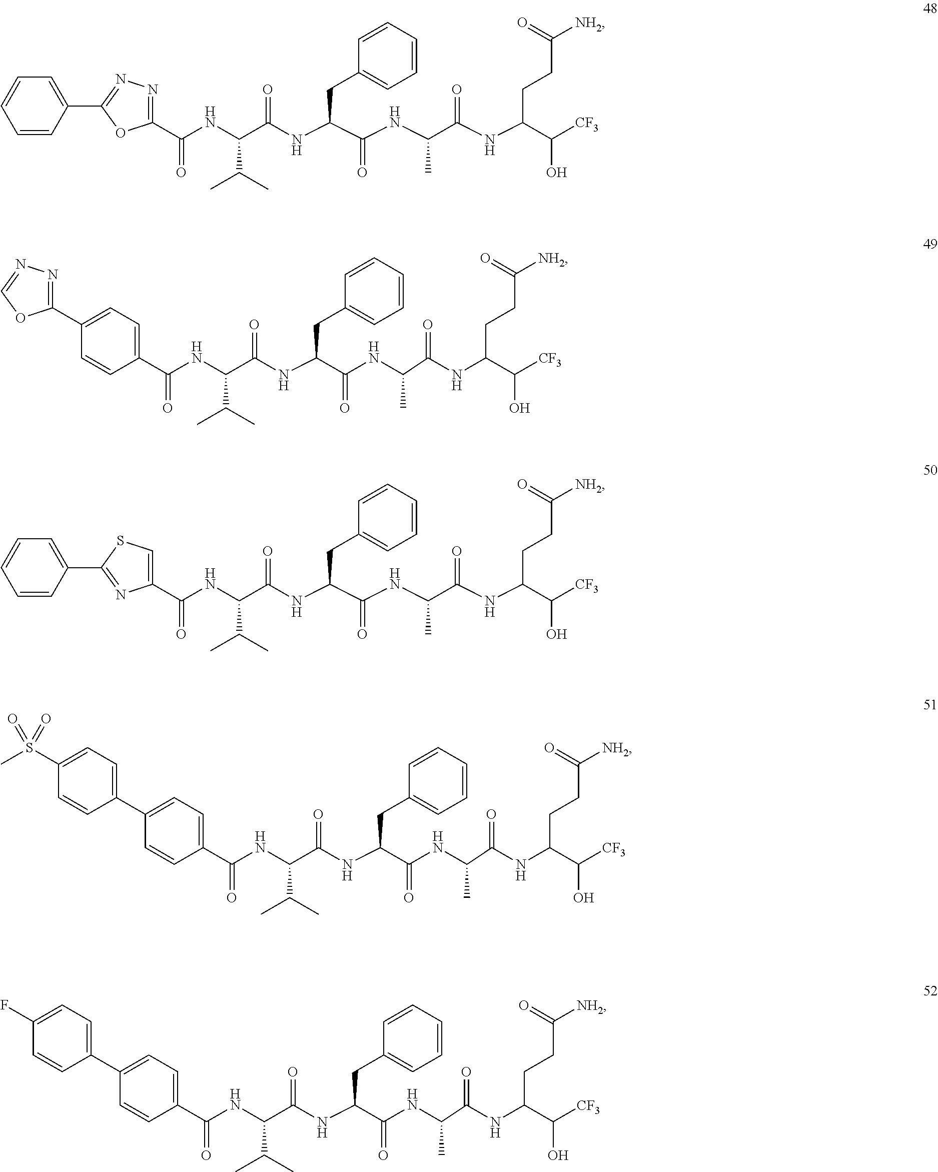

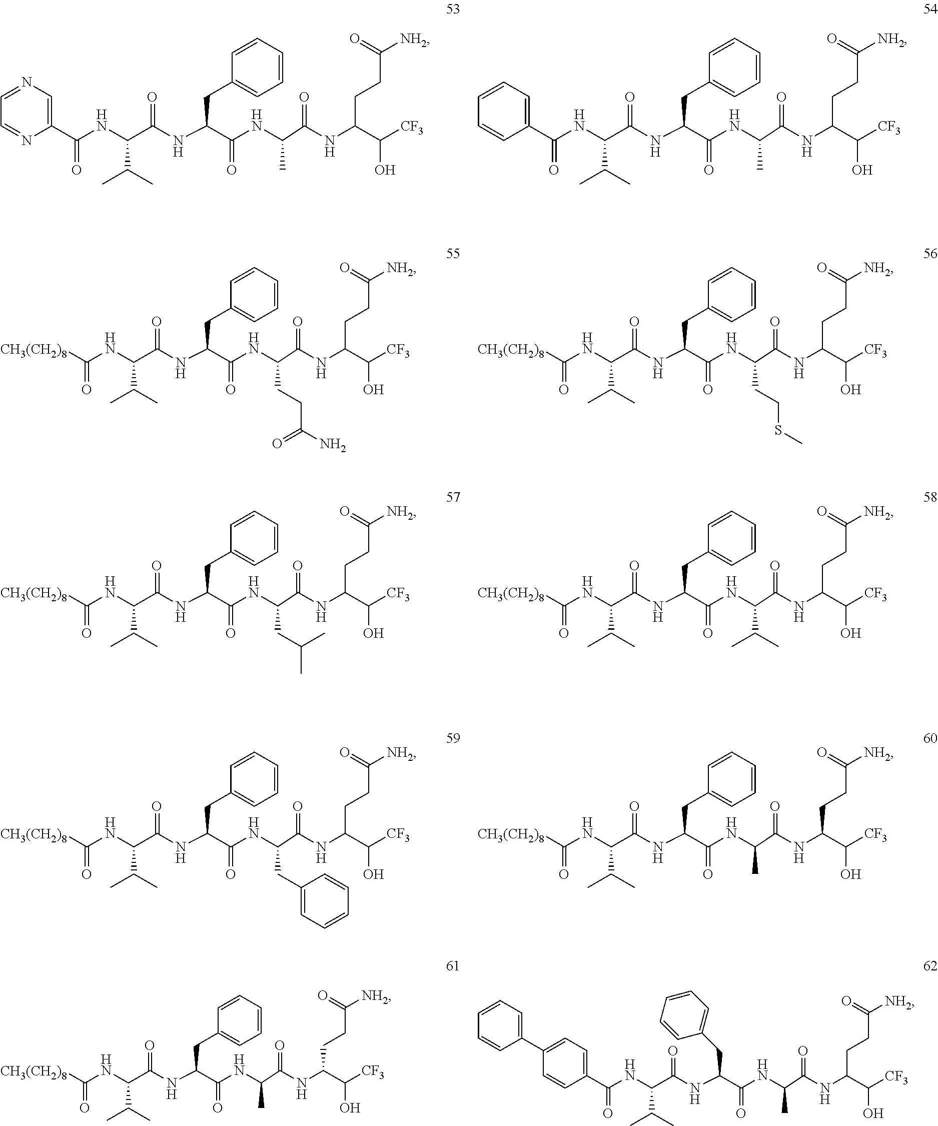

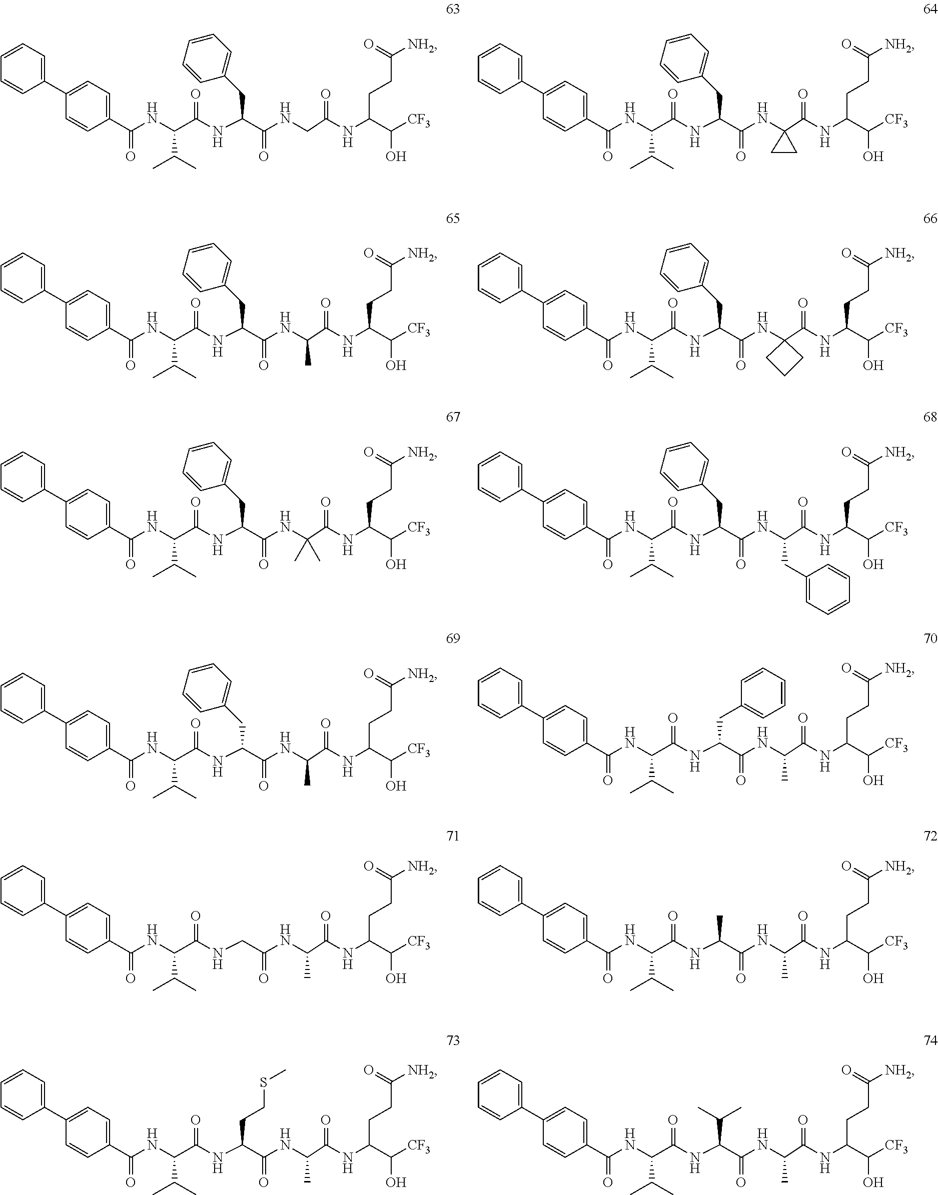

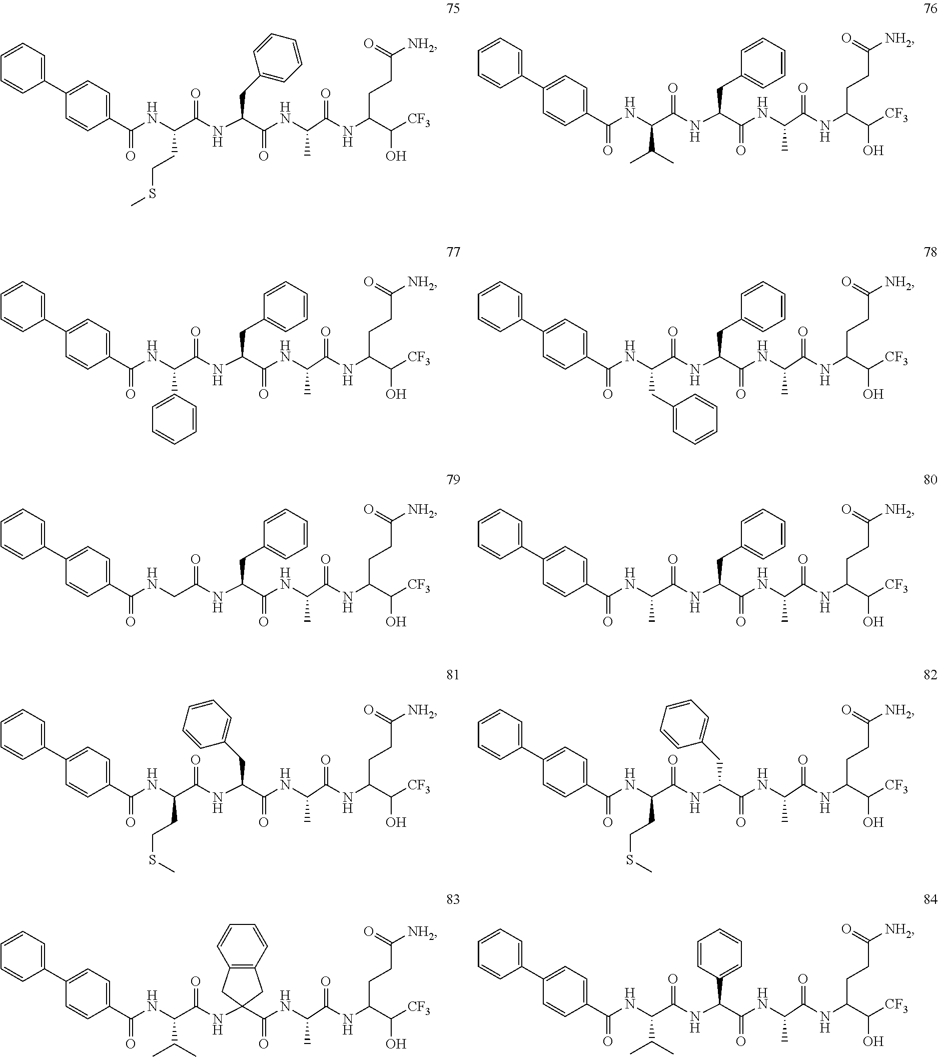

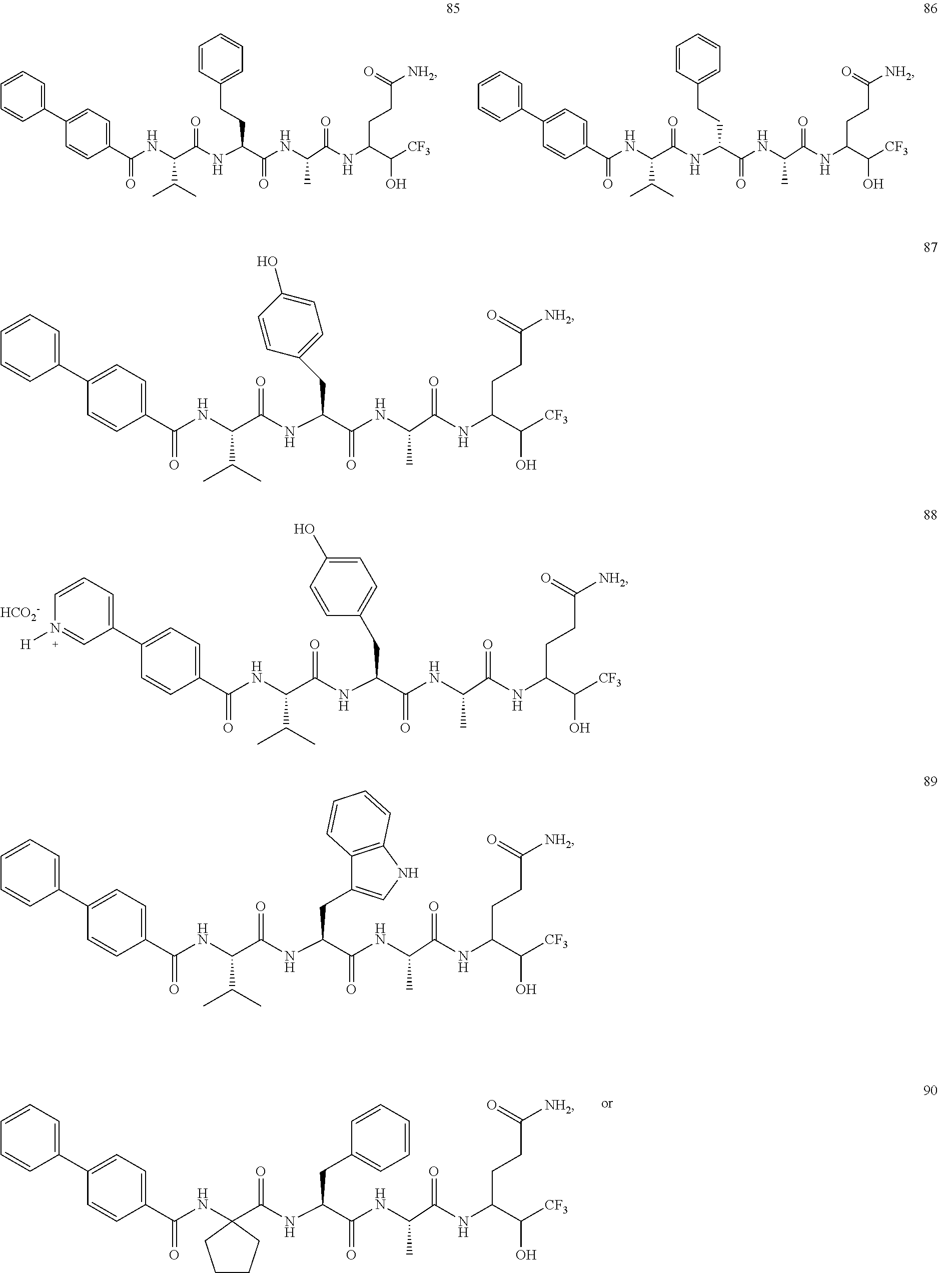

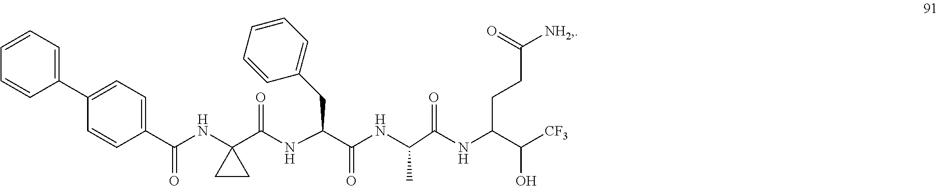 [SMILES: C[C@H](NC(=O)[C@H](Cc1ccccc1)NC(=O)C1(NC(=O)c2ccc(-c3ccccc3)cc2)CC1)C(=O)NC(CCC(N)=O)C(O)C(F)(F)F]

In a specific embodiment, the compound of the invention is not compound 1; 2; 17; mixture of 21a and 21b; 25 and/or 26.

In accordance with another aspect of the present invention, there is provided a pharmaceutical composition comprising at least one of the compounds of the present invention. In a specific embodiment of the composition, the composition further comprises at least one other compound of the present invention. In another specific embodiment, the composition further comprises at least one other active ingredient which improve a patient's lipid profile. In another specific embodiment, the composition further comprises a pharmaceutical carrier or excipient.

In accordance with another aspect of the present invention, there is provided a compound or a composition of the present invention for use as a medicament. In a specific embodiment, the compound or composition is for the manufacture of a medicament. In another specific embodiment, said medicament is for preventing or treating a low density lipid-cholesterol-related disease or disorder in a subject, with the proviso that the compound is not:

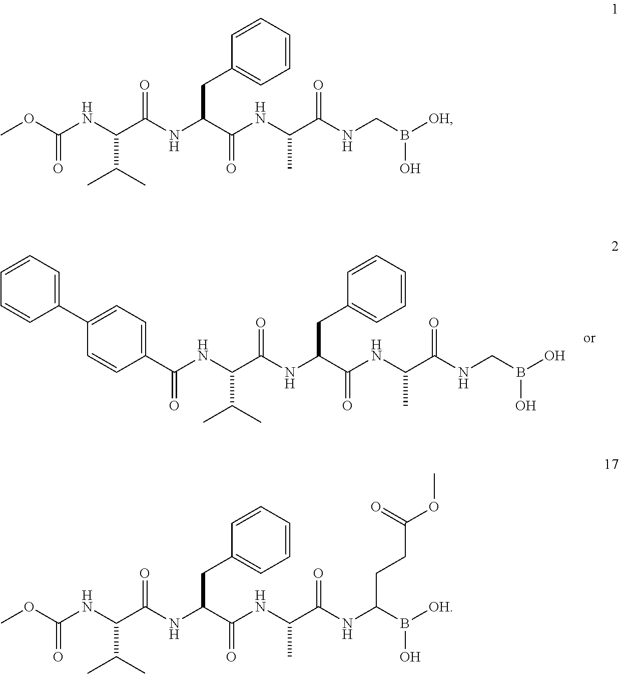

In another specific embodiment of the compound or composition, the low density lipid-cholesterol-related disease or disorder is hypercholesterolemia.

In accordance with another aspect of the present invention, there is provided a method for preventing or treating an LDL-cholesterol-related disease or disorder, comprising administering to a subject in need thereof, a therapeutically effective amount of the compound or the composition of the present invention (e.g., composition comprising the compound of the present invention), with the proviso that the compound is not:

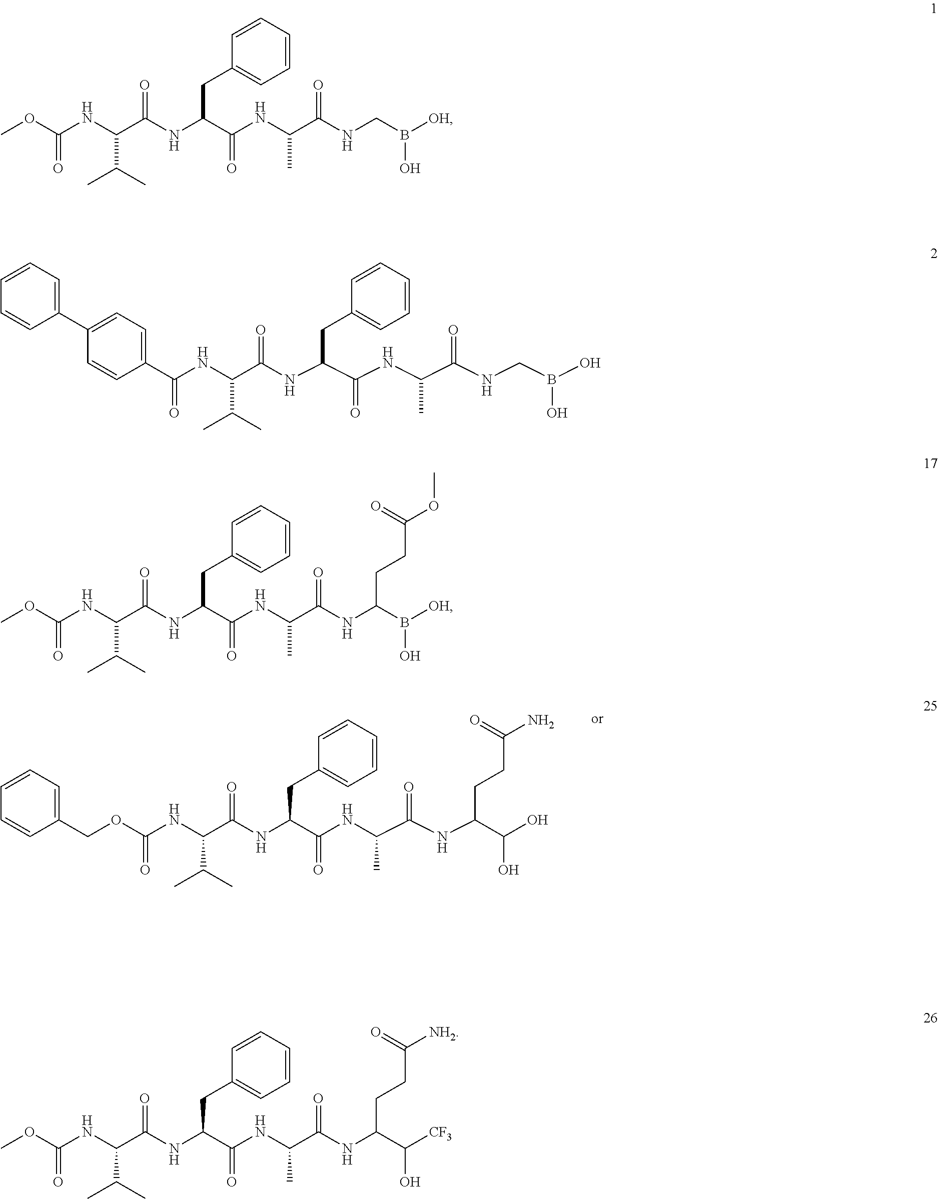

In a specific embodiment of the method, the low density lipid-cholesterol-related disease or disorder is hypercholesterolemia.

In accordance with another aspect of the present invention, there is provided a use of a compound or composition of the present invention, as a medicament. In a specific embodiment, the use of the compound or composition of the present invention is for preventing or treating a low density lipid-cholesterol-related disease or disorder in a subject, with the proviso that the compound is not:

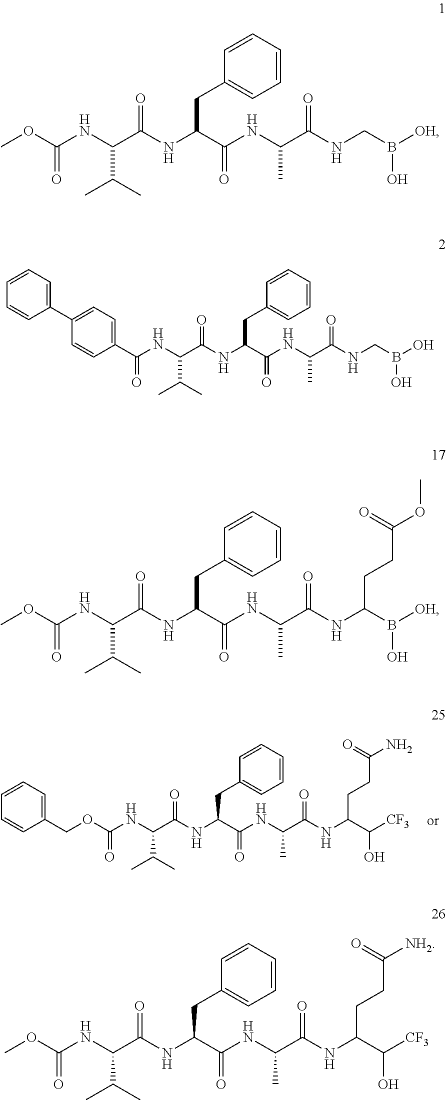

In a specific embodiment, the use is for the manufacture of a medicament for preventing or treating a low density lipid-cholesterol-related disease in a subject, with the proviso that the compound is not:

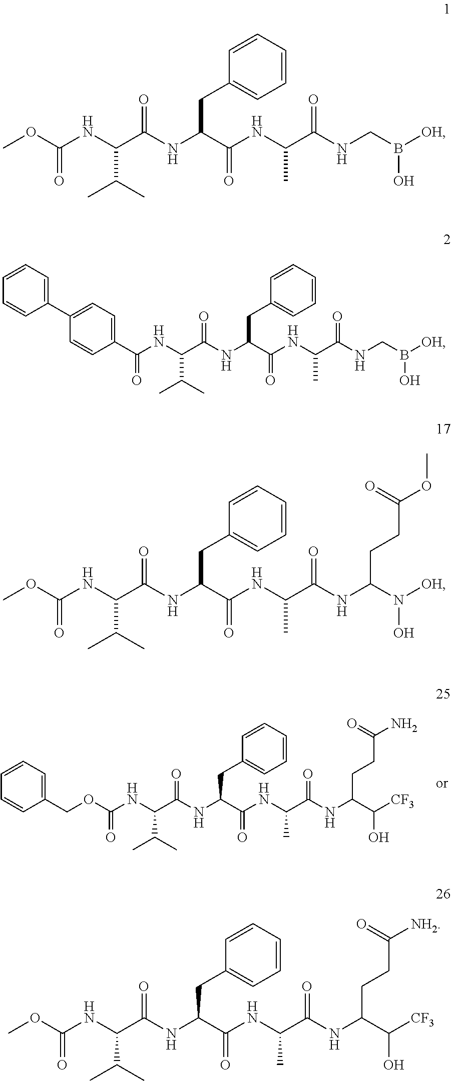

In another specific embodiment of the use, the low density lipid-cholesterol-related disease or disorder is hypercholesterolemia.

In accordance with another aspect of the present invention, there is provided a kit for preventing or treating a low density lipid-cholesterol-related disease or disorder in a subject comprising (i) at least one of the compounds or composition of the present invention, and (ii) (a) at least one other active ingredient which improve a patient's lipid profile; (b) at least one other compound or composition of the present invention; (c) a container for the compound and/or active ingredients; and/or (d) instructions to use the compound for preventing or treating the low density lipid-cholesterol-related disease or disorder in the subject, with the proviso that the compound is not:

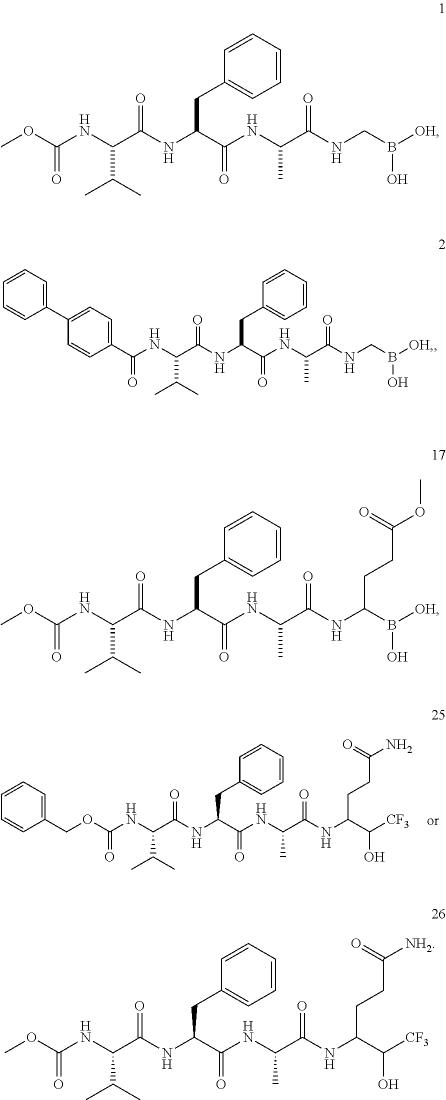

In a specific embodiment of the kit, the low density lipid-cholesterol-related disease or disorder is hypercholesterolemia.

### BRIEF DESCRIPTION OF THE DRAWINGS

In the appended drawings:

FIG. 1 shows (A) the effect of 19 compounds on PCSK9 secretion, as part of the initial screening. HepG2 cells stably expressing WT PCSK9(+V5) were incubated for 24 h in culture media alone (−), or containing either DMSO control (DMSO) or 100 μM of the indicated compounds. The PCSK9 levels in the recovered media and cell fractions were quantified by ELISA and expressed as ng PCSK9/ml fraction. The inhibitory effect of the indicated compounds on PCSK9 secretion was estimated from the decrease of the media/cell ratio relative to DMSO control. The stars identify compounds having an inhibitory activity; and (B) the structure of compounds a to j;

FIG. 2 shows the dose dependent effect of 3 compounds on PCSK9 secretion. HepG2 cells stably expressing WT PCSK9(+V5) were incubated for 24 h in the absence (DMSO) or presence of increasing concentrations of each indicated compound. The culture media and cells were recovered and the level of PCSK9 in each fraction was quantified by ELISA. The level of PCSK9 in cells was corrected for the amount of total protein, which was measured by the Bio-Rad DC protein assay. At each concentration, the inhibitory effect of the indicated compounds on PCSK9 secretion was estimated from the decrease of the media/cell ratio relative to DMSO control;

FIG. 3 shows the cell toxicity assay for 5 compounds relative to DMSO control. HepG2 cells stably expressing WT PCSK9(+V5) were incubated for 24 h in the absence (DMSO) or presence of increasing concentrations of each indicated compound. The level of toxicity was measured by a MTT cell toxicity assay;

FIG. 4 shows the effect of a PCSK9 inhibitory compound on the level of LDLR at the surface of HepG2 naïve cells. Cells were incubated for 20 h in the absence (DMSO) or presence of 33.3 μM of compound 19. Non-permeabilized cells were analyzed by immunofluorescence. LDLR was stained using a human LDLR Ab (green labeling). Three different fields are shown for each DMSO control and compound 19 condition. Note the increase of LDLR at the cell surface induced by compound 19;

FIG. 5 shows the effect of PCSK9 inhibitory compounds on the activity of cell surface LDLR, as measured by Dil-LDL uptake in HepG2 naïve cells (A) and HEK293 naïve cells (B), or on total LDLR levels, as measured by Western blot analysis in HepG2 naïve cells (C). The negative control is compound i. HepG2 naïve cells, which express endogenous PCSK9 (A) or HEK293 naïve cells, which lack PCSK9 expression (B), were incubated for 6 h in the absence (DMSO) or presence of 33.3 μM of the indicated compounds prior to the addition of Dil-LDL that was followed by an additional 18 h-incubation. For each condition, Dil-LDL uptake (fluorescence of Dil fluorescent probe) was corrected for the total number of cells (fluorescence of CyQuant™ GR dye) and expressed as % activity of the DMSO control. Data represents an average of 2 to 5 (A) or 2 to 3 (B) independent experiments performed in triplicate. (C) Western blot analysis of total LDLR in HepG2 naïve cells incubated for 24 h in the absence (Cnt_{—}0) or presence of increasing concentrations of compound 19. Total LDLR levels normalized to 1-actin are plotted for each condition as % control (Cnt). Quantification of protein bands was obtained using Image J™ software; and

FIG. 6 shows the in vivo inhibition of PCSK9. Selected inhibitors are tested in heterozygote Ldlr^{+/-} mice that exhibit “humanized” LDLc profiles (LDLc×2-3) and generated by intercrossing mice having WT and Ldlr^{-/-} backgrounds. These mice express either normal levels of mouse PCSK9 (Pcsk9^{+/+}), no PCSK9 (Pcsk9^{-/-}) and/or one or five copies of transgene encoding human PCSK9-D374Y from its own human promoter (TgDY). The human WT form of PCSK9 is also tested.

### DETAILED DESCRIPTION OF THE INVENTION

PCSK9 also known as neural apoptosis-regulated convertase 1 (NARC-1), is a proteinase K-Hke subtilase of 692 amino acids in human (NP_{—}777596.2), and comprises a signal peptide (1-30) followed by a prosegment (residues 31-152), a catalytic domain (residues 153-454) and a C-terminal Cys-His-rich domain (CHRD; residues 455-692). PCSK9 is expressed in cells capable of proliferation and differentiation such as hepatocytes, kidney mesenchymal cells, intestinal ileum, colon epithelia and embryonic brain telencephalic neurons (Seidah et al., 2003, Proc. Natl. Acad. Sci. USA 100:928-933).

Following translocation in the ER, the prosegment of PCSK9 is autocatalytically cleaved at the VFAQ₁₅₂↓SIP site. In PCs, the prosegment (pro) is an intramolecular chaperone/inhibitor that is usually removed intracellularly to yield a fully active protease. Different from other PCs, PCSK9 is secreted as a stable non-covalent complex [pro=PCSK9]. Accordingly, enhanced degradation of the LDLR induced by PCSK9 does not require the catalytic activity of the mature PCSK9 form. In human and mouse plasma, both full-length PCSK9 (153-692) and a truncated form PCSK9-ΔN218 (219-692) can be detected. The latter, which has no activity on LDLR, is likely generated by Furin and/or PC5, since they cleave PCSK9 ex vivo at RFHR218↓.

In the studies disclosed herein, the present inventors have generated and identified inhibitory compounds directed against human PCSK9 and shown that these compounds inhibit PCSK9 activity (e.g., PCSK9 secretion, induced LDLR degradation in the human hepatocarcinoma-derived cell lines such as HepG2).

### Compounds

The present invention relates to compounds of Formula (I):

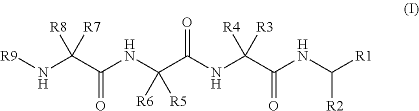

or a pharmaceutically acceptable salt, hydrate, solvate, or racemic mixture or stereoisomer thereof, wherein:

### R1 is —CH(OH)Rₐ or —B(OR_{b})(OR_{c});

R2 is —H, —CH₂R_{d}, —CHR_{d}(Rₑ), —CH₂(CH₂)ₘC(O)Rⱼ, —CH₂(CH₂)ₘC(O)N(R_{d})Rₑ or —CH₂(CH₂)ₘS(O)ₙN(R_{d})Rₑ;
R3, R4, R5, R6, R7 and R8 are identical or different, and are independently hydrogen or one of the following groups: a C1-6 alkyl, a C1-6 haloalkyl, a C1-6 thioalkyl, a C1-6 aminoalkyl, an alkenyl, an alkynyl, a cycloalkyl, an heterocyclyl, an aryl, and an heteroaryl group, wherein said group is optionally substituted with one or more C1-6 alkyl, C3-8 cycloalkyl, C1-6 haloalkyl, aryl, an heteroaryl, —CN, —C(O)N(R_{f})R_{g}, —C(O)OR_{f}, —C(R_{f})(R_{g})ORₕ, —OR_{f}, —OC(O)OR_{f}, —OC(O)NR_{f}(R_{g}), —SR_{f}, —S(O)ₙR_{f}, —S(O)ₙN(R_{f})R_{g}, —S(O)ₙN(R_{f})C(O)R_{g}, —N(R_{f})R_{g}, —N(R_{f})C(O)R_{g}, —N(R_{f})C(O)OR_{g}, —N(R_{f})C(O)N(R_{g})(Rₕ), —N(R_{f})S(O)ₙR_{g}, and —N(R_{f})S(O)N(R_{g})Rₕ substituents;
when (R3 and R4) or (R5 and R6) or (R7 and R8) are not hydrogen, the bracketed pairs can also be linked with —C(O)—, —CO₂—, —C(O)N(H)—, —C(O)N(Rₓ)—, —O—, —NH—, —N(Rₓ)—, —S—, —S(O)—, —S(O)ₙN(H)—, —S(O)ₙN(Rₓ)— radicals to form cyclic structures;
R9 is RᵢC(O)—, RᵢS(O)—, RᵢOC(O)—, RᵢNHC(O)—, RᵢNHS(O)—, Rₖ(Rₗ)NC(O)—, Rₗ(Rₗ)NS(O)ₙ—; RₘORᵢC(O)—, RₘC(O)RᵢC(O)— or one or more amino acids residues;
Rₐ is C1-3 alkyl, C1-2 fluoroalkyl or cyclopropyl;
R_{b} and R_{c} are identical or different, and are independently H or C1-6 alkyl, or can be connected together to form a cyclic 5- or 6-membered ring structure, or fused with additional aliphatic or aromatic ring systems, the cyclic 5- or 6-membered ring structure or aliphatic or aromatic ring systems being optionally substituted with one or more C1-6 alkyl and/or C1-6 haloalkyl substituents;
R_{d} and Rₑ are identical or different, and are independently H or one of the following groups: a C1-3 alkyl, a C1-3 haloalkyl or a C3-4 cycloalkyl group, or can be connected together directly or with —C(O)—, —C(O)O—, —C(O)N(Rₓ)— —O—, —N(Rₓ)—, —S—, —S(O)—, or —S(O)ₙN(Rₓ)— radicals to form cyclic 3-8 membered ring structures; R_{f}, R_{g}, Rₕ, Rₖ and Rₗ are identical or different, and are independently H or one of the following groups: a C1-6 alkyl, a C1-6 haloalkyl or a C3-4 cycloalkyl group, or can be connected together directly or with —C(O)—, —C(O)O—, —C(O)N(Rₓ)— —O—, —N(Rₓ)—, —S—, —S(O)— or —S(O)ₙN(Rₓ)— radicals to form cyclic 3-8 membered ring structures;
Rᵢ is a C1-10 alkyl, a C1-10 heteroalkyl, a C3-8 cycloalkyl, a C1-10 haloalkyl, a heterocyclyl, an aryl, a heteroaryl, a C1-10 alkyl-C3-8 cycloalkyl, a C1-10 alkyl-heterocyclyl, a C1-10 alkyl-aryl, a C1-10 alkyl-heteroaryl, a C1-10 heteroalkyl-C3-8 cycloalkyl, a C1-10 heteroalkyl-heterocyclyl, a C1-10 heteroalkyl-aryl or a C1-10 heteroalkyl-heteroaryl group, wherein the group is optionally substituted with one or more of a halogen, C1-6 aminoalkyl, C1-6 heteroalkyl, C1-6 alkyl, C3-8 cycloalkyl, C1-6 haloalkyl, aryl, heteroaryl and heterocyclyl groups;
Rₘ is a C1-10 alkyl, a C1-10 heteroalkyl, a C3-8 cycloalkyl, a C1-10 haloalkyl, a heterocyclyl, an aryl or a heteroaryl group, wherein the group is optionally substituted with one or more of a halogen, C1-6 aminoalkyl, C1-6 heteroalkyl, C1-6 alkyl, C3-8 cycloalkyl, C1-6 haloalkyl, aryl, heteroaryl and heterocyclyl groups;

### R_{y} is OR_{d} or N(R_{d})(Rₑ);

Rₓ is H, C1-6 alkyl, C1-6 haloalkyl, C3-4 cycloalkyl, —C(O)R_{y}, —C(O)OR_{y}, —C(O)NH₂, —C(O)NH(R_{y}) or —C(O)NHS(O)ₙR_{y};
R_{y} is C1-6 alkyl, C1-6 haloalkyl or C3-4 cycloalkyl;
m is an integer of value 0 or 1; and
n is an integer of value 1 or 2,
provided that:
1) when R1 is —CH(OH)Rₐ, R2 is —CHR_{d}(Rₑ), —CH₂(CH₂)ₘC(O)Rⱼ, —CH₂(CH₂)ₘC(O)N(R_{d})Rₑ or —CH₂(CH₂)ₘS(O)ₙN(R_{d})Rₑ; and
2) when R1 is —B(OR_{b})(ORₑ), R2 is —H, —CH₃, —CHR_{d}(Rₑ), —CH₂(CH₂)ₘC(O)Rⱼ, —CH₂(CH₂)ₘC(O)N(R_{d})Rₑ or —CH₂(CH₂)ₘS(O)ₙN(R_{d})Rₑ.

As recited above, the present invention encompasses additional amino acid residues in R9. One or more amino acid residues in R9 can facilitate active transport of the compounds of the present invention across cell membranes (see Koren, E.; Torchilin, V. P. Cell Penetrating Peptides: Breaking Through to the Other Side. Trends Mol. Med. 2012, 18(7), 385-93 and references therein.
Specific embodiments of the current invention include, but are not limited to the following:
(2S)-3-Methyl-N-[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoy)ethyl]-2-(phenylformamido)butanamide; 5.
benzyl N-[(1S)-2-methyl-1-{[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoyl}ethyl]carbamoyl}propyl]carbamate; 7.
tert-ButylN-[(1S)-2-methyl-1-{[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoyl}ethyl]carbamoyl}propyl]carbamate; 6.
(2S)-3-methyl-N-[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoyl}ethyl]-2-({4-[3-(trifluoromethyl)-3H-diazirin-3-yl]phenyl}formamido)butanamide; 8a.
(2S)-3-methyl-N-[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoyl}ethyl]-2-({3-[3-(trifluoromethyl)-3H-diazirin-3-yl]phenyl}formamido)butanamide; 8b.
(2S)-3-methyl-N-[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoyl}ethyl]-2-(2-{4-[3-(trifluoromethyl)-3H-diazirin-3-yl]phenyl}acetamido)butanamide; 8c.
(2S)-3-methyl-N-[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoyl}ethyl]-2-(2-{3-[3-(trifluoromethyl)-3H-diazirin-3-yl]phenyl}acetamido)butanamide; 8d.
(2S)-2-[(2-fluorophenyl)formamido]-3-methyl-N-[(1S)-2-phenyl-1-{[(1S)-1-({[(1S,2S,6R,8S)-2,9,9-trimethyl-3,5- dioxa-4-boratricyclo[6.1.1.0^{2,6}]decan-4-yl]methyl}carbamoyl)ethyl]carbamoyl}ethyl]butanamide; 9.
{[(2S)-2-[(2S)-2-[(2S)-3-Methyl-2-{2-[4-(trifluoromethoxy)phenyl]acetamido}butanamido]-3-phenylpropanamido]propanamido]methyl}boronic acid; 3.
{[(2S)-2-[(2S)-2-[(2S)-2-decanamido-3-methylbutanamido]-3-phenylpropanamido]propanamido]methyl}boronic acid; 4.
{[(2S)-2-[(2S)-2-[(2S)-3-Methyl-2-[(4-phenylphenyl)formamido]butanamido]-3-phenylpropanamido]propanamido]methyl}boronic acid; 2.
{[(2S)-2-[(2S)-2-[(2S)-2-[(Methoxycarbonyl)amino]-3-methylbutanamido]-3-phenylpropanamido]propanamido]methyl}boronic acid; 1.
[(1R/S)-3-Carbamoyl-1-[(2S)-2-[(2S)-2-[(2S)-2-[(methoxycarbonyl)amino]-3-methylbutanamido]-3-phenylpropanamido]propanamido]propyl]boronic acid; 18.
[(1R/S)-4-Methoxy-1-[(2S)-2-[(2S)-2-[(2S)-2-[(methoxycarbonyl)amino]-3-methylbutanamido]-3-phenylpropanamido]propanamido]-4-oxobutyl]boronic acid; 17.
Methyl N-[(1S)-1-{[(1R/S)-1-{[(1S)-1-{[(1S)-3-carbamoyl-1-[(1S,2S,6R,8)-2,9,9-trimethyl-3,5-dioxa-4-boratricyclo[6.1.1.0^{2,6}]decan-4-yl]propyl]carbamoyl}ethyl]carbamoyl}-2-phenylehtyl]carbamoyl}-2-methylpropyl]carbamate; 16.
Methyl (4R/S)-4-[(2S)-2-[(2S)-2-[(2S)-2[(Methoxycarbonyl)amino]-3-methylbutanamido]-3-phenylpropanamido]-2-phenylacetamido]-4-[(1S,2S,6R,8S)-2,9,9-trimethyl-3,5-dioxa-4-boratricyclo[6.1.1.0^{2,6}]decan-4-yl]butanoate; 15.
Methyl N-[(1S)-2-methyl-1-{[(1S)-2-phenyl-1-{[(S)-phenyl({[(1S,2S,6R,8S)-2,9,9-trimethyl-3,5-dioxa-4-boratricyclo[6.1.1.0^{2,6}]decan-4-yl]methyl}carbamoyl)methyl]carbamoyl}ethyl]carbamoyl}propyl]carbamate; 14.
Methyl N-[(1S)-2-methyl-1-{[(1S)-1-{[(1S)-3-methyl-1-({[(1S,2S,6R,8S)-2,9,9-trimethyl-3,5-dioxa-4-boratricyclo[6.1.1.0^{2,6}]decan-4-yl]methyl}carbamoyl)butyl]carbamoyl}-2-phenylethyl]carbamoyl}propyl]carbamate; 13.
Methyl N-[(1S)-2-methyl-1-{[(1S)-1-{[(1S)-3-(methylsulfanyl)-1-({[(1S,2S,6R,8S)-2,9,9-trimethyl-3,5-dioxa-4-boratricyclo[6.1.1.0^{2,6}]decan-4-yl]methyl}carbamoyl)propyl]carbamoyl}-2-phenylethyl]carbamoyl}propyl]carbamate; 12.
Methyl N-[(1S)-2-methyl-1-{[(1S)-2-phenyl-1-{[(1S)-2-phenyl-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoyl}ethyl]carbamoyl}propyl]carbamate; 11.
Methyl N-[(1S)-2-methyl-1-{[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)propyl]carbamoyl}ethyl]carbamoyl}propyl]carbamate; 10.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)decanamide; 19.
N-((2S)-1-(((2S)-1-(((2S)-1-(((3S)-6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)decanamide; 20a.
N-((2S)-1-(((2S)-1-(((2S)-1-(((3R)-6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)decanamide; 20b.
N-((2S)-1-(((2S)-1-(((2S)-1-((6-Amino-1-fluoro-2-hydroxy-6-oxohexan-3-yl)amino)-1-oxopropan-2-yl)amino)-1-oxo-3-phenylpropan-2-yl)amino)-3-methyl-1-oxobutan-2-yl)decanamide; 21a and 21b.
N-((2)-1-(((2)-1-(((2)-1-((6-Amino-2-hydroxy-6-oxohexan-3-yl)amino)-1-oxopropan-2-yl)amino)-1-oxo-3-phenylpropan-2-yl)amino)-3-methyl-1-oxobutan-2-yl)decanamide; 22a and 22b.
6,6,6-Trifluoro-5-hydroxy-4-((S)-2-((S)-2-((S)-3-methyl-2-(2-(4-(trifluoromethoxy)phenyl)acetamido)butanamido)-3-phenylpropanamido)propanamido)hexanamide; 23.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-[1,1′-biphenyl]-4-carboxamide; 24.
Benzyl ((2S)-1-(((2S)-1-(((2S)-1-((6-amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)carbamate; 25.
Methyl ((2S)-1-(((2S)-1-(((2S)-1-((6-amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)carbamate; 26.
tert-Butyl ((2S)-1-(((2S)-1-(((2S)-1-((6-amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)carbamate; 27.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4-isopropylbenzamide; 28.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4-(3-(trifluoromethyl)-3H-diazirin-3-yl)benzamide; 29.
6,6,6-Trifluoro-5-hydroxy-4-((S)-2-((S)-2-((S)-3-methyl-2-(2-(4-(3-(trifluoromethyl)-3H-diazirin-3-yl)phenyl)acetamido)butanamido)-3-phenylpropanamido)propanamido)hexanamide; 30.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-3-(3-(trifluoromethyl)-3H-diazirin-3-yl)benzamide; 31.
6,6,6-Trifluoro-5-hydroxy-4-((S)-2-((S)-2-((S)-3-methyl-2-(2-(3-(3-(trifluoromethyl)-3H-diazirin-3-yl)phenyl)acetamido)butanamido)-3-phenylpropanamido)propanamido)hexanamide; 32.
4-((S)-2-((S)-2-((S)-2-([1,1′-Biphenyl]-4-sulfonamido)-3-methylbutanamido)-3-phenylpropanamido)propanamido)-6,6,6-trifluoro-5-hydroxyhexanamide; 33.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-5-methyl-1-phenyl-1H-pyrazole-4-carboxamide; 34.
6,6,6-Trifluoro-5-hydroxy-4-((S)-2-((S)-2-((S)-3-methyl-2-(2-phenylacetamido)butanamido)-3-phenylpropanamido)propanamido)hexanamide; 35.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-6-phenylnicotinamide; 36)
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-2-(pyridin-4-yl)thiazole-4-carboxamide; 37.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4-(pyridin-3-yl)benzamide; 38.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4-(hydroxymethyl)benzamide; 39.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-1-phenyl-1H-pyrazole-4-carboxamide; 40.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4-morpholinobenzamide; 41.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4-(morpholine-4-carbonyl)benzamide; 42.
6,6,6-Trifluoro-5-hydroxy-4-((S)-2-((S)-2-((S)-3-methyl-2-(3-phenylpropiolamido)butanamido)-3-phenylpropanamido)propanamido)hexanamide; 43.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)nicotinamide; 44.
6,6,6-Trifluoro-5-hydroxy-4-((S)-2-((S)-2-((S)-3-methyl-2-(2-phenoxyacetamido)butanamido)-3-phenylpropanamido)propanamido)hexanamide; 45.
4-((6S,9S,12S)-9-Benzyl-6-isopropyl-12-methyl-4,7,10-trioxo-1-phenyl-2-oxa-5,8,11-triazatridecan-13-amido)-6,6,6-trifluoro-5-hydroxyhexanamide; 46.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-1H-indole-5-carboxamide; 47.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-5-phenyl-1,3,4-oxadiazole-2-carboxamide; 48.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4-(1,3,4-oxadiazol-2-yl)benzamide; 49.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-2-phenylthiazole-4-carboxamide; 50.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4′-(methylsulfonyl)-[1,1′-biphenyl]-4-carboxamide; 51.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4′-fluoro-[1,1′-biphenyl]-4-carboxamide; 52.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)pyrazine-2-carboxamide; 53.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)benzamide; 54.
2S)—N′-(6-Amino-1,1,1-trifluoro-2-hydroxy-6-oxohexan-3-yl)-2-((S)-2-((S)-2-decanamido-3-methylbutanamido)-3-phenylpropanamido)pentanediamide; 55.
N-((2S)-1-(((2S)-1-(((2S)-1-((6-Amino-1,1,1-trifluoro-2-hydroxy-6-oxohexan-3-yl)amino)-4-(methylthio)-1-oxobutan-2-yl)amino)-1-oxo-3-phenylpropan-2-yl)amino)-3-methyl-1-oxobutan-2-yl)decanamide; 56.
N-((2S)-1-(((2S)-1-(((2S)-1-((6-Amino-1,1,1-trifluoro-2-hydroxy-6-oxohexan-3-yl)amino)-4-methyl-1-oxopentan-2-yl)amino)-1-oxo-3-phenylpropan-2-yl)amino)-3-methyl-1-oxobutan-2-yl)decanamide; 57)
N-((2S)-1-(((2S)-1-(((2S)-1-((6-amino-1,1,1-trifluoro-2-hydroxy-6-oxohexan-3-yl)amino)-3-methyl-1-oxobutan-2-yl)amino)-1-oxo-3-phenylpropan-2-yl)amino)-3-methyl-1-oxobutan-2-yl)decanamide; 58.
N-((2S)-1-(((2S)-1-(((2S)-1-((6-Amino-1,1,1-trifluoro-2-hydroxy-6-oxohexan-3-yl)amino)-1-oxo-3-phenylpropan-2-yl)amino)-1-oxo-3-phenylpropan-2-yl)amino)-3-methyl-1-oxobutan-2-yl)decanamide; 59.
N-((2S)-1-(((2S)-1-(((2R)-1-(((3S)-6-amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)decanamide; 60.
N-((2S)-1-(((2S)-1-(((2R)-1-(((3R)-6-amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)decanamide; 61.
N-((2S)-1-(((2S)-1-(((2R)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-[1,1′-biphenyl]-4-carboxamide; 62.
N-((2S)-1-(((2S)-1-((2-((6-Amino-1,1,1-trifluoro-2-hydroxy-6-oxohexan-3-yl)amino)-2-oxoethyl)amino)-1-oxo-3-phenylpropan-2-yl)amino)-3-methyl-1-oxobutan-2-yl)-[1,1′-biphenyl]-4-carboxamide; 63.
N-((2S)-1-(((2S)-1-((1-((6-Amino-1,1,1-trifluoro-2-hydroxy-6-oxohexan-3-yl)carbamoyl)cyclopropyl)amino)-1-oxo-3-phenylpropan-2-yl)amino)-3-methyl-1-oxobutan-2-yl)-[1,1′-biphenyl]-4-carboxamide; 64.
N-((2S)-1-(((2S)-1-(((2R)-1-(((3S)-6-amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-[1,1′-biphenyl]-4-carboxamide; 65.
N-((2S)-1-(((2S)-1-((1-(((3S)-6-Amino-1,1,1-trifluoro-2-hydroxy-6-oxohexan-3-yl)carbamoyl)cyclobutyl)amino)-1-oxo-3-phenylpropan-2-yl)amino)-3-methyl-1-oxobutan-2-yl)-[1,1′-biphenyl]-4-carboxamide; 66.
N-((2S)-1-(((2S)-1-((1-((6-Amino-1,1,1-trifluoro-2-hydroxy-6-oxohexan-3-yl)amino)-2-methyl-1-oxopropan-2-yl)amino)-1-oxo-3-phenylpropan-2-yl)amino)-3-methyl-1-oxobutan-2-yl)-[1,1′-biphenyl]-4-carboxamide; 67.
N-((2S)-1-(((2S)-1-(((2S)-1-((6-Amino-1,1,1-trifluoro-2-hydroxy-6-oxohexan-3-yl)amino)-1-oxo-3-phenylpropan-2-yl)amino)-1-oxo-3-phenylpropan-2-yl)amino)-3-methyl-1-oxobutan-2-yl)-[1,1′-biphenyl]-4-carboxamide; 68.
N-((2S)-1-(((2R)-1-(((2R)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-[1,1′-biphenyl]-4-carboxamide; 69.
N-((2S)-1-(((2R)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-[1,1′-biphenyl]-4-carboxamide; 70.
N-((2S)-1-((2-(((2S)-1-((6-Amino-1,1,1-trifluoro-2-hydroxy-6-oxohexan-3-yl)amino)-1-oxopropan-2-yl)amino)-2-oxoethyl)amino)-3-methyl-1-oxobutan-2-yl)-[1,1′-biphenyl]-4-carboxamide; 71.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxopropan-2-yl)amino)-3-methyl-1-oxobutan-2-yl)-[1,1′-biphenyl]-4-carboxamide; 72.
N-((2S)-1-(((2S)-1-(((2S)-1-((6-Amino-1,1,1-trifluoro-2-hydroxy-6-oxohexan-3-yl)amino)-1-oxopropan-2-yl)amino)-4-(methylthio)-1-oxobutan-2-yl)amino)-3-methyl-1-oxobutan-2-yl)-[1,1′-biphenyl]-4-carboxamide; 73.
N-((2S)-1-(((2S)-1-(((2S)-1-((6-Amino-1,1,1-trifluoro-2-hydroxy-6-oxohexan-3-yl)amino)-1-oxopropan-2-yl)amino)-3-methyl-1-oxobutan-2-yl)amino)-3-methyl-1-oxobutan-2-yl)-[1,1′-biphenyl]-4-carboxamide; 74.
N-((5S,8S,11S)-17-amino-8-benzyl-11-methyl-6,9,12,17-tetraoxo-14-(2,2,2-trifluoro-1-hydroxyethyl)-2-thia-7,10,13-triazaheptadecan-5-yl)-[1,1′-biphenyl]-4-carboxamide; 75.
N-((2R)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-[1,1′-biphenyl]-4-carboxamide; 76.
N-((1S)-2-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-2-oxo-1-phenylethyl)-[1,1′-biphenyl]-4-carboxamide; 77.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-1-oxo-3-phenylpropan-2-yl)-[1,1′-biphenyl]-4-carboxamide; 78.
N-(2-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-2-oxoethyl)-[1,1′-biphenyl]-4-carboxamide; 79.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-1-oxopropan-2-yl)-[1,1′-biphenyl]-4-carboxamide; 80.
N-((5R,8S,11S)-17-amino-8-benzyl-11-methyl-6,9,12,17-tetraoxo-14-(2,2,2-trifluoro-1-hydroxyethyl)-2-thia-7,10,13-triazaheptadecan-5-yl)-[1,1′-biphenyl]-4-carboxamide; 81.
N-((5R,8R,11S)-17-Amino-8-benzyl-11-methyl-6,9,12,17-tetraoxo-14-(2,2,2-trifluoro-1-hydroxyethyl)-2-thia-7,10,13-triazaheptadecan-5-yl)-[1,1′-biphenyl]-4-carboxamide; 82.
2-((S)-2-([1,1′-Biphenyl]-4-carboxamido)-3-methylbutanamido)-N-((2S)-1-((6-amino-1,1,1-trifluoro-2-hydroxy-6-oxohexan-3-yl)amino)-1-oxopropan-2-yl)-2,3-dihydro-1H-indene-2-carboxamide; 83.
N-((2S)-1-(((1S)-2-(((2S)-1-((6-Amino-1,1,1-trifluoro-2-hydroxy-6-oxohexan-3-yl)amino)-1-oxopropan-2-yl)amino)-2-oxo-1-phenylethyl)amino)-3-methyl-1-oxobutan-2-yl)-[1,1′-biphenyl]-4-carboxamide; 84.
N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-4-phenylbutan-2-yl)amino)-3-methyl-1-oxobutan-2-yl)-[1,1′-biphenyl]-4-carboxamide; 85.
N-((2S)-1-(((2R)-1-(((2S)-1-((6-Amino-1,1,1-trifluoro-2-hydroxy-6-oxohexan-3-yl)amino)-1-oxopropan-2-yl)amino)-1-oxo-4-phenylbutan-2-yl)amino)-3-methyl-1-oxobutan-2-yl)-[1,1′-biphenyl]-4-carboxamide; 86.
N-((2S)-1-(((2S)-1-(((2S)-1-((6-Amino-1,1,1-trifluoro-2-hydroxy-6-oxohexan-3-yl)amino)-1-oxopropan-2-yl)amino)-3-(4-hydroxyphenyl)-1-oxopropan-2-yl)amino)-3-methyl-1-oxobutan-2-yl)-[1,1′-biphenyl]-4-carboxamide; 87.
3-(4-(((2S)-1-(((2S)-1-(((2S)-1-((6-Amino-1,1,1-trifluoro-2-hydroxy-6-oxohexan-3-yl)amino)-1-oxopropan-2-yl)amino)-3-(4-hydroxyphenyl)-1-oxopropan-2-yl)amino)-3-methyl-1-oxobutan-2-yl)carbamoyl)phenyl)pyridin-1-ium formate; 88.
N-((2S)-1-(((2S)-1-(((2S)-1-((6-Amino-1,1,1-trifluoro-2-hydroxy-6-oxohexan-3-yl)amino)-1-oxopropan-2-yl)amino)-3-(1H-indol-3-yl)-1-oxopropan-2-yl)amino)-3-methyl-1-oxobutan-2-yl)-[1,1′-biphenyl]-4-carboxamide; 89.
N-(1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)carbamoyl)cyclopentyl)-[1,1′-biphenyl]-4-carboxamide; 90.
N-(1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)carbamoyl)cyclopropyl)-[1,1′-biphenyl]-4-carboxamide; 91.

Compounds listed above are identified by numbers (in bold). These numbers also refer to the specific examples where each of the above listed compounds is further described. If there were discrepancies between structures and names used herein for compounds of the present invention, structures will prevail.

### DEFINITIONS

### Chemical Groups

As used herein, the term “alkyl” refers to a monovalent straight or branched chain, saturated or unsaturated aliphatic hydrocarbon radical having a number of carbon atoms in the specified range. Thus, for example, “C1-6 alkyl” (or “C1-C6 alkyl”) refers to any of the hexyl alkyl and pentyl alkyl isomers as well as n-, iso-, sec- and t-butyl, n- and iso-propyl, ethyl and methyl. As another example, “C1-4 alkyl” refers to n-, iso-, sec- and t-butyl, n- and isopropyl, ethyl and methyl. As another example, “C1-3 alkyl” refers to n-propyl, isopropyl, ethyl and methyl. Alkyl include unsaturated aliphatic hydrocarbon including alkyne (R—C≡C—R); and/or alkene (R—C═C—R).

The term “halogen” (or “halo”) refers to fluorine, chlorine, bromine and iodine (alternatively referred to as fluoro, chloro, bromo, and iodo). The term “haloalkyl” refers to an alkyl group as defined above in which one or more of the hydrogen atoms have been replaced with a halogen (i.e., F, Cl, Br and/or I). Thus, for example, “C1-6 haloalkyl” (or “C1-C6 haloalkyl”) refers to a C1 to C6 linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.).

The term “heteroalkyl” is given its ordinary meaning in the art and refers to alkyl groups as described herein in which one or more carbon atoms is replaced with a heteroatom (e.g., oxygen, nitrogen, sulfur, or derivatives thereof, and the like). Examples of heteroalkyl groups include, but are not limited to, alkoxy, alkyl-substituted amino, thiol such as methionine side group. Up to two heteroatoms may be consecutive. When a prefix such as C2-6 is used to refer to a heteroalkyl group, the number of carbons (2-6, in this example) is meant to include the heteroatoms as well.

The term “aminoalkyl” refers to an alkyl group as defined above in which one or more of the hydrogen or carbon atoms has been replaced with an nitrogen or amino derivative. Thus, for example, “C1-6 aminoalkyl” (or “C1-C6 aminoalkyl”) refers to a C1 to C6 linear or branched alkyl group as defined above with one or more amino derivatives (e.g., NH, amide, diazirin, etc.).

The term “thioalkyl” refers to an alkyl group as defined above in which one or more of the hydrogen or carbon atoms has been replaced with a sulfur atom or thiol derivative. Thus, for example, “C1-6 aminoalkyl” (or “C1-C6 aminoalkyl”) refers to a C1 to C6 linear or branched alkyl group as defined above with one or more sulfur atoms or thiol derivatives (e.g., S, SH, etc.).

Aminoalkyl and thioalkyls are specific embodiments of and encompassed by the term “heteroalkyl” or substituted alkyl depending on the heteroatom replaces a carbon atom or an hydrogen atom.

The term “cycloalkyl” refers to saturated alicyclic hydrocarbon consisting of saturated 3-8 membered rings optionally fused with additional (1-3) aliphatic (cycloalkyl) or aromatic ring systems, each additional ring consisting of a 3-8 membered ring. It includes without being so limited cyclopropane, cyclobutane, cyclopentane, and cyclohexane.

The term “heterocyclyl” refers to (i) a 4- to 7-membered saturated heterocyclic ring containing from 1 to 3 heteroatoms independently selected from N, O and S, or (ii) is a heterobicyclic ring (e.g., benzocyclopentyl). Examples of 4- to 7-membered, saturated heterocyclic rings within the scope of this invention include, for example, azetidinyl, piperidinyl, morpholinyl, thiomorpholinyl, thiazolidinyl, isothiazolidinyl, oxazolidinyl, isoxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, azepanyl, diazepanyl, tetrahydropyranyl, tetrahydrothiopyranyl, and dioxanyl. Examples of 4- to 7-membered, unsaturated heterocyclic rings within the scope of this invention include mono-unsaturated heterocyclic rings corresponding to the saturated heterocyclic rings listed in the preceding sentence in which a single bond is replaced with a double bond (e.g., a carbon-carbon single bond is replaced with a carbon-carbon double bond).

The term “C(O)” refers to carbonyl. The terms “S(O)₂” and “SO₂” each refer to sulfonyl. The term “S(O)” refers to sulfinyl.

The term “aryl” refers to aromatic (unsaturated) compounds consisting of 3-8 membered rings, optionally fused with additional (1-3) aliphatic (cycloalkyl) or aromatic ring systems, each additional ring consisting of 3-8 membered ring. In a specific embodiment, it refers to phenyl, benzocyclopentyl, or naphthyl. The aryl of particular interest is phenyl. The term “heteroaryl” refers to (i) a 3-, 4-, 5- or 6-membered heteroaromatic ring containing from 1 to 3 heteroatoms independently selected from N, O and S, or (ii) is a heterobicyclic ring selected from quinolinyl, isoquinolinyl, and quinoxalinyl. Suitable 3-, 4-, 5- and 6-membered heteroaromatic rings include, for example, diazirin, pyridyl (also referred to as pyridinyl), pyrrolyl, diazine (e.g.i, pyrazinyl, pyrimidinyl, pyridazinyl), triazinyl, thienyl, furanyl, imidazolyl, pyrazolyl, triazolyl, oxazolyl, iso-oxazolyl, oxadiazolyl, oxatriazolyl, thiazolyl, isothiazolyl, and thiadiazolyl. Heteroaryls of particular interest are pyrrolyl, imidazolyl, pyridyl, pyrazinyl, quinolinyl (or quinolyl), isoquinolinyl (or isoquinolyl), and quinoxalinyl. Suitable heterobicyclic ring include indolyl.

As used herein, and unless otherwise specified, the terms “alkyl”, “haloalkyl”, “aminoalkyl”, “cycloalkyl”, “heterocyclyl”, “aryl”, “heteroalkyl” and “heteroaryl” and the terms designating their specific embodiments (e.g., butyl, fluoropropyl, aminobutyl, cyclopropane, morpholine, phenyl, pyrazole, etc.) encompass the substituted (i.e. in the case of haloalkyl and aminoalkyl, in addition to their halogen and nitrogen substituents, respectively) and unsubstituted embodiments of these groups. Hence for example, the term “phenyl” encompasses unsubstituted phenyl as well as fluorophenyl, hydroxyphenyl, methylsulfonyl phenyl (or biphenyl), trifluoromethyl-diazirin-phenyl, isopropyl-phenyl, trifluorohydroxy-phenyl. Similarly, the term pyrazole, encompass unsubstituted pyrazole as well as methylpyrazole. The one or more substituents may be an amine, halogen, hydroxyl, C1-6 aminoalkyl, C1-6 heteroalkyl, C1-6 alkyl, C3-8 cycloalkyl, C1-6 haloalkyl, aryl, heteroaryl and heterocyclyl groups (etc.).

It is understood that the specific rings listed above are not a limitation on the rings which can be used in the present invention. These rings are merely representative.

Unless expressly stated to the contrary in a particular context, any of the various cyclic rings and ring systems described herein may be attached to the rest of the compound at any ring atom (i.e., any carbon atom or any heteroatom) provided that a stable compound results.

Unless expressly stated to the contrary, all ranges cited herein are inclusive. For example, a heteroaromatic ring described as containing from “1 to 4 heteroatoms” means the ring can contain 1, 2, 3 or 4 heteroatoms. It is also understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, and 4 heteroatoms.

As another example, an aryl or heteroaryl described as optionally substituted with “from 1 to 4 substituents” is intended to include as aspects thereof, an aryl or heteroaryl substituted with 1 to 4 substituents, 2 to 4 substituents, 3 to 4 substituents, 4 substituents, 1 to 3 substituents, 2 to 3 substituents, 3 substituents, 1 to 2 substituents, 2 substituents, and 1 substituent.

When any variable (e.g., XA or XB) occurs more than one time in any constituent or in Formula I or in any other formula depicting and describing compounds of the present invention, its definition on each occurrence is independent of its definition at every other occurrence. Also, combinations of substituents and/or variables are permissible only if such combinations result in stable compounds.

Unless expressly stated to the contrary, substitution by a named substituent is permitted on any atom in a ring (e.g., cycloalkyl, heterocyclyl, aryl, or heteroaryl) provided such ring substitution is chemically allowed and results in a stable compound.

### Salts, Esters, Hydrates and Solvates

The compounds of the present invention include pharmacologically acceptable salts and ester derivatives thereof as well as hydrates or solvates thereof and all stereoisomeric forms of the referenced compounds. The compounds and pharmacologically acceptable esters thereof of the present invention can form pharmacologically acceptable salts if necessary.

Salts

The terms “pharmacologically acceptable salt” refer to a salt to which the compounds of the present invention can be converted that retains the desired biological activity of the parent compound and does not impart any undesired toxicological effects (see e.g., Berge, S. M. et al., 1977 J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. Preferred examples of such a salt include alkali metal salts such as a sodium salt, a potassium salt, a lithium salt, magnesium or calcium salts; alkaline earth metal salts such as a calcium salt and a magnesium salt; metal salts such as an aluminum salt, an iron salt, a zinc salt, a copper salt, a nickel salt and a cobalt salt; amine salts such as inorganic salts including an ammonium salt; organic salts or ammonium salts such as a t-octylamine salt, a dibenzylamine salt, a morpholine salt, a glucosamine salt, a phenylglycine alkyl ester salt, an ethylenediamine salt, an N-methylglucamine salt, a guanidine salt, a diethylamine salt, a triethylamine salt, a dicyclohexylamine salt, an N,N′-dibenzylethylenediamine salt, a chloroprocaine salt, a procaine salt, a diethanolamine salt, an N-benzylphenethylamine salt, a piperazine salt, a tetramethylammonium salt and a tris(hydroxymethyl)aminomethane salt; inorganic acid salts such as hydrohalic acid salts such as a hydrofluoride, a hydrochloride, a hydrobromide or a hydroiodide, a nitrate, a perchlorate, a sulfate or a phosphate; lower alkanesulfonates such as a methanesulfonate, trifluoromethanesulfonate or an ethanesulfonate; arylsulfonates such as a benzenesulfonate or a p-toluenesulfonate and the like, which are non-toxic to living organisms; organic acid salts such as an acetate, a malate, adipate, a fumarate, a succinate, a citrate, alginate, ascorbate, benzoate, benzenesulfonate, bisulfate, butyrate, camphorate, camphorsulfonate, cinnamate, cyclopentanepropionate, digluconate, dodecylsulfate, ethanesulfonate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, sulfonate, methanesulfonate, trifluoromethanesulfonates, ethanesulfonates 2-naphthalenesulfonate, nicotinate, nitrate, oxalate, pamoate, pectinate, persulfate, 3-phenylpropionate, picrate, pivalate, propionate, tartrate, thiocyanate, tosylate, trifluoroacetic acid, undecanoate, a tartrate, an oxalate or a maleate; and amino acid salts such as a glycine salt, a lysine salt, an arginine salt, an omithine salt, histidine, a glutamate or an aspartate salt. Additionally, basic nitrogen containing groups may be quaternized with such agents as lower alkyl halides such as methyl, ethyl, propyl, and butyl chlorides, bromides and iodides; dialkyl sulfates including dimethyl, diethyl, and dibutyl sulfate; and diamyl sulfates, long chain halides such as decyl, lauryl, myristyl and strearyl chlorides, bromides and iodides, aralkyl halides including benzyl and phenethyl bromides, and others. For further example, see S. M. Berge, et al. “Pharmaceutical Salts,” J. Pharm. Sci. 1977, 66, 1-19. Such salts can be formed quite readily by those skilled in the art using standard techniques.

Preferred examples of the salts formed with an acidic group present in the compounds of the present invention include metal salts such as alkali metal salts (e.g., sodium salts, potassium salts and lithium salts), alkali earth metal salts (e.g., calcium salts and magnesium salts), aluminum salts and iron salts; amine salts such as inorganic amine salts (e.g., ammonium salts) and organic amine salts (e.g., t-octylamine salts, dibenzylamine salts, morpholine salts, glucosamine salts, phenylglycinealkyl ester salts, ethylenediamine salts, N-methylglucamine salts, guanidine salts, diethylamine salts, triethylamine salts, dicyclohexylamine salts, N,N′-dibenzylethylenediamine salts, chloroprocaine salts, procaine salts, diethanolamine salts. N-benzylphenethylamine salts, piperazine salts, tetramethylammonium salts and tris(hydroxymethyl)aminomethane salts; and amino acid salts such as glycine salts, lysine salts, arginine salts, ornithine salts, glutamates and aspartates.

All salts are intended to be pharmaceutically acceptable salts within the scope of the invention and all salts are considered equivalent to the free forms of the corresponding compounds for purposes of the invention.

### Esters

Physiologically/pharmaceutically acceptable esters are also useful as active medicaments. The term “pharmaceutically acceptable esters” embraces esters of the compounds of the present invention, in which hydroxy groups (e.g., in carboxylic acid) have been converted to the corresponding esters and may act as a prodrug which, when absorbed into the bloodstream of a warm-blooded animal, may cleave in such a manner as to release the drug form and permit the drug to afford improved therapeutic efficacy. Such esters can be formed with inorganic or organic acids such as nitric acid, sulphuric acid, phosphoric acid, citric acid, formic acid, maleic acid, acetic acid, succinic acid, tartaric acid, methanesulphonic acid, p-toluenesulphonic acid and the like, which are non-toxic to living organisms. Further examples are the esters with aliphatic or aromatic acids such as acetic acid or with aliphatic alcohol (e.g., alkyl esters, including methyl, ethyl, propyl, isopropyl, butyl, isobutyl or pentyl esters, and the like) or aromatic alcohols (e.g., benzyl ester).

Esters can be prepared from their corresponding acids or salts by a variety of methods known to those skilled in the art, such as, for example, by first transforming the acid to the acid chloride and then reacting the acid chloride with a suitable alcohol. Other suitable methods for making esters are described in Kemp and Vellaccio, 1980.

Where esters of the invention have a basic group, such as an amino group, the compound can be converted to a salt by reacting it with an acid, and in the case where the esters have an acidic group, such as a sulfonamide group, the compound can be converted to a salt by reacting it with a base. The compounds of the present invention encompass such salts.

Salts and esters of the compounds of the present invention may be prepared by known method by employing appropriate starting materials or intermediate compounds that are readily available and/or are described herein.

Generally, a desired salt of a compound of this invention can be prepared in situ during the final isolation and purification of a compound by means well known in the art. For example, a desired salt can be prepared by separately reacting the purified compound in its free base or free acid form with a suitable organic or inorganic acid, or suitable organic or inorganic base, respectively, and isolating the salt thus formed. In the case of basic compounds, for example, the free base is treated with anhydrous HCl in a suitable solvent such as THF, and the salt isolated as a hydrochloride salt. In the case of acidic compounds, the salts may be obtained, for example, by treatment of the free acid with anhydrous ammonia in a suitable solvent such as ether and subsequent isolation of the ammonium salt. These methods are conventional and would be readily apparent to one skilled in the art.

The compounds of this invention may be esterified by a variety of conventional procedures including reacting the appropriate anhydride, carboxylic acid or acid chloride with the alcohol group of a compound of this invention. The appropriate anhydride is reacted with the alcohol in the presence of a base to facilitate acylation such as 1,8-bis[dimethylamino]naphthalene or N,N-dimethylaminopyridine. Or, an appropriate carboxylic acid can be reacted with the alcohol in the presence of a dehydrating agent such as dicyclohexylcarbodiimide, 1-[3-dimethylaminopropyl]-3-ethylcarbodiimide or other water soluble dehydrating agents which are used to drive the reaction by the removal of water, and, optionally, an acylation catalyst. Esterification can also be effected using the appropriate carboxylic acid in the presence of trifluoroacetic anhydride and, optionally, pyridine, or in the presence of N,N-carbonyldiimidazole with pyridine. Reaction of an acid chloride with the alcohol can be carried out with an acylation catalyst such as 4-DMAP or pyridine.

One skilled in the art would readily know how to successfully carry out these as well as other known methods of etherification of alcohols.

### Prodrugs and Solvates

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of the present invention or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of the present invention or a pharmaceutically acceptable salt, hydrate or solvate of the compound contains a carboxylic acid functional group, a prodrug can comprise an ester formed by the replacement of the hydrogen atom of the acid group with a group such as, for example, (C1-C8)alkyl, (C2-C12)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C1-C2)alkylamino(C2-C3)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a compound of the present invention contains an alcohol functional group, a prodrug can be formed by the replacement of the hydrogen atom of the alcohol group with a group such as, for example, (C1-C6)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of the present invention incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl, —C(OH)C(O)OY1 wherein Y1 is H, (C₁-C₆)alkyl or benzyl, —C(OY₂)Y₃ wherein Y₂ is (C₁-C₄) alkyl and Y₃ is (C₁-C₆)alkyl, carboxy (C₁-C₆)alkyl, amino(C₁-C₄)alkyl or mono-N- or di-N,N—(C₁-C₆)alkylaminoalkyl, —C(Y4)Y5 wherein Y₄ is H or methyl and Y₅ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. A “Hydrate” is a solvate wherein the solvent molecule is H₂O.

Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

### Hydrates

As used herein the terms, “pharmaceutically acceptable hydrate” refer to the compounds of the instant invention crystallized with one or more molecules of water to form a hydrated form.

### Stereoisomers, Diastereomers, Enantiomers, Racemates, Tautomers

The compounds of the present invention have asymmetric carbon atoms/chiral centers and, as a result of the selection of substituents and substituent patterns, can contain additional chiral centers, and thus can occur as mixtures of stereoisomers (racemates), or as individual diastereomers, or enantiomers. The present invention embraces all of these forms.

Diastereomers (sometimes called diastereoisomers) are stereoisomers that are not enantiomers. Diastereomerism occurs when two or more stereoisomers of a compound have different configurations at one or more (but not all) of the equivalent (related) stereocenters and are not mirror images of each other. When two diastereoisomers differ from each other at only one stereocenter they are epimers. Each stereocenter gives rise to two different configurations and thus to two different stereoisomers.

Diastereomers differ from enantiomers in that the latter are pairs of stereoisomers which differ in all stereocenters and are therefore mirror images of one another. Enantiomers of a compound with more than one stereocenter are also diastereomers of the other stereoisomers of that compound that are not their mirror image. Diastereomers have different physical properties and different reactivity, unlike enantiomers. Diastereomers of the present invention include tomatidine and 3 alpha-hydroxy-tomatidine for example.

To the extent substituents and substituent patterns provide for the existence of tautomers (e.g., keto-enol tautomers) in the compounds of the invention, all tautomeric forms of these compounds, whether present individually or in mixtures, are within the scope of the present invention. Compounds of the present invention having a hydroxy substituent on a carbon atom of a heteroaromatic ring are understood to include compounds in which only the hydroxy is present, compounds in which only the tautomeric keto form (i.e., an oxo substitutent) is present, and compounds in which the keto and enol forms are both present.

For purposes of this Specification, “pharmaceutically acceptable tautomer” means any tautomeric form of any compound of the present invention.

The purification of enantiomers and the separation of isomeric mixtures of a compound of the present invention may be accomplished by standard techniques known in the art.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject). The compounds of the present invention are limited to stable compounds embraced by Formula I.

Synthetic methods for preparing the compounds of the present invention are illustrated in the following general procedures, schemes, and examples. Starting materials are commercially available or may be prepared according to procedures known in the art or as illustrated herein. The compounds of the invention are illustrated by means of the specific examples shown below. However, these specific examples are not to be construed as forming the only genus that is considered as the invention. These examples further illustrate details for the preparation of the compounds of the present invention. Those skilled in the art will readily understand that known variations of the conditions and processes of the following preparative procedures can be used to prepare these compounds.

All temperatures are in degrees Celsius. Mass spectra (MS) were measured by electrospray ion-mass spectroscopy (ESI) on an Agilent 6120 Quadrapole™ MS coupled to an Agilent 1100™ series HPLC instrument. NMR spectra were recorded on a Varian Mercury spectrometer at 400 MHz for ¹H and 376 MHz for ¹⁹F.

For the purposes of this specification, the following abbreviations have the indicated meanings:
AcOH=acetic acid
Alk=alkyl
Ar=aryl
atm=atmosphere
BINAP=2,2′-bis(diphenylphosphino)-1,1′-binaphthalene
Boc=tert-butoxycarbonyl
n-BuLi=n-butyllithium
Cbz=carboxybenzyl
CH₂Cl₂=dichloromethane
DBU=1,8-diazabicyclo[5.4.0]undec-7-ene
DEAD=diethyl azodicarboxylate
DIPEA=N,N-diisopropylethylamine
DMAP=4-(dimethylamino)pyridine
DMF=N,N-dimethylformamide
DMSO=dimethyl sulfoxide
ESI=electrospray ionization
Et₃N=triethylamine
Et₂O=diethylether
EtOAc or EA=ethyl acetate
EtOH=ethyl alcohol
h=hour(s)
H₂=hydrogen
HATU=O-(7-azabenzotriazol-1-yl)-N,N,N′,N′-tetramethyluronium hexafluorophosphate
HCl=hydrochloric acid
HPLC=High Pressure Liquid Chromatography
iPrOH=2-propanol
KF=potassium fluoride
LC-MS=Liquid Chromatography Mass Spectrometry
LiOH=lithium hydroxide
MeCN=acetonitrile
MeMgBr=methylmagnesium bromide
MeOH=methyl alcohol
MeTHF=2-methyltetrahydrofuran
MgSO₄=magnesium sulfate
min=minute(s)
MS=mass spectroscopy
MTBE=methyl tert-butyl ether
N₂=nitrogen
NaBH₄=sodium borohydride
NaHCO₃=sodium bicarbonate
NaOH=sodium hydroxide
Na₂SO₄=sodium sulfate
NH₃=ammonia
NH₄Cl=ammonium chloride
NH₄OH=ammonium hydroxide
NMP=N-methyl 2-pyrrolidinone
NMR=nuclear magnetic resonance spectroscopy
Moc=methoxycarbonyl
P=pressure
Pd/C=palladium on charcoal
PG=protecting group
Ph=phenyl
Pyr=pyridine
rbf=round bottom flask
R_{f}=retention factor on silica gel
rt=room temperature
TBDMS=tert-butyldimethylsilyl
Ts=toluene-4-sulfonyl
TFA=trifluoroacetic acid
TFAA=trifluoroacetic anhydride
TFA-NHS=trifluoroacetic N-hydroxysuccinimide
THF=tetrahydrofuran
TLC=thin layer chromatography
TMEDA=N,N,N′,N′-tetramethylethylenediamine
T3P=Propylphosphonic anhydride

The preparation of trifluoroacetic N-hydroxysuccinimide (TFA-NHS) is described in the literature: Sohn, C. H.; Lee, J. E.; Sweredoski, M. J.; Graham, R. L. J.; Smith, G. T.; Hess, S.; Czerwieniec, G.; Loo, J. A.; Deshaies, R. J.; Beauchamp, J. L. J. Am. Chem. Soc. 2012, 134, 2672-2680.

The preparation of (S)-3-tert-butoxycarbonyl-4-(2-benzyloxycarbonylethyl)oxazolidin-5-one is described in the literature: Aurelio, L.; Brownlee, R. T. C.; Hughes, A. B.; Sleebs, B. E. Aust. J. Chem. 2000, 53, 425-433. (R)-3-tert-butoxycarbonyl-4-(2-benzyloxycarbonylethyl)oxazolidin-5-one can also be prepared in an analogous manner starting from N-Boc-D-glutamic acid 5-benzyl ester.

### Pharmaceutical Compositions

In another aspect, the present invention provides a composition, e.g., a pharmaceutical composition, containing one or a combination of compounds of the present invention, formulated together with a pharmaceutically acceptable carrier and/or excipient.

Pharmaceutical compositions of the invention also can be administered in combination therapy, i.e., combined with other agents. For example, the combination therapy can include at least one other cholesterol-reducing agent. Examples of therapeutic agents that can be used in combination therapy are described in greater detail below.

As used herein, “pharmaceutically acceptable carrier” or “pharmaceutically acceptable excipient” includes any and all solvents, dispersion media, coatings, antibacterial and antifungal agents, isotonic and absorption delaying agents, and the like that are physiologically compatible. The carrier should be suitable for oral, intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

### Carrier/Excipients

A pharmaceutical composition of the invention may include a pharmaceutically acceptable antioxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers or excipients include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition.

Prolonged absorption of the injectable compositions can be brought about by including in the composition an agent that delays absorption for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

Examples of pharmaceutical preparations for oral administration include tablets for internal use (such as uncoated tablets, sugar-coated tablets, coating tablets, enteric coated tablets and chewable tablets), tablets administered to oral cavity (such as buccal preparations, sublingual tablets, troches and adhesive tablets), powders, capsules (such as hard capsules and soft capsules), granules (such as coated granules, pills, troches, liquids preparations or pharmaceutically acceptable sustained release pharmaceutical preparations). Specific examples of liquid preparations capable of being orally administered are solutions for internal use, shake mixtures, suspensions, emulsions, syrups, dry syrups, elixirs, infusion and decoction and lemonades.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

### Doses and Dosage Regimen

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

For administration of the compounds, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per day, week, once every two weeks, once every three weeks, once every four weeks, once a month, once every 3 months or once every three to 6 months.

In some methods, two or more compounds with different activity are administered simultaneously, in which case the dosage of each compound administered falls within the ranges indicated. The compound is usually administered on multiple occasions. Intervals between single dosages can be, for example, weekly, monthly, every three months or yearly. Intervals can also be irregular as indicated by measuring blood levels of compound in the patient. In some methods, dosage is adjusted to achieve a plasma concentration of the compound of about 1-1000 μg/ml and in some methods about 25-300 μg/ml.

Alternatively, a compound can be administered as a sustained release formulation, in which case less frequent administration is required. Dosage and frequency vary depending on the half-life of the compound in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response (e.g., decreased plasma LDL/cholesterol levels) for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective amount” or “effective amount” or “therapeutically effective dosage” of compounds of the invention can result in a lowering of LDL-C level in a subject, a decrease in severity of at least one disease symptom (e.g., a decrease in plasma LDL-cholesterol, or a decrease in a symptom of a LDL-cholesterol-related disorder), an increase in frequency and duration of disease symptom-free periods, or a prevention of impairment or disability due to the disease affliction in the subject.

### Routes of Administration

A composition of the present invention can be administered by one or more routes of administration using one or more of a variety of methods known in the art. As will be appreciated by the skilled artisan, the route and/or mode of administration will vary depending upon the desired results. Routes of administration for compounds of the invention include oral, intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. Alternatively, a compound of the invention can be administered by a nonparenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978. Therapeutic compositions can be administered with medical devices known in the art. In certain embodiments, the compound of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. The liposomes may comprise one or more moieties which are selectively transported into specific cells, tissues or organs, thus enhance targeted drug delivery (see, e.g., V. V. Ranade, 1989 J. Clin. Pharmacol. 29:685). In an embodiment, the compound of the invention can be formulated to be delivered to the liver (i.e., to hepatocytes).

### Functional Characterization of the Compounds

The functional characteristics of the compounds of the present invention can be tested in vitro and in vivo. For example, compounds can be tested for the ability to inhibit PCSK9 proteolytic activity, PCSK9-dependent effects on LDLR (e.g., LDLR mediated uptake of LDL-C), PCSK9-dependent LDLR degradation including the interaction of PCSK9 to LDLR, and to decrease LDL-C in vivo.

PCSK9 binding to LDLR can be detected by surface plasmon resonance (SPR) (using BIAcore®) by immobilizing LDLR to a solid support and detecting soluble PCSK9 binding to the LDLR. Alternatively, PCSK9 can be immobilized, and LDLR binding can be detected. PCSK-9/LDLR binding can also be analyzed by ELISA (e.g., by detecting PCSK9 binding to immobilized LDLR), by fluorescence resonance energy transfer (FRET), or phage display. To perform FRET, fluorophore-labeled PCSK9 binding to LDLR in solution can be detected (see, for example, U.S. Pat. No. 5,631,169). PCSK9 binding to LDLR has been detected by coimmunoprecipitation (Lagace et al., 2006 J. Clin. Inv. 116(11):2995-3005). For example, to examine PCSK9-LDLR binding in this manner, HepG2 cells are cultured in sterol-depleted medium for 18 hours. Purified PCSK9 is added to the medium in the presence of 0.1 mM chloroquine and the cells are incubated for one hour. Cells are lysed in mild detergent (1% digitonin w/vol). PCSK9 or LDLR is immunoprecipitated from cell lysates, separated by SDS-PAGE, and immunoblotted to detect the presence of coimmunoprecipitated LDLR or PCSK9, respectively (Lagace et al., 2006, supra).

These assays may be conducted with a mutant form of PCSK9 that binds to LDLR with a higher affinity (e.g., hPCSK9 D374Y, Lagace et al., 2006, supra). Hepatocytes express LDLR on the cell surface. Addition of purified PCSK9 to cultured hepatocyte cells (e.g., HepG2 cells, ATCC HB-8065, HuH7 cells, primary human or mouse hepatocytes) produces a decrease in LDLR expression in a dose- and time-dependent manner (Lagace et al., 2006 supra). Compounds of the invention can be tested for the ability to increase LDLR levels in hepatocytes. For example, HepG2 cells are cultured in sterol-depleted medium (DMEM supplemented with 100 U/ml penicillin, 100 μg/ml streptomycin sulfate, and 1 g/1 glucose, 5% (vol/vol) newborn calf lipoprotein-deficient serum (NCLPDS), 10 μM sodium compactin, and 50 μM sodium mevalonate) for 18 hours to induce LDLR expression. Purified PCSK9 (5 μg/ml) is added to the medium. LDLR levels in cells harvested at 0, 0.5, 1, 2, and 4 hours after addition of PCSK9 are determined (Lagace et al., 2006, supra). LDLR levels can be determined by flow cytometry, FRET, immunoblotting, or other means. LDL-C uptake by cells (e.g., HepG2 cells, HuH7 cells) can be measured using fluorescently-labeled LDL-C, Dil-LDL (3,3′-dioctadecylindocarbocyanine-low density lipoprotein) as described by Stephan and Yurachek (1993, J. Lipid Res. 34: 325-330). Briefly, cells are incubated in culture with Dil-LDL (20-100 μg protein/ml) at 37° C. for 2 hours. Cells are washed, lysed, and the concentration of internalized Dil-LDL is quantitated using a spectrofluorometer. LDL-C uptake can be measured in cells contacted with a PCSK9 inhibitory compound (prior to, and/or during the period in which Dil-LDL is present in the cell culture).

Transgenic mice overexpressing human PCSK9 in liver have increased levels of plasma LDL-C relative to non-transgenic mice (Lagace et al., 2006 supra). See also Maxwell and Breslow, 2004 Proc. Natl. Acad. Sci. USA, 101:7100, describing overexpression of PCSK9 using an adenovirus vector in mice. PCSK9^{-/-} mice have been produced (Rashid et al., 2005 Proc. Natl. Acad. Sci. 102(5):5374-5379). These mice can be genetically modified to express a mouse or a human PCSK9 transgene. Compounds can be tested in any of these models, or in animals which are not genetically modified, for the ability to clear or reduce total cholesterol and/or LDL-C.

The kinetics of LDL clearance from plasma can be determined by injecting animals with [¹²⁵I]-labelled LDL, obtaining blood samples at 0, 5, 10, 15, and 30 minutes after injection, and quantitating [¹²⁵I]-LDL in the samples (Rashid et al., 2005 supra). The rate of LDL clearance is increased in PCSK9^{-/-} mice relative to wild type mice (Rashid et al., 2005 supra). Increased LDL clearance in animals administered a compound indicates that the agent inhibits PCSK9 activity in vivo.

Decreases in total plasma cholesterol, plasma triglycerides, and/or LDL-C in response to treatment with a compound are indicative of therapeutic efficacy against PCKS9 activity. Cholesterol and lipid profiles can be determined by colorimetric, gas-liquid chromatographic, or enzymatic means using commercially available kits.

Methods/assays to determine PCSK9 activity are described below. As used herein the terms “PCSK9 activity” and “PCSK9 function” refer to detectable (direct or indirect) enzymatic, biochemical or cellular activity attributable to PCSK9. Without being so limited, such activities include the effect of PCSK9 on reducing the level of LDLR at the cell surface, on reducing plasma LDL-C and/or the PCSK9 proteinase activity itself (e.g., PCSK9 secretion).

### In Vitro Analysis of PCSK9 Secretion and Processing.

The secretion and/or the proPCSK9 to PCSK9 processing are tested using ELISA assay or a biosynthetic approach using the measurement of PCSK9 in cells and media by Western blot. A decrease in PCSK9 secretion or response in the presence of compound is indicative that the compound inhibits a PCSK9 activity. For statistical significance each experiment is performed in triplicate. “Dose-dependent” responses of compound(s) are performed.

### In Vitro Analysis of PCSK9-Dependant LDLR Degradation.

Compound is also tested for its ability to inhibit the LDLR enhanced degradation by PCSK9 on mouse or human hepatocyte cell lines such as HepG2 or HuH7. The assay consists in the addition of wild type (WT), mutants or chimeric PCSK9, either following transfection or directly to the culture supernatants in the presence or absence of the tested compound. “Dose-dependent” responses of compound(s) are performed. For statistical significance each “dose-responses” experiment is done in duplicate or triplicate for 4 to 6 different dosages.

The inhibition of the PCSK9 activity is evidence by an increase of the LDLR protein expression and/or at the cell surface, as evidenced by:
Western blot analysis of cell lysates for the total LDLR;
FACS analysis for cell surface for LDLR;
Fluorescent Dil-LDL incorporation monitoring the cell surface activity of LDLR.

The Dil-LDL fluorescent uptake assay consists in the fluorescence measurement of the Dil-LDL cellular incorporation via LDLR internalization (a measurement of cell surface LDLR activity). The cells are incubated in a 96-well format in the presence or absence of different doses of tested compound for 2 h, and then Dil-LDL was added for an additional 2 h. The inhibition of the PCSK9 activity is detected by an increase in the Dil-LDL fluorescence.

a. WT PCSK9.

The assay consists in the addition of wild type (WT) PCSK9, either as conditioned media from transfected cells or purified, and added to the culture supernatants, in the presence or absence of the tested compound. The dose routinely chosen for PCSK9 added extracellularly is 1 ug/ml.

b. Mutants PCSK9 (Gain of Function).

In order to further characterize whether the tested compound can inhibit the function of a gain of function mutation, the cells are incubated with purified mutant proteins, in the presence or absence of different doses of tested compound. Purified PCSK9 mutants are PCSK9-D374Y, for example (exhibiting a ˜25-fold higher affinity towards LDLR) or S127R (showing an increase stability of PCSK9). The dose routinely chosen for PCSK9 and its gain-of-function natural mutant D374Y, added extracellularly are 1 μg/ml and 0.2 μg/ml. Others PCSK9 mutants are similarly used. The assay could also be conducted using culture medium harvested from cells transfected with gain of function PCSK9 mutants.

c. Chimeric PCSK9.

Chimeric protein fusing the PCSK9 with the transmembrane and cytosolic domains of the cell surface angiotensin converting enzyme 2 (PCSK9-ACE2) is tested for measuring the activity of the tested compound on the PCSK9 extracellular pathway activity. Alternatively, chimeric protein fusing PCSK9 with the transmembrane and cytosolic domains of the Lamp-1 which directly traffic the protein to the endosomes/lysosomes (PCSK9-Lamp1) is tested for measuring the activity of the tested compound on the PCSK9 intracellular pathway activity. The stable cells expressing the chimeric PCSK9-ACE2 or PCSK9-Lamp1 are available and are be incubated in the presence or absence of different doses of tested compound. Chimeric protein also included PCSK9 with a V5 tag.

d. Primary Human Hepatocytes.

The PCSK9 inhibitory compounds are tested on mouse and human primary hepatocytes in order to measure their effect on cell surface LDLR. The advantage of using the mouse primary hepatocytes is that it also measures the specificity of the compound in the context of a wild type or knockout mouse expressing or lacking PCSK9, respectively. HepG2 and HuH7 cells that no longer express PCSK9 endogenously (e.g., under a shRNA knockdown) are also used for similar drug specificity purpose.

PCSK9 Contained in Conditioned Media from Transfected Cells.

Human wild type PCSK9 (PCSK9-WT) and gain-of-function (PCSK9-D374Y) proteins are produced by over-expression in HEK293 cells or Huh7 cells. Briefly, HEK293 or Huh7 cell lines are grown in Dulbecco's modified Eagle's medium with 10% fetal bovine serum (Invitrogen) and maintained at 37° C. under 5% CO2. HEK293 cells are transfected with jetPRIME™ (Polyplus transfection), and Huh7 cells are transfected with Lipofectamine™ 2000 (Invitrogen), according to the manufacturer's protocol. Twenty-four hours post-transfection, cells are washed and incubated in serum-free medium. Conditioned media containing secreted human PCSK9-D374Y or PCSK9-WT proteins are collected 24 hours later. The level of PCSK9 proteins in conditioned media is quantified by enzyme-linked immunosorbent assay (ELISA), as described previously (Dubuc G, Tremblay M, Paré G, Jacques H, Hamelin J, Benjannet S, Boulet L, Genest J, Bernier L, Seidah N G, Davignon J. 2010. A new method for measurement of total plasma PCSK9: clinical applications. J Lipid Res. 51:140-149).

Human HepG2 Cells Stably Expressing PCSK9.

Human HepG2 cells (ATCC, HB-8065) were transfected with the plasmid construct (human PCSK9 tagged at the C-terminus with V5) using the Fugene™ HD transfection reagent (Roche). Namely, HepG2 cells were transfected in a 35 mm culture dish using Fugene™ HD optimised protocol for HepG2 cells (2 ug DNA for 7 ul Fugene™ HD, following the manufacturer's instructions). 72 hours post transfection, the cells are trypsinised and transferred onto a 100 mm dish containing the selection medium: DMEM (high glucose+sodium pyruvate) (Wisent)/10% FBS and 600 ug/ml G418 (Wisent). After 10 days of selection, a cell pool expressing the gene of interest is obtained. See also Seidah, NG, Benjannet, S, Wickham, L, Marcinkiewicz, J, Jasmin, S B, Stifani, S, Baska, A, Prat, A and Chretien, M. The secretory proprotein convertase neural apoptosis-regulated convertase 1 (NARC-1): liver regeneration and neuronal differentiation. Proc. Natl. Acad. Sci. U.S.A, 100:928-933, 2003; Benjannet, S., Rhainds, D., Essalmani, R., Mayne, J., Wickham, L., Jin, W., Asselin, M. C., Hamelin, J., varret, M., Allard, D., Trillard, M., Abifadel, M., Tebon, A., Attie, A. D., Rader, D. J., Boileau, C., Brissette, L., Chretien, M., Prat, A., and Seidah, N. G. NARC-1/PCSK9 and its natural mutants: zymogen cleavage and effects on the low density lipoprotein (LDL) receptor and LDL cholesterol. J. Biol. Chem., 279: 48865-48875, 2004; and Benjannet, S., Rhainds, D., Hamelin, J., Nassoury, N., and Seidah, N. G. The proprotein convertase PCSK9 is inactivated by furin and/or PC5/6A: Functional consequences of natural mutations and post-translational modifications. J. Biol. Chem., 281:30561-30572, 2006.

PCSK9 Secretion Assay: Detection of Secreted PCSK9 by ELISA.

In 12-well plates (Greiner BioOne™), HepG2 cells stably expressing PCSK9(+V5) were seeded at a density of 1×10⁶/well in complete media and incubated for 20 hours, at 37° C. Cells were rinsed once with 2 ml/well of D-PBS (+Calcium, +Magnesium) (Wisent) followed by addition of 0.5 ml/well incubation media (+0.07% BSA) containing the inhibitory compounds at various concentrations (11, 33, 100 μM) or DMSO control (0.4% final) and overnight (24 hours) incubation. 24 hours-conditioned media was collected, centrifuged at 130×g for 5 minutes to remove cell debris, and the supernatant removed for PCSK9 quantification by ELISA (100 μl of 1:30 dilution). Cells were washed once with ice-cold D-PBS (+Calcium, +Magnesium) (Wisent), then lysed with 250 ul/well of radioimmunoprecipitation assay buffer (RIPA) (50 mM Tris-HCl, pH 7.8, 150 mM NaCl, 1% Nonident P-40, 0.5% sodium deoxycholate, 0.1% SDS) containing a mixture of protease inhibitors (Roche Applied Science) for 30 min on ice, and pelleted at 11,300×g for 5 minutes. Supernatant was removed for cellular PCSK9 quantification by ELISA (100 μl of 1:20 dilution) and total protein determination by the Bio-Rad™ DC Protein assay (Bio-Rad) (4 μl cell lysate in duplicate) (Dubuc G, Tremblay M, Pare G, Jacques H, Hamelin J, Benjannet S, Boulet L, Genest J, Bernier L, Seidah N G, Davignon J. 2010. A new method for measurement of total plasma PCSK9: clinical applications. J Lipid Res. 51:140-149). The ratio of PCSK9 concentration of media (M) over cells (C) was determined and compounds which decreased M/C by >30% were considered active.

Detection by Western Blot.

Cells are washed 3× in phosphate-buffered saline (PBS) and lysed in complete RIPA buffer (50 mM Tris/HCl, pH 8.0, 1% (v/v) Nonidet P40, 0.5% sodium deoxycholate, 150 mM NaCl and 0.1% (v/v) SDS) supplemented with 1× Complete Protease Inhibitor Mixture (Roche Applied Science). Proteins are separated by 8% SDS-polyacrylamide gel electrophoresis and blotted on polyvinylidene difluoride (PVDF, Perkin Elmer) membranes (GE Healthcare), which were blocked for 1 h in TBS-T (50 mM Tris-HCl, pH 7.5, 150 mM NaCl, 0.1% Tween-20) containing 5% nonfat dry milk. Membranes are then incubated 3 h in 5% nonfat milk with either a polyclonal hPCSK9 antibody (1:2500), a human LDLR antibody (1:1000, R&D Systems), or a rabbit P-Actin antibody (1:2500, Sigma). Appropriate horseradish peroxidase-conjugated secondary antibody (1:10,000, Sigma) is used for detection with enhanced chemiluminescence using the ECL plus kit (GE Healthcare).

Fluorescence-Activated Cell Sorting (FACS) Quantification of Cell Surface LDLR Levels.

HuH7 cells are incubated for 4 h or 18 h at 37° C. with various PCSK9 constructs in the presence or absence of the added compound(s), and then washed 3× with calcium/magnesium free Dulbecco's phosphate-buffered saline (DPBS) containing 0.5% bovine serum albumin (Sigma) and 1 g/l glucose (solution A). Cells are then incubated 5 min at 37□C with 500 μl of 1× Versene™ solution (Invitrogen) and layered on 5 ml of solution A. Cells are then centrifuged for 5 min at 1000 rpm and re-suspended in 1 ml of solution A containing 1:100 of monoclonal LDLR antibody C7 directed against human LDLR (mAb-C7, Santa Cruz Biotechnology) for 40 min. Cells are washed once with 5 ml of solution A, centrifuged and re-suspended for 20 min in 1 ml of PBS containing 1:250 of Alexa Fluor 647 donkey anti-mouse (Molecular Probes). Cells are washed and re-suspended in 300 μl of PBS 0.2% of propidium iodide (PI). Viable cells (PI-negative) are then analyzed by FACS for both PI and Alexa Fluor 647 using the FACS BD LSR (BD Biosciences).

Immunofluorescence of LDLR in Human Huh7 Cells.

HepG2 naïve cells were plated on Poly-L-Lysine-coated (50 ug/ml) round microscope cover slips 1.12 mm thickness (Fisherbrand 12CIR #1) that were placed in a 24-well cell culture plate. Seeding was performed in DMEM complete media (10% FBS) and 24 hours later the media was swapped for media (+0.07% BSA) containing 33.3 μM of the different inhibitor compounds (300 μl/well). A volume of DMSO equivalent to the volume of inhibitor compound was also diluted in 0.07% BSA containing media and used as a negative control. After a 20 hour incubation period, the cells were washed 3× with PBS and then fixed for 10 min with 3.7% paraformaldehyde. Immunofluorescence of human LDLR (green labeling) was performed under non-permeabilizing conditions. After an additional 3 washes with PBS, cells were blocked for 30 min with 1% BSA, followed by overnight incubation at 4° C. with primary antibody (1:200 goat polyclonal anti-hLDLR in 1% BSA, R&D Systems). Following a final 3 washes with PBS, antigen-antibody complexes were revealed by 1-hour incubation at room temperature with Alexa fluor-tagged secondary antibody and mounted in ProLong Gold Antifade Reagent (Molecular Probes, Invitrogen). Immunofluorescence analyses were performed with a confocal miscroscope (Zeiss LSM-710).

Dil-LDL Uptake Cell-Based Assay for PCSK9 Activity.

HepG2 naive cells and HEK293 naive cells were plated in 96-well plates (CellBind black plate with clear bottom (Corning; Cat #3340)) at a density of 25,000 cells/well, in complete media (DMEM high glucose (+sodium pyruvate for HepG2) (Wisent)+10% FBS). After 20 hours, cells were washed for 30 min with serum free DMEM media (100 μl/well), the wash media was removed and replaced with 100 μl/well of incubation media (DMEM high glucose (+sodium pyruvate for HepG2) (Wisent)+0.07% BSA (Sigma-Aldrich)) containing compound at various concentrations (11, 33, 100 μM) or DMSO control (0.4% final). Each condition was prepared in triplicates. After 6 hours incubation at 37° C., Dil-LDL (Biomedical Technologies (Cat #BT-904)) was added to the cell media (5 μl) at 5 μg/ml final concentration, and cells returned to tissue culture incubator for another 18 hours (a total of 24 hours incubation with the compounds). After 2 washes (200 μl/well) with ice-cold D-PBS (Wisent) and aspiration of the final wash, plates were scanned (bottom read) on a SpectraMax GeminiEM™ plate reader (Molecular Devices). For each well, raw Dil-LDL uptake was measured as the average fluorescence intensity (RFU) (ex: 520 nm/em: 575 nm, cutoff: 550 nm) of 9 readings in 3 different points in the well. Dil-LDL uptake in each well was corrected for total number of cells by performing a CyQuant™ cell assay (Invitrogen; Cat #C7026). Namely, after recording the Dil fluorescence, the plate was frozen overnight at −80° C. The next day, the plates were thawed at room temperature, the cells lysed according to the manufacturer's protocol, and the number of cells/well was determined by measuring the fluorescence (RFU) of the CyQuant green fluorescent dye bound to the cellular nucleic acids (ex: 485 nm/em: 538 nm, cutoff: 515 nm). For each condition, Dil-RFU was divided by CyQuant-RFU. Corrected dil-LDL uptake is reported as % DMSO control and was obtained from triplicate wells.

Transfections, Biosynthetic Analyses, Immunoprecipitations of PCSK9.

Transfections are done with 3×10⁵ HEK293 cells using Effectene™ (Qiagen) and a total of 0.5 μg of cDNAs. Alternatively, 5×10⁵ HuH7 or 6×10⁵ HepG2 cells are transfected with a total of 4 μg of cDNAs in Lipofectamine™ 2000 (Invitrogen). Two days post-transfection, HEK293 cells are washed and then incubated for various times with either 250 μCi/ml [35S]Met/Cys (PerkinElmer Life Sciences). The cells are lysed in modified RIPA buffer (150 mM NaCl, 50 mM Tris-HCl, pH 7.5), 1% Nonidet P-40, 0.5% sodium deoxycholate, 0.1% SDS, and a protease inhibitor mixture (Roche Applied Science), after which the lysates and media are prepared for immunoprecipitation. The antibodies used are the anti-V5 mAb (Invitrogen, 1:500), and proprietary rabbit anti-PCSK9 31-454 (A-03). Immunoprecipitates are resolved by SDS-PAGE on 8% Tricine gels and autoradiographed. These experiments are repeated at least three times. Quantitation is performed on a Storm Imager™ (Amersham Biosciences) by using the ImageQuant™ version 5.2 software. Biosynthesis is a sensitive method that allows for testing whether the compounds affect proPCSK9 to PCSK9 activation in the endoplasmic reticulum and hence the secretion of active PCSK9 complexed with its prosegment into the medium. Cells engineered to express endogenously and stably PCSK9 (e.g. PCSK9-V5) WT or mutated variants are incubated with the compound(s) and the different PCSK9 forms present in the media and cell lysates are analysed by Western blot using sensitive human PCSK9 antibodies.

MTT Toxicity Assay.

HepG2 stable cells over-expressing hPCSK9(+V5) were seeded in complete DMEM (high glucose+sodium pyruvate) (Wisent)+10% FBS media in 96-well plates (Greiner BioOne) at a density of 1×10⁵ cells/well (100 μl) and incubated for 20 hours. Cells were rinsed once with 100 ul/well of serum free DMEM media, followed by addition of 100 μl/well of DMEM (high glucose+sodium pyruvate) (Wisent)+0.07% BSA (Sigma-Aldrich) media containing compound at various concentrations (11, 33, 100 μM) or DMSO control (0.4% final) followed by overnight (24 hours) incubation. The MTT assay (Promega) consisted of addition of 20 μl/well MTT reagent and incubation for 45 minutes at 37° C., following the manufacturer's instructions. Absorbance was recorded at 490 nm and corrected for background due to nonspecific absorbance (690 nm).

### Methods of Using the Compounds

PCSK9 has been implicated in cholesterol homeostasis, as it appears to have a specific role in cholesterol biosynthesis or uptake. In a study of cholesterol-fed rats, it was reported that PCSK9 was downregulated in a similar manner to other genes involved in cholesterol biosynthesis, (Maxwell et al., 2003 J Lipid Res. 44:2109-2119). PCSK9 expression has been found to be upregulated by statins in a manner attributed to the cholesterol-lowering effects of the drugs (Dubuc et al., 2004, Arterioscler Thromb Vasc Biol. 24: 1454-1459). Adenoviral expression of PCSK9 results in a time-dependent increase in circulating low density lipoprotein (LDL) cholesterol (LDL-C) (Benjannet et al., 2004 J. Biol. Chem. 279:48865-48875), and mice with PCSK9 gene deletions have increased levels of hepatic LDL receptors (LDLR) and clear LDL-C from the plasma more rapidly (Rashid et al., 2005 supra). Medium from HepG2 cells which are transiently transfected with PCSK9 is found to reduce the amount of cell surface LDLRs and internalization of LDL-C when transferred to untransfected HepG2 cells (Cameron et al., 2006 Human Mol. Genet. 15:1551-1558). Additionally, purified PCSK9 added to the medium of HepG2 cells reduced the number of cell-surface LDLRs in a dose- and time-dependent manner (Lagace et al., 2006, supra).

A number of mutations in the gene PCSK9 have been associated with autosomal dominant hypercholesterolemia (ADH), an inherited metabolism disorder which is characterized by marked elevations of LDL-C particles in the plasma that can lead to premature cardiovascular failure (e.g., Abifadel et al, 2003 Nat. Genetics 34:154-156; Tirnms et al, 2004 Hum. Genetics 114:349-353; Leren, 2004 Clin. Genet. 65:419-422).

Increased expression or upregulation of PCSK9 is associated with increased plasma levels of LDL-C, and inhibition or lack of expression of PCSK9 is associated with low LDL-C plasma levels. Lower levels of LDL-C associated with sequence variations in PCSK9 confer protection against coronary heart disease (Cohen, et al, 2006 N. Engl. J. Med. 354:1264-1272).

The compounds described herein have in vitro and in vivo therapeutic utilities. For example, these molecules can be administered to cells in culture, e.g., in vitro or ex vivo, or in a subject in need thereof, e.g., in vivo, to treat, prevent or diagnose a variety of disorders associated with PCSK9 activity/function.

Compounds of the present invention are particularly suitable for treating human patients having, or at risk for, elevated cholesterol or a condition associated with elevated cholesterol (e.g., LDL cholesterol), including a lipid disorder (e.g., hyperlipidemia, hypercholesterolemia, xanthomatosis). Compounds of the present invention may also be suitable for treating human patients having ateriosclerotic conditions (e.g., atherosclerosis), coronary artery disease, cardiovascular disease, stroke, ischemia, peripheral vascular diseases, and prophylactically for patients at risk for these disorders, e.g., due to the presence of one or more risk factors (e.g., hypertension, cigarette smoking, diabetes, obesity, or hyperhomocysteinemia).

As used herein the terms “LDL-cholesterol-related disease or disorder” refer to a disease or condition resulting in part from a high level of circulating LDL-cholesterol in the blood stream. Without being so limited, LDL-cholesterol-related diseases or disorders include hyperlipidemia, hypercholesterolemia, xanthomatosis and cardiovascular diseases such as ateriosclerotic conditions (e.g., atherosclerosis), coronary artery disease, stroke, ischemia and peripheral vascular diseases.

As used herein, the terms “treat/treating/treatment” and “prevent/preventing/prevention”, refer to eliciting the desired biological response, i.e. a therapeutic and prophylactic effect, respectively. In accordance with the subject invention, the therapeutic effect comprises a decrease/reduction in the progress of the LDL-cholesterol-related disease or disorder or in the severity of associated symptoms or a complete cure of the LDL-cholesterol-related disease or disorder and/or associated symptoms. In accordance with the invention, a prophylactic effect may comprise a delay or decrease in the onset of, progression of, or the severity of LDL-cholesterol-related disease or disorder and associated symptoms (e.g., decreased plasma LDL/cholesterol levels), following administration of a compound or composition of the present invention. In an embodiment, the compound or composition of the present invention, comprising a compound of Formula (1) treats or prevent the LDL-cholesterol-related disease or disorder in a subject. The methods, compositions formulations and uses described herein are suitable for both humans and animals (including birds), preferably mammals.

### Combination Therapy

When compounds are administered together with another agent, the two can be administered sequentially in either order or simultaneously (in the same composition or in different compositions). In some embodiments, a compound is administered to a subject who is also receiving therapy with a second agent useful for treating the disease/condition (e.g., a second cholesterol-reducing agent). Examples of active ingredients that may be administered in combination with of the present invention include, but are not limited to, other compounds which improve a patient's lipid profile, such as (a) HMG-CoA reductase inhibitors, (e.g., statins, including lovastatin, simvastatin, fluvastatin, rosuvastatin, pravastatin, rivastatin, atorvastatin, itavastatin, pitavastatin, cerivastatin and other statins). Statins inhibit cholesterol synthesis by blocking HMGCoA, a key enzyme in cholesterol biosynthesis; (b) cholesterol absorption inhibitors, such as stanol esters, beta-sitosterol, sterol glycosides such as tiqueside; and azetidinones, such as ezetimibe; (c) inhibitors of cholesterol ester transport protein (CETP) (e.g., anacetrapib or dalcetrapib) which are now in clinical trials to increase HDL and decrease LDL cholesterol; (d) niacin and related compounds, such as nicotinyl alcohol, nicotinamide, and nicotinic acid or a salt thereof; (e) bile acid sequestrants (cholestyramine, colestipol (e.g., colestipol hydrochloride), dialkylaminoalkyl derivatives of a cross-linked dextran, Colestid®, LoCholest®. Bile acid sequestrants interrupt the recycling of bile acids from the intestine to the liver; (f) acyl CoA:cholesterol acyltransferase (ACAT) inhibitors, such as avasimibe and melinamide, and including selective ACAT-1 and ACAT-2 inhibitors and dual inhibitors; (g) PPARy agonists, such as gemfibrozil and fenofibric acid derivatives (fibrates), including clofibrate, fenofibrate, bezafibrate, ciprofibrate, and etofibrate; (h) microsomal triglyceride transfer protein (MTP)/ApoB secretion inhibitors; (i) anti-oxidant vitamins, such as vitamins C and E and beta carotene; (j) thyromimetics; (k) LDL receptor inducers, (I) platelet aggregation inhibitors, for example glycoprotein Ib/IIIa fibrinogen receptor antagonists and aspirin; (m) vitamin B 12 (also known as cyanocobalamin); (n) folic acid or a pharmaceutically acceptable salt or ester thereof, such as the sodium salt and the methylglucamine salt; (o) FXR and LXR ligands, including both inhibitors and agonists; (p) agents that enhance ABCA1 gene expression; (q) ileal bile acid transporters and (r) long chain alpha, omego-dicarboxylic acids.

A combination therapy regimen may be additive, or it may produce synergistic results (e.g., reductions in cholesterol greater than expected for the combined use of the two agents). In some embodiments, combination therapy with a compound and a cholesterol-reducing agent (e.g., a statin, fibrates, ezetimibe or a combination thereof) produces synergistic results (e.g., synergistic reductions in cholesterol). In some subjects, this can allow reduction in the dosage of the cholesterol-reducing agent to achieve the desired cholesterol levels. Compounds may be useful for subjects who are intolerant to therapy with another cholesterol-reducing agent, or for whom therapy with another cholesterol-reducing agent has produced inadequate results (e.g., subjects who experience insufficient LDL-C reduction on statin therapy).

A compound described herein can be administered to a subject with elevated cholesterol (e.g., LDL-cholesterol) (e.g., a human subject with total plasma cholesterol levels of 200 mg/dl or greater, a human subject with LDL-C levels of 160 mg/dl or greater).

### Kits

The present invention also encompasses kits comprising at least one compound of the present invention. For example, the kit can comprise one or more compounds preventing and/or treating LDL-cholesterol-related disease or disorder. The kit may optionally include one or more control samples and a device (e.g., syringe, etc.). The kit may optionally include one or more other active ingredient which improves a patient's lipid profile. The compounds or ingredients can be packaged in a suitable container. The kit can further comprise instructions for using the kit (e.g., to use the compound for preventing or treating the low density lipid-cholesterol-related disease or disorder in the subject).

The invention having been fully described, it is further illustrated by the following examples and claims, which are illustrative and are not meant to be further limiting. Those skilled in the art will recognize or be able to ascertain using no more than routine experimentation, numerous equivalents to the specific procedures described herein. Such equivalents are within the scope of the present invention and claims. The contents of all references, including issued patents and published patent applications, cited throughout this application are hereby incorporated by reference.

### MODE(S) FOR CARRYING OUT THE INVENTION

The present invention is illustrated in further details by the following non-limiting examples.

### Intermediate 1

### (S)-Methyl 2-((S)-2-amino-3-phenylpropanamido)propanoate trifluoroacetate

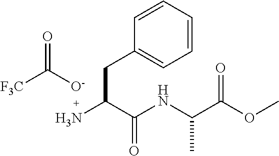 [SMILES: O=C([O-])C(F)(F)F ; COC(=O)[C@H](C)NC(=O)[C@@H](N)Cc1ccccc1]

Step 1: DIPEA (46 mL, 260 mmol, 3.5 eq.) was added dropwise over 10 min to a stirred solution of N-Boc-L-phenylalanine (20 g, 74 mmol), L-alanine methyl ester hydrochloride (12.6 g, 90.5 mmol, 1.2 eq.) and HATU (42.9 g, 113 mmol, 1.5 eq.) in DMF (250 mL) at 0° C. followed by warming to rt. After 18 h of stirring, the reaction mixture was poured into a cold saturated solution of NaHCO₃ and extracted with 1:1 EtOAc:hexanes (2×). The combined organic fractions were washed with water and brine, dried (MgSO₄), filtered and concentrated under reduced pressure. The residue was purified on silica gel eluting with an increasing proportion of EtOAc in hexanes.

Step 2: A stirred solution of the product from Step 1 (14.8 g, 42.2 mmol) in CH₂Cl₂ (141 mL) at 0° C. was treated with TFA (32.5 mL, 422 mmol, 10 eq.) dropwise. The reaction mixture was subsequently warmed to rt and stirred for an additional 3 h before being concentrated to dryness. The residue was triturated with Et₂O and the solid was collected by filtration and dried under high vacuum to afford the title compound.

### Intermediate 2

### (2S)-2-[(2S)-2-[(2S)-2-[(Methoxycarbonyl)amino]-3-methylbutanamido]-3-phenylpropanamido]propanoic acid

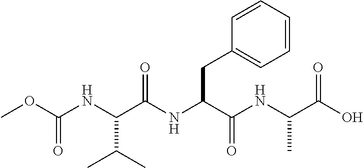 [SMILES: COC(=O)N[C@H](C(=O)N[C@@H](Cc1ccccc1)C(=O)N[C@@H](C)C(=O)O)C(C)C]

Step 1: Collidine (3.0 mL, 23 mmol, 2.5 eq.) was added dropwise over 10 min to a stirred solution of N-methoxycarbonyl-L-valine (1.60 g, 9.13 mmol), Intermediate 1 (3.49 g, 9.59 mmol, 1.05 eq.) and HATU (3.82 g, 10.0 mmol, 1.1 eq.) in DMF (30 mL) at 0° C. with stirring at this temperature for 1 h and then at rt for 18 h. The mixture was then recooled to 000° C. and a saturated aqueous solution of NaHCO₃ (50 mL) was added slowly followed by the addition of Et₂O (50 mL). The mixture was stirred vigorously for 20 min and the solid product was collected by filtration, washed with separate portions of water and Et₂O, and dried under suction and high vacuum.

Step 2: 1 M LiOH (10.65 mL, 10.65 mmol, 1.25 eq.) was added dropwise to a stirred suspension of the product from Step 1 (3.47 g, 8.52 mmol) in a mixture of THF (40 mL) and MeOH (20 mL) followed by slow warming to rt over several hours. The vessel contents were then recooled to 000° C., additional 1 M LiOH (3.4 mL, 3.4 mmol, 0.4 eq.) was added dropwise and the mixture was stirred at rt for an additional 2 h prior to acidification to pH 4 with 1 M HCl at 0° C. and extraction with ethyl acetate (2×). The combined organic fractions were washed with water, half saturated brine, dried (MgSO₄), filtered and concentrated under reduced pressure to afford the title compound which was used without further purification.

### Intermediate 3

### 2,5-Dioxopyrrolidin-1-yl(2S)-2-[(2S)-2-[(2S)-2-[(methoxycarbonyl)amino]-3-methylbutanamido]-3-phenylpropanamido]propanoate

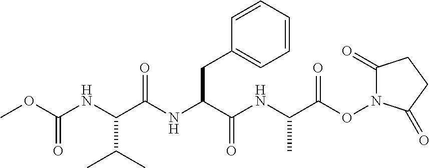 [SMILES: COC(=O)N[C@H](C(=O)N[C@@H](Cc1ccccc1)C(=O)N[C@@H](C)C(=O)ON1C(=O)CCC1=O)C(C)C]

DIPEA (0.33 mL, 1.9 mmol, 3.0 eq.) was added to a solution of Intermediate 2 (0.25 g, 0.64 mmol) and TFA-NHS (0.40 g, 1.9 mmol, 3.0 eq.) in DMF (4.5 mL) at −78° C. The reaction mixture was then warmed slowly to 000° C. and stirred for 2 h at this temperature before being partitioned between water and EtOAc. The layers were separated and the aqueous layer was extracted with EtOAc (2×). The combined organic layers were washed with half saturated brine, dried (MgSO₄), filtered and concentrated under reduced pressure to give the title compound that was used in subsequent reactions without further purification.

### Intermediate 4

### Methyl (2S)-2-[(2S)-2-[(2S)-2-amino-3-methylbutanamido]-3-phenylpropanamido]propanoate hydrochloride

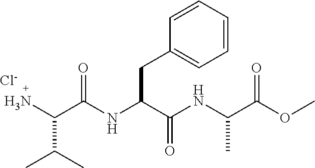 [SMILES: [Cl-] ; COC(=O)[C@H](C)NC(=O)[C@H](Cc1ccccc1)NC(=O)[C@@H](N)C(C)C]

Step 1: DIPEA (6.7 mL, 38 mmol, 3.5 eq.) was added dropwise over 10 min to a stirred solution of Intermediate 1 (4.0 g, 11 mmol), N-Boc-L-Valine (2.62 g, 12.0 mmol, 1.1 eq.) and HATU (5.0 g, 13 mmol, 1.2 eq.) in DMF (35 mL) at 0° C. After stirring overnight at rt, the vessel contents were poured into a mixture of ice (100 mL), saturated aqueous NaHCO₃ (100 mL) and Et₂O (200 mL) and the mixture was stirred for 30 min. The solid product was collected by filtration, washed with water and 1:1 Et₂O/hexanes and was dried under suction and high vacuum.

Step 2: TFA (7.3 mL, 95 mmol, 10 eq.) was added dropwise to a stirred solution of the product from Step 1 (4.28 g, 9.52 mmol) in CH₂Cl₂ (30 mL) at 000° C. The reaction vessel contents were then held at rt for 5 h before being concentrated to dryness. The residue was triturated with Et₂O and the solid product was collected by filtration and dried under high vacuum.

Step 3: 4 M HCl in dioxane (14 mL, 56 mmol, 5.9 eq.) was added to a stirred suspension of the product from Step 2 (4.40 g, 9.52 mmol) in MeOH (20 mL) with stirring overnight at rt. After concentrating the mixture to dryness, Et₂O was added with stirring for 20 min and the solid product was collected by filtration and dried under high vacuum to afford the title compound.

### Intermediate 5

### Methyl (4R/S)-4-azido-4-[(1S,2S,6R,8S)-2,9,9-trimethyl-3,5-dioxa-4-boratricyclo[6.1.1.0^{2,6}]decan-4-yl]butanoate

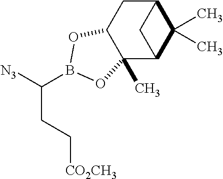

### Reaction Sequence:

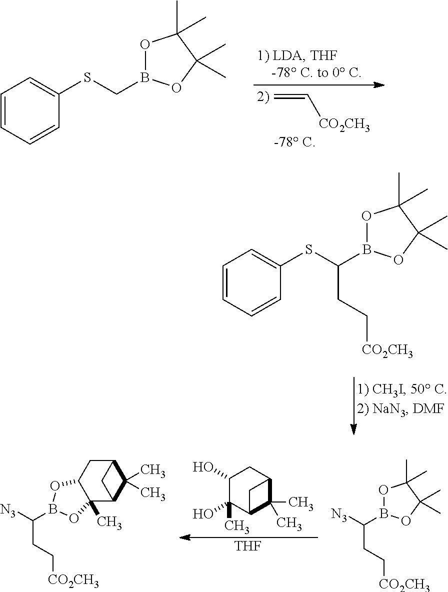

Step 1: DIPEA (6.2 mL, 44 mmol, 1.1 eq.) and THF (40 mL) were placed in a flame-dried rbf flushed with N₂. The temperature was cooled to −78° C. and n-BuLi (2.5 M in hexanes, 17 mL, 42 mmol, 1.0 eq.) was added dropwise to the reaction vessel over a period of 2 min. The reaction mixture was then warmed to 000° C. and stirred for 5 min before being cooled back to −78° C. A solution of 4,4,5,5,-tetramethyl-2-phenylsulfanylmethyl-1,3,2-dioxaborolane (10 mL, 42 mmol) in THF (10 mL) was added dropwise to the reaction mixture followed by stirring at 000° C. After 1 h at this temperature, the reaction vessel contents were recooled to −78° C. and methyl acrylate (7.6 mL, 85 mmol, 2.0 eq.) was introduced dropwise. After 6 h at −78° C., the reaction was quenched by pouring into 200 mL of 10% citric acid aqueous solution. The layers were separated and the aqueous layer was extracted with Et₂O (3×). The organics were combined, washed with half saturated brine, dried (MgSO₄), filtered and concentrated under reduced pressure. The product was isolated by flash chromatography on silica gel eluting with an increasing proportion of EtOAc in hexanes.

Step 2: The product from Step 1 (1.80 g, 5.35 mmol), iodomethane (6.6 mL, 110 mmol, 20 eq.) and acetonitrile (8 mL) were heated together in a N₂ flushed sealed tube at 50° C. After 24 h at this temperature, the vessel contents were concentrated under reduced pressure. The residue was used directly in the next step.

Step 3: The product from Step 2 (1.89 g, 5.35 mmol) was dissolved in DMF (11 mL) and treated with sodium azide (0.70 g, 11 mmol, 2.0 eq.) with stirring overnight at rt. The reaction mixture was then partitioned between EtOAc and water. The layers were separated and the aqueous layer extracted with EtOAc (2×). The combined organics were washed with half saturated brine (3×), dried (MgSO₄), filtered and concentrated under reduced pressure. The residue was used directly in the next step.

Step 4: A solution of the product from Step 3 (1.43 g, 5.35 mmol) and (1S,2S,3R,5S)-(+)-pinanediol (1.00 g, 5.90 mmol, 1.1 eq.) in THF (21 mL) was stirred at rt for 18 h. The reaction vessel contents were concentrated to dryness and the residue was purified by flash chromatography on silica gel eluting with an increasing proportion of EtOAc in hexanes to afford the title compound.

### Intermediate 6

### (4R/S)-4-Azido-4-[(1S,2S,6R,8S)-2,9,9-trimethyl-3,5-dioxa-4-boratricyclo[6.1.1.0^{2,6}]decan-4-yl]butanamide

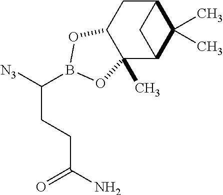

Intermediate 5 (0.30 g, 0.94 mmol) was stirred with 7.0 M NH₃ in MeOH (4 mL, 30 mmol, 30 eq.) for 42 h at rt in a N₂ flushed sealed tube. The vessel contents were then concentrated under reduced pressure and the residue was purified by flash chromatography on silica gel eluting with an increasing proportion of MeOH in EtOAc to afford the title compound.

### Intermediate 7a

### 4-(3-(Trifluoromethyl)-3H-diazirin-3-yl)benzoic acid

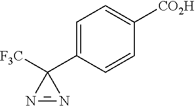 [SMILES: O=C(O)c1ccc(C2(C(F)(F)F)N=N2)cc1]

### Intermediate 7b

### 3-(3-(Trifluoromethyl)-3H-diazirin-3-yl)benzoic acid

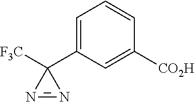 [SMILES: O=C(O)c1cccc(C2(C(F)(F)F)N=N2)c1]

### Reaction Sequence:

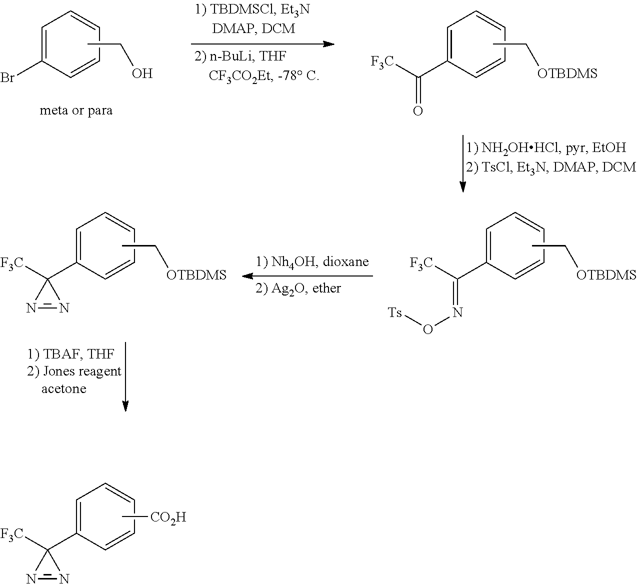

Step 1: Et₃N (11 mL, 80 mmol, 1.5 eq.) and DMAP (0.65 g, 5.3 mmol, 0.1 eq.) were added to a stirred solution of 4-bromobenzyl alcohol (to prepare 7a) or 3-bromobenzyl alcohol (to prepare 7b) (10.00 g, 53.46 mmol) in CH₂Cl₂ (60 mL) at 0° C. followed by tert-butyldimethylsilyl chloride (8.86 g, 58.8 mmol, 1.1 eq.) in a portion-wise manner. The reaction vessel contents were then warmed to rt for 3 h. Water was added and the mixture was stirred until all solids had dissolved. The mixture was transferred to a separatory funnel and extracted with CH₂Cl₂ (3×). The combined organic fractions were washed with a saturated aqueous solution of NH₄Cl and water, and then were dried (MgSO₄), filtered and concentrated under reduced pressure. The residue was used directly in the next step.

Step 2: n-BuLi (2.5 M in hexanes, 24 mL, 60 mmol, 1.1 eq.) was added dropwise over 30 min to a stirred solution of the product from Step 1 (15.89 g, 52.74 mmol) in THF (175 mL) at −78° C. The reaction was stirred at this temperature for 30 min prior to the dropwise introduction of a solution of ethyl trifluoroacetate (7.6 mL, 63 mmol, 1.2 eq.) in THF (30 mL) over 30 min. The reaction was stirred for an additional 90 min at −78° C. before being partitioned between EtOAc and a saturated solution of NH₄Cl. The phases were separated and the aqueous layer was extracted with EtOAc (2×). The combined organic fractions were washed with a saturated solution of brine, dried (MgSO₄), filtered and concentrated under reduced pressure. The residue was purified by distillation under high vacuum (P=10 mmHg) collecting the fraction boiling between 160-170° C.

Step 3: The product from Step 2 (8.0 g, 25 mmol) and hydroxylamine hydrochloride (1.92 g, 27.6 mmol, 1.1 eq.) were heated together at reflux in a mixture of pyridine (12 mL) and EtOH (6 mL). After 3 h, the solvents were removed under reduced pressure and the residue was partitioned between EtOAc and water. The phases were separated and the aqueous layer was extracted with EtOAc (2×). The combined organic fractions were washed with a saturated solution of brine, dried (MgSO₄), filtered and concentrated under reduced pressure. The residue was purified on silica gel with an increasing proportion of EtOAc in hexanes.

Step 4: p-Toluenesulfonyl chloride (3.29 g, 17.2 mmol, 1.15 eq.) was added in a portion-wise manner to a stirred solution of the product from Step 3 (5.0 g, 15 mmol), Et₃N (2.5 mL, 18 mmol, 1.2 eq.) and DMAP (0.183 g, 1.5 mmol, 0.10 eq.) in CH₂Cl₂ (26 mL) at 000° C. Following completion of the reaction as judged by TLC, the mixture was concentrated under reduced pressure and the residue was partitioned between EtOAc and water. The layers were separated and the organic phase was washed with additional water and then with brine before being dried (MgSO₄), filtered and concentrated under reduced pressure. The residue was used without additional purification in the next step.

Step 5: Concentrated NH₄OH (68 mL) was added to a stirred solution of the product from Step 4 (6.85 g, 14.1 mmol) in dioxane (68 mL) at 10° C. in a thick-walled glass tube. The tube was capped and the contents were stirred at rt for 48 h. Water and EtOAc were then added and the layers were separated. The aqueous layer was extracted with EtOAc (2×) and the combined organic fractions were washed with a saturated solution of brine, dried (MgSO₄), filtered and concentrated under reduced pressure. The residue was purified on silica gel with an increasing proportion of EtOAc in hexanes.

Step 6: A solution of the product from Step 5 (1.8 g, 5.4 mmol) in Et₂O (54 mL) was stirred with Ag₂O (2.50 g, 10.9 mmol, 2 eq.) at rt. After 4 h, a second portion of Ag₂O was added (2.50 g, 10.9 mmol, 2 eq.) and the mixture was stirred at rt overnight. Hexanes (100 mL) were then added and the mixture filtered through a silica gel pad. The pad was washed with 9:1 hexanes/EtOAc and the filtrate was concentrated under reduced pressure. The residue was used directly in the next step.

Step 7: TBAF (1.0 M in THF, 6.2 mL, 6.2 mmol, 1.2 eq.) was added dropwise to a solution of the product from Step 6 (1.70 g, 5.15 mmol) in THF (25 mL) at 0° C. followed by stirring at rt for 1 h. The mixture was then partitioned between EtOAc and water and the phases were separated. The aqueous phase was extracted with EtOAc (2×) and the combined organics were washed with a saturated solution of brine, dried (MgSO4), filtered and concentrated under reduced pressure. The residue was purified on silica gel with an increasing proportion of EtOAc in hexanes.

Step 8: Jones reagent (2.5 M, 0.56 mL, 1.4 mmol, 2.0 eq.) was added to a solution of the product from Step 6 (0.15 g, 0.69 mmol) in acetone (7.0 mL) with stirring at 0° C. for 1 h. The mixture was then diluted with EtOAc and washed with water in a separatory funnel until no color was visible in the aqueous layer. The organic layer was washed with brine, dried over MgSO₄, filtered and concentrated under reduced pressure to afford the title compound.

### Intermediate 7c

### 2-(4-(3-(trifluoromethyl)-3H-diazirin-3-yl)phenyl)acetic acid

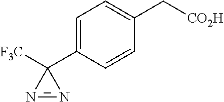 [SMILES: O=C(O)Cc1ccc(C2(C(F)(F)F)N=N2)cc1]

### Intermediate 7d

### 2-(3-(3-(trifluoromethyl)-3H-diazirin-3-yl)phenyl)acetic acid

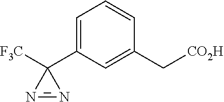 [SMILES: O=C(O)Cc1cccc(C2(C(F)(F)F)N=N2)c1]

### Reaction Sequence:

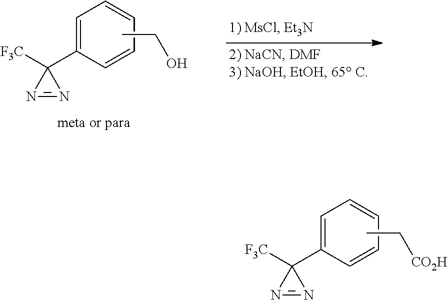

Step 1: MsCl (0.22 mL, 2.8 mmol, 1.5 eq.) was added dropwise to a solution of the para- or meta-product (400 mg, 1.85 mmol) from step 7 in the preparation of Intermediate 7a or 7b and Et₃N (0.52 mL, 3.7 mmol, 2 eq.) in Et₂O (19 mL) at 0° C. with stirring at this temperature for 15 min and then at rt. After 2 h, the mixture was filtered, the solid material was washed with ether, and the filtrate was concentrated in vacuo. The residue was used in the next step without further purification.

Step 2: The product from Step 1 (535 mg, 1.82 mmol) and NaCN (134 mg, 2.70 mmol, 1.5 eq.) were stirred together in DMF (10 mL) at rt overnight. The mixture was subsequently partitioned between water and Et₂O and the layers were separated. The organic phase was washed with water, then with brine, dried (MgSO₄), filtered and concentrated. The residue was purified by flash chromatography on silica gel eluting with 1/4 EtOAc/hexanes.

Step 3: The product from Step 2 (200 mg, 0.89 mmol) was stirred with 1 M NaOH (4.5 mL, 5 eq.) in EtOH (4.5 mL) at 65° C. overnight. After cooling to rt, the reaction was quenched by the addition of 1 M HCl (5 mL) and the mixture was partitioned between water and Et₂O. The layers were separated and the aqueous phase was extracted with additional Et₂O (2×). The organics were combined, washed with brine, dried (MgSO₄), filtered and concentrated. The residue was purified by flash chromatography on silica gel eluting with 1/9 EtOH/hexanes containing 1% AcOH to afford the title compound as a pale yellow solid.

### Intermediate 8

### 4-Amino-6,6,6-trifluoro-5-hydroxyhexanamide hydrochloride

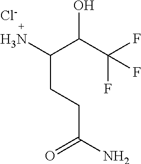 [SMILES: [Cl-] ; NC(=O)CCC(N)C(O)C(F)(F)F]

### Reaction Sequence:

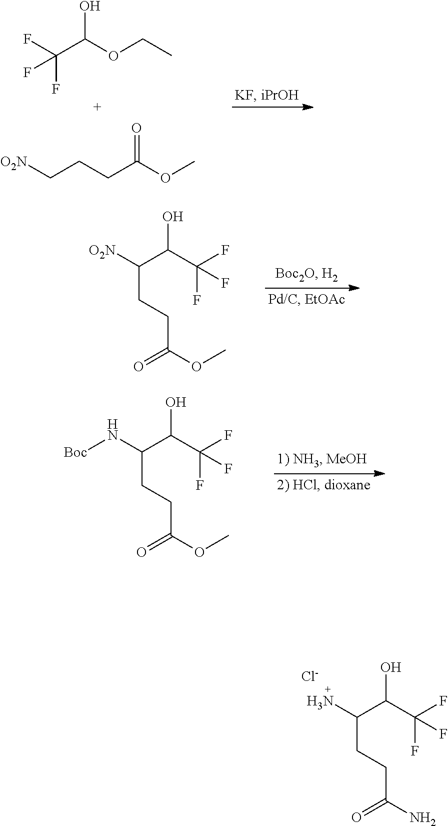

Step 1: Trifluoroacetaldehyde ethyl hemiacetal (90%, 15 mL, 110 mmol, 1.8 eq.), methyl 4-nitrobutyrate (8.0 mL, 62 mmol), potassium fluoride (3.65 g, 62.8 mmol) and i-PrOH (20 mL) were placed in a sealed reaction vessel and the mixture was stirred at rt for 20 h. The mixture was then partitioned between EtOAc (200 mL) and a 1:1 mixture of 5% (w/w) aqueous citric acid and brine (300 mL) and the layers were separated. The organic phase was washed with additional brine (200 mL) and the combined aqueous layers were extracted with EtOAc (200 mL). The organic layers were combined, dried over MgSO₄, filtered and concentrated by rotary evaporation under reduced pressure while keeping the bath temperature at 20° C. The residue was purified by column chromatography on silica gel by eluting with 20% EtOAc/hexanes.

Step 2: A mixture of the product from Step 1 (13.0 g, 52.9 mmol), di-tert-butyl dicarbonate (15.0 g, 68.7 mmol, 1.3 eq.) and 10% Pd/C (4.00 g) was placed in a septum-capped rbf under vacuum and degassed EtOAc (100 mL) was introduced by syringe. The mixture was then placed under an atmosphere of H₂ via a latex balloon and the reaction vessel was sonicated in an ultrasonic bath for 2 min followed by stirring of the contents at rt for 24 h. Following completion of the reaction as judged by TLC, the vessel was flushed with N₂ and CH₂Cl₂ was added. The suspension was filtered through a Celite pad and the pad was washed thoroughly with a mixture of EtOAc and CH₂Cl₂. The filtrate was concentrated in vacuo and the residue was treated with 1:4 mixture of Et₂O/heptane. After standing for 4 h, the solid product was collected by suction filtration and washed with a small amount of 1:4 Et₂O/heptane to afford the desired diasteromer in >10:1 diastereomeric purity.

Step 3: A solution of NH₃ in MeOH (7.0 M, 30 mL, 200 mmol, 20 eq.) was added to the product from Step 2 (3.0 g, 9.5 mmol) at 0° C. under N₂ in a thick walled glass tube and the entrance was sealed with a Teflon screw cap followed by heating of the contents at 35° C. for 5 days. The mixture was then concentrated under reduced pressure and the residue was stirred with Et₂O overnight. The solid product was collected by suction filtration and dried under high vacuum.

Step 4: A solution of HCl in 1,4-dioxane (4 M, 1.2 mL, 5 mmol, 5 eq.) was added dropwise to a suspension of the product from Step 3 (0.30 g, 1.0 mmol) in CH₂Cl₂ (2 mL) at 0° C. followed by stirring at rt until the reaction was determined to be complete by LCMS (3 h). The reaction mixture was then concentrated to dryness under reduced pressure to afford the title compound. The material was stored under a N₂ atmosphere at −15° C. and was used in subsequent reactions without further purification.

### Intermediate 9

### (4S)-4-Amino-6,6,6-trifluoro-5-hydroxyhexanamide hydrochloride

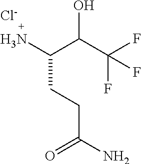 [SMILES: [Cl-] ; NC(=O)CC[C@H](N)C(O)C(F)(F)F]

### Reaction Sequence:

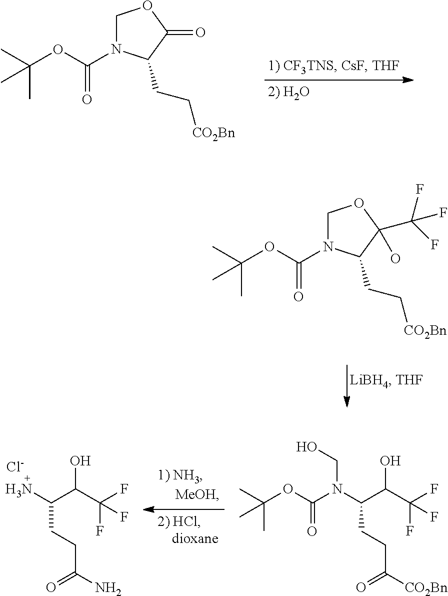 [SMILES: CC(C)(C)OC(=O)N1COC(=O)[C@@H]1CCC(=O)OCc1ccccc1 ; CC(C)(C)OC(=O)N1COC(O)(C(F)(F)F)[C@@H]1CCC(=O)OCc1ccccc1 ; [Cl-] ; NC(=O)CC[C@H](N)C(O)C(F)(F)F ; CC(C)(C)OC(=O)N(CO)[C@@H](CCC(=O)C(=O)OCc1ccccc1)C(O)C(F)(F)F]

Step 1: A solution of (S)-3-tert-butoxycarbonyl-4-(2-benzyloxycarbonylethyl)oxazolidin-5-one (4.97 g, 14.2 mmol) in dry THF (30 mL) was treated with CF₃TMS (2.5 mL, 17 mmol, 1.2 eq.) and CsF (0.34 g, 2.2 mmol, 0.15 eq.) under an atmosphere of N₂. The reaction vessel was then sonicated in an ultrasonic bath and the progress of the reaction was monitored by TLC. After 1 h, water (2.5 mL) was added and sonication was continued until hydrolysis of the intermediate silyl ether was judged to be complete by TLC (30 min). EtOAc was added and the mixture was washed with water and brine. The aqueous phases were extracted with EtOAc and the combined organic layers were dried over Na₂SO₄/MgSO₄, filtered and concentrated under vacuum and the residue was used directly in the next step.

Step 2: A solution of LiBH₄ in THF (2.0 M, 6.5 mL, 13 mmol, 1.05 eq.) was added dropwise to a solution of the product from Step 1 (5.28 g, 12.6 mmol) in THF (50 mL) at a rate sufficient to maintain internal temperature below 30° C. Following completion of the reaction as judged by TLC (5 min), the reaction vessel was cooled to 0° C. and the reaction was quenched by the addition of ice water. The reaction vessel contents were partitioned between EtOAc and water and the layers were separated. The aqueous phase was extracted with additional EtOAc (2×) and the combined organics were washed with brine, dried over MgSO₄/Na₂SO₄, filtered and concentrated under vacuum to afford the product as a 2.2:1 mixture of diastereomers. These were separated by repeated flash chromatography on silica gel eluting with an increasing proportion of EtOAc (0-30%) in toluene. The desired diastereomer corresponded to the more polar compound and was the minor isomer produced in the reaction.

Step 3: A solution of NH₃ in MeOH (7.0 M, 15 mL, 100 mmol, 110 eq.) was added to the product from Step 2 (0.38 g, 0.90 mmol) at 0° C. under N₂ in a thick walled glass tube and the entrance was sealed with a Teflon screw cap followed by heating of the contents at 45° C. for 48 h. The mixture was then concentrated under reduced pressure and the residue was azeotroped with ether/heptane (2×) prior to trituration with Et₂O for 1 h. The solid product was collected by suction filtration and dried under high vacuum.

Step 4: A solution of HCl in 1,4-dioxane (4 M, 0.70 mL, 2.8 mmol, 28 eq.) was added dropwise to a suspension of the product from Step 3 (30 mg, 0.10 mmol) in CH₂Cl₂ (0.7 mL) at 0° C. followed by stirring at rt until the reaction was determined to be complete by LCMS (2 h). The reaction mixture was then concentrated to dryness under reduced pressure to afford the title compound. The material was utilized immediately in subsequent reactions without additional purification. The above sequence can also be utilized to prepare (4R,5S)- and (4R,5R)-4-amino-6,6,6-trifluoro-5-hydroxyhexanamide hydrochloride from (R)-3-tert-butoxycarbonyl-4-(2-benzyloxycarbonylethyl)oxazolidin-5-one.

### Intermediate 10

### 4-Amino-6-fluoro-5-hydroxyhexanamide hydrochloride

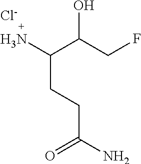 [SMILES: [Cl-] ; NC(=O)CCC(N)C(O)CF]

### Reaction Sequence:

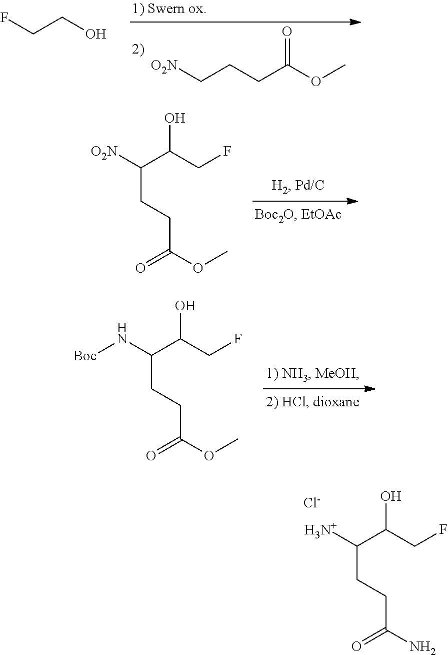

Step 1: A solution of DMSO (8.7 mL, 120 mmol, 3 eq.) in CH₂Cl₂ (15 mL) was added slowly to a solution of oxalyl chloride (4.2 mL, 48 mmol, 1.2 eq.) in CH₂Cl₂ (50 mL) at −78° C. followed by stirring at this temperature for 30 min. A solution of 2-fluoroethanol (2.4 mL, 41 mmol) in CH₂Cl₂ (20 mL) was then introduced dropwise and the mixture was warmed slowly to −40° C. over 1 h followed by recooling to −78° C. prior to the dropwise addition of Et₃N (28.5 mL, 204 mmol, 5 eq.). The formation of insoluble material necessitated shaking to the reaction vessel by hand to ensure homogeneity was maintained over the course of the addition. The suspension was then warmed to rt and stirred for an additional 1.5 h, after which methyl 4-nitrobutyrate (4 mL, 31.2 mmol, 0.77 eq.) was added to the reaction vessel while cooling in a water bath at rt. The mixture was stored at −15° C. overnight and then stirred at rt for 2.5 h prior to partitioning of the mixture between EtOAc (100 mL) and an aqueous solution prepared from 10% citric acid and brine. The aqueous phase was extracted with EtOAc (100 mL) and the combined organic layers were washed with brine, dried over MgSO₄, filtered and concentrated by rotary evaporation under reduced pressure at a bath temperature of 20° C. The residue was subjected to flash chromatography on silica gel eluting with an increasing proportion of EtOAc (0-50%) in hexanes to afford the product as a mixture of diastereomers.

Step 2: The product from Step 1 (4.01 g, 20.0 mmol), di-tert-butyl dicarbonate (5.23 g, 24.0 mmol, 1.25 eq.) and 10% Pd/C (1.00 g) were placed under vacuum in a septum capped rbf and degassed EtOAc (50 mL) was introduced via syringe. The mixture was then placed under an atmosphere of H₂ via a latex balloon and the reaction vessel contents were sonicated for 2 min followed by stirring of the contents at rt for 24 h. Sonication of the reaction vessel contents was then repeated for another 2 min period and the mixture was stirred for an additional 24 h at rt. Following completion of the reaction as judged by TLC, the vessel was flushed with N₂ and CH₂Cl₂ was added. The suspension was filtered through a Celite pad and the pad was washed thoroughly with a mixture of EtOAc and CH₂Cl₂. The filtrate was concentrated in vacuo and the residue was purified by flash chromatography on silica gel eluting with an increasing proportion of EtOAc (10-50%) in hexanes. The major diastereomer eluted first (less polar) and the minor diastereomer eluted second (more polar). Both diastereomers were purified further by trituration with Et₂O/hexanes to afford the products in >10:1 diastereomeric purity as determined by ¹H NMR.

Step 3: A solution of NH₃ in MeOH (7.0 M, 6.0 mL, 42 mmol, 30 eq.) was added to the products isolated in Step 2 (400 mg, 1.43 mmol) at 0° C. under N₂ in a thick walled glass tube and the entrance was sealed with a Teflon screw cap followed by heating of the contents at 40° C. for 3 d. The mixture was then concentrated to dryness under reduced pressure and the residue was triturated with Et₂O. The solid products were collected by suction filtration and dried under vacuum.

Step 4: A solution of HCl in 1,4-dioxane (4.0 M, 1.0 mL, 4.0 mmol, 14 eq.) was added dropwise to a suspension of the products from Step 3 (75 mg, 0.28 mmol) in CH₂Cl₂ (1 mL) at 0° C. followed by stirring at rt until the reaction was determined to be complete by LCMS (2 h). The reaction mixture was then concentrated to dryness under reduced pressure to afford the title compound which was used immediately in subsequent reactions without further purification.

### Intermediate 11

### 4-Amino-5-hydroxyhexanamide

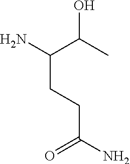

### Reaction Sequence:

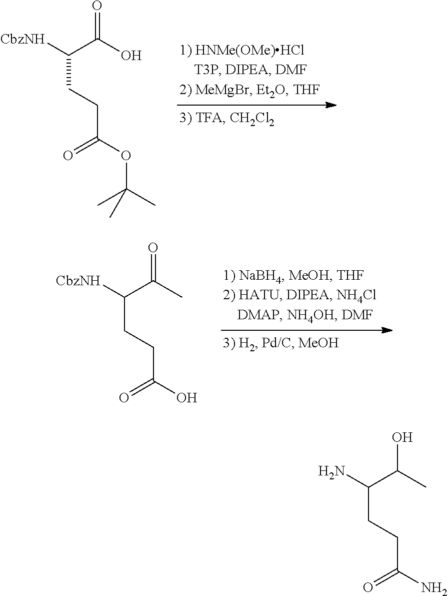

Step 1: T3P (50 wt. % in EtOAc, 21 mL, 35 mmol, 1.2 eq.) and DIPEA (12.5 mL, 71.8 mmol, 2.4 eq.) were added in succession to a mixture of N-Cbz-L-glutamic acid (10.0 g, 29.6 mmol) and N,O-dimethylhydroxylamine hydrochloride (3.46 g, 35.5 mmol, 1.2 eq.) in DMF (15 mL) at 0° C. followed by stirring at rt for 1 h. The reaction vessel contents were then poured into a cold aqueous 0.5 M HCl and extracted with EtOAc (2×). The combined organics were washed with a cold 0.5 M HCl and then with brine. The combined organics were dried over MgSO₄ and filtered through a pad of silica gel. The pad was washed with additional EtOAc and the filtrate was concentrated to dryness. The residue was used directly in the next step.

Step 2: A solution of 3.0 M MeMgBr in Et₂O (34 mL, 100 mmol, 3.0 eq.) was added dropwise to a solution of the product from Step 1 (12.9 g, 34.0 mmol) in THF (100 mL) at −78° C. Following completion of the addition, the mixture was warmed to rt and stirred at this temperature until the reaction was determined to be complete (2 h) by LCMS. The reaction vessel contents were then poured into a cold aqueous 0.5 M HCl and extracted with EtOAc (2×). The combined organics were washed with cold aqueous 0.5 M HCl and brine, and then were dried over MgSO₄, filtered and concentrated under vacuum to dryness. The ketone product was isolated by flash chromatography on silica gel eluting with an increasing proportion of EtOAc (25-50%) in hexanes.

Step 3: TFA (10 mL, 130 mmol, 5.4 eq.) was added dropwise to a stirred solution of the product from Step 2 (8.08 g, 24.1 mmol) in CH₂Cl₂ (2 mL) at −30° C. The reaction vessel contents were then warmed slowly to rt and stirred at this temperature for 3 h before being concentrated to dryness. The residue containing the product was azeotroped with toluene (3×), dried under high vacuum and used directly in the next step.

Step 4: NaBH₄ (170 mg, 4.44 mmol, 2.5 eq.) and MeOH (1 mL) were added in succession to a solution of the product from Step 3 (500 mg, 1.79 mmol) in THF (4 mL) at −78° C. followed by warming to 0° C. with stirring at this temperature for 45 min. The reaction vessel contents were then partitioned between 10% (w/w) citric acid aqueous solution and EtOAc, and the layers were separated. The aqueous phase was extracted with additional EtOAc and the combined organics were washed with brine, dried over Na₂SO₄, filtered, treated with DIPEA (0.62 mL) and concentrated by rotary evaporation under reduced pressure at a bath temperature of 20° C. The residue was dissolved in DMF (5 mL) at 0° C. and treated with DMAP (22 mg, 0.18 mmol, 0.1 eq.), HATU (817 mg, 2.15 mmol, 1.2 eq.), DIPEA (1.87 mL, 10.7 mmol, 6 eq.) and NH₄Cl (481 mg, 9.0 mmol, 5 eq.) followed by warming to rt and stirring overnight. Concentrated aqueous NH₄OH (1.8 mL, 25 mmol, 14 eq.) and DMF (5 mL) were then added with continued stirring at rt for 3 d. The reaction vessel contents were then poured into brine (50 mL) and extracted with EtOAc (50 mL×2). The combined organic layers were dried over MgSO₄, filtered and concentrated to dryness, and the residue was triturated with a mixture of Et₂O/EtOAc. The solid product was collected by suction filtration and dried under high vacuum.

Step 5: A mixture of the product from Step 4 (0.27 g, 0.95 mmol), 10% Pd/C (100 mg) was placed in a rubber septum sealed flask under high vacuum. Degassed MeOH (5 mL) was then added via syringe and the mixture was placed under a N₂ atmosphere supplied by a latex balloon. The reaction vessel was immersed into an ultrasonic bath and sonicated for 2 min. The N₂ atmosphere was purged by delivery of H₂ by latex balloon and the mixture was stirred at rt. After 5 h the reaction was judged to be complete by TLC analysis. The H₂ atmosphere was replaced with N₂, CH₂Cl₂ was added and the suspension was filtered through a Celite pad which was subsequently washed with a mixture of MeOH/CH₂Cl₂. Concentration of the filtrate in vacuo and trituration of the residue with MeOH in EtOAc afforded the title compound.

### Preparation of Intermediates 12-14

### Reaction Sequence:

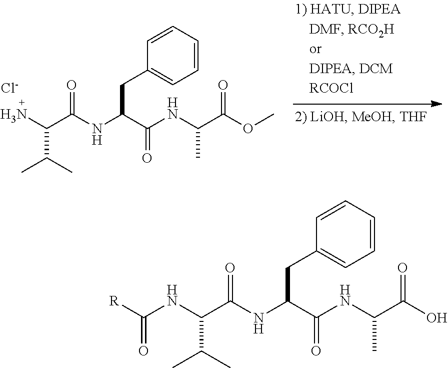

### Intermediate 12

### (S)-2-((S)-2-((S)-2-Decanamido-3-methylbutanamido)-3-phenylpropanamido)propanoic acid

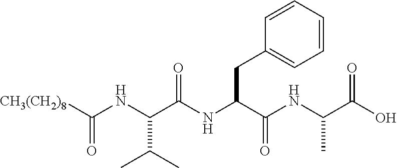

Step 1: Decanoyl chloride (0.73 mL, 3.6 mmol, 1.1 eq.) and Et₃N (1.6 mL, 12 mmol, 3.5 eq.) were added dropwise in succession to a stirred suspension of Intermediate 4 (1.25 g, 3.24 mmol) in THF (15 mL) at 0° C. followed by slow warming to rt. After stirring overnight, additional THF (70-80 mL) was added to facilitate stirring of the mixture and a second portion of decanoyl chloride (0.10 mL, 0.50 mmol, 0.15 eq.) was added with continued stirring at rt until the reaction was judged to be complete (24 h) by LCMS. The reaction vessel contents were then poured into a cold mixture of aqueous 0.5 M HCl, EtOAc and hexanes. The solid product was collected by gravity filtration, washed with water and 1:1 Et₂O/hexanes before being dried under high vacuum.

Step 2: An aqueous solution of 1 M LiOH (9 mL, 9 mmol, 3 eq.) was added to a stirred suspension of the product from Step 1 (1.47 g, 2.92 mmol) in MeOH (9 mL) and THF (18 mL) at 0° C. followed by warming to rt. After 4 h, additional MeOH (18 mL) was added and the suspension was stirred overnight at rt. LCMS analysis indicated that the reaction had not reached completion after 24 h. The mixture was recooled to 0° C. and an additional portion of 1 M LiOH (3 mL, 3 mmol, 1 eq.) was added followed by stirring at rt for a second 24 h period. At this point, the reaction still had not yet reached completion due to the low solubility of the starting ester in the reaction media. The addition of a third portion of MeOH and 1 M LiOH (3 mL, 3 mmol, 1 eq.) was necessary to push the reaction to completion. The mixture was subsequently filtered to remove solids and the filtrate was acidified to pH 1 with 1 M HCl followed by concentration to dryness. The solid residue was suspended in water, collected by suction filtration and washed with water and Et₂O. Drying of the solid under suction and high vacuum provided the title compound.

### Intermediate 13

### (S)-2-((S)-2-((S)-3-Methyl-2-(2-(4-(trifluoromethoxy)phenyl)acetamido)butanamido)-3-phenylpropanamido)propanoic acid

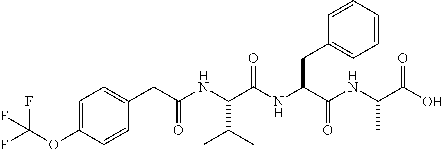 [SMILES: CC(C)[C@H](NC(=O)Cc1ccc(OC(F)(F)F)cc1)C(=O)N[C@@H](Cc1ccccc1)C(=O)N[C@@H](C)C(=O)O]

Step 1: DIPEA (0.51 mL, 2.9 mmol, 3.5 eq.) was added dropwise over 10 min to a solution of Intermediate 4 (0.32 g, 0.83 mmol), 4-(trifluoromethoxy)phenylacetic acid (0.20 g, 0.91 mmol, 1.1 eq.) and HATU (0.38 g, 1.0 mmol, 1.2 eq.) in DMF (3 mL) at 0° C. followed by slow warming to rt with stirring overnight. The reaction vessel contents were then diluted with Et₂O and poured into a mixture of ice and saturated aqueous NaHCO₃. This mixture was stirred for 30 min until the ice had melted and the solid product was collected by suction filtration, washed with water and Et₂O and dried under high vacuum.

Step 2: An aqueous solution of 1 M LiOH (3 mL, 3 mmol, 4 eq.) was added to a rapidly stirred suspension of the product from Step 1 (0.40 g, 0.73 mmol) in MeOH (3 mL) and THF (6 mL) at 0° C. After 15 min at this temperature, the reaction vessel was warmed to rt and the progress of the reaction was monitored by LC-MS. After 2 h, the mixture was cooled to 0° C. and acidified to pH 1 by the addition of an aqueous solution of 0.05 M HCl (100 mL) with stirring for an additional 1 h at rt. The solid product was collected by suction filtration, washed with water and dried under suction and high vacuum to afford the title compound.

### Intermediate 14

### (S)-2-((S)-2-((S)-2-([1,1′-Biphenyl]-4-ylcarboxamido)-3-methylbutanamido)-3-phenylpropanamido)propanoic acid

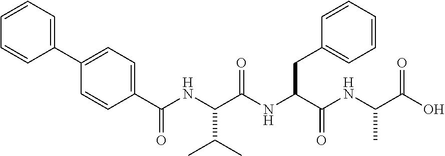 [SMILES: CC(C)[C@H](NC(=O)c1ccc(-c2ccccc2)cc1)C(=O)N[C@@H](Cc1ccccc1)C(=O)N[C@@H](C)C(=O)O]

Step 1: DIPEA (0.42 mL, 2.4 mmol, 3.5 eq.) was added dropwise over 10 min to a solution of Intermediate 4 (0.32 g, 0.83 mmol, 1.2 eq.), biphenyl-4-carboxylic acid (145 mg, 0.732 mmol) and HATU (316 mg, 0.832 mmol, 1.2 eq.) in DMF (3 mL) at 0° C. followed by stirring at rt for 3 d. The mixture was then diluted with EtOAc and poured into a mixture of ice and a saturated aqueous solution of NaHCO₃ with stirring at rt until the ice melted (ca. 30 min). The solid product was collected by suction filtration, washed successively with water and EtOAc before being dried under suction and high vacuum.

Step 2: An aqueous solution of 1 M LiOH (3 mL, 3 mmol, 5 eq.) was added to a suspension of the product from Step 1 (0.33 g, 0.62 mmol) in MeOH (3 mL) and THF (6 mL) at 0° C. followed by rapid stirring at rt until the reaction was judged to be complete (3 h) by LCMS. The reaction vessel contents were then recooled to 0° C. and acidified by the addition of an aqueous solution of 0.05 M HCl (100 mL) followed by stirring at rt for 1 h. The solid product was collected by suction filtration, washed with water and Et₂O, and dried under suction and high vacuum to afford the title compound.

### General Sequence 1:

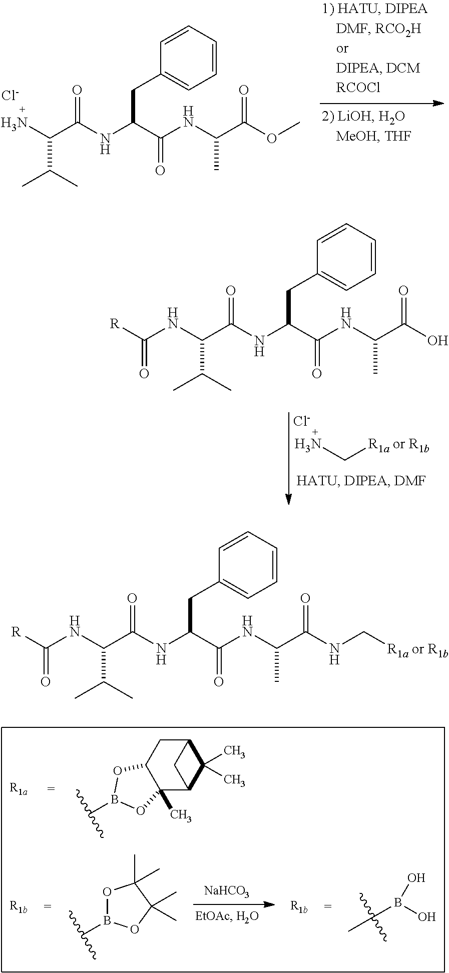

Step 1 (for RCO₂H): DIPEA (3.5 eq) was added to a stirred suspension of Intermediate 4 (0.83 mmol), HATU (1.2 eq.) and the appropriate acid (1.1 eq.) in DMF (3.0 mL) at 0° C. with slow warming to rt and stirring overnight. Saturated NaHCO₃ aqueous solution and EtOAc were then added and the solid product was collected by suction filtration, washed with water and EtOAc and under high vacuum.

Step 1 (for RC(O)Cl): The appropriate acid chloride (1.1 eq.) was added portionwise to a stirred suspension of Intermediate 4 (1.3 mmol) and DIPEA (2.2 eq.) in CH₂Cl₂ (13 mL) at 0° C. followed by slow warming to rt and stirring overnight. Additional CH₂Cl₂ and water were added to the reaction vessel and the solid product was isolated by suction filtration, washed with CH₂Cl₂ and water and dried under high vacuum.

Step 2: 1 M Aqueous LiOH (4 eq.) was added dropwise to a stirred suspension of the product from Step 1 (0.73 mmol) in MeOH (3 mL) and THF (6 mL) at 000° C. After 15 min, the reaction was warmed to rt and stirred for an additional 3 h. Crushed ice was then added and the mixture was acidified to pH 3 with 1 M HCl. After stirring for an additional 1 h, the solid product was isolated by suction filtration and dried under high vacuum. Analogs that failed to precipitate under these conditions were isolated by an extractive workup with EtOAc (3×). The combined organics were washed with brine, dried (Na₂SO₄), filtered and concentrated and the residue was used directly in the next step.

Step 3: DIPEA (2.5 eq.) was added to a stirred suspension of the product from Step 2 (0.14 mmol), HATU (1.1 eq.) and aminomethylboronic acid pinacolester hydrochloride (1.1 eq.) or boro-Gly-(+)-pinanediol hydrochloride (1.1 eq.) in DMF (1 mL) at 0° C. followed by slow warming to rt with stirring overnight. Half-saturated NaHCO₃ aqueous solution and EtOAc were then added and the mixture was agitated in an ultrasonic bath for 1 h. The solid product was isolated by suction filtration and dried under high vacuum.

### General Sequence 2:

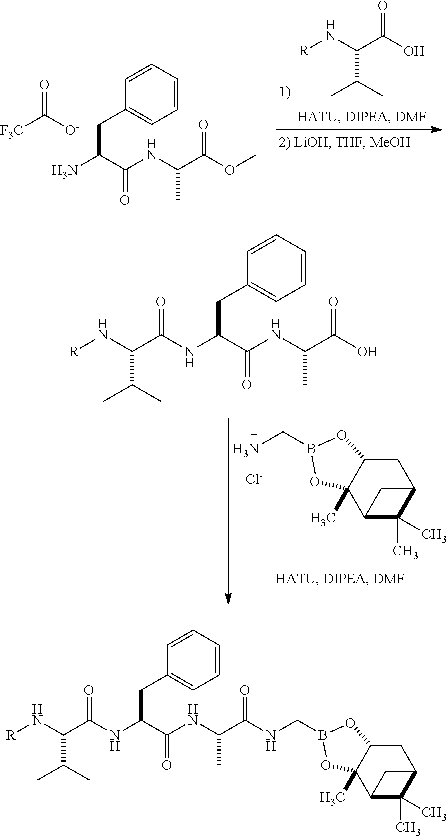

Step 1: DIPEA (38 mmol, 3.5 eq.) was added dropwise over 10 min to a solution of Intermediate 1 (11 mmol), an L-valine derivative (12 mmol, 1.1 eq.) and HATU (13 mmol, 1.2 eq.) in DMF (35 mL) at 0° C. followed by slow warming to rt with stirring overnight. The mixture was then poured into a mixture of crushed ice (100 mL), saturated aqueous NaHCO₃ (100 mL) and Et₂O (200 mL) and stirred for 30 min until the ice melted. The solid product was collected by suction filtration, washed with water and 1:1 Et₂O/hexanes, and dried under high vacuum.

Step 2: The product from Step 1 (3.33 mmol) was suspended in a mixture of THF (20 mL) and MeOH (10 mL) and treated with 1 M LiOH (4.3 mL, 4.3 mmol, 1.3 eq.) at 0° C. followed by slow warming to rt with stirring overnight. The mixture was then recooled to 000° C., acidified to pH 4 with 1 M HCl, and extracted with EtOAc (3×). The combined organics were dried (MgSO₄), filtered and concentrated and the residue was used directly in the next step.

Step 3: DIPEA (2.0 mmol, 2.5 eq.) was added dropwise to a stirred suspension of the product from Step 2 (0.90 mmol), boro-Gly-(+)-pinanediol hydrochloride (1.1 mmol, 1.2 eq.) and HATU (1.03 mmol, 1.1 eq.) in DMF (4.5 mL) at 0° C. followed by slow warming to rt. After stirring overnight, the mixture was diluted with half saturated NaHCO₃ aqueous solution and extracted with EtOAc (3×). The combined organics were washed with half saturated brine (2×), dried (MgSO₄), filtered and concentrated under reduced pressure. The residue was stirred with Et₂O and the solid material was collected by filtration and dried under high vacuum to afford the final product.

### General Sequence 3

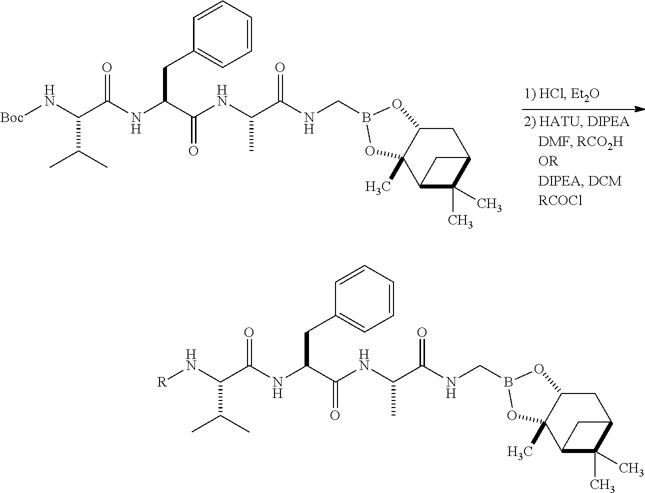

Step 1: The final product from General Sequence 2 with R=Boc (0.51 mmol) was treated at 0° C. with a solution of 2 M HCl in Et₂O (20 mmol, 40 eq.) with stirring for 18 h at this temperature followed by concentration under reduced pressure. The residue was used directly in the next step.

Step 2 (for RCO₂H): DIPEA (0.27 mmol, 3.0 eq.) was added dropwise to a stirred suspension of the product from Step 1 (0.089 mmol), HATU (0.11 mmol, 1.2 eq.) and the appropriate carboxylic acid (1.1 eq.) in DMF (0.89 mL) at 0° C. followed by slow warming to rt. After stirring overnight, EtOAc and half saturated NaHCO₃ aqueous solution were added and the solid product was collected by suction filtration, washed with water and dried under high vacuum.

Step 2 (for RCOCl): DIPEA (0.13 mmol, 3.0 eq.) was added to a stirred suspension of the product from Step 1 (0.042 mmol) in CH₂Cl₂ (0.4 mL) at 0° C. followed by the portion-wise introduction of the appropriate acid chloride (0.064 mmol, 1.5 eq.) The reaction was warmed slowly to rt and stirred overnight. Thereafter, the reaction was diluted with EtOAc and half saturated NaHCO₃ and the layers were separated. The aqueous layer was extracted with EtOAc (2×) and the combined organic layers were washed with half saturated brine (2×), dried (MgSO₄) and concentrated under reduced pressure. The residue was triturated with Et₂O and the solid product was isolated by filtration and dried under high vacuum.

### General Sequence 4

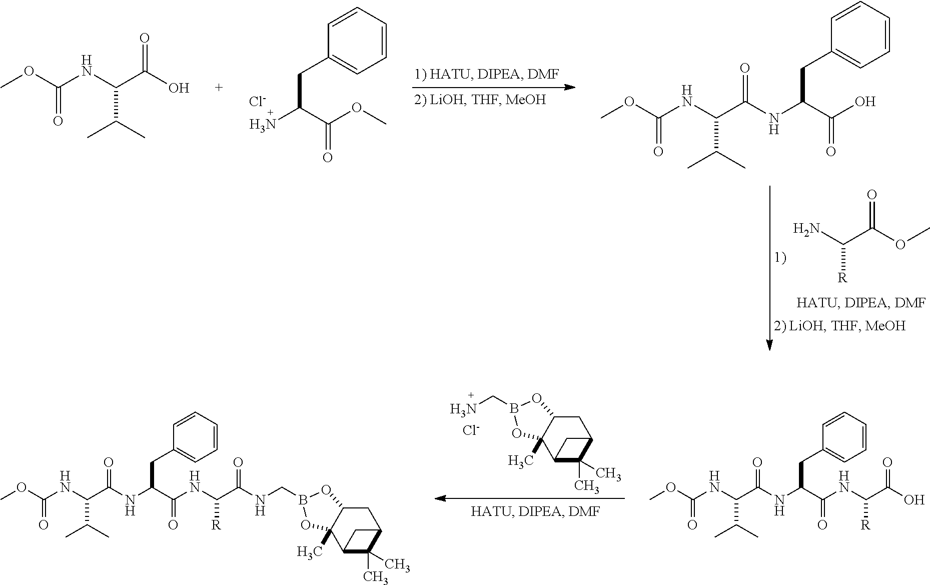

Step 1: HATU (11.9 g, 31.4 mmol, 1.1 eq.) and DIPEA (14.9 mL, 85.5 mmol, 3.0 eq.) were added to a solution of (S)-2-((methoxycarbonyl)amino)-3-methylbutanoic acid (5.00 g, 28.5 mmol) and L-phenylalanine methyl ester hydrochloride (6.15 g, 28.5 mmol, 1.0 eq.) in DMF (100 mL) at 0° C. followed by slow warming to rt with stirring overnight. The mixture was subsequently diluted with EtOAc and washed with aqueous solutions of 10% HCl, saturated NaHCO₃ and brine. The organic layer was separated, dried over MgSO₄, filtered and concentrated under reduced pressure and the residue was used directly in next step.

Step 2: Aqueous 1 M LiOH (60 mL) was added to a solution of the product from Step 1 (9.58 g, 28.5 mmol) in THF (120 mL) and MeOH (60 mL) at 000° C. After stirring at rt for 3 h, the mixture was acidified to pH 2-3 with 1 M HCl and extracted with EtOAc. The organic layer was separated, washed with brine, dried over MgSO₄, filtered and concentrated under reduced pressure. The residue was triturated with a mixture EtOAc and hexanes and the solid product was collected by suction filtration and dried under high vacuum.

Step 3: HATU (1.12 mmol, 1.2 eq.) and DIPEA (3.7 mmol, 4.0 eq.) were added sequentially to a stirred mixture of the product from Step 2 (0.93 mmol) and the appropriate protected amino acid (0.93 mmol) in DMF (5 mL) at 0° C. followed by slow warming to rt and stirring at this temperature for 3 h. The mixture was then partitioned between EtOAc and 10% HCl aqueous solution. The organic phase was separated and washed with saturated NaHCO₃ and brine, dried over MgSO₄, filtrated and concentrated under reduced pressure. The residue was purified by flash chromatography on silica gel eluting with an increasing proportion of MeOH in CH₂Cl₂.

Step 4: A stirred suspension of the product from Step 3 (0.47 mmol) in THF (2 mL) and MeOH (1 mL) at 0° C. was treated with aqueous 1 M LiOH (0.95 mmol, 2.0 eq.), warmed slowly to rt and stirred for 3 h. The mixture was then acidified with 1 M HCl to pH 2-3 and extracted with EtOAc. The organic layer was separated, washed with brine, dried over MgSO₄, filtered and concentrated under reduced pressure. The residue was triturated with a mixture of EtOAc and Et₂O and the solid product was isolated by suction filtration and dried under high vacuum.

Step 5: A stirred suspension of the product from Step 4 (0.11 mmol) and boro-Gly-(+)-pinanediol hydrochloride (0.12 mmol, 1.1 eq.) in DMF (1 mL) at 0° C. was treated with HATU (0.13 mmol, 1.2 eq.) and DIPEA (0.44 mmol, 4.0 eq.) followed by slow warming to rt and stirring overnight. The reaction vessel contents were subsequently partitioned between EtOAc and half saturated NaHCO₃ aqueous solution. The EtOAc layer was separated and the aqueous layer extracted with additional EtOAc (2×). The combined organic layers were dried over MgSO₄, filtered and concentrated under reduced pressure. The residue was triturated with water and the solid product was isolated by suction filtration and dried under high vacuum.

### General Sequence 5

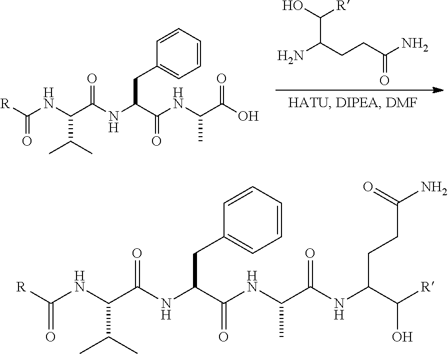

DIPEA (3.5 eq.) was added dropwise to a stirred suspension of the appropriate carboxylic acid (Intermediate 12,13 or 14), the appropriate amine (Intermediate 8, 9, 10 or 11, 1.2-1.4 eq.) and HATU (1.2-1.3 eq.) in DMF (0.1 M) at 0° C. followed by slow warming to rt and stirring overnight. The reaction mixture was then diluted with EtOAc and stirred with an ice-cold, half-saturated NaHCO₃ aqueous solution. The solid amide product was isolated by filtration, washed with EtOAc and water, followed by drying under suction and high vacuum. When the product was soluble in EtOAc, the layers were separated and the aqueous phase was extracted with additional EtOAc. The combined organics were washed with an aqueous solution of 5% LiCl and saturated brine, and then were dried over MgSO₄, filtered and concentrated under reduced pressure. The residue was triturated with Et₂O and the solid product was collected by filtration and dried under high vacuum.

Compounds described in Examples 1 to 24 are identified by the example numbers. Hence the compound disclosed in Example 1 is designated compound 1.

### Example 1 (1)

### {[(2S)-2-[(2S-[(2S)-2-[(Methoxycarbonyl)amino]-3-methylbutanamido]-3-phenylpropanamido]propanamido]methyl}boronic acid

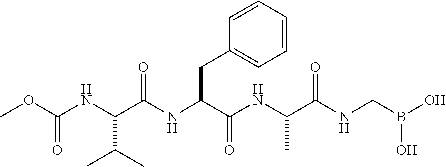 [SMILES: COC(=O)N[C@H](C(=O)N[C@@H](Cc1ccccc1)C(=O)N[C@@H](C)C(=O)NCB(O)O)C(C)C]

2,4,6-Collidine (0.14 mL, 1.1 mmol, 2.1 eq.) was added dropwise to a suspension of Intermediate 2 (0.20 g, 0.51 mmol), HATU (0.194 g, 0.510 mmol) and aminomethylboronic acid pinacol ester hydrochloride (0.099 g, 0.51 mmol) in DMF (2.9 mL) at 0° C. with slow warming to rt and stirring overnight. The vessel contents were subsequently partitioned between half-saturated NaHCO₃ aqueous solution and EtOAc. The organic layer was separated and the aqueous phase was extracted with EtOAc (2×). The combined organics were dried (MgSO₄), filtered and concentrated under reduced pressure. The residue was triturated with mixture of EtOAc/Et₂O/water with agitation in an ultrasonic bath for 1 h. The solid material was collected by suction filtration and dried under high vacuum to afford the title compound.

¹H NMR (methanol-d4): δ 7.31-7.18 (5H), 4.63 (1H), 4.50 (1H), 3.80 (1H, d), 3.64 (3H), 3.20 (1H), 2.94 (1H), 2.34 (2H), 1.92 (1H), 1.42 (3H), 0.83-0.75 (6H). MS ESI: 473.3 [M+Na]⁺

Examples 2-5 were prepared using General Sequence 1:

### Example 2 (2)

### {[(2S)-2-[(2S)-2-[(2S)-3-Methyl-2-[(4-phenylphenyl)formamido]butanamido]-3-phenylpropanamido]propanamido]methyl}boronic acid

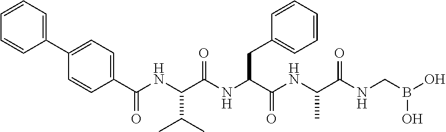 [SMILES: CC(C)[C@H](NC(=O)c1ccc(-c2ccccc2)cc1)C(=O)N[C@@H](Cc1ccccc1)C(=O)N[C@@H](C)C(=O)NCB(O)O]

¹H NMR (methanol-d4): δ 7.89 (2H), 7.73 (2H), 7.68 (2H), 7.47 (2H), 7.38 (1H), 7.28-7.22 (2H), 7.21-7.13 (2H), 7.09 (1H), 4.69 (1H), 4.39 (1H), 4.32 (1H), 3.23 (1H), 2.93 (1H), 2.35-2.22 (2H), 2.13 (1H), 1.41-1.31 (3H), 1.00-0.88 (6H). MS ESI: 595.4 [M+Na]⁺

### Example 3 (3)

### {[(2S)-2-[(2S)-2-[(2S)-3-Methyl-2-{2-[4-(trifluoromethoxy)phenyl]acetamido}butanamido]-3-phenylpropanamido]propanamido]methyl}boronic acid

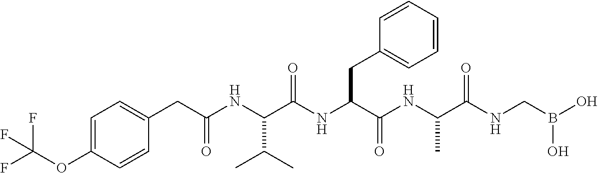 [SMILES: CC(C)[C@H](NC(=O)Cc1ccc(OC(F)(F)F)cc1)C(=O)N[C@@H](Cc1ccccc1)C(=O)N[C@@H](C)C(=O)NCB(O)O]

¹H NMR (DMSO-d₆): δ 8.26 (1H), 8.16 (1H), 8.20-8.12 (2H) 7.38-7.32 (2H), 7.30-7.12 (7H), 4.53 (1H), 4.30 (1H), 4.10 (1H), 3.55 (1H), 3.45 (1H), 3.04 (1H), 2.77 (1H), 2.27 (1H), 1.89 (1H), 1.22-1.18 (3H), 0.74-0.72 (6H). MS ESI: 617.3 [M+Na]⁺

### Example 4 (4)

### {[(2S)-2-[(2S)-2-[(2S)-2-Decanamido-3-methylbutanamido]-3-phenylpropanamido]propanamido]methyl}boronicacid

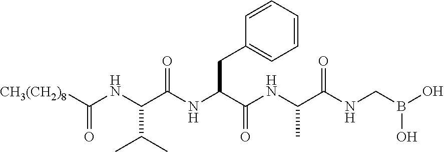

¹H NMR (methanol-d4): δ 7.29-7.16 (5H), 4.59 (1H), 4.48 (1H), 4.04 (1H), 3.15 (1H), 2.94 (1H), 2.33 (2H), 2.26-2.14 (2H), 1.95 (1H), 1.56 (2H), 1.40 (3H), 1.35-1.20 (12H), 0.91-0.81 (9H). MS ESI: 569.4 [M+Na]⁺

### Example 5 (5)

### (2S)-3-Methyl-N-[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoyl}ethyl]-2-(phenylformamido)butanamide

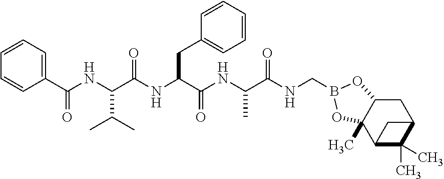

¹H NMR (DMSO-d₆): δ 8.22-8.16 (2H), 8.11-8.03 (2H), 7.84 (2H), 7.55 (1H), 7.50-7.45 (2H), 7.26-7.09 (5H), 4.57 (1H), 4.29 (1H), 4.20 (2H), 3.05 (1H), 2.77 (1H), 2.37 (2H), 2.22 (1H), 2.10-1.99 (2H), 1.86 (1H), 1.80 (1H), 1.67 (1H), 1.28-1.17 (10H), 0.83 (3H), 0.79 (3H), 0.75 (3H). MS ESI: 653.5 [M+Na]⁺

Examples 6 and 7 were prepared using General Sequence 2:

### Example 6 (6)

### tert-Butyl N-[(1S)-2-methyl-1-{[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoyl}ethyl]carbamoyl}propyl]carbamate

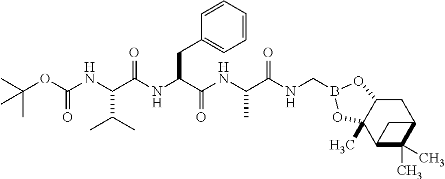

¹H NMR (DMSO-d₆): δ 8.26 (1H), 8.08 (1H), 7.87 (1H), 7.19-7.16 (5H), 6.64 (1H), 4.58 (1H), 4.29 (1H), 4.19 (1H), 3.68 (1H), 3.02 (1H), 2.74 (1H), 2.38 (2H), 2.22 (1H), 2.06 (1H), 1.87 (1H), 1.84-1.73 (2H), 1.68 (1H), 1.36 (9H), 1.31-1.14 (10H), 0.79 (3H), 0.73-0.61 (6H). MS ESI: 627.5 [M+H]⁺

### Example 7 (7)

### benzyl N-[(1S)-2-methyl-1-{[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoyl}ethyl]carbamoyl}propyl]carbamate

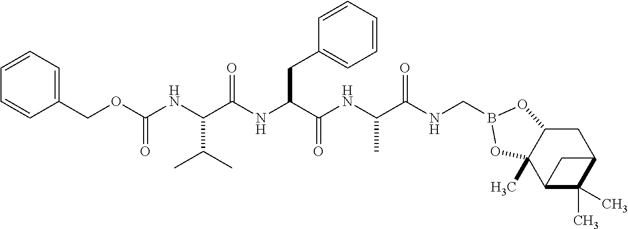

¹H NMR (DMSO-d6): δ 8.21 (1H), 8.09 (1H), 7.98 (1H), 7.40-7.29 (5H), 7.27-7.12 (6H), 5.02 (2H), 4.56 (1H), 4.29 (1H), 4.19 (1H), 3.80 (1H), 3.04 (1H), 2.76 (1H), 2.38 (2H), 2.22 (1H), 2.06 (1H), 1.89-1.76 (3H), 1.68 (1H), 1.30-1.15 (10H), 0.79 (3H), 0.72 (6H). MS ESI: 683.4 [M+Na]⁺

Examples 8a, 8b, 8c, 8d and 9 were prepared using General Sequence 3:

### Example 8a (8a)

(2S)-3-methyl-N-[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoyl}ethyl]-2-({4-[3-(trifluoromethyl)-3H-diazirin-3-yl]phenyl}formamido)butanamide

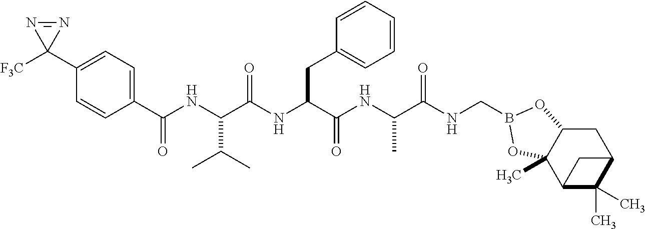

¹H NMR (DMSO-d₆): δ 8.36 (1H), 8.16 (1H), 8.10-8.04 (2H), 7.93 (2H), 7.38 (2H), 7.24-7.05 (5H), 4.55 (1H), 4.32-4.14 (3H), 3.03 (1H), 2.76 (1H), 2.36 (2H), 2.20 (1H), 2.08-1.96 (2H), 1.86 (1H), 1.78 (1H), 1.66 (1H), 1.29-1.13 (10H), 0.84-0.72 (9H). MS ESI: 739.4 [M+H]⁺

### Example 8b (8b)

(2S)-3-methyl-N-[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoyl}ethyl]-2-({3-[3-(trifluoromethyl)-3H-diazirin-3-yl]phenyl}formamido)butanamide

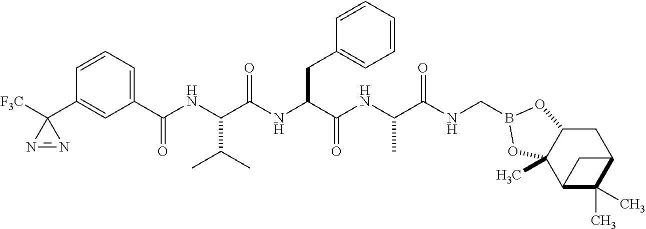

¹H NMR (DMSO-d₆): δ 8.46 (1H), 8.16-8.05 (3H), 8.00 (1H), 7.65-7.60 (2H), 7.55 (1H), 7.24-7.19 (2H), 7.17-7.05 (3H), 4.54 (1H), 4.31-4.15 (3H), 3.04 (1H), 2.76 (1H), 2.39-2.34 (2H), 2.20 (1H), 2.09-1.97 (2H), 1.86 (1H), 1.79 (1H), 1.67 (1H), 1.30-1.11 (10H), 0.86-0.74 (9H). MS ESI: 739.4 [M+H]⁺

### Example 8c (8c)

(2S)-3-methyl-N-[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoyl}ethyl]-2-(2-{4-[3-(trifluoromethyl)-3H-diazirin-3-yl]phenyl}acetamido)butanamide

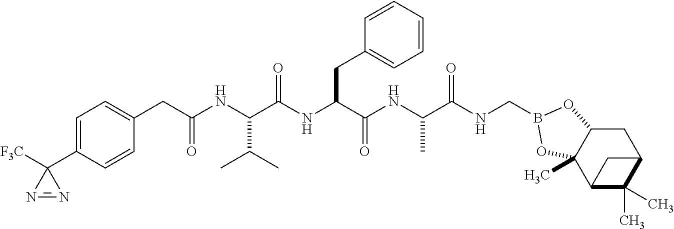

¹H NMR (DMSO-d₆): δ 8.14-8.04 (3H), 7.39-7.33 (2H), 7.24-7.10 (8H), 4.51 (1H), 4.28 (1H), 4.18 (1H) 4.09 (1H), 3.60-3.44 (2H), 3.01 (1H), 2.75 (1H), 2.38-2.34 (2H), 2.21 (1H), 2.05 (1H), 1.92-1.83 (2H), 1.79 (1H), 1.66 (1H), 1.29-1.09 (10H), 0.81-0.65 (9H). MS ESI: 753.5 [M+H]⁺

### Example 8d (8d)

(2S)-3-methyl-N-[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoyl}ethyl]-2-(2-{3-[3-(trifluoromethyl)-3H-diazirin-3-yl]phenyl}acetamido)butanamide

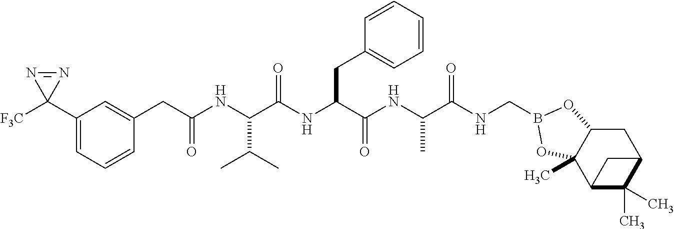

¹H NMR (DMSO-d₆): δ 8.15-8.03 (3H), 7.44-7.36 (2H), 7.23-7.09 (8H), 4.56-4.42 (2H), 4.30-4.07 (2H), 3.63-3.39 (2H), 3.02 (1H), 2.76 (1H), 2.38-2.33 (2H), 2.20 (1H), 2.04 (1H), 1.93-1.82 (2H), 1.78 (1H), 1.66 (1H), 1.28-1.09 (10H), 0.80-0.75 (3H), 0.75-0.67 (6H). MS ESI: 753.4 [M+H]⁺

### Example 9 (9)

### (2S)-2-[(2-fluorophenyl)formamido]-3-methyl-N-[(1S)-2-phenyl-1-{[(1S)-1-({[(1S,2S,6R,8S)-2,9,9-trimethyl-3,5- dioxa-4-boratricyclo[6.1.1.0^{2,6}]decan-4-yl]methyl}carbamoyl)ethyl]carbamoyl}ethyl]butanamide

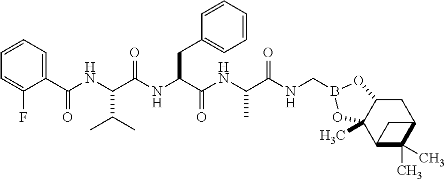

¹H NMR (DMSO-d₆): δ 8.25 (1H), 8.14-8.08 (2H), 8.03 (1H), 7.64-7.51 (2H), 7.33-7.11 (7H), 4.60 (1H), 4.30 (2H), 4.18 (1H), 3.05 (1H), 2.75 (1H), 2.38 (2H), 2.20 (1H), 2.10-1.93 (2H), 1.87 (1H), 1.79 (1H), 1.67 (1H), 1.29-1.18 (10H), 0.82-0.74 (9H). MS ESI: 649.4 [M+H]⁺

Examples 10-14 were prepared using General Sequence 4:

### Example 10 (10)

### Methyl N-[(1S)-2-methyl-1-{[(1S)-2-phenyl-1-{[(1S)-1-({[(1S,2S,6R,8S)-2,9,9-trimethyl-3,5-dioxa-4-boratricyclo[6.1.1.0^{2,6}]decan-4-yl]methyl}carbamoyl)propyl]carbamoyl}ethyl]carbamoyl}propyl]carbamate

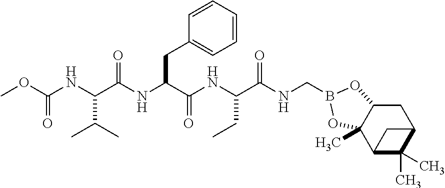

¹H NMR (DMSO-d₆): δ 8.11-8.04 (2H), 7.98 (1H), 7.27-7.21 (4H), 7.17 (1H), 7.05 (1H), 4.58 (1H), 4.22-4.14 (2H), 3.77 (1H), 3.52 (3H), 3.02 (1H), 2.78 (1H), 2.37 (2H), 2.22 (1H), 2.06 (1H), 1.89-1.77 (3H), 1.71-1.61 (2H), 1.52 (1H), 1.31-1.24 (4H), 1.23-1.20 (3H), 0.85-0.77 (6H), 0.74-0.69 (6H). MS ESI: 599.4 [M+H]⁺

### Example 11 (11)

### Methyl N-[(1S)-2-methyl-1-{[(1S)-2-phenyl-1-{[(1S)-2-phenyl-1-({[(1S,2S,6R,8S)-2,9,9-trimethyl-3,5-dioxa-4-boratricyclo[6.1.1.0^{2,6}]decan-4-yl]methyl}carbamoyl)ethyl]carbamoyl}ethyl]carbamoyl}propyl]carbamate

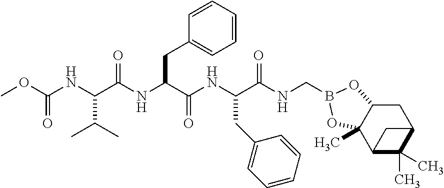

¹H NMR (DMSO-d₆): δ 8.23 (1H), 8.09 (1H), 7.88 (1H), 7.29-7.12 (10H), 7.03 (1H), 4.59-4.45 (2H), 4.24 (1H), 3.76 (1H), 3.53 (3H), 3.00-2.68 (4H), 2.39 (2H), 2.24 (1H), 2.09 (1H), 1.90 (1H), 1.86-1.78 (2H), 1.71 (1H), 1.34-1.26 (4H), 1.25-1.20 (3H), 0.81 (3H), 0.77-0.62 (6H). MS ESI: 661.5 [M+H]⁺

### Example 12 (12)

### Methyl N-[(1S)-2-methyl-1-{[(1S)-1-{[(1S)-3-(methylsulfanyl)-1-({[(1S,2S,6R,8S)-2,9,9-trimethyl-3,5-dioxa-4-boratricyclo[6.1.1.0^{2,6}]decan-4-yl]methyl}carbamoyl)propyl]carbamoyl}-2-phenylethyl]carbamoyl}propyl]carbamate

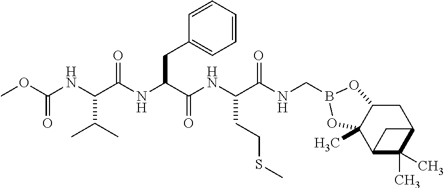

¹H NMR (DMSO-d₆): δ 8.13 (1H), 8.03 (1H), 7.95 (1H), 7.27-7.15 (5H), 7.06 (1H), 4.54 (1H), 4.34 (1H), 4.22 (1H), 3.77 (1H), 3.52 (3H), 3.02 (1H), 2.79 (1H), 2.47-2.32 (4H), 2.22 (1H), 2.06 (1H), 2.01 (3H), 1.93-1.74 (5H), 1.69 (1H), 1.30-1.21 (7H), 0.79 (3H), 0.72 (6H). MS ESI: 645.4 [M+H]⁺

### Example 13 (13)

### Methyl N-[(1S)-2-methyl-1-{[(1S)-1-{[(1S)-3-methyl-1-({[(1S,2S,6R,8S)-2,9,9-trimethyl-3,5-dioxa-4-boratricyclo[6.1.1.0² 6]decan-4-yl]methyl}carbamoyl)butyl]carbamoyl}-2-phenylethyl]carbamoyl}propyl]carbamate

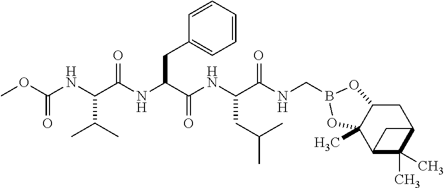

¹H NMR (DMSO-d₆): δ 8.12 (1H), 8.07 (1H, s), 8.00 (1H), 7.26-7.14 (5H), 7.04 (1H), 4.56 (1H), 4.31 (1H), 4.18 (1H), 3.79 (1H), 3.52 (3H), 3.01 (1H), 2.78 (1H), 2.30-2.36 (2H), 2.21 (1H), 2.05 (1H), 1.89-1.76 (3H), 1.68 (1H), 1.56 (1H), 1.48-1.39 (2H), 1.30-1.24 (4H), 1.22 (3H), 0.86 (3H), 0.85 (3H), 0.76 (3H), 0.72 (6H). MS ESI: 627.5 [M+H]⁺

### Example 14 (14)

### Methyl N-[(1S)-2-methyl-1-{[(1S)-2-phenyl-1-{[(S)-phenyl({[(1S,2S,6R,8S)-2,9,9-trimethyl-3,5-dioxa-4-boratricyclo[6.1.1.0^{2,6}]decan-4-yl]methyl}carbamoyl)methyl]carbamoyl}ethyl]carbamoyl}propyl]carbamate

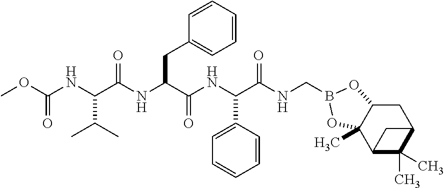

¹H NMR (DMSO-d₆): δ 8.66 (0.5H), 8.53 (0.5H), 8.44 (0.5H), 8.32 (0.5H), 8.05 (0.5H H), 7.97 (0.5H), 7.92 (1H), 7.42-7.00 (10H), 5.47 (1H), 4.68 (1H), 4.27 (1H), 3.77 (1H), 3.52 (3H), 2.95 (1H), 2.76 (1H), 2.58-2.42 (2H overlapped), 2.23 (1H), 2.07 (1H), 1.94-1.77 (3H), 1.65 (1H), 1.32-1.19 (7H), 0.79 (3H), 0.75-0.63 (6H). MS ESI: 647.4 [M+H]⁺

### Example 15 (15)

### Methyl (4R/S)-4-[(2S)-2-[(2S)-2-[(2S)-2-[(Methoxycarbonyl)amino]-3-methylbutanamido]-3-phenylpropanamid]-2-phenylacetamido]-4-[(1S,2S,6R,8S)-2,9,9-trimethyl-3,5-dioxa-4-boratricyclo[6.1.1.0² 6]decan-4-yl]butanoate

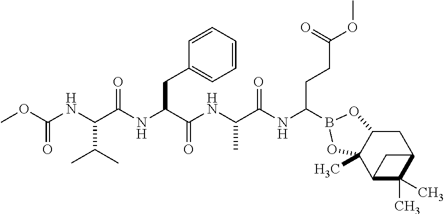

Intermediate 5 (0.027 g, 0.084 mmol), Intermediate 3 (0.062 g, 0.13 mmol, 1.5 eq) and platinum oxide (0.002 g, 0.008 mmol, 0.1 eq.) were placed in an rbf and sealed with a septum. The flask was purged (vacuum, N₂) and degassed THF (2.5 mL) was added. The mixture was then stirred under a H₂ atmosphere (P=1 atm) provided via a latex balloon. After 18 h, EtOAc and a saturated aqueous solution of NaHCO₃ were added. The layers were separated and the aqueous layer extracted with additional EtOAc (2×). The combined organic layers were dried over MgSO₄, filtered and concentrated under reduced pressure. The residue was purified by flash chromatography on silica gel eluting with an increasing proportion of EtOAc in hexanes to afford the title compound as a mixture of diastereomers.

¹H NMR (Acetone-d₆): δ 7.89 (0.5H), 7.77 (0.5H), 7.61-7.51 (2H), 7.31-7.17 (5H), 6.52 (0.5H), 6.48 (0.5H), 4.58 (1H), 4.45 (1H), 4.17 (1H), 3.87 (1H), 3.61-3.55 (6H), 3.21 (2H), 2.97 (1H), 2.68 (1H), 2.56-2.32 (2H), 2.31-2.17 (2H), 1.94-1.69 (5H), 1.44 (1H), 1.34-1.23 (9H), 0.86-0.81 (9H). MS ESI: 671.5 [M+H]⁺

### Example 16 (16)

### Methyl N-[(1S)-1-{[(1R/S)-1-{[(1S)-1-{[(1S)-3-carbamoyl-1-[(1S,2S,6R,8S)-2,9,9-trimethyl-3,5-dioxa-4-boratricyclo[6.1.1.0^{2,6}]decan-4-yl]propyl]carbamoyl}ethyl]carbamoyl}-2-phenylethyl]carbamoyl}-2-methylpropyl]carbamate

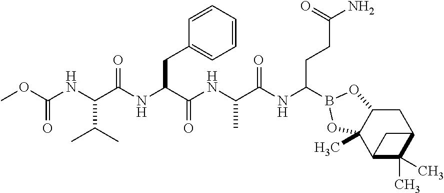

Intermediate 6 (0.12 g, 0.39 mmol), Intermediate 3 (0.30 g, 0.61 mmol, 1.6 eq) and platinum oxide (0.009 g, 0.04 mmol, 0.1 eq.) were placed in an rbf sealed with a septum. The flask was purged (vacuum then N₂) and degassed THF (2.5 mL) was added. The mixture was then stirred under a H₂ atmosphere (P=1 atm) delivered via a latex balloon. After 18 h, methanol and silica gel were added to the reaction vessel and the mixture was concentrated. The residue was poured onto the top of a silica gel column pre-packed with EtOAc. Flash chromatography on silica gel eluting with an increasing proportion of MeOH in EtOAc, concentration of the fractions containing the desired material, trituration of the residue with EtOAc and collection of the solid product by suction filtration followed by drying under high vacuum gave the title compound as a mixture of diastereomers.

¹H NMR (methanol-d4): δ 7.30-7.18 (5H), 4.59 (1H), 4.49 (1H), 4.18 (1H), 3.77 (1H), 3.63 (3H), 3.20 (1H), 2.95 (1H), 2.64 (1H), 2.37-2.10 (4H), 1.97-1.70 (6H), 1.42 (3H), 1.37-1.31 (4H), 1.27 (3H), 0.86 (3H), 0.82-0.77 (6H). MS ESI: 656.5 [M+H]⁺

### Example 17 (17)

### [(1R/S)-4-Methoxy-1-[(2S)-2-[(2S)-2-[(2S)-2-[(methoxycarbonyl)amino]-3-methylbutanamido]-3-phenylpropanamido]propanamido]-4-oxobutyl]boronic acid

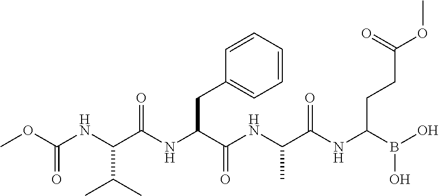 [SMILES: COC(=O)CCC(NC(=O)[C@H](C)NC(=O)[C@H](Cc1ccccc1)NC(=O)[C@@H](NC(=O)OC)C(C)C)B(O)O]

Example 15 (0.010 g, 0.015 mmol) was stirred with polymer-supported benzeneboronic acid (3 mmol/g loading, 0.058 g) in 1 mL of 1:1 acetonitrile/water for 18 h at rt. The resin was removed by filtration and was washed with water and acetonitrile. The filtrate was concentrated and additional portions of polymer-supported benzeneboronic acid (3 mmol/g, 0.058 g) and acetonitrile (5 mL) were added with stirring at rt for 18 h. The second portion of resin was removed by filtration and was washed with water and acetonitrile. Concentration of the filtrate in vacuo to remove acetonitrile, freeze drying of the residual aqueous solution, and trituration of the residue with Et₂O afforded the title compound.

¹H NMR (MeOH-d₄): δ 7.31-7.17 (5H), 4.58 (1H), 4.47 (1H), 3.80 (1H), 3.67-3.61 (6H), 3.19 (1H), 2.95 (1H), 2.58 (1H), 2.46-2.39 (2H), 1.93 (1H), 1.86-1.65 (2H), 1.43 (3H), 0.84-0.76 (6H). MS ESI: 559.3 [M+Na]⁺

### Example 18 (18)

### [(1R/S)-3-Carbamoyl-1-[(2S)-2-[(2S)-2-[(2S)-2-[(methoxycarbonyl)amino]-3-methylbutanamido]-3-phenylpropanamido]propanamido]propyl]boronic acid

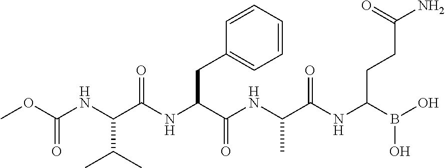 [SMILES: COC(=O)N[C@H](C(=O)N[C@@H](Cc1ccccc1)C(=O)N[C@@H](C)C(=O)NC(CCC(N)=O)B(O)O)C(C)C]

A sample of the final product from example 16 (0.020 g, 0.030 mmol) was suspended in a mixture of Et₂O (2.5 mL), water (1 mL) and EtOAc (0.5 mL). Phenylboronic acid (0.036 g, 0.30 mmol, 10 eq.) was added and the mixture was stirred at rt for 18 h. The phases were separated and the aqueous layer was washed with EtOAc (5×) and the collected organics were discarded. The aqueous layer was freeze dried and the residue was triturated with EtOAc to afford the title compound.

¹H NMR (DMSO-d₆+5% H₂O): δ 8.08-7.96 (2H), 7.65 (1H), 7.37 (1H), 7.28-7.12 (5H), 7.04 (1H), 6.69 (1H), 4.55 (1H), 4.30 (1H), 3.75 (1H), 3.51 (3H), 2.99 (1H), 2.75 (1H), 2.56 (1H), 2.02-1.95 (2H), 1.79 (1H), 1.69-1.40 (2H), 1.20-1.12 (3H), 0.68 (6H). MS ESI: 544.3 [M+Na]⁺

The following examples were prepared according to General Sequence 5:

### Example 19 (19)

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)decanamide

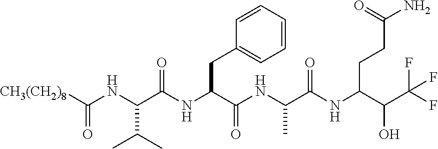

Prepared as a mixture of diastereomers from Intermediate 12 (0.10 g, 0.20 mmol), Intermediate 8 (57 mg, 0.24 mmol, 1.2 eq.), HATU (91 mg, 0.24 mmol, 1.2 eq.) and DIPEA (0.12 mL, 0.69 mmol, 3.5 eq.) in DMF (4 mL).

¹H NMR (DMSO-d₆): δ 8.02-7.87 (3H), 7.71 (1H), 7.25-7.12 (6H), 6.71 (1H), 4.52 (1H), 4.21 (1H), 4.07 (1H), 3.97-3.77 (2H), 3.03 (1H), 2.78 (1H), 2.20-1.93 (4H), 1.93-1.81 (2H), 1.64 (1H), 1.49-1.38 (2H), 1.29-1.15 (15H), 0.85 (3H), 0.73 (6H). ¹⁹F NMR (DMSO-d₆): δ −74.65, −74.72. ESI-MS: 672.5 [M+H⁺]

### Example 20a (20a)

### N-((2S)-1-(((2S)-1-(((2S)-1-(((3S)-6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)decanamide

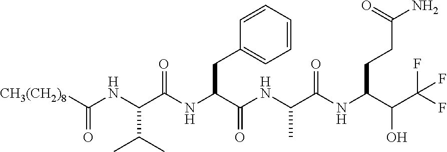

Prepared from Intermediate 12 (40 mg, 0.082 mmol), Intermediate 9 (23 mg, 0.1 mmol, 1.2 eq.), HATU (38 mg, 0.1 mmol, 1.2 eq.) and DIPEA (50 μL, 0.29 mmol, 3.5 eq.) in DMF (2 mL). The solid product was purified by dissolution in hot EtOH, treatment with activated charcoal and filtration through a Celite pad. The pad was washed with additional hot EtOH and the filtrate was concentrated under reduced pressure to afford the title compound as a solid following trituration with EtOAc containing a few drops of MeOH.

¹H NMR (DMSO-d₆): δ 8.01 (1H), 7.98 (1H), 7.92 (1H), 7.72 (1H), 7.25-7.12 (6H), 6.71 (1H), 6.53 (1H), 4.52 (1H), 4.21 (1H), 4.07 (1H), 3.93 (1H), 3.82 (1H), 3.03 (1H), 2.78 (1H), 2.19-1.97 (4H), 1.97-1.81 (2H), 1.60 (1H), 1.49-1.39 (2H), 1.29-1.16 (12H), 1.18 (3H), 0.85 (3H), 0.73 (6H). ¹⁹F NMR (DMSO-d₆): δ −74.65. ESI-MS: 672.5 [M+H⁺].

### Example 20b (20b)

### N-((2S)-1-(((2S)-1-(((2S)-1-(((3R)-6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)decanamide

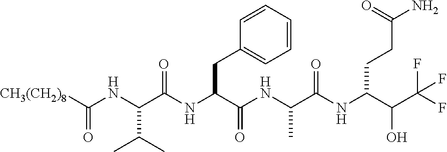

Prepared as for 20a from Intermediate 12 and ent-Intermediate 9.

### Examples 21a (21a), 21b (21b)

### N-((2S)-1-(((2S)-1-(((2S)-1-((6-Amino-1-fluoro-2-hydroxy-6-oxohexan-3-yl)amino)-1-oxopropan-2-yl)amino)-1-oxo-3-phenylpropan-2-yl)amino)-3-methyl-1-oxobutan-2-yl)decanamide

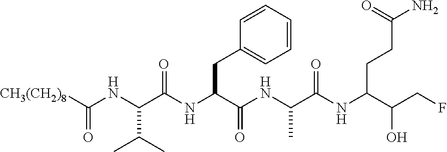

Compound 21a was prepared from Intermediate 12 (100 mg, 0.20 mmol), the minor diastereomer of Intermediate 10 (56 mg, 0.28 mmol, 1.4 eq.), HATU (100 mg, 0.26 mmol, 1.3 eq.) and DIPEA (0.12 mL, 0.69 mmol, 3.5 eq.) in DMF (3 mL). The title compound was isolated as a mixture of two diastereomers by flash chromatography on silica gel eluting with an increasing proportion of MeOH (5% to 10%) in CH₂Cl₂. Compound 21b was prepared in an analogous manner utilizing the major diastereomer of Intermediate 10.

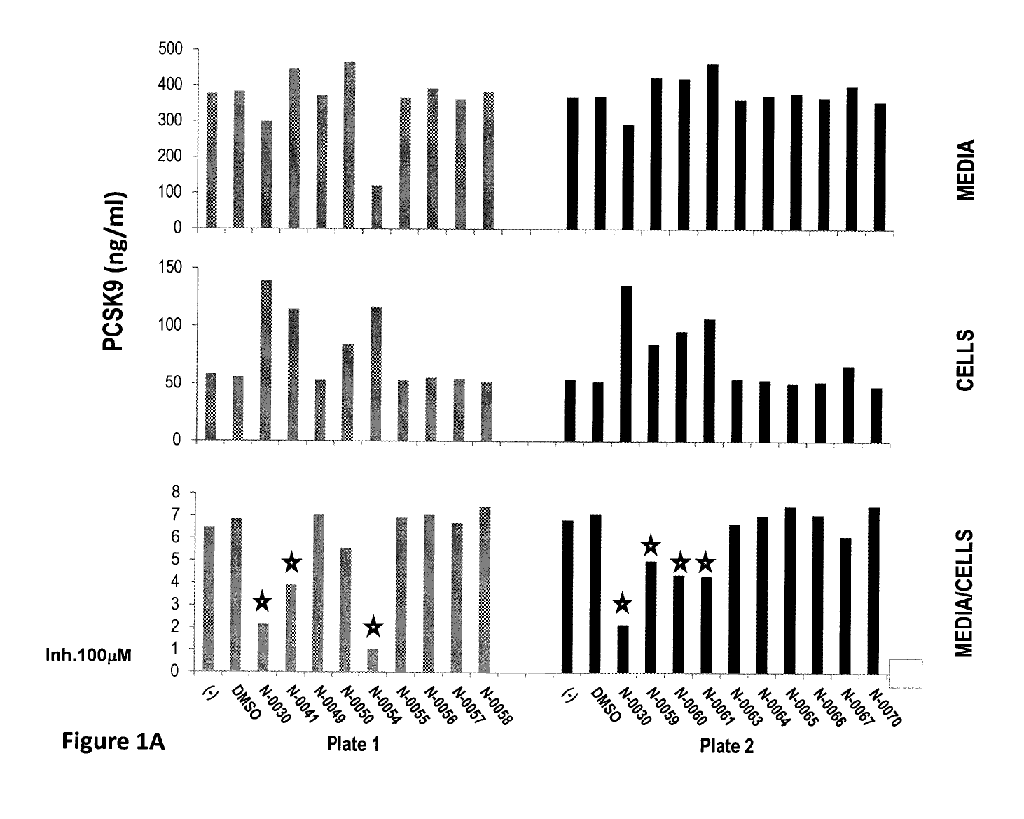

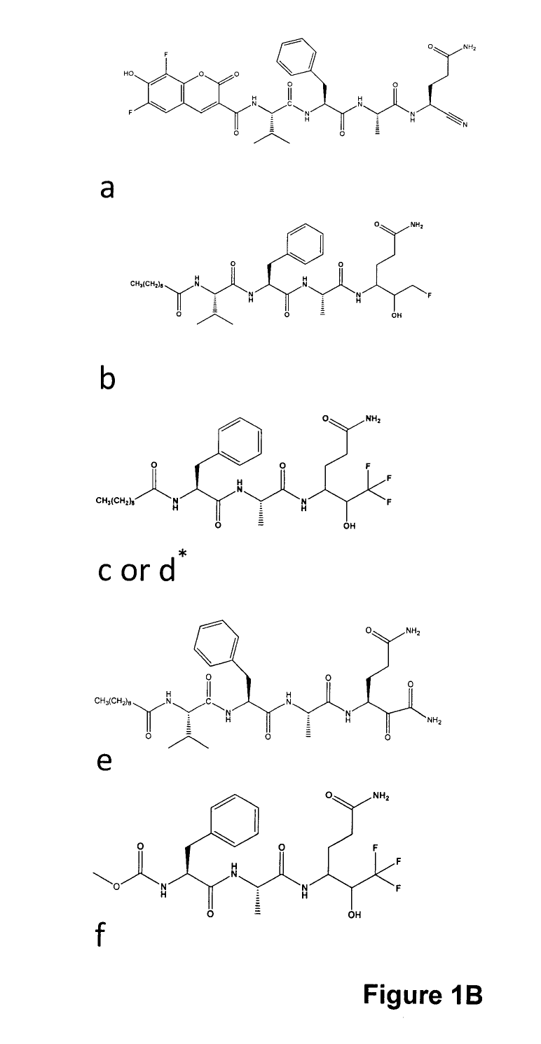

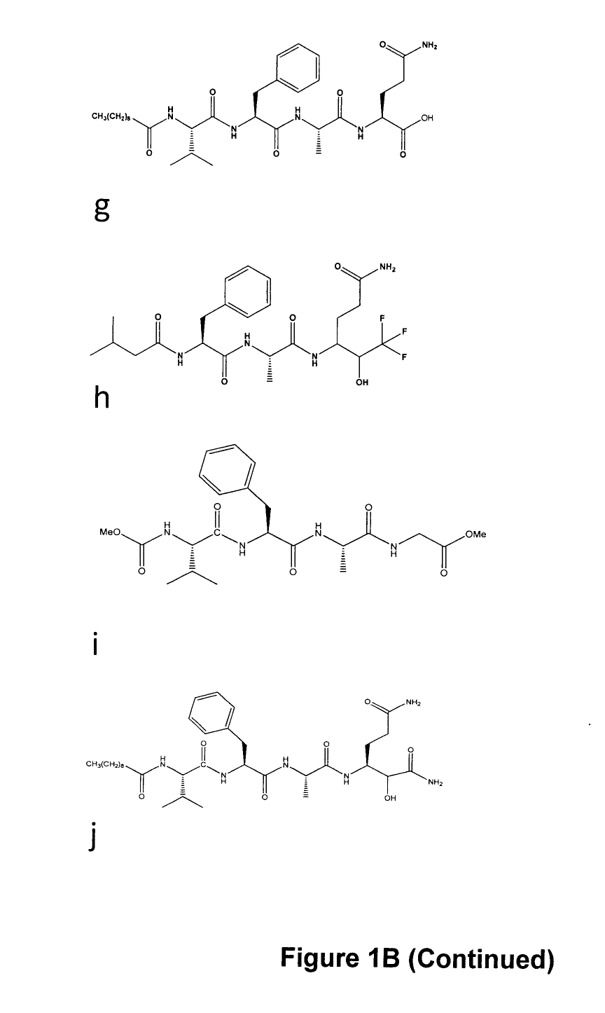

Compound b (see FIG. 1) is a mixture of compounds 21a and 21b.

21a: ¹H NMR (DMSO-d₆): δ 8.01-7.93 (2H), 7.74-7.62 (2H), 7.24-7.13 (6H), 6.69 (1H), 5.28 (1H), 4.52 (1H, m), 4.44-4.03 (4H), 3.66-3.46 (2H), 3.03 (1H), 2.79 (1H), 2.19-1.82 (6H), 1.59-1.38 (3H), 1.30-1.16 (15H), 0.85 (3H), 0.74 (6H). ¹⁹F NMR (DMSO-d6): δ 3.84, 3.18. ESI-MS: 636.5 [M+H]⁺

21b: ¹H NMR (DMSO-d₆): δ 8.04-7.95 (2H), 7.74-7.67 (1H), 7.55 (0.5H), 7.48 (0.5H), 7.26-7.12 (6H), 6.71 (1H), 4.52 (1H), 4.37-4.04 (4H), 3.80-3.67 (2H), 3.03 (1H), 2.78 (1H), 2.20-1.92 (4H), 1.87 (1H), 1.76-1.58 (2H), 1.50-1.40 (2H), 1.29-1.17 (15H), 0.85 (3H), 0.77-0.70 (6H). ESI-MS: 636.5 [M+H]⁺

### Examples 22a (22a) and 22b (2b)

### N-((2S)-1-(((2S)-1-((-1-((6-Amino-2-hydroxy-6-oxohexan-3-yl)amino)-1-oxopropan-2-yl)amino)-1-oxo-3-phenylpropan-2-yl)amino)-3-methyl-1-oxobutan-2-yl)decanamide

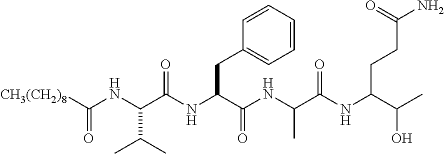

Prepared from Intermediate 12 (100 mg, 0.204 mmol), Intermediate 11 (40 mg, 0.27 mmol, 1.3 eq.), HATU (91 mg, 0.24 mmol, 1.2 eq.) and DIPEA (0.12 mL, 0.69 mmol, 3.5 eq.) in DMF (3 mL). The two diastereomeric products were separable into by flash chromatography on silica gel eluting with an increasing proportion of MeOH (5% to 10%) in CH₂Cl₂. Compound 22a was the first eluting (less polar) diastereomer and Compound 22b was the second eluting (more polar) diastereomer. Both products were isolated as solids following concentration of the appropriate fractions and trituration of the residue with water and Et₂O.

22a: ¹H NMR (DMSO-d₆): δ 8.15 (1H), 7.96 (1H), 7.73 (1H), 7.45 (1H), 7.26-7.14 (6H), 6.69 (1H), 4.56 (1H), 4.40 (1H), 4.16 (1H), 4.10 (1H), 3.50-3.41 (2H), 2.95 (1H), 2.84 (1H), 2.20-2.02 (2H), 2.02-1.82 (4H), 1.56-1.40 (3H), 1.30-1.17 (12H), 1.07 (3H), 0.99 (3H), 0.85 (3H), 0.764 (3H), 0.748 (3H). ESI-MS: 618.5 [M+H]⁺.

22b: ¹H NMR (DMSO-d₆): δ 8.00 (1H), 7.93 (1H), 7.71 (1H), 7.52 (1H), 7.25-7.12 (6H), 6.68 (1H), 4.63 (1H), 4.53 (1H), 4.21 (1H), 4.07 (1H), 3.52-3.39 (2H), 3.03 (1H), 2.78 (1H), 2.19-1.81 (6H), 1.51-1.39 (3H), 1.29-1.18 (15H), 0.98 (3H), 0.85 (3H), 0.74 (6H). ESI-MS: 618.5 [M+H]⁺.

### Example 23 (23)

### 6,6,6-Trifluoro-5-hydroxy-4-((S)-2-((S)-2-((S)-3-methyl-2-(2-(4-(trifluoromethoxy)phenyl)acetamido)butanamido)-3-phenylpropanamido)propanamido)hexanamide

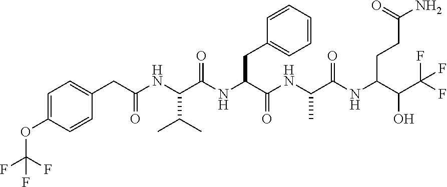 [SMILES: CC(C)[C@H](NC(=O)Cc1ccc(OC(F)(F)F)cc1)C(=O)N[C@@H](Cc1ccccc1)C(=O)N[C@@H](C)C(=O)NC(CCC(N)=O)C(O)C(F)(F)F]

Prepared from Intermediate 13 (108 mg, 0.20 mmol), Intermediate 8 (57 mg, 0.24 mmol, 1.2 eq.), HATU (91 mg, 0.24 mmol, 1.2 eq.), DIPEA (0.12 mL, 0.69 mmol, 3.5 eq.) in DMF (2 mL). The title product was isolated as a mixture of diastereomers by flash chromatography on silica gel eluting with an increasing proportion of MeOH (5% to 10%) in CH₂Cl₂ followed by trituration with water and Et₂O.

¹H NMR (DMSO-d₆): δ 8.11-8.05 (2H), 7.99 (0.4H), 7.92-7.90 (1.6H), 7.36 (2H), 7.27 (2H), 7.24-7.11 (6H), 6.72 (1H), 6.50 (1H), 4.53 (1H), 4.22 (1H), 4.10 (1H), 3.96-3.78 (2H), 3.57 (1H), 3.46 (1H), 3.02 (1H), 2.77 (1H), 2.10-1.82 (4H), 1.64 (1H), 1.20 (1.8H), 1.16 (1.2H), 0.73 (6H). ¹⁹F NMR (DMSO-d₆): δ −57.2 (both diastereomers), −74.6 (one diastereomer), −74.71 (other diastereomer). ESI-MS: 720.3 [M+H⁺].

### Example 24 (24)

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-[1,1′-biphenyl]-4-carboxamide

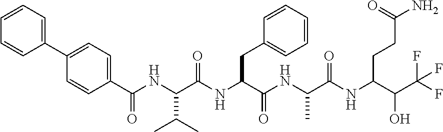 [SMILES: CC(C)[C@H](NC(=O)c1ccc(-c2ccccc2)cc1)C(=O)N[C@@H](Cc1ccccc1)C(=O)N[C@@H](C)C(=O)NC(CCC(N)=O)C(O)C(F)(F)F]

Prepared as a mixture of diastereomers from Intermediate 14 (70 mg, 0.14 mmol), Intermediate 8 (46 mg, 0.18 mmol, 1.3 eq.), HATU (62 mg, 0.16 mmol, 1.2 eq.), DIPEA (85 μL, 0.49 mmol, 3.5 eq.) in DMF (1.4 mL).

¹H NMR (DMSO-d₆): δ 8.27 (1H), 8.11-8.00 (2H), 7.97-7.86 (3H), 7.78 (2H), 7.74 (2H), 7.50 (2H), 7.42 (1H), 7.26-7.09 (6H), 6.71 (1H), 6.52 (1H), 4.60 (1H), 4.26-4.19 (2H), 3.98-3.77 (2H), 3.05 (1H), 2.79 (1H), 2.12-1.82 (4H), 1.63 (1H), 1.23 (1.5H), 1.19 (1.5H), 0.84 (3H, d), 0.762 (1.5H), 0.752 (1.5H). ¹⁹F NMR (DMSO-d₆): δ −74.64, −74.69. ESI-MS: 698.4 [M+H⁺].

Schemes 1 through 9 and the following text describe the general experimental procedures that were utilized to prepare compound examples 25-91. All reactions sensitive to moisture, atmospheric oxygen and/or carbon dioxide were performed under an anhydrous nitrogen atmosphere in solvents that were pre-dried over molecular sieves and degassed by several freeze-thaw cycles under hi-vacuum. Modifications to the described procedures, where necessary, are included with the description of the specific examples to which they apply. Final compounds were isolated as beige to colorless powders. Compounds prepared from intermediate 8 were generally isolated as 1:1 mixture of diastereomers, unless indicated otherwise.

General Procedure for Amine to Carboxylic Acid Coupling with HATU, DIPEA:

DIPEA (3.5 eq.) was added dropwise to a stirred mixture of the appropriate amine (1.2 eq.), HATU (1.2 eq.) and the appropriate carboxylic acid at a concentration of 0.2 M in DMF at −20° C. followed by slow warming to 0° C. and stirring at rt overnight. The reaction vessel contents were then diluted with EtOAc and stirred with an ice-cold, half-saturated NaHCO₃ aqueous solution. The solid amide product was isolated by filtration, washed with water and EtOAc, and dried under suction and hi-vacuum. In cases where the product was soluble in EtOAc, the layers were separated and the aqueous phase was extracted with additional EtOAc. The combined organics were washed with aqueous solutions of 5-10% LiCl or 1:1 saturated brine/water, followed by drying over MgSO₄. The organics were filtered and concentrated under reduced pressure and the residue was triturated with ether containing 0-50% EtOAc. The solid product was collected by filtration and dried under high vacuum. In cases where purification by trituration was insufficient to reach a final purity of >90-95%, the isolated material was subjected to recrystallization or by flash chromatography on silica gel eluting with an increasing proportion of EtOAc or MeOH in CH₂Cl₂.

### General Procedure for Amine to Carboxylic Acid Coupling Using Isobutyl Chloroformate:

(Shieh, Wen-Chung; Carlson, John A.; Shore, Michael E.; Tetrahedron Lett. 1999, 40, 7167-70.)

A 0.35 M solution of isobutyl chloroformate (1.1 eq.) in CH₂Cl₂ or THF was added slowly over a period of 1 h to a mixture of an appropriate amine hydrochloride (1.1 eq.), N-methylmorpholine (2.2 eq.) and an appropriate carboxylic acid at a concentration of 0.1 M in CH₂Cl₂ or THF at 00° C. Stirring at this temperature was continued for an additional 15 min prior to quenching of the reaction by the addition of saturated aqueous sodium bicarbonate solution and EtOAc. The solid amide product was collected by suction filtration and washed sequentially with water, EtOAc and ether. Drying under suction and hi-vacuum typically afforded the desired compound as a colorless to beige powder. Soluble products were isolated by an extractive workup procedure (EtOAc (2×), saturated aqueous NaHCO₃ and NaCl washes, Na₂SO₄ to dry the organic layer, filtration, concentration in vacuo). In cases where purification by trituration (typically with ether or with 1:1 EtOAc/ether) was insufficient to reach a final purity of >90-95%, the product was purified by recrystallization (in the specified solvent mixture) or by flash chromatography on silica gel eluting with an increasing proportion of EtOAc or MeOH in CH₂Cl₂, or EtOAc in hexanes.

General Procedure for Ester Saponification with Lithium Hydroxide:

Aqueous LiOH (1 M, 1.5-2 eq.) was added dropwise to the appropriate methyl or ethyl ester at a concentration of 0.1 M in a 2:1 mixture of MeOH and THF at 000° C. After 15 min at this temperature, the reaction was warmed to rt and stirred until the reaction was judged complete by LCMS or TLC analysis. Crushed ice was then added and the mixture was acidified to pH 3-4 with 1 M HCl. After stirring for an additional 1 h, the solid product was isolated by suction filtration and dried under high vacuum. Products that failed to precipitate under these conditions were isolated by an extraction with EtOAc (2×), after which the combined organics were washed with brine, dried (MgSO₄), filtered and concentrated, and the residue was used directly in the next step.

General Procedure for Boc Deprotection with 4 M HCl in Dioxane:

A 4 M solution of HCl in 1,4-dioxane (10-20 eq.) was added slowly to the appropriate Boc-protected amine at a concentration of 0.5 M in CH₂Cl₂ at 0° C. The resulting mixture was then stirred at rt and the reaction progress was monitored by LCMS or TLC. When necessary, additional HCl in dioxane was added to drive the reaction to completion. The reaction mixture was then concentrated to dryness under reduced pressure to afford the desired deprotected amine hydrochloride salt which was used directly in the next reaction without further purification.

### General Procedure for Benzyl Ester or Cbz-Deprotection Via Hydrogenolysis:

A measured volume of deoxygenated MeOH to achieve a substrate concentration of 0.1-0.2 M was added via cannula to the appropriate Cbz-protected amine or benzyl ester and 10% Pd/C (30 wt %) in a septum-capped rbf under vacuum. The reaction vessel contents were then placed under an atmosphere of H₂ via a latex balloon and the reaction vessel was agitated in an ultrasonic bath for 2 min. The mixture was stirred at rt until the reaction was judged to be complete by LCMS or TLC analysis whereupon the vessel was flushed with N₂ and the reaction mixture was diluted with an equal volume of CH₂Cl₂. The suspension was filtered through a Celite pad and the pad was washed thoroughly with a 1:1 mixture of MeOH and CH₂Cl₂. The filtrate was concentrated and dried under hi-vacuum and the residue was used directly in the next step.

### General Procedure for R9 Introduction Using Acyl or Sulfonyl Chlorides:

The appropriate acyl or sulfonyl chloride (1.1 eq.) was added portion-wise to a stirred mixture of DIPEA (2.2 eq.) or Et₃N (2.2 eq.) and the appropriate amine or amine hydrochloride at a concentration of 0.1-0.2 M in CH₂Cl₂ at 0° C. followed by slow warming to rt with stirring until the reaction was judged complete by LCMS or TLC analysis. Additional CH₂Cl₂ and dilute aqueous HCl were added to the reaction vessel and the solid product was isolated by suction filtration, washed with CH₂Cl₂ and water and dried under high vacuum. Non-solid or soluble products were isolated by an extractive workup with CH₂Cl₂ or EtOAc followed by washing of the combined organic extracts with saturated solutions of NaHCO₃ and brine, drying over MgSO₄ and concentration under reduced pressure. Trituration of the residue with Et₂O typically generated a solid product that was isolated by filtration and dried under high vacuum.

### General Procedure for Preparation of N-Acyl Amino Acids:

A suitable acyl chloride (1 eq.) was added in one portion at rt to a 3:2:6 mixture of aqueous NaOH (1 M, 2 eq.), THF and Et₂O containing an appropriate amino acid followed by rapid stirring overnight. The acylated product was isolated by extraction with EtOAc (2×) following adjustment of the mixture to pH 2-3 with 10% aqueous HCl. The combined organics were washed with brine, dried over Na₂SO₄ and concentrated to dryness. Recrystallization of the residue from CH₂Cl₂/Et₂O afforded the desired N-acyl amino acid in suitable purity for use in the subsequent reaction.

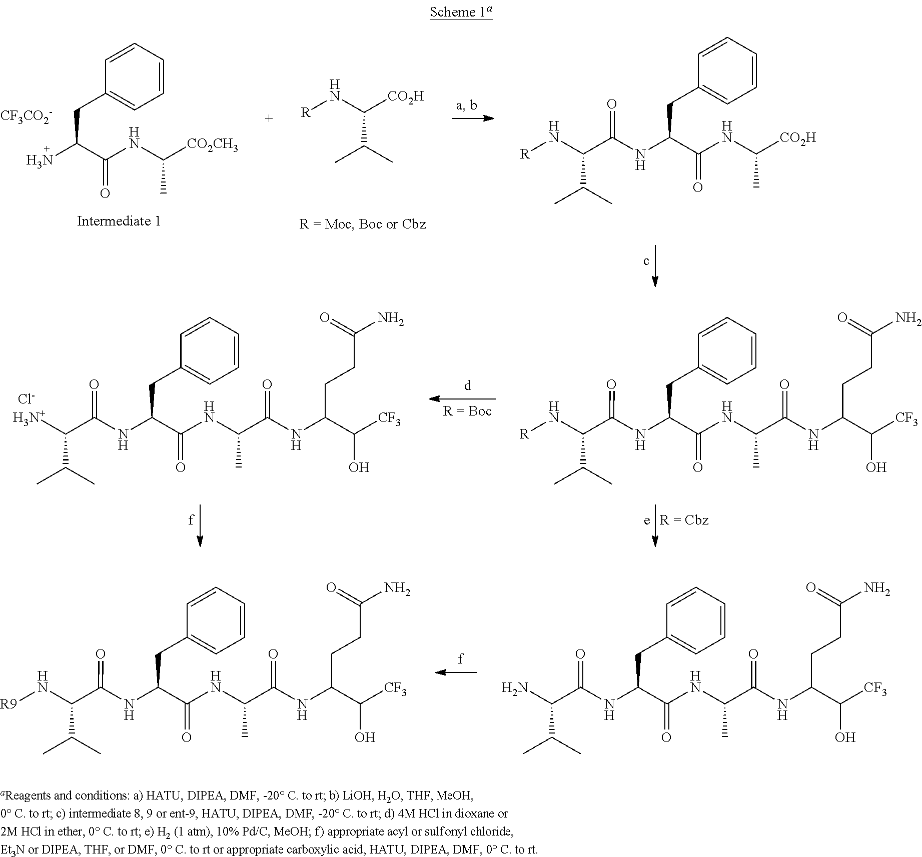

### Compound Examples Prepared According to Scheme 1

### Example 25

### Benzyl ((2S)-1-(((2S)-1-(((2S)-1-((6-amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)carbamate

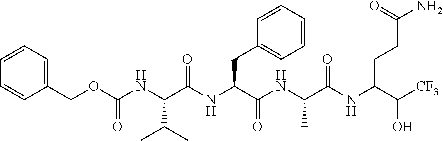 [SMILES: CC(C)[C@H](NC(=O)OCc1ccccc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.10 (0.5H), 8.00 (1.5H), 7.90 (1H), 7.40-7.08 (12H), 6.72 (1H), 6.50 (1H), 5.00 (2H), 4.55 (1H), 4.20 (1H), 3.99-2.65 (3H), 3.00 (1H), 2.72 (1H), 2.12-1.75 (4H), 1.60 (1H), 1.18 (3H), 0.70 (6H); ¹⁹F NMR (DMSO-d₆): δ −74.65, −74.69; MS ESI: 652.4 [M+H]⁺

### Example 26

### Methyl ((2S)-1-(((2S)-1-(((2S)-1-((6-amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)carbamate

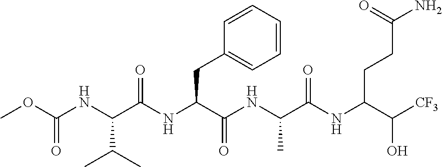 [SMILES: COC(=O)N[C@H](C(=O)N[C@@H](Cc1ccccc1)C(=O)N[C@@H](C)C(=O)NC(CCC(N)=O)C(O)C(F)(F)F)C(C)C]

¹H NMR (DMSO-d₆): δ 8.12 (0.5H), 8.05 (0.5H); 7.94 (1H); 7.63 (0.5H), 7.51 (0.5H); 7.35-7.12 (6H), 7.03 (1H), 6.72 (1H), 6.56 (0.5H), 6.52 (0.5H), 4.56 (1H), 4.28 (1H), 4.15-3.98 (2H), 3.73 (1H), 3.50 (3H), 3.00 (1H), 2.75 (1H), 2.04 (2H), 1.82 (1H), 1.85-1.62 (2H), 1.20 (1.5H), 1.17 (1.5H), 0.72 (6H); ¹⁹F NMR (DMSO-d₆): δ −75.25, −75.37; MS ESI: 576.3 [M+H]⁺

### Example 27

### tert-Butyl ((2S)-1-(((2S)-1-(((2S)-1-((6-amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)carbamate

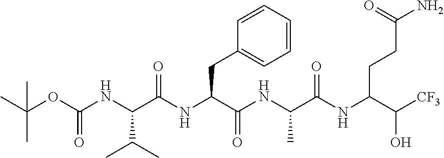 [SMILES: CC(C)[C@H](NC(=O)OC(C)(C)C)C(=O)N[C@@H](Cc1ccccc1)C(=O)N[C@@H](C)C(=O)NC(CCC(N)=O)C(O)C(F)(F)F]

¹H NMR (DMSO-d₆): δ 8.12 (0.5H), 8.04 (0.5H), 7.86 (2H), 7.24-7.08 (6H), 6.69 (1H), 6.62 (1H), 6.47 (1H), 4.58 (1H), 4.20 (1H), 3.96-3.75 (2H), 3.66 (1H), 3.00 (1H), 2.73 (1H), 2.10-1.80 (3H), 1.77 (1H), 1.61 (1H), 1.38 (9H), 1.18 (3H), 0.64 (6H); ¹⁹F NMR (DMSO-d₆): δ −74.66, −74.69; MS ESI: 618.4 [M+H]⁺

### Example 28

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4-isopropylbenzamide

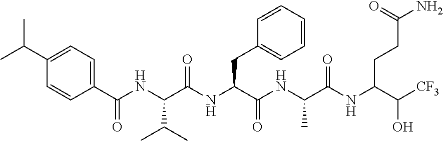 [SMILES: CC(C)c1ccc(C(=O)N[C@H](C(=O)N[C@@H](Cc2ccccc2)C(=O)N[C@@H](C)C(=O)NC(CCC(N)=O)C(O)C(F)(F)F)C(C)C)cc1]

¹H NMR (DMSO-d₆): δ 8.14-7.93 (3H), 7.84 (1H), 7.75 (2H), 7.30 (2H), 7.25-7.01 (6H), 6.70 (1H), 6.46 (1H), 4.58 (1H), 4.25-4.12 (2H), 3.98-3.72 (2H), 3.02 (1H), 2.93 (1H), 2.74 (1H), 2.11-1.78 (4H), 1.60 (1H), 1.20 (9H), 0.80 (3H), 0.70 (3H); ¹⁹F NMR (DMSO-d₆): δ −74.65, −74.70; MS ESI: 664.4 [M+H]⁺.

### Example 29

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4-(3-(trifluoromethyl)-3H-diazirin-3-yl)benzamide

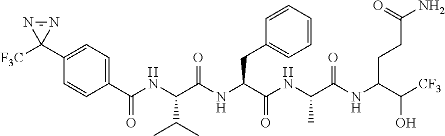 [SMILES: CC(C)[C@H](NC(=O)c1ccc(C2(C(F)(F)F)N=N2)cc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.38 (1H), 8.10-7.80 (5H), 7.37 (2H), 7.24-7.02 (6H), 6.67 (1H), 6.45 (1H), 4.55 (1H), 4.20 (2H), 3.97-3.72 (2H), 3.01 (1H), 2.77 (1H), 2.12-1.80 (4H), 1.60 (1H), 1.20 (3H), 0.80 (3H), 0.75 (3H); ¹⁹F NMR (DMSO-d₆): δ −64.90, −74.66, −74.70; MS ESI: 730.4 [M+H]⁺

### Example 30

### 6,6,6-Trifluoro-5-hydroxy-4-((S)-2-((S)-2-((S)-3-methyl-2-(2-(4-(3-(trifluoromethyl)-3H-diazirin-3-yl)phenyl)acetamido)butanamido)-3-phenylpropanamido)propanamido)hexanamide

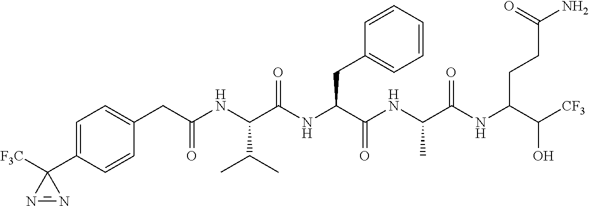 [SMILES: CC(C)[C@H](NC(=O)Cc1ccc(C2(C(F)(F)F)N=N2)cc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.08 (2H), 7.86 (2H), 7.37 (2H), 7.30-7.02 (8H), 6.68 (1H), 6.44 (1H), 4.50 (1H), 4.20 (1H), 4.10 (1H), 3.98-3.75 (2H), 3.36 (1H), 3.24 (1H), 3.00 (1H), 2.73 (1H), 2.12-1.80 (4H), 1.60 (1H), 1.18 (3H), 0.68 (6H); MS ESI: 744.4 [M+H]⁺

### Example 31

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-3-(3-(trifluoromethyl)-3H-diazirin-3-yl)benzamide

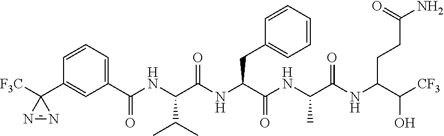 [SMILES: CC(C)[C@H](NC(=O)c1cccc(C2(C(F)(F)F)N=N2)c1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.46 (1H), 8.16 (1H), 8.00 (1H), 7.98-7.84 (2H), 7.60 (2H), 7.52 (1H), 7.27-7.02 (6H), 6.69 (1H), 6.47 (1H), 4.55 (1H), 4.21 (2H), 3.97-3.75 (2H), 3.01 (1H), 2.77 (1H), 2.12-1.80 (4H), 1.64 (1H), 1.18 (3H), 0.80 (6H); MS ESI: 730.3 [M+H]⁺

### Example 32

### 6,6,6-Trifluoro-5-hydroxy-4-((S)-2-((S)-2-((S)-3-methyl-2-(2-(3-(3-(trifluoromethyl)-3H-diazirin-3-yl)phenyl)acetamido)butanamido)-3-phenylpropanamido)propanamido)hexanamide

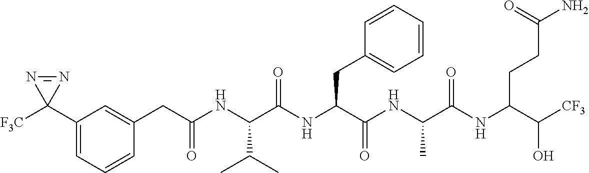 [SMILES: CC(C)[C@H](NC(=O)Cc1cccc(C2(C(F)(F)F)N=N2)c1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.20-8.05 (2H), 8.00-7.90 (2H), 7.40 (2H), 7.26-7.08 (8H), 6.71 (1H), 6.59 (1H), 4.51 (1H), 4.20 (1H), 4.09 (1H), 3.98-3.75 (2H), 3.60 (1H), 3.42 (1H), 3.00 (1H), 2.75 (1H), 2.10-1.80 (4H), 1.60 (1H), 1.18 (3H), 0.70 (6H); MS ESI: 744.4 [M+H]⁺

### Example 33

### 4-((S)-2-((S)-2-((S)-2-([1,1′-Biphenyl]-4-sulfonamido)-3-methylbutanamido)-3-phenylpropanamido)propanamido)-6,6,6-trifluoro-5-hydroxyhexanamide

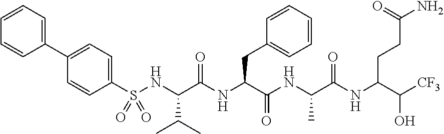 [SMILES: CC(C)[C@H](NS(=O)(=O)c1ccc(-c2ccccc2)cc1)C(=O)N[C@@H](Cc1ccccc1)C(=O)N[C@@H](C)C(=O)NC(CCC(N)=O)C(O)C(F)(F)F]

Extractive workup in the final step was performed with 9:1 CH₂Cl₂:MeOH as the organic phase; final purification was accomplished by flash chromatography on silica gel eluting with 2-6% MeOH/CH₂Cl₂.

¹H NMR (DMSO-d₆): δ 8.10 (1H), 7.95 (1H), 7.90-7.75 (2H), 7.72-7.60 (6H), 7.47 (2H), 7.40 (1H), 7.23-7.07 (6H), 6.70 (1H), 6.46 (1H), 4.30 (1H), 4.18 (1H), 3.95-3.75 (2H), 3.53 (1H), 2.89 (1H), 2.63 (1H), 2.06-1.71 (4H), 1.58 (1H), 1.13 (3H), 0.70 (6H); ¹⁹F NMR (DMSO-d₆): δ −74.66, −74.71; MS ESI: 734.4 [M+H]⁺

### Example 34

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-5-methyl-1-phenyl-1H-pyrazole-4-carboxamide

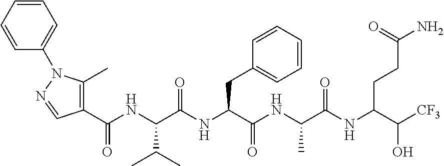 [SMILES: Cc1c(C(=O)N[C@H](C(=O)N[C@@H](Cc2ccccc2)C(=O)N[C@@H](C)C(=O)NC(CCC(N)=O)C(O)C(F)(F)F)C(C)C)cnn1-c1ccccc1]

¹H NMR (DMSO-d₆): δ 8.20 (1H), 8.10-7.95 (2H), 7.90 (1H), 7.82 (1H), 7.60-7.40 (5H), 7.24-7.05 (6H), 6.70 (1H), 6.54 (1H), 4.57 (1H), 4.20 (2H), 3.98-3.76 (2H), 3.02 (1H), 2.80 (1H), 2.47 (3H), 2.13-1.80 (4H), 1.62 (1H), 1.20 (3H), 0.80 (6H); ¹⁹F NMR (DMSO-d₆): δ −74.64, −74.69; MS ESI: 702.4 [M+H]⁺

### Example 35

### 6,6,6-Trifluoro-5-hydroxy-4-((S)-2-((S)-2-((S)-3-methyl-2-(2-phenylacetamido)butanamido)-3-phenylpropanamido)propanamido)hexanamide

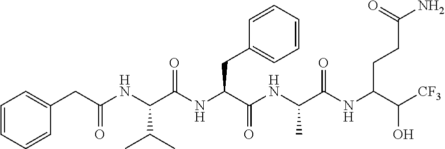 [SMILES: CC(C)[C@H](NC(=O)Cc1ccccc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.05 (1H), 8.00-7.82 (3H), 7.30-7.08 (11H), 6.70 (1H), 6.50 (1H), 4.53 (1H), 4.22 (1H), 4.10 (1H), 3.97-3.76 (2H), 3.53 (1H), 3.40 (1H), 3.01 (1H), 2.77 (1H), 2.10-1.80 (4H), 1.62 (1H), 1.20 (3H), 0.72 (6H); ¹⁹F NMR (DMSO-d₆): δ −74.64, −74.69; MS ESI: 636.4 [M+H]⁺

### Example 36

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-6-phenylnicotinamide

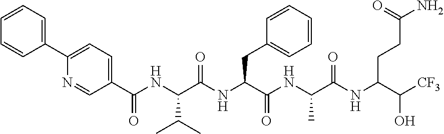 [SMILES: CC(C)[C@H](NC(=O)c1ccc(-c2ccccc2)nc1)C(=O)N[C@@H](Cc1ccccc1)C(=O)N[C@@H](C)C(=O)NC(CCC(N)=O)C(O)C(F)(F)F]

¹H NMR (DMSO-d₆): δ 9.07 (1H), 8.62 (1H), 8.26 (1H), 8.20-7.97 (5H), 7.90 (1H), 7.58-7.42 (3H), 7.25-7.02 (6H), 6.70 (1H), 6.60 (1H), 4.57 (1H), 4.22 (2H), 3.99-3.75 (2H), 3.03 (1H), 2.80 (1H), 2.12-1.80 (4H), 1.62 (1H), 1.20 (3H), 0.80 (6H); MS ESI: 699.4 [M+H]⁺

### Example 37

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-2-(pyridin-4-yl)thiazole-4-carboxamide

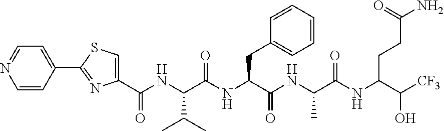 [SMILES: CC(C)[C@H](NC(=O)c1csc(-c2ccncc2)n1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.76 (2H), 8.49 (1H), 8.38 (1H), 8.15-7.82 (5H), 7.11-7.00 (6H), 6.70 (1H), 6.50 (1H), 4.59 (1H), 4.33 (1H), 4.22 (1H), 3.97-3.74 (2H), 3.02 (1H), 2.77 (1H), 2.15-1.80 (4H), 1.62 (1H), 1.20 (3H), 0.80 (6H); ¹⁹F NMR (DMSO-d₆): δ −74.64, −74.66; MS ESI: 706.3 [M+H]⁺

### Example 38

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4-(pyridin-3-yl)benzamide

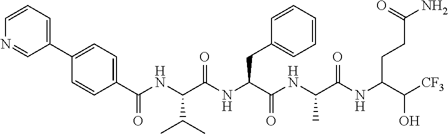 [SMILES: CC(C)[C@H](NC(=O)c1ccc(-c2cccnc2)cc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.97 (1H), 8.60 (1H), 8.31 (1H), 8.20-7.70 (8H), 7.50 (1H), 7.12-7.00 (6H), 6.70 (1H), 6.50 (1H), 4.60 (1H), 4.22 (2H), 4.07-3.71 (2H), 3.01 (1H), 2.79 (1H), 2.17-1.78 (4H), 1.61 (1H), 1.18 (3H), 0.79 (6H); ¹⁹F NMR (DMSO-d₆): δ −74.62, −74.65; MS ESI: 699.4 [M+H]⁺

### Example 39

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4-(hydroxymethyl)benzamide

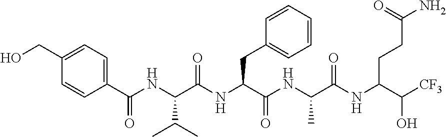 [SMILES: CC(C)[C@H](NC(=O)c1ccc(CO)cc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.12 (1H), 8.08 (1.5H), 8.0 (0.5H), 7.86 (1H), 7.80 (2H), 7.40 (2H), 7.24-7.04 (6H), 6.70 (1H), 6.50 (1H), 5.30 (1H), 4.59 (1H), 4.56 (2H), 4.20 (2H), 3.97-3.76 (2H), 3.03 (1H), 2.75 (1H), 2.10-1.80 (4H), 1.60 (1H), 1.19 (3H), 0.80 (3H), 0.70 (3H); ¹⁹F NMR (DMSO-d₆): δ −74.64, −74.69; MS ESI: 652.3 [M+H]⁺

### Example 40

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-1-phenyl-1H-pyrazole-4-carboxamide

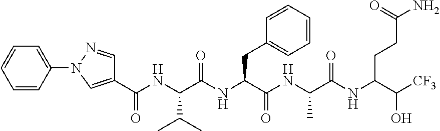 [SMILES: CC(C)[C@H](NC(=O)c1cnn(-c2ccccc2)c1)C(=O)N[C@@H](Cc1ccccc1)C(=O)N[C@@H](C)C(=O)NC(CCC(N)=O)C(O)C(F)(F)F]

¹H NMR (DMSO-d₆): δ 9.04 (1H), 8.19 (1H), 8.16 (1H), 8.00-7.83 (3H), 7.82 (2H), 7.54 (2H), 7.38 (1H), 7.23-7.02 (6H), 6.71 (1H), 6.51 (1H), 4.55 (1H), 4.23-4.18 (2H), 3.98-3.77 (2H), 3.04 (1H), 2.80 (1H), 2.14-1.80 (4H), 1.62 (1H), 1.20 (3H), 0.80 (6H); ¹⁹F NMR (DMSO-d₆): δ −74.65, −74.70; MS ESI: 688.3 [M+H]⁺

### Example 41

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4-morpholinobenzamide

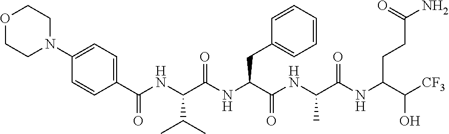 [SMILES: CC(C)[C@H](NC(=O)c1ccc(N2CCOCC2)cc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.08-7.98 (2H), 7.98-7.82 (2H), 7.76 (2H), 7.26-7.06 (6H), 6.97 (2H), 6.70 (1H), 6.55 (1H), 4.56 (1H), 4.25-4.08 (2H), 3.98-3.78 (2H), 3.72 (4H), 3.20 (4H), 3.02 (1H), 2.77 (1H), 2.14-1.80 (4H), 1.60 (1H), 1.20 (3H), 0.80 (3H), 0.70 (3H); ¹⁹F NMR (DMSO-d₆): δ −74.62, −74.65; MS ESI: 707.4 [M+H]⁺

### Example 42

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4-(morpholine-4-carbonyl)benzamide

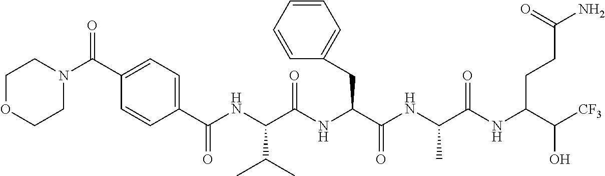 [SMILES: CC(C)[C@H](NC(=O)c1ccc(C(=O)N2CCOCC2)cc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.31 (1H), 8.15-8.10 (1.5H), 8.01 (0.5H), 7.97-7.82 (3H), 7.50 (2H), 7.27-7.04 (6H), 6.70 (1H), 6.52 (1H), 4.60 (1H), 4.20 (2H), 4.00-3.77 (2H), 3.75-3.42 (8H), 3.03 (1H), 2.78 (1H), 2.15-1.80 (4H), 1.63 (1H), 1.20 (3H), 0.83 (3H), 0.77 (3H); ¹⁹F NMR (DMSO-d₆): δ −74.64, −74.69; MS ESI: 735.4 [M+H]⁺

### Example 43

### 6,6,6-Trifluoro-5-hydroxy-4-((S)-2-((S)-2-((S)-3-methyl-2-(3-phenylpropiolamido)butanamido)-3-phenylpropanamido)propanamido)hexanamide

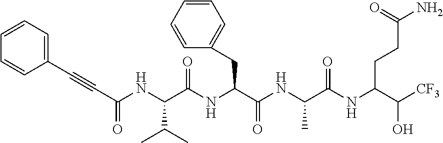 [SMILES: CC(C)[C@H](NC(=O)C#Cc1ccccc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.74 (1H), 8.14 (1H), 8.06 (0.5H), 8.00 (0.5H), 7.92 (1H), 7.59 (2H), 7.54-7.40 (3H), 7.27-7.17 (6H), 6.70 (1H), 6.52 (1H), 4.66 (1H), 4.31-4.11 (2H), 3.98-3.72 (2H), 3.02 (1H), 2.78 (1H), 2.12-1.80 (4H), 1.62 (1H), 1.18 (3H), 0.78 (6H); ¹⁹F NMR (DMSO-d₆): δ −74.64, −74.69; MS ESI: 646.4 [M+H]⁺

### Example 44

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)nicotinamide

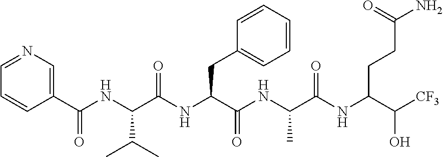 [SMILES: CC(C)[C@H](NC(=O)c1cccnc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.99 (1H), 8.70 (1H), 8.47 (1H), 8.17 (1H), 8.10 (1H), 8.01 (0.5H), 7.98 (0.5H), 7.90 (1H), 7.50 (1H), 7.24-7.02 (6H), 6.70 (1H), 6.48 (1H), 4.58 (1H), 4.20 (2H), 3.97-3.75 (2H), 3.03 (1H), 2.78 (1H), 2.12-1.80 (4H), 1.63 (1H), 1.20 (3H), 0.85 (3H), 0.77 (3H); ¹⁹F NMR (DMSO-d₆): δ −74.62, −74.68; MS ESI: 623.3 [M+H]⁺

### Example 45

### 6,6,6-Trifluoro-5-hydroxy-4-((S)-2-((S)-2-((S)-3-methyl-2-(2-phenoxyacetamido)butanamido)-3-phenylpropanamido)propanamido)hexanamide

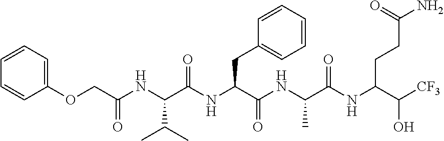 [SMILES: CC(C)[C@H](NC(=O)COc1ccccc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.20 (1H), 8.03 (0.5H), 7.98 (0.5H), 7.92 (1H), 7.72 (1H), 7.30-7.06 (8H), 6.99-6.84 (3H), 6.68 (1H), 6.53 (1H), 4.60-4.45 (3H), 4.27-4.13 (2H), 3.98-3.76 (2H), 3.03 (1H), 2.76 (1H), 2.15-1.80 (4H), 1.62 (1H), 1.20 (3H), 0.70 (6H); ¹⁹F NMR (DMSO-d₆): δ −74.60, −74.66; MS ESI: 652.4 [M+H]⁺

### Example 46

### 4-((6S,9S,12S)-9-Benzyl-6-isopropyl-12-methyl-4,7,10-trioxo-1-phenyl-2-oxa-5,8,11-triazatridecan-13-amido)-6,6,6-trifluoro-5-hydroxyhexanamide

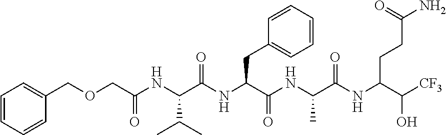 [SMILES: CC(C)[C@H](NC(=O)COCc1ccccc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.23 (1H), 8.07 (0.5H), 8.00 (0.5H), 7.90 (1H), 7.42-7.02 (12H), 6.70 (1H), 6.50 (1H), 4.56 (1H), 4.50 (2H), 4.26-4.11 (2H), 3.99-3.72 (4H), 3.03 (1H), 2.72 (1H), 2.12-1.90 (4H), 1.61 (1H), 1.20 (3H), 0.72 (6H); ¹⁹F NMR (DMSO-d₆): δ −74.64, −74.69; MS ESI: 666.4 [M+H]⁺

### Example 47

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-1H-indole-5-carboxamide

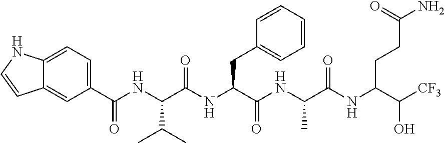 [SMILES: CC(C)[C@H](NC(=O)c1ccc2[nH]ccc2c1)C(=O)N[C@@H](Cc1ccccc1)C(=O)N[C@@H](C)C(=O)NC(CCC(N)=O)C(O)C(F)(F)F]

¹H NMR (DMSO-d₆): δ 11.31 (1H), 8.17-7.95 (4H), 7.84 (1H), 7.60 (1H), 7.40 (2H), 7.14-7.01 (6H), 6.70 (1H), 6.58-6.40 (2H), 4.60 (1H), 4.24-4.10 (2H), 3.98-3.72 (2H), 3.02 (1H), 2.68 (1H), 2.15-1.80 (4H), 1.60 (1H), 1.20 (3H), 0.82 (3H), 0.70 (3H); ¹⁹F NMR (DMSO-d₆): δ −74.62, −74.69; MS ESI: 661.3 [M+H]⁺

### Example 48

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-5-phenyl-1,3,4-oxadiazole-2-carboxamide

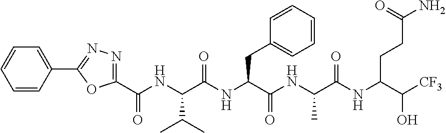 [SMILES: CC(C)[C@H](NC(=O)c1nnc(-c2ccccc2)o1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.87 (1H), 8.31 (1H), 8.15-8.00 (3H), 7.90 (1H), 7.70-7.52 (3H), 7.27-6.98 (6H), 6.70 (1H), 6.46 (1H), 4.60 (1H), 4.23 (2H), 3.98-3.72 (2H), 3.04 (1H), 2.73 (1H), 2.14-1.80 (4H), 1.60 (1H), 1.20 (3H), 0.82 (3H), 0.73 (3H); ¹⁹F NMR (DMSO-d₆): δ −74.67, −74.70; MS ESI: 690.3 [M+H]⁺

### Example 49

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4-(1,3,4-oxadiazol-2-yl)benzamide

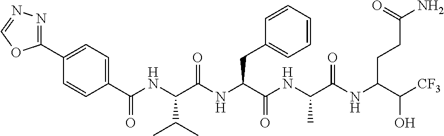 [SMILES: CC(C)[C@H](NC(=O)c1ccc(-c2nnco2)cc1)C(=O)N[C@@H](Cc1ccccc1)C(=O)N[C@@H](C)C(=O)NC(CCC(N)=O)C(O)C(F)(F)F]

¹H NMR (DMSO-d₆): δ 9.40 (1H), 8.43 (1H), 8.18-7.95 (5.5H), 7.96-7.80 (1.5H), 7.27-7.00 (6H), 6.70 (1H), 6.50 (1H), 4.58 (1H), 4.27-4.14 (2H), 3.98-3.75 (2H), 3.02 (1H), 2.79 (1H), 2.14-1.80 (4H), 1.60 (1H), 1.18 (3H), 0.82 (3H), 0.76 (3H); ¹⁹F NMR (DMSO-d₆): δ −74.63, −74.68; MS ESI: 690.4 [M+H]⁺

### Example 50

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-2-phenylthiazole-4-carboxamide

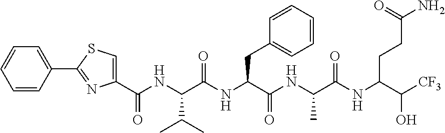 [SMILES: CC(C)[C@H](NC(=O)c1csc(-c2ccccc2)n1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.38 (1H), 8.32 (1H), 8.10 (0.5H), 8.05 (0.5H), 8.00 (3H), 7.92 (1H), 7.60-7.50 (3H), 7.23 (2H), 7.15 (3H), 7.05 (1H), 6.69 (1H), 6.50 (1H), 4.60 (1H), 4.53 (1H), 4.21 (1H), 3.97-3.72 (2H), 3.04 (1H), 2.77 (1H), 2.14-1.80 (4H), 1.60 (1H), 1.20 (3H), 0.80 (6H); ¹⁹F NMR (DMSO-d₆): δ −74.64, −74.68; MS ESI: 705.3 [M+H]⁺

### Example 51

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4′-(methylsulfonyl)-[1,1′-biphenyl]-4-carboxamide

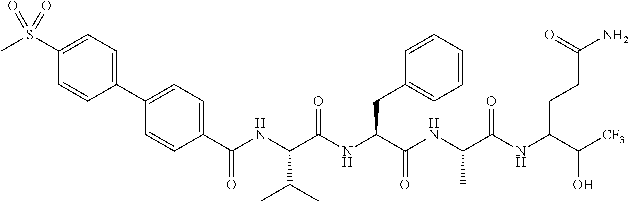 [SMILES: CC(C)[C@H](NC(=O)c1ccc(-c2ccc(S(C)(=O)=O)cc2)cc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.33 (1H), 8.13-7.93 (8H), 7.92-7.80 (3H), 7.27-7.04 (6H), 6.70 (1H), 6.48 (1H), 4.60 (1H), 4.22 (2H), 3.98-3.77 (2H), 3.25 (3H), 3.04 (1H), 2.77 (1H), 2.12-1.80 (4H), 1.61 (1H), 1.20 (3H), 0.82 (3H), 0.77 (3H); ¹⁹F NMR (DMSO-d₆): δ −74.64, −74.69; MS ESI: 776.3 [M+H]⁺

### Example 52

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-4′-fluoro-[1,1′-biphenyl]-4-carboxamide

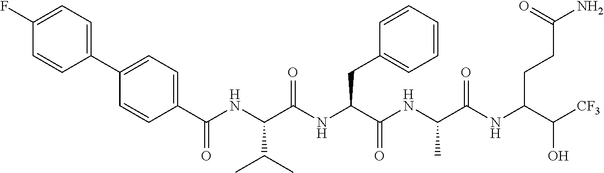 [SMILES: CC(C)[C@H](NC(=O)c1ccc(-c2ccc(F)cc2)cc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.26 (1H), 8.14-8.00 (2H), 7.99-7.82 (3H), 7.80-7.68 (4H), 7.30 (2H), 7.26-7.02 (6H), 6.70 (1H), 6.50 (1H), 4.60 (1H), 4.30-4.10 (2H), 3.99-3.72 (2H), 3.03 (1H), 2.78 (1H), 2.16-1.77 (4H), 1.63 (1H), 1.20 (3H), 0.80 (3H), 0.70 (3H); ¹⁹F NMR (DMSO-d₆): δ −74.64, −74.69, −115.0; MS ESI: 716.3 [M+H]⁺

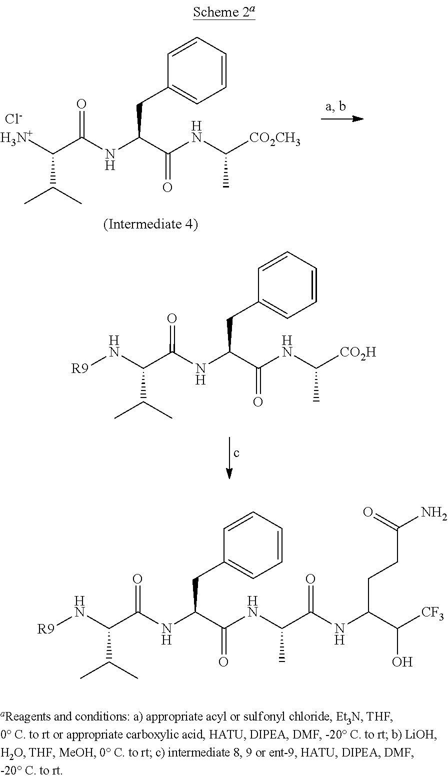

### Compound Examples Prepared According to Scheme 2

### Example 53

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)pyrazine-2-carboxamide

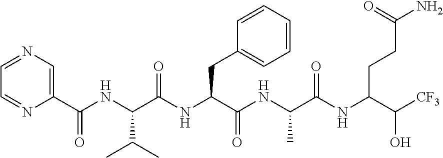 [SMILES: CC(C)[C@H](NC(=O)c1cnccn1)C(=O)N[C@@H](Cc1ccccc1)C(=O)N[C@@H](C)C(=O)NC(CCC(N)=O)C(O)C(F)(F)F]

¹H NMR (DMSO-d₆): δ 9.19 (1H), 8.90 (1H), 8.73 (1H), 8.45-8.35 (2H), 8.13 (0.5H), 8.03 (0.5H), 7.90 (1H), 7.27-7.10 (5H), 7.05 (1H), 6.70 (1H), 6.50 (1H), 4.60 (1H), 4.35 (1H), 4.20 (1H), 3.98-3.72 (2H), 3.02 (1H), 2.71 (1H), 2.14-1.80 (4H), 1.60 (1H), 1.20 (3H), 0.77 (6H); ¹⁹F NMR (DMSO-d₆): δ −74.64, −74.68; MS ESI: 624.4 [M+H]⁺

### Example 54

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)benzamide

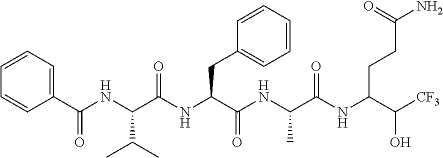 [SMILES: CC(C)[C@H](NC(=O)c1ccccc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.20 (1H), 8.07 (1.5H), 8.00 (0.5H), 7.88 (1H), 7.80 (2H), 7.50 (1H), 7.45 (2H), 7.23-7.02 (6H), 6.70 (1H), 6.50 (1H), 4.56 (1H), 4.24-4.12 (2H), 3.96-3.76 (2H), 3.01 (1H), 2.78 (1H), 2.10-1.80 (4H), 1.61 (1H), 1.18 (3H), 0.80 (3H), 0.72 (3H); ¹⁹F NMR (DMSO-d₆): δ −74.65, −74.70; MS ESI: 622.4 [M+H]⁺

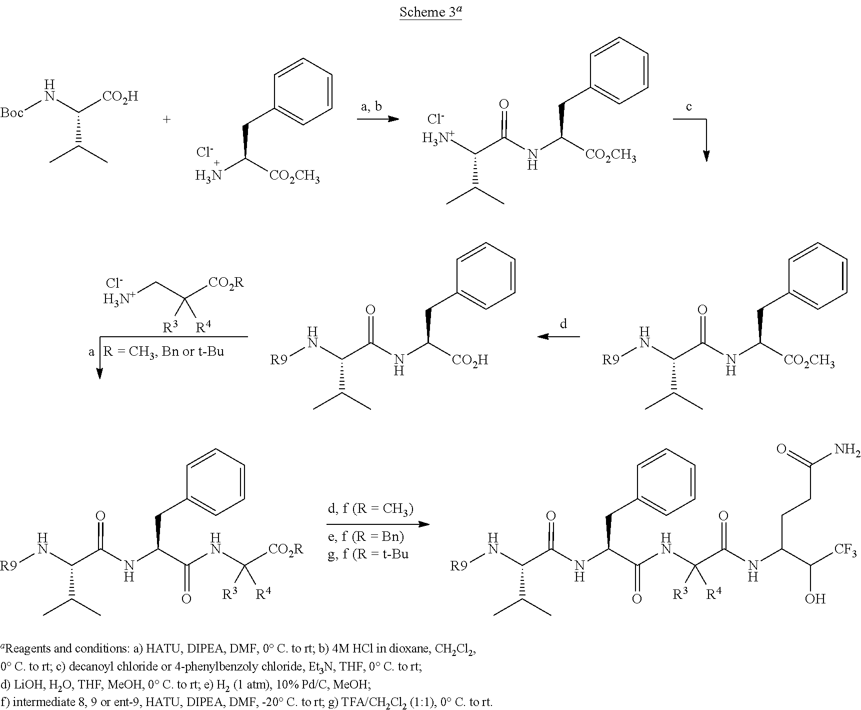

### Compound Examples Prepared According to Scheme 3

### Example 55

### (2S)—N¹-(6-Amino-1,1,1-trifluoro-2-hydroxy-6-oxohexan-3-yl)-2-((S)-2-((S)-2-decanamido-3-methylbutanamido)-3-phenylpropanamido)pentanediamide

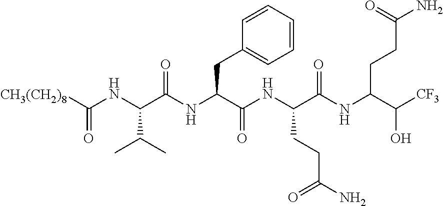

Compound was purified by recrystallization from ethanol/water with decolourization using activated charcoal.

¹H NMR (DMSO-d₆): δ 8.01-7.86 (3H), 7.71 (1H), 7.26-7.05 (7H), 6.80-6.64 (2H), 6.44 (1H), 4.55 (1H), 4.20 (1H), 4.03 (1H), 3.98-3.80 (2H), 3.01 (1H), 2.80 (1H), 2.20-1.94 (6H), 1.94-1.55 (5H), 1.50-1.30 (2H), 1.30-1.10 (12H), 0.82 (3H), 0.75 (6H); ¹⁹F NMR (DMSO-d₆): δ −74.61, −74.70; MS ESI: 729.5 [M+H]⁺

### Example 56

### N-((2S)-1-(((2S)-1-(((2S)-1-((6-Amino-1,1,1-trifluoro-2-hydroxy-6-oxohexan-3-yl)amino)-4-(methylthio)-1-oxobutan-2-yl)amino)-1-oxo-3-phenylpropan-2-yl)amino)-3-methyl-1-oxobutan-2-yl)decanamide

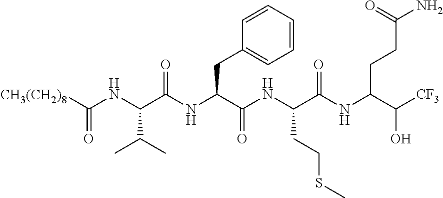

¹H NMR (DMSO-d₆): δ 8.10-7.82 (3H), 7.75 (1H), 7.23-7.08 (6H), 6.70 (1H), 6.50 (1H), 4.52 (1H), 4.30 (1H), 4.04 (1H), 3.98-3.80 (2H), 3.00 (1H), 2.80 (1H), 2.44-2.30 (2H), 2.20-1.55 (9H), 2.00 (3H), 1.52-1.30 (2H), 1.30-1.10 (12H), 0.81 (3H), 0.73 (6H); MS ESI: 732.5 [M+H]⁺

### Example 57

### N-((2S)-1-(((2S)-1-(((2S)-1-((6-Amino-1,1,1-trifluoro-2-hydroxy-6-oxohexan-3-yl)amino)-4-methyl-1-oxopentan-2-yl)amino)-1-oxo-3-phenylpropan-2-yl)amino)-3-methyl-1-oxobutan-2-yl)decanamide

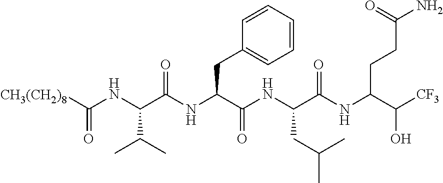

¹H NMR (DMSO-d₆): δ 7.98 (1H), 7.95 (0.5H), 7.83 (1.5H), 7.73 (1H), 7.23-7.06 (6H), 6.70 (1H), 6.47 (1H), 4.53 (1H), 4.44 (1H), 4.05 (1H), 3.96-3.78 (2H), 3.00 (1H), 2.80 (1H), 2.20-1.80 (6H), 1.70-1.33 (6H), 1.30-1.10 (12H), 0.83 (9H), 0.73 (6H); MS ESI: 714.5 [M+H]⁺

### Example 58

### N-((2S)-1-(((2S)-1-(((2S)-1-((6-amino-1,1,1-trifluoro-2-hydroxy-6-oxohexan-3-yl)amino)-3-methyl-1-oxobutan-2-yl)amino)-1-oxo-3-phenylpropan-2-yl)amino)-3-methyl-1-oxobutan-2-yl)decanamide

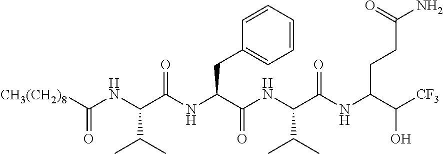

¹H NMR (DMSO-d₆): δ 8.04-7.87 (2H), 7.79-7.70 (2H), 7.23-7.04 (6H), 6.70 (1H), 6.44 (1H), 4.55 (1H), 4.19-4.01 (2H), 3.90 (1H), 3.80 (1H), 2.98 (1H), 2.78 (1H), 2.20-1.76 (7H), 1.63 (1H), 1.50-1.32 (2H), 1.30-1.13 (12H), 0.80 (9H), 0.75 (6H); ¹⁹F NMR (DMSO-d₆): δ −74.61, −74.69; MS ESI: 700.5 [M+H]⁺

### Example 59

### N-((2S)-1-(((2S)-1-(((2S)-1-((6-Amino-1,1,1-trifluoro-2-hydroxy-6-oxohexan-3-yl)amino)-1-oxo-3-phenylpropan-2-yl)amino)-1-oxo-3-phenylpropan-2-yl)amino)-3-methyl-1-oxobutan-2-yl)decanamide

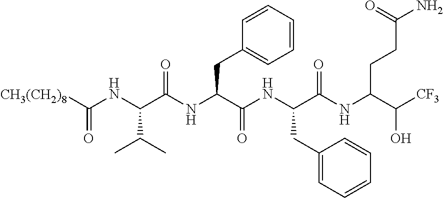

¹H NMR (DMSO-d₆): δ 8.03 (1H), 8.00 (1H), 7.90 (1H), 7.70 (1H), 7.30-7.07 (11H), 6.72 (1H), 6.47 (1H), 4.60-4.42 (2H), 4.05 (1H), 4.00-3.74 (2H), 3.00-2.82 (2H), 2.80-2.62 (2H), 2.20-1.97 (3H), 1.97-1.73 (3H), 1.62 (1H), 1.52-1.32 (2H), 1.30-1.15 (12H), 0.82 (3H), 0.70 (6H); MS ESI: 748.5 [M+H]⁺

### Example 60

### N-((2S)-1-(((2S)-1-(((2R)-1-(((3S)-6-amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)decanamide

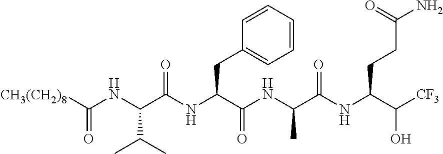

Single diastereomer; prepared using intermediate 9.

¹H NMR (DMSO-d₆): δ 8.07 (1H), 7.95 (1H), 7.78 (1H), 7.70 (1H), 7.25-7.10 (6H), 6.70 (1H), 6.40 (1H), 4.43 (1H), 4.17 (1H), 4.09 (1H), 3.96-3.79 (2H), 2.94 (1H), 2.81 (1H), 2.20-1.80 (6H), 1.68 (1H), 1.51-1.38 (2H), 1.30-1.14 (12H), 1.05 (3H), 0.83 (3H), 0.74 (6H); ¹⁹F NMR (DMSO-d₆): δ −74.67; MS ESI: 672.5 [M+H]⁺

### Example 61

### N-((2S)-1-(((2S)-1-(((2R)-1-(((3R)-6-amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)decanamide

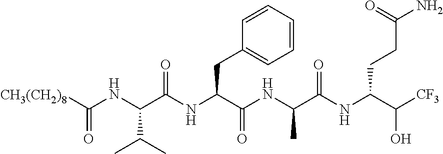

Single diastereomer; prepared using intermediate ent-9; purified by slow recrystallization from EtOAcl/EtOH.

¹H NMR (DMSO-d₆): δ 8.10 (1H), 7.97 (1H), 7.81 (1H), 7.64 (1H), 7.25-7.10 (6H), 6.66 (1H), 6.43 (1H), 4.42 (1H), 4.18 (1H), 4.10 (1H), 3.96-3.77 (2H), 2.95 (1H), 2.80 (1H), 2.20-1.80 (6H), 1.62 (1H), 1.44-1.36 (2H), 1.30-1.14 (12H), 1.02 (3H), 0.82 (3H), 0.74 (6H); ¹⁹F NMR (DMSO-d₆): δ −74.62; MS ESI: 672.5 [M+H]⁺

### Example 62

### N-((2S)-1-(((2S)-1-(((2R)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-[1,1′-biphenyl]-4-carboxamide

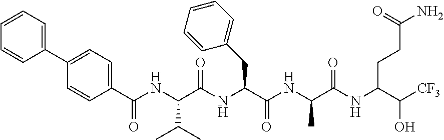 [SMILES: CC(C)[C@H](NC(=O)c1ccc(-c2ccccc2)cc1)C(=O)N[C@@H](Cc1ccccc1)C(=O)N[C@H](C)C(=O)NC(CCC(N)=O)C(O)C(F)(F)F]

¹H NMR (DMSO-d₆): δ 8.32-8.16 (2H), 8.03 (1H), 7.99-7.89 (2H), 7.83-7.62 (5H), 7.55-7.43 (2H), 7.40 (1H), 7.24-7.01 (6H), 6.70 (1H), 6.42 (1H), 4.50 (1H), 4.32-4.12 (2H), 3.99-3.77 (2H), 2.96 (1H), 2.81 (1H), 2.27-1.78 (4H), 1.62 (1H), 1.03 (3H), 0.81 (6H); ¹⁹F NMR (DMSO-d₆): δ −74.66, −74.69; MS ESI: 698.4 [M+H]⁺

### Example 63

### N-((2S)-1-(((2S)-1-((2-((6-Amino-1,1,1-trifluoro-2-hydroxy-6-oxohexan-3-yl)amino)-2-oxoethyl)amino)-1-oxo-3-phenylpropan-2-yl)amino)-3-methyl-1-oxobutan-2-yl)-[1,1′-biphenyl]-4-carboxamide

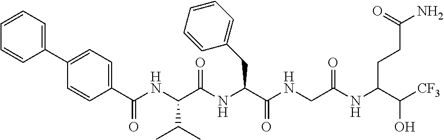 [SMILES: CC(C)[C@H](NC(=O)c1ccc(-c2ccccc2)cc1)C(=O)N[C@@H](Cc1ccccc1)C(=O)NCC(=O)NC(CCC(N)=O)C(O)C(F)(F)F]

¹H NMR (DMSO-d₆): δ 8.30-8.20 (2H), 8.10 (1H), 7.91 (2H), 7.84 (1H), 7.80-7.64 (4H), 7.47 (2H), 7.40 (1H), 7.25-7.01 (6H), 6.70 (1H), 6.50 (1H), 4.60 (1H), 4.23 (1H), 4.00-3.80 (2H), 3.79-3.59 (2H), 3.01 (1H), 2.81 (1H), 2.16-1.79 (4H), 1.62 (1H), 0.83 (3H), 0.78 (3H); ¹⁹F NMR (DMSO-d₆): δ −74.63, −74.68; MS ESI: 684.4 [M+H]⁺

### Example 64

### N-((2S)-1-(((2S)-1-((1-((6-Amino-1,1,1-trifluoro-2-hydroxy-6-oxohexan-3-yl)carbamoyl)cyclopropyl)amino)-1-oxo-3-phenylpropan-2-yl)amino)-3-methyl-1-oxobutan-2-yl)-[1,1′-biphenyl]-4-carboxamide

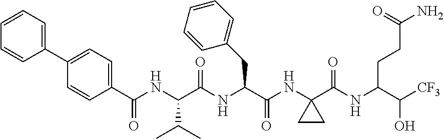 [SMILES: CC(C)[C@H](NC(=O)c1ccc(-c2ccccc2)cc1)C(=O)N[C@@H](Cc1ccccc1)C(=O)NC1(C(=O)NC(CCC(N)=O)C(O)C(F)(F)F)CC1]

Isolated as a 2.4:1 mix of diastereomers.

¹H NMR (DMSO-d₆): δ 8.21 (0.7H), 8.15 (0.3H), 8.10 (0.3H), 8.00 (0.7H), 7.98 (0.3H), 7.90 (0.7H), 7.70-7.58 (2H), 7.50-7.36 (4H), 7.18 (2H), 7.10 (2H), 6.97-6.75 (6H), 6.40 (0.3H), 6.38 (0.7H), 6.00 (1H), 4.00 (1H), 3.90 (1H), 3.67-3.50 (2H), 2.70-2.50 (2H), 1.86-1.30 (5H), 0.94-0.72 (1.7H), 0.72-0.48 (6.3H), 0.30 (0.7H), 0.13 (0.3H), 0.02 (1H); ¹⁹F NMR (DMSO-d₆): δ −74.62 (minor diastereomer), −74.84 (major diastereomer); MS ESI: 710.4 [M+H]⁺

### Example 65

### N-((2S)-1-(((2S)-1-(((2R)-1-(((3S)-6-amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-[1,1′-biphenyl]-4-carboxamide

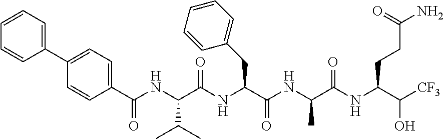 [SMILES: CC(C)[C@H](NC(=O)c1ccc(-c2ccccc2)cc1)C(=O)N[C@@H](Cc1ccccc1)C(=O)N[C@H](C)C(=O)N[C@@H](CCC(N)=O)C(O)C(F)(F)F]

Single diastereomer; prepared using intermediate 9.

¹H NMR (DMSO-d₆): δ 8.30 (1H), 8.20 (1H), 8.03 (1H), 7.97 (2H), 7.81 (1H), 7.76-7.68 (4H), 7.50 (2H), 7.40 (1H), 7.24-7.05 (6H), 6.70 (1H), 6.40 (1H), 4.50 (1H), 4.33-4.16 (2H), 3.97-3.79 (2H), 2.95 (1H), 2.82 (1H), 2.10-1.78 (4H), 1.70 (1H), 1.05 (3H), 0.82 (3H), 0.78 (3H); ¹⁹F NMR (DMSO-d₆): δ −74.66; MS ESI: 698.4 [M+H]⁺

### Example 66

### N-((2S)-1-(((2S)-1-((1-(((3S)-6-Amino-1,1,1-trifluoro-2-hydroxy-6-oxohexan-3-yl)carbamoyl)cyclobutyl)amino)-1-oxo-3-phenylpropan-2-yl)amino)-3-methyl-1-oxobutan-2-yl)-[1,1′-biphenyl]-4-carboxamide

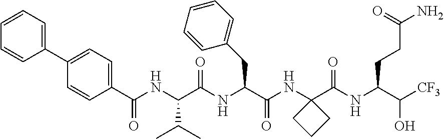 [SMILES: CC(C)[C@H](NC(=O)c1ccc(-c2ccccc2)cc1)C(=O)N[C@@H](Cc1ccccc1)C(=O)NC1(C(=O)N[C@@H](CCC(N)=O)C(O)C(F)(F)F)CCC1]

Single diastereomer; prepared using intermediate 9.

¹H NMR (DMSO-d₆): δ 8.40 (1H), 8.35 (2H), 7.97 (2H), 7.73 (4H), 7.50 (2H), 7.40 (1H), 7.30-7.12 (6H), 7.06 (1H), 6.70 (1H), 6.36 (1H), 4.43 (1H), 4.23 (1H), 3.92 (1H), 3.81 (1H), 3.06-2.88 (2H), 2.52 (1H), 2.24 (1H), 2.13-1.53 (9H), 0.90 (3H), 0.84 (3H); ¹⁹F NMR (DMSO-d₆): δ −74.62; MS ESI: 724.4 [M+H]⁺

### Example 67

### N-((2S)-1-(((2S)-1-((1-((6-Amino-1,1,1-trifluoro-2-hydroxy-6-oxohexan-3-yl)amino)-2-methyl-1-oxopropan-2-yl)amino)-1-oxo-3-phenylpropan-2-yl)amino)-3-methyl-1-oxobutan-2-yl)-[1,1′-biphenyl]-4-carboxamide

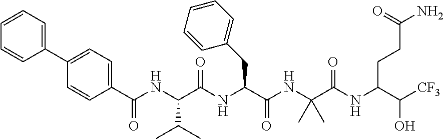 [SMILES: CC(C)[C@H](NC(=O)c1ccc(-c2ccccc2)cc1)C(=O)N[C@@H](Cc1ccccc1)C(=O)NC(C)(C)C(=O)NC(CCC(N)=O)C(O)C(F)(F)F]

Purified by flash chromatography on silica gel eluting 0-4% MeOH in EtOAc.

¹H NMR (DMSO-d₆): δ 8.31-8.18 (2H), 7.99-7.88 (3H), 7.80-7.64 (4H), 7.47 (2H), 7.42-7.27 (2H), 7.25-7.00 (6H), 6.70 (1H), 6.36 (1H), 4.46 (1H), 4.24 (1H), 3.98-3.80 (2H), 3.01 (1H), 2.82 (1H), 2.18-1.80 (4H), 1.62 (1H), 1.32-1.20 (6H), 0.95-0.75 (6H); ¹⁹F NMR (DMSO-d₆): δ −74.57, −74.67; MS ESI: 712.4 [M+H]⁺

### Example 68

### N-((2S)-1-(((2S)-1-(((2S)-1-((6-Amino-1,1,1-trifluoro-2-hydroxy-6-oxohexan-3-yl)amino)-1-oxo-3-phenylpropan-2-yl)amino)-1-oxo-3-phenylpropan-2-yl)amino)-3-methyl-1-oxobutan-2-yl)-[1,1′-biphenyl]-4-carboxamide

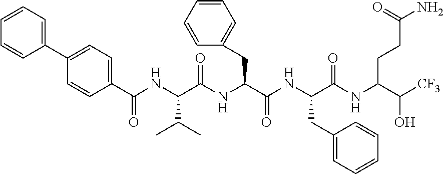 [SMILES: CC(C)[C@H](NC(=O)c1ccc(-c2ccccc2)cc1)C(=O)N[C@@H](Cc1ccccc1)C(=O)N[C@@H](Cc1ccccc1)C(=O)NC(CCC(N)=O)C(O)C(F)(F)F]

¹H NMR (DMSO-d₆): δ 8.27 (1H), 8.13 (2H), 8.00 (1H), 7.94 (2H), 7.76 (4H), 7.50 (2H), 7.42 (1H), 7.28-7.02 (11H), 6.72 (1H), 6.55 (1H), 4.62-4.46 (2H), 4.23 (1H), 3.99-3.77 (2H), 3.05-2.88 (2H), 2.86-2.63 (2H), 2.13- 1.74 (4H), 1.65 (1H), 0.83 (3H), 0.74 (3H); ¹⁹F NMR (DMSO-d₆): δ −74.69, −74.72; MS ESI: 774.4 [M+H]⁺

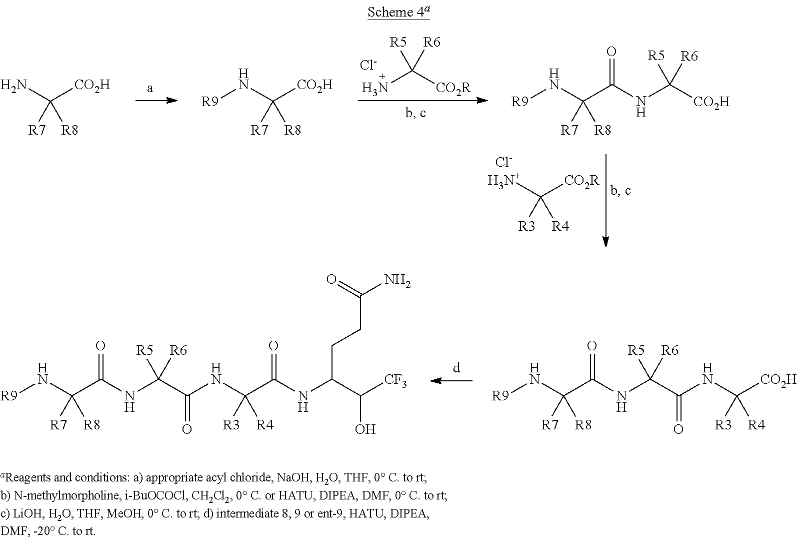

### Compound Examples Prepared According to Scheme 4

### Example 69

### N-((2S)-1-(((2R)-1-(((2R)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-[1,1′-biphenyl]-4-carboxamide

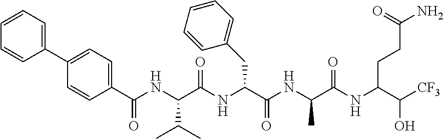 [SMILES: CC(C)[C@H](NC(=O)c1ccc(-c2ccccc2)cc1)C(=O)N[C@H](Cc1ccccc1)C(=O)N[C@H](C)C(=O)NC(CCC(N)=O)C(O)C(F)(F)F]

¹H NMR (DMSO-d₆): δ 8.42 (1H), 8.30 (1H), 8.14 (1H), 7.95 (2H), 7.80 (0.5H), 7.73 (4.5H), 7.46 (2H), 7.40 (1H), 7.24 (2H), 7.20-7.05 (4H), 6.70 (1H), 6.45 (1H), 4.56 (1H), 4.22 (1H), 4.17 (1H), 3.97-3.74 (2H), 3.12 (1H), 2.68 (1H), 2.10-1.80 (4H), 1.60 (1H), 1.35-1.20 (3H), 0.75 (3H), 0.58 (3H); ¹⁹F NMR (DMSO-d₆): δ −74.63, −74.75; MS ESI: 698.4 [M+H]⁺

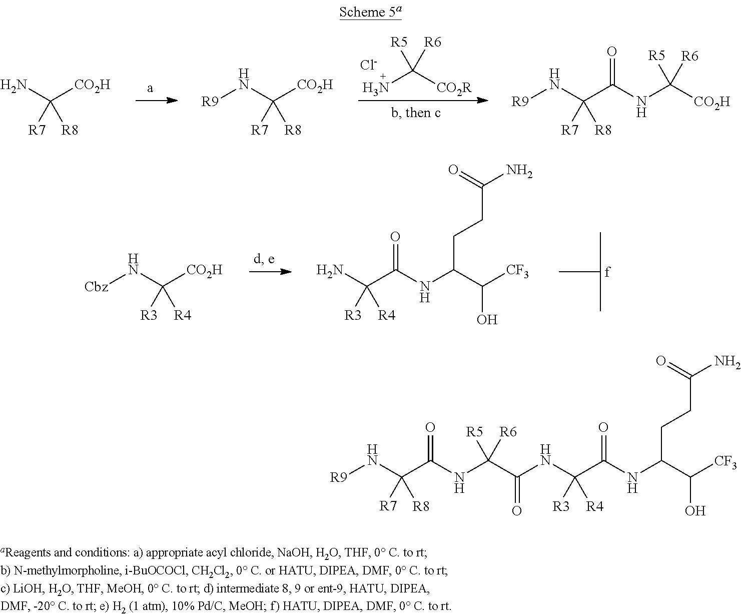

### Compound Examples Prepared According to Scheme 5

### Example 70

### N-((2S)-1-(((2R)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-[1,1′-biphenyl]-4-carboxamide

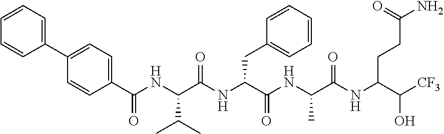 [SMILES: CC(C)[C@H](NC(=O)c1ccc(-c2ccccc2)cc1)C(=O)N[C@H](Cc1ccccc1)C(=O)N[C@@H](C)C(=O)NC(CCC(N)=O)C(O)C(F)(F)F]

Isolated as a 4:1 mixture of diastereomers.

¹H NMR (DMSO-d₆): δ 8.48 (1H), 8.36 (1H), 8.05 (1H), 7.97 (2H), 7.88 (1H), 7.76 (4H), 7.50 (2H), 7.40 (1H), 7.23 (2H), 7.20-7.06 (4H), 6.70 (1H), 6.53 (1H), 4.52 (1H), 4.22 (1H), 4.17 (1H), 3.96 (1H), 3.83 (1H), 3.05 (1 H), 2.76 (1H), 2.18-1.82 (4H), 1.61 (1H), 1.18 (3H), 0.78 (3H), 0.57 (3H); ¹⁹F NMR (DMSO-d₆): δ −74.63; MS ESI: 698.4 [M+H]⁺

### Example 71

### N-((2S)-1-((2-(((2S)-1-((6-Amino-1,1,1-trifluoro-2-hydroxy-6-oxohexan-3-yl)amino)-1-oxopropan-2-yl)amino)-2-oxoethyl)amino)-3-methyl-1-oxobutan-2-yl)-[1,1′-biphenyl]-4-carboxamide

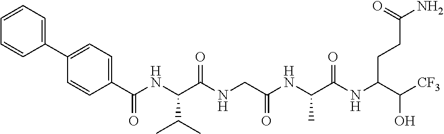 [SMILES: CC(C)[C@H](NC(=O)c1ccc(-c2ccccc2)cc1)C(=O)NCC(=O)N[C@@H](C)C(=O)NC(CCC(N)=O)C(O)C(F)(F)F]

¹H NMR (DMSO-d₆): δ 8.44-8.30 (2H), 8.00 (2H), 7.90 (2H), 7.76 (4H), 7.48 (2H), 7.40 (1H), 7.18 (1H), 6.68 (1H), 6.53 (1H), 4.23 (2H), 3.99-3.80 (2H), 3.76 (2H), 2.20-1.80 (4H), 1.62 (1H), 1.20 (3H), 0.97 (6H); ¹⁹F NMR (DMSO-d₆): δ −74.64. −74.66; MS ESI: 608.4 [M+H]⁺

### Example 72

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxopropan-2-yl)amino)-3-methyl-1-oxobutan-2-yl)-[1,1′-biphenyl]-4-carboxamide

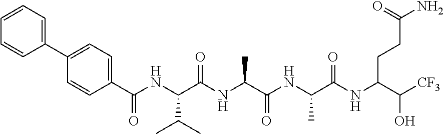 [SMILES: CC(C)[C@H](NC(=O)c1ccc(-c2ccccc2)cc1)C(=O)N[C@@H](C)C(=O)N[C@@H](C)C(=O)NC(CCC(N)=O)C(O)C(F)(F)F]

¹H NMR (DMSO-d₆): δ 8.32 (1H), 8.18 (1H), 8.00-7.80 (4H), 7.72 (4H), 7.48 (2H), 7.40 (1H), 7.17 (1H), 6.68 (1H), 6.56 (1H), 4.30 (2H), 4.20 (1H), 3.98-3.73 (2H), 2.20-1.80 (4H), 1.60 (1H), 1.25-1.10 (6H), 0.93 (6H); ¹⁹F NMR (DMSO-d₆): δ −74.53, −74.54; MS ESI: 622.4 [M+H]⁺

### Example 73

### N-((2S)-1-(((2S)-1-(((2S)-1-((6-Amino-1,1,1-trifluoro-2-hydroxy-6-oxohexan-3-yl)amino)-1-oxopropan-2-yl)amino)-4-(methylthio)-1-oxobutan-2-yl)amino)-3-methyl-1-oxobutan-2-yl)-[1,1′-biphenyl]-4-carboxamide

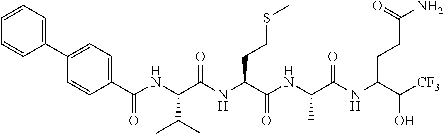 [SMILES: CSCC[C@H](NC(=O)[C@@H](NC(=O)c1ccc(-c2ccccc2)cc1)C(C)C)C(=O)N[C@@H](C)C(=O)NC(CCC(N)=O)C(O)C(F)(F)F]

¹H NMR (DMSO-d₆): δ 8.39 (1H), 8.16 (1H), 8.00-7.85 (4H), 7.75 (4H), 7.47 (2H), 7.40 (1H), 7.19 (0.5H), 7.11 (0.5H), 6.70 (1H), 4.38 (1H), 4.30-4.14 (2H), 3.96-3.76 (2H), 2.42 (2H), 2.20-1.72 (6H), 2.00 (3H), 1.60 (1H), 1.20 (3H), 0.94 (6H); ¹⁹F NMR (DMSO-d₆): δ −74.63, −74.69; MS ESI: 682.4 [M+H]⁺

### Example 74

### N-((2S)-1-(((2S)-1-(((2S)-1-((6-Amino-1,1,1-trifluoro-2-hydroxy-6-oxohexan-3-yl)amino)-1-oxopropan-2-yl)amino)-3-methyl-1-oxobutan-2-yl)amino)-3-methyl-1-oxobutan-2-yl)-[1,1′-biphenyl]-4-carboxamide

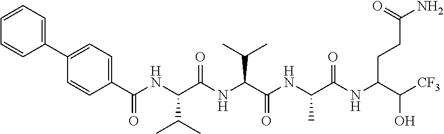 [SMILES: CC(C)[C@H](NC(=O)c1ccc(-c2ccccc2)cc1)C(=O)N[C@H](C(=O)N[C@@H](C)C(=O)NC(CCC(N)=O)C(O)C(F)(F)F)C(C)C]

¹H NMR (DMSO-d₆): δ 8.40 (1H), 8.03-7.80 (5H), 7.74 (4H), 7.46 (2H), 7.40 (1H), 7.20 (0.5H), 7.06 (0.5H), 6.65 (1H), 6.47 (1H), 4.33 (1H), 4.30-4.10 (2H), 3.95-3.72 (2H), 2.21-1.79 (5H), 1.60 (1H), 1.23-1.10 (3H), 0.92 (6H), 0.83 (6H); ¹⁹F NMR (DMSO-d₆): δ −74.66, −74.70; MS ESI: 650.4 [M+H]⁺

### Example 75

### N-((5S,8S,11S)-17-amino-8-benzyl-11-methyl-6,9,12,17-tetraoxo-14-(2,2,2-trifluoro-1-hydroxyethyl)-2-thia-7,10,13-triazaheptadecan-5-yl)-[1,1′-biphenyl]-4-carboxamide

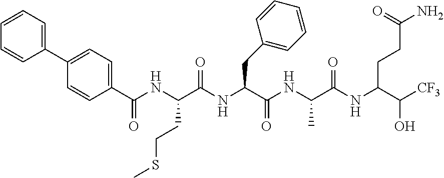 [SMILES: CSCC[C@H](NC(=O)c1ccc(-c2ccccc2)cc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.54 (1H), 8.03 (2H), 7.98 (2H), 7.89 (1H), 7.75 (4H), 7.48 (2H), 7.40 (1H), 7.23-7.05 (6H), 6.70 (1H), 6.48 (1H), 4.58 (1H), 4.53 (1H), 4.20 (1H), 4.00-3.76 (2H), 3.03 (1H), 2.80 (1H), 2.40 (2H), 2.15-1.80 (5H), 2.02 (3H), 1.62 (1H), 1.21 (3H); ¹⁹F NMR (DMSO-d₆): δ −74.64, −74.66; MS ESI: 730.4 [M+H]⁺

### Example 76

### N-((2R)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-3-methyl-1-oxobutan-2-yl)-[1,1′-biphenyl]-4-carboxamide

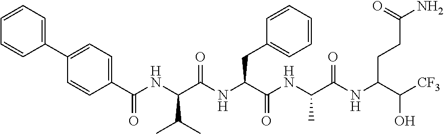 [SMILES: CC(C)[C@@H](NC(=O)c1ccc(-c2ccccc2)cc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.44 (1H), 8.31 (1H), 8.17 (1H), 7.95 (2H), 7.87 (0.5H), 7.80 (0.5H), 7.77 (4H), 7.44 (2H), 7.40 (1H), 7.24 (2H), 7.20-7.07 (4H), 6.70 (1H), 4.57 (1H), 4.22 (1H), 4.18 (1H), 3.96-3.75 (2H), 3.13 (1H), 2.73 (1H), 2.10-1.80 (4H), 1.60 (1H), 1.25 (3H), 0.77 (3H), 0.58 (3H); ¹⁹F NMR (DMSO-d₆): δ −74.63, −74.75; MS ESI: 698.4 [M+H]⁺

### Example 77

### N-((1S)-2-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-2-oxo-1-phenylethyl)-[1,1′-biphenyl]-4-carboxamide

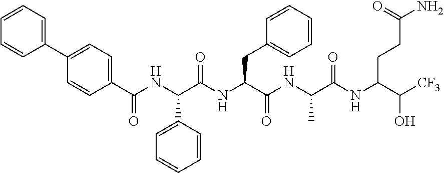 [SMILES: C[C@H](NC(=O)[C@H](Cc1ccccc1)NC(=O)[C@@H](NC(=O)c1ccc(-c2ccccc2)cc1)c1ccccc1)C(=O)NC(CCC(N)=O)C(O)C(F)(F)F]

¹H NMR (DMSO-d₆): δ 8.81 (1H), 8.41 (1H), 8.10-7.92 (3H), 7.86 (1H), 7.75 (4H), 7.50 (2H), 7.40 (1H), 7.38-7.00 (11H), 6.70 (1H), 6.48 (1H), 5.63 (1H), 4.60 (1H), 4.20 (1H), 3.98-3.78 (2H), 3.09 (1H), 2.82 (1H), 2.17-1.80 (3H), 1.61 (1H), 1.20 (3H); ¹⁹F NMR (DMSO-d₆): δ −74.64, −74.67; MS ESI: 732.4 [M+H]⁺

### Example 78

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-1-oxo-3-phenylpropan-2-yl)-[1,1′-biphenyl]-4-carboxamide

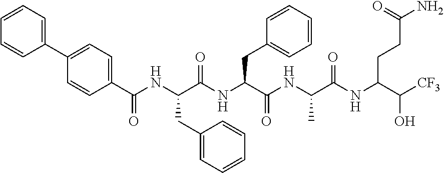 [SMILES: C[C@H](NC(=O)[C@H](Cc1ccccc1)NC(=O)[C@H](Cc1ccccc1)NC(=O)c1ccc(-c2ccccc2)cc1)C(=O)NC(CCC(N)=O)C(O)C(F)(F)F]

¹H NMR (DMSO-d₆): δ 8.60 (1H), 8.17 (2H), 7.90 (1H), 7.84 (2H), 7.72 (4H), 7.50 (2H), 7.40 (1H), 7.33-7.09 (11H), 6.70 (1H), 6.49 (1H), 4.63 (1H), 4.58 (1H), 4.22 (1H), 4.00-3.78 (2H), 3.03 (2H), 2.95 (1H), 2.82 (1H), 2.18-1.80 (3H), 1.61 (1H), 1.21 (3H); ¹⁹F NMR (DMSO-d₆): δ −74.62, −74.65; MS ESI: 746.4 [M+H]⁺

### Example 79

### N-(2-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-2-oxoethyl)-[1,1′-biphenyl]-4-carboxamide

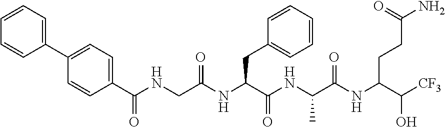 [SMILES: C[C@H](NC(=O)[C@H](Cc1ccccc1)NC(=O)CNC(=O)c1ccc(-c2ccccc2)cc1)C(=O)NC(CCC(N)=O)C(O)C(F)(F)F]

¹H NMR (DMSO-d₆): δ 8.79 (1H), 8.21-8.12 (2H), 7.94 (2H), 7.87-7.63 (5H), 7.45 (2H), 7.40 (1H), 7.26-7.07 (6H), 6.70 (1H), 6.47 (1H), 4.57 (1H), 4.21 (1H), 4.00-3.70 (3H), 3.02 (1H), 2.80 (1H), 2.15-1.80 (4H), 1.62 (1H), 1.21 (3H); ¹⁹F NMR (DMSO-d₆): δ −74.62, −74.68; MS ESI: 656.4 [M+H]⁺

### Example 80

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)amino)-1-oxopropan-2-yl)-[1,1′-biphenyl]-4-carboxamide

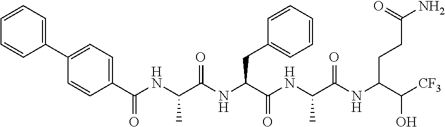 [SMILES: C[C@H](NC(=O)c1ccc(-c2ccccc2)cc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.57 (1H), 8.03-7.95 (4H), 7.82 (1H), 7.77 (4H), 7.50 (2H), 7.40 (1H), 7.22-7.05 (6H), 6.70 (1H), 6.50 (1H), 4.50 (1H), 4.40 (1H), 4.20 (1H), 4.00-3.77 (2H), 3.02 (1H), 2.81 (1H), 2.16-1.80 (3H), 1.61 (1H), 2.22 (6H); ¹⁹F NMR (DMSO-d₆): δ −74.65, −74.68; MS ESI: 670.4 [M+H]⁺

### Example 81

### N-((5R,8S,11S)-17-amino-8-benzyl-11-methyl-6,9,12,17-tetraoxo-14-(2,2,2-trifluoro-1-hydroxyethyl)-2-thia-7,10,13-triazaheptadecan-5-yl)-[1,1′-biphenyl]-4-arboxamide

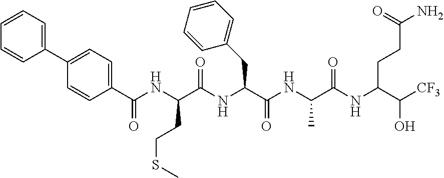 [SMILES: CSCC[C@@H](NC(=O)c1ccc(-c2ccccc2)cc1)C(=O)N[C@@H](Cc1ccccc1)C(=O)N[C@@H](C)C(=O)NC(CCC(N)=O)C(O)C(F)(F)F]

¹H NMR (DMSO-d₆): δ 8.52 (1H), 8.35 (1H), 8.17 (1H), 7.96 (2H), 7.90 (0.5H), 7.72 (4.5H), 7.48 (2H), 7.40 (1H), 7.24-7.04 (6H), 6.70 (1H), 6.50 (1H), 4.59 (1H), 4.51 (1H), 4.21 (1H), 3.98-3.78 (2H), 3.10 (1H), 2.73 (1H), 2.38-2.15 (2H), 2.12-1.80 (3H), 2.00 (3H), 1.80-1.48 (3H), 1.24 (3H); ¹⁹F NMR (DMSO-d₆): δ −74.62, −74.72; MS ESI: 730.4 [M+H]⁺

### Example 82

### N-((5R,8R,11S)-17-Amino-8-benzyl-11-methyl-6,9,12,17-tetraoxo-14-(2,2,2-trifluoro-1-hydroxyethyl)-2-thia-7,10,13-triazaheptadecan-5-yl)-[1,1′-biphenyl]-4-carboxamide

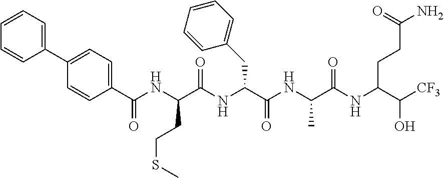 [SMILES: CSCC[C@@H](NC(=O)c1ccc(-c2ccccc2)cc1)C(=O)N[C@H](Cc1ccccc1)C(=O)N[C@@H](C)C(=O)NC(CCC(N)=O)C(O)C(F)(F)F]

¹H NMR (DMSO-d₆): δ 8.60 (0.5H), 8.56 (0.5H), 8.18-8.05 (2H), 7.98 (2H), 7.85 (0.5H), 7.75 (4.5H), 7.48 (2H), 7.40 (1H), 7.23-7.11 (6H), 6.77 (0.5H), 6.67 (0.5H), 6.45 (1H), 4.50 (2H), 4.20 (1H), 3.99-3.78 (2H), 2.98 (1H), 2.84 (1H), 2.44 (2H), 2.15-1.52 (6H), 2.00 (3H), 1.11 (3H); ¹⁹F NMR (DMSO-d₆): δ −74.62, −74.65; MS ESI: 730.3 [M+H]⁺

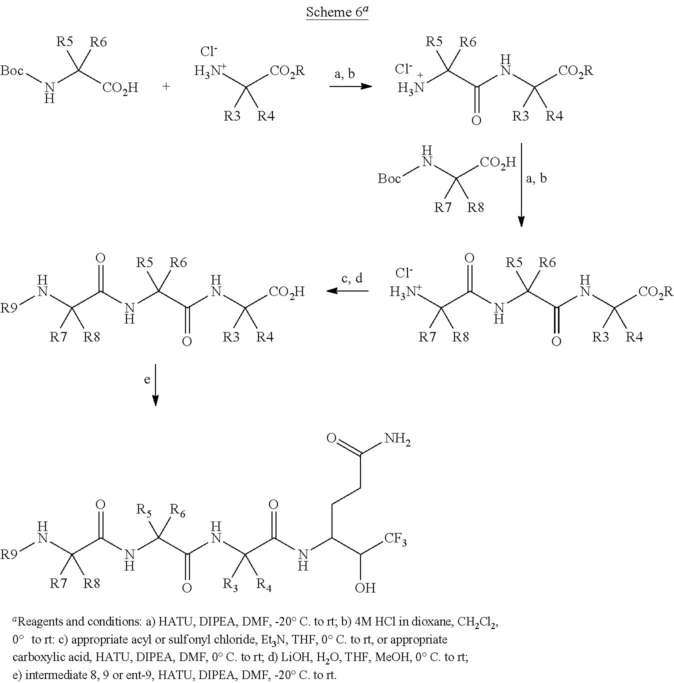

### Compound Examples Prepared According to Scheme 6

### Example 83

### 2-((S)-2-([1,1′-Biphenyl]-4-carboxamido)-3-methylbutanamido)-N-((2S)-1-((6-amino-1,1,1-trifluoro-2-hydroxy-6-oxohexan-3-yl)amino)-1-oxopropan-2-yl)-2,3-dihydro-1H-indene-2-carboxamide

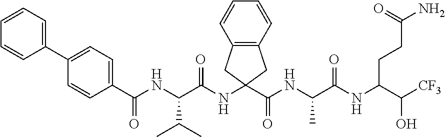 [SMILES: CC(C)[C@H](NC(=O)c1ccc(-c2ccccc2)cc1)C(=O)NC1(C(=O)N[C@@H](C)C(=O)NC(CCC(N)=O)C(O)C(F)(F)F)Cc2ccccc2C1]

¹H NMR (DMSO-d₆): δ 8.72 (1H), 8.48 (1H), 7.99 (2H), 7.75 (5H), 7.50 (3H), 7.40 (1H), 7.22-7.00 (5H), 6.70 (0.5H), 6.63 (0.5H), 6.45 (1H), 4.16 (1H), 4.01 (1H), 3.98-3.73 (2H), 3.58 (1H), 3.39 (1H), 3.24 (2H), 2.18-1.80 (4H), 1.62 (1H), 1.18 (3H), 0.94 (3H), 0.77 (3H); ¹⁹F NMR (DMSO-d₆): δ −74.60, −74.66; MS ESI: 710.4 [M+H]⁺

### Example 84

### N-((2S)-1-(((1S)-2-(((2S)-1-((6-Amino-1,1,1-trifluoro-2-hydroxy-6-oxohexan-3-yl)amino)-1-oxopropan-2-yl)amino)-2-oxo-1-phenylethyl)amino)-3-methyl-1-oxobutan-2-yl)-[1,1′-biphenyl]-4-carboxamide

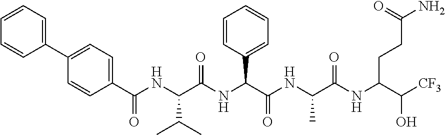 [SMILES: CC(C)[C@H](NC(=O)c1ccc(-c2ccccc2)cc1)C(=O)N[C@H](C(=O)N[C@@H](C)C(=O)NC(CCC(N)=O)C(O)C(F)(F)F)c1ccccc1]

¹H NMR (DMSO-d₆): δ 8.42 (3H), 7.96 (2H), 7.85 (0.5H), 7.76 (4.5H), 7.50 (2H), 7.40 (3H), 7.36-7.20 (3H), 7.15 (0.5H), 7.04 (0.5H), 6.69 (0.5H), 6.63 (0.5H), 6.48 (1H), 5.58 (1H), 4.40 (1H), 4.22 (1H), 3.85 (1H), 3.75 (1H), 2.17 (1H), 2.05-1.75 (3H), 1.57 (1H), 1.20 (3H), 0.90 (6H); ¹⁹F NMR (DMSO-d₆): δ −74.67, −74.70; MS ESI: 684.3 [M+H]⁺

### Example 85

### N-((2S)-1-(((2S)-1-(((2S)-1-((6-Amino-1,1,1-trifluoro-2-hydroxy-6-oxohexan-3-yl)amino)-1-oxopropan-2-yl)amino)-1-oxo-4-phenylbutan-2-yl)amino)-3-methyl-1-oxobutan-2-yl)-[1,1′-biphenyl]-4-carboxamide

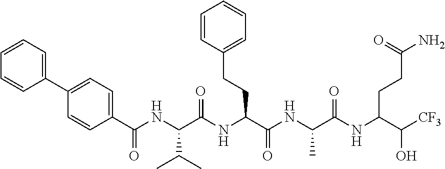 [SMILES: CC(C)[C@H](NC(=O)c1ccc(-c2ccccc2)cc1)C(=O)N[C@@H](CCc1ccccc1)C(=O)N[C@@H](C)C(=O)NC(CCC(N)=O)C(O)C(F)(F)F]

¹H NMR (DMSO-d₆): δ 8.60 (1H), 8.22 (1H), 8.03-7.86 (4H), 7.76 (4H), 7.50 (2H), 7.40 (1H), 7.28-7.07 (6H), 6.70 (1H), 6.60 (1H), 4.40-4.15 (3H), 4.06-3.72 (2H), 2.60 (2H), 2.20 (1H), 2.15-1.76 (5H), 1.61 (1H), 1.20 (3H), 0.97 (6H); ¹⁹F NMR (DMSO-d₆): δ −74.64, −74.71; MS ESI: 712.4 [M+H]⁺

### Example 86

### N-((2S)-1-(((2R)-1-(((2S)-1-((6-Amino-1,1,1-trifluoro-2-hydroxy-6-oxohexan-3-yl)amino)-1-oxopropan-2-yl)amino)-1-oxo-4-phenylbutan-2-yl)amino)-3-methyl-1-oxobutan-2-yl)-[1,1′-biphenyl]-4-carboxamide

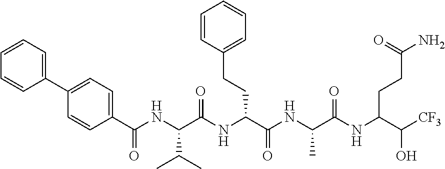 [SMILES: CC(C)[C@H](NC(=O)c1ccc(-c2ccccc2)cc1)C(=O)N[C@H](CCc1ccccc1)C(=O)N[C@@H](C)C(=O)NC(CCC(N)=O)C(O)C(F)(F)F]

¹H NMR (DMSO-d₆): δ 8.57-8.42 (2H), 8.00 (2H), 7.93 (1H), 7.84 (1H), 7.74 (4H), 7.49 (2H), 7.40 (1H), 7.25 (2H), 7.16 (4H), 6.70 (1H), 6.48 (1H), 4.22 (3H), 3.98-3.75 (2H), 2.61 (1H), 2.51 (1H), 2.21-1.72 (6H), 1.61 (1H), 1.20 (3H), 1.00 (6H); ¹⁹F NMR (DMSO-d₆): δ −74.63, −74.65; MS ESI: 712.4 [M+H]⁺

### Example 87

### N-((2S)-1-(((2S)-1-(((2S)-1-((6-Amino-1,1,1-trifluoro-2-hydroxy-6-oxohexan-3-yl)amino)-1-oxopropan-2-yl)amino)-3-(4-hydroxyphenyl)-1-oxopropan-2-yl)amino)-3-methyl-1-oxobutan-2-yl)-[1,1′-biphenyl]-4-carboxamide

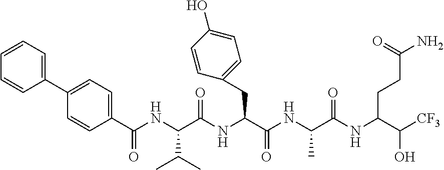 [SMILES: CC(C)[C@H](NC(=O)c1ccc(-c2ccccc2)cc1)C(=O)N[C@@H](Cc1ccc(O)cc1)C(=O)N[C@@H](C)C(=O)NC(CCC(N)=O)C(O)C(F)(F)F]

Purified by RP-HPLC (stationary phase: Phenomenex C12 Synergi 4u Max-RP; mobile phase A: 0.1% formic acid in H₂O; mobile phase B: 0.1% formic acid in CH₃CN; gradient 18-58% B in A; run time=20 min.

¹H NMR (DMSO-d₆): δ 9.12 (1H), 8.29 (1H), 8.06-7.82 (5H), 7.75 (4H), 7.50 (2H), 7.40 (1H), 7.20 (1H), 7.00 (2H), 6.69 (1H), 6.62-6.44 (3H), 4.50 (1H), 4.20 (2H), 4.00-3.78 (2H), 2.92 (1H), 2.67 (1H), 2.18-1.80 (4H), 1.62 (1H), 1.20 (3H), 0.80 (6H); ¹⁹F NMR (DMSO-d₆): δ −74.64, −74.69; MS ESI: 714.4 [M+H]⁺

### Example 88

### 3-(4-(((2S)-1-(((2S)-1-(((2S)-1-((6-Amino-1,1,1-trifluoro-2-hydroxy-6-oxohexan-3-yl)amino)-1-oxopropan-2-yl)amino)-3-(4-hydroxyphenyl)-1-oxopropan-2-yl)amino)-3-methyl-1-oxobutan-2-yl)carbamoyl)phenyl)pyridin-1-ium formate

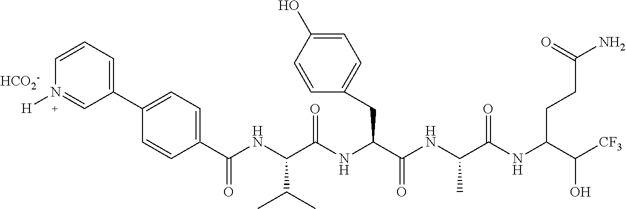

Purified by RP-HPLC (stationary phase: Phenomenex C12 Synergi 4u Max-RP 8A 50×20 mm; mobile phase A: 0.1% formic acid in H₂O; mobile phase B: 0.1% formic acid in CH₃CN; gradient 12-52% B in A; run time=20 min.

¹H NMR (DMSO-d₆): δ 9.20 (1H), 8.94 (1H), 8.60 (1H), 8.50 (1H), 8.38 (1H), 8.14 (1H), 8.08-7.87 (5H), 7.83 (2H), 7.51 (1H), 7.19 (0.5H), 7.15 (0.5H), 7.00 (2H), 6.70 (1H), 6.62 (1H), 6.66 (2H), 4.48 (1H), 4.20 (2H), 3.96-3.74 (2H), 2.91 (1H), 2.57 (1H), 2.11-1.80 (4H), 1.61 (1H), 1.19 (3H), 0.85 (3H), 0.77 (3H); ¹⁹F NMR (DMSO-d₆): δ −74.64, −74.70; MS ESI: 715.4 [M+H]⁺

### Example 89

### N-((2S)-1-(((2S)-1-(((2S)-1-((6-Amino-1,1,1-trifluoro-2-hydroxy-6-oxohexan-3-yl)amino)-1-oxopropan-2-yl)amino)-3-(1H-indol-3-yl)-1-oxopropan-2-yl)amino)-3-methyl-1-oxobutan-2-yl)-[1,1′-biphenyl]-4-carboxamide

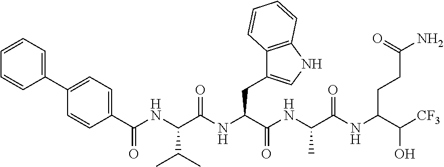 [SMILES: CC(C)[C@H](NC(=O)c1ccc(-c2ccccc2)cc1)C(=O)N[C@@H](Cc1c[nH]c2ccccc12)C(=O)N[C@@H](C)C(=O)NC(CCC(N)=O)C(O)C(F)(F)F]

¹H NMR (DMSO-d₆): δ 10.79 (1H), 8.28 (1H), 8.13 (1H), 8.05-7.80 (4H), 7.75 (4H), 7.58 (1H), 7.50 (2H), 7.41 (1H), 7.30 (1H), 7.18 (2H), 7.04 (1H), 6.95 (1H), 6.73 (1H), 6.50 (1H), 4.61 (1H), 4.30-4.15 (2H), 3.99-3.77 (2H), 3.14 (1H), 2.98 (1H), 2.15-1.80 (4H), 1.65 (1H), 1.30-1.10 (3H), 0.87 (3H), 0.80 (3H); ¹⁹F NMR (DMSO-d₆): δ −74.62, −74.65; MS ESI: 737.4 [M+H]⁺

Purified by RP-HPLC (stationary phase: Phenomenex C12 Synergi 4u Max-RP 8A 50×20 mm; mobile phase A: 0.1% formic acid in H₂O; mobile phase B: 0.1% formic acid in CH₃CN; gradient 20-60% B in A; run time=20 min.

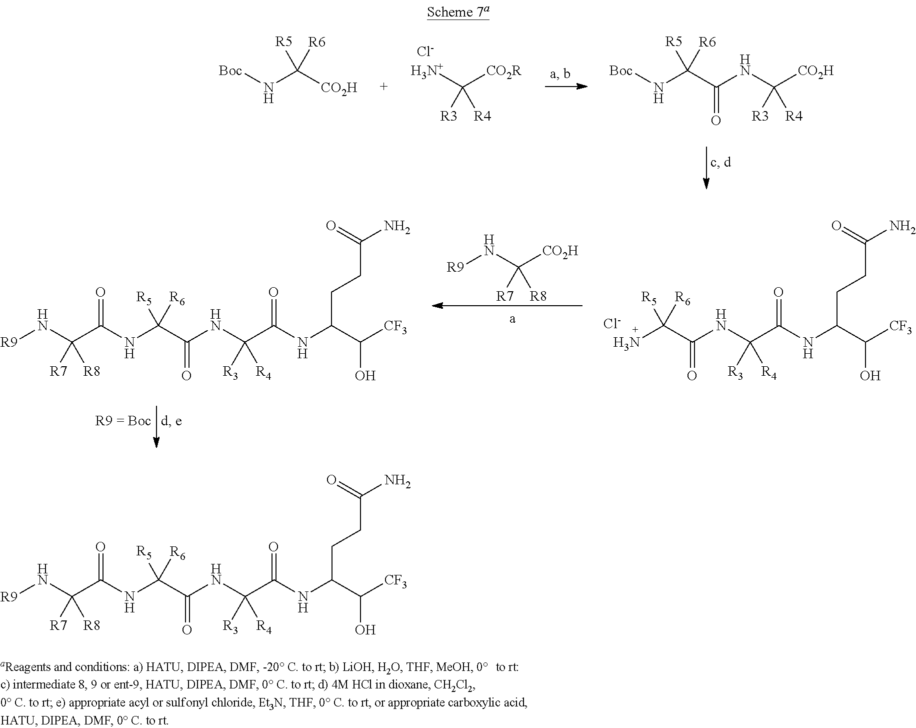

### Compound Examples Prepared According to Scheme 7

### Example 90

### N-(1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)carbamoyl)cyclopentyl)-[1,1′-biphenyl]-4-carboxamide

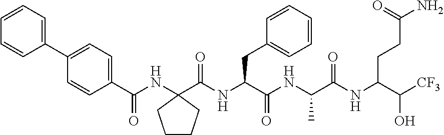 [SMILES: C[C@H](NC(=O)[C@H](Cc1ccccc1)NC(=O)C1(NC(=O)c2ccc(-c3ccccc3)cc2)CCCC1)C(=O)NC(CCC(N)=O)C(O)C(F)(F)F]

¹H NMR (DMSO-d₆): δ 8.66 (0.5H), 8.61 (0.5H), 8.00 (2H), 7.86-7.60 (6H), 7.58-7.36 (4H), 7.13 (6H), 6.68 (1H), 6.45 (1H), 4.44 (1H), 4.15 (1H), 3.99-3.78 (2H), 3.10 (1H), 2.87 (1H), 2.25 (1H), 2.17-1.74 (5H), 1.73-1.40 (6H), 1.23 (3H); ¹⁹F NMR (DMSO-d₆): δ −74.62, −74.67; MS ESI: 710.4 [M+H]⁺

### Example 91

### N-(1-(((2S)-1-(((2S)-1-((6-Amino-1,1,1-trifluoro-2-hydroxy-6-oxohexan-3-yl)amino)-1-oxopropan-2-yl)amino)-1-oxo-3-phenylpropan-2-yl)carbamoyl)cyclopropyl)-[1,1′-biphenyl]-4-carboxamide

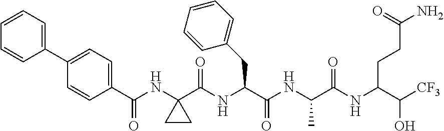 [SMILES: C[C@H](NC(=O)[C@H](Cc1ccccc1)NC(=O)C1(NC(=O)c2ccc(-c3ccccc3)cc2)CC1)C(=O)NC(CCC(N)=O)C(O)C(F)(F)F]

¹H NMR (DMSO-d₆): δ 9.10 (1H), 8.02-7.90 (3H), 7.85-7.66 (6H), 7.50 (2H), 7.40 (1H), 7.12 (6H), 6.70 (1H), 6.50 (1H), 4.53 (1H), 4.20 (1H), 4.00-3.78 (2H), 3.02 (1H), 2.90 (1H), 2.18-1.79 (3H), 1.61 (1H), 1.40-1.15 (5H), 1.03 (1H), 0.93 (1H); ¹⁹F NMR (DMSO-d₆): δ −74.63, −74.71; MS ESI: 682.3 [M+H]⁺.

Compounds of the invention were then screened using biological PCSK9-based assays.

### Example 92

### Initial Screening

HepG2 cells stably expressing WT PCSK9(+V5) were incubated overnight (24 hours) in the absence (DMSO) or presence of 10 μM, 100 μM or increasing concentrations (11, 33, 100 μM) of a tested compound. 24 hours-conditioned media was collected, centrifuged, and the supernatant removed for PCSK9 quantification by ELISA (100 μl of 1:30 dilution). Cells were lysed with 250 ul/well of radioimmunoprecipitation assay buffer (RIPA) (50 mM Tris-HCl, pH 7.8, 150 mM NaCl, 1% Nonident P-40, 0.5% sodium deoxycholate, 0.1% SDS) containing a mixture of protease inhibitors (Roche Applied Science), and pelleted at 11,300×g for 5 minutes. Supernatant was removed for cellular PCSK9 quantification by ELISA (100 μl of 1:20 dilution) and total protein determination by the Bio-Rad DC Protein assay (Bio-Rad) (Dubuc G, Tremblay M, Pare G, Jacques H, Hamelin J, Benjannet S, Boulet L, Genest J, Bernier L, Seidah N G, Davignon J. 2010. A new method for measurement of total plasma PCSK9: clinical applications. J Lipid Res. 51:140-149). Over 165 compounds were subjected to a first screen at 100 μM by PCSK9 secretion assay.

Certain compounds identified to reduce the PCSK9-media/cell ratio by at least 30% versus DMSO control (30% inhibition) in this assay were further tested for their ability to inhibit PCSK9 secretion in a dose dependent manner. At least 24 compounds were identified to inhibit PCSK9 secretion in a dose-dependent fashion (compounds described in examples 1 to 24).

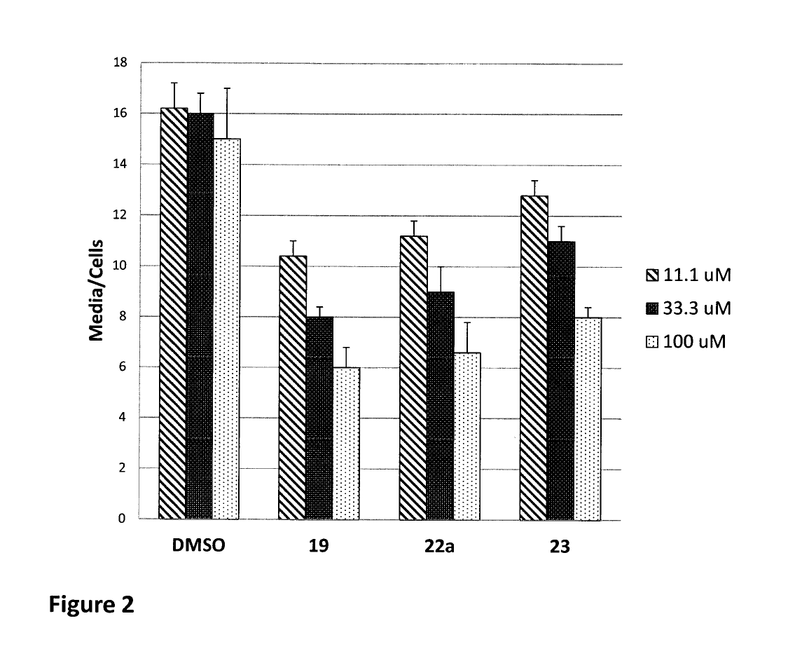

FIG. 1 and Table I present a selection of the tested compounds. FIG. 2 illustrate the dose-dependent inhibition of PCSK9 secretion in HepG2 cells stably expressing PCSK9(+V5) for a selection of the tested compounds, namely 19, 22a and 23, which reduce the PCSK9-media/cell between 30% and 50% versus DMSO control, at a dose of 33.3 μM.

TABLE I
PCSK9 secretion for selected compounds
tested at a concentration of 10 μM

| | Secretion Inhibition |
|---|---|
| Example # | (% Activity)^{a} |
| 24 | 88 |
| 29 | 83 |
| 52 | 79 |
| 58 | 81 |
| 59 | 82 |
| 60 | 73 |
| 66 | 92 |
| 70 | 74 |
| 75 | 100 |

The % activity was calculated using the following formula:

$$\text{*\%} Activity = \{ 1 - \frac{{( {[ PCSK 9 ]}_{media} {\text{/} [ PCSK 9 ]}_{cells} )}_{inhibitor}}{{( {[ PCSK 9 ]}_{media} {\text{/} [ PCSK 9 ]}_{cells} )}_{DMSO}} \} \times 100 \%$$

### Example 93

### Cytotoxicity Assays

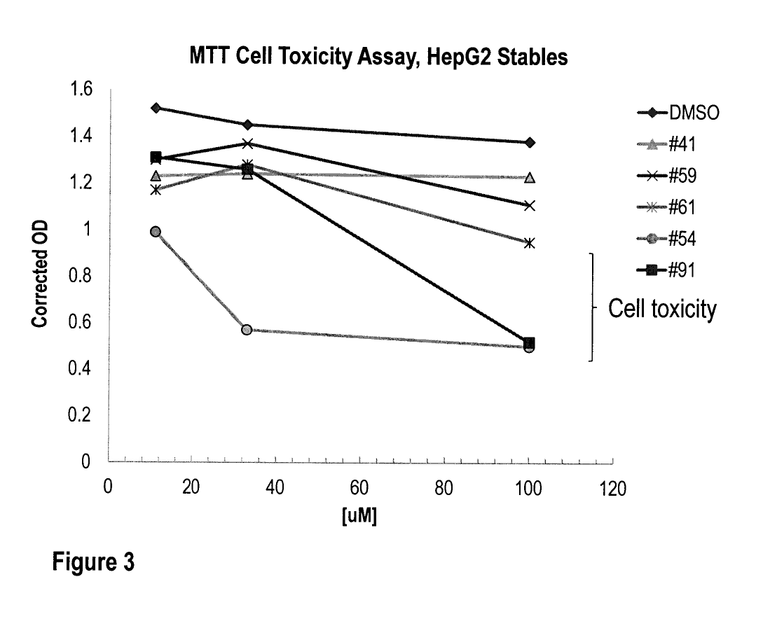

Following the initial screening, the cytotoxicity of compounds was tested using either a MTT toxicity assay (3-[4,5-dimethylthiazol-2-yl]-2,5 diphenyl tetrazolium bromide) according to manufacturer's protocol (Promega) (for see FIG. 3) or using a cell viability assay (See Table II).

For the MTT toxicity assay, HepG2 stable cells over-expressing WT PCSK9(+V5) were seeded in 96-well plates (Greiner BioOne) and incubated for 20 hours. Following a brief wash, cells were incubated overnight (24 hours) in the presence of increasing concentrations (11, 33, 100 μM) of compounds or in the presence of DMSO (control). The MTT assay (Promega) consisted of addition of 20 μl/well MTT reagent and incubation for 45 minutes at 37° C., following the manufacturer's instructions. Absorbance was recorded at 490 nm and corrected for background due to nonspecific absorbance (690 nm). Compounds with a low toxicity at 33.3 μM or less were selected as exemplified in FIG. 3 for compounds 19, 22a, 23, 4 and 17.

For the cell viability assay, the cell density of HepG2 cells was measured in the presence of 33 μM of compounds or in the presence of DMSO (control) during the PCSK9 secretion assay described in Example 92. Table II below presents compounds with a low toxicity at 33.3 μM or less.

TABLE II
Cell viability in the presence of selected compounds
at a concentration of 33 μM presented as a percentage
of that obtained in the absence thereof (DMSO)

| | Cell viability |
|---|---|
| Example # | (% DMSO Cnt) |
| 59 | 83 |
| 58 | 93 |
| 29 | 88 |
| 60 | 80 |

The activity of selected PCSK9 inhibitory compounds on PCSK9 was further characterized, using a variety of assays including Western blot analyses and Biosynthetic analysis for the effect on PCSK9 autoprocessing and secretion. The effect on LDLR degradation and activity of the selected PCSK9 inhibitory compounds was characterized using a series of assays, including immunofluorescence assay, Dil-LDL uptake assay and FACS analysis for cell-surface LDLR, and Western blot analyses for total LDLR.

### Example 94

### Immunofluorescence Assay

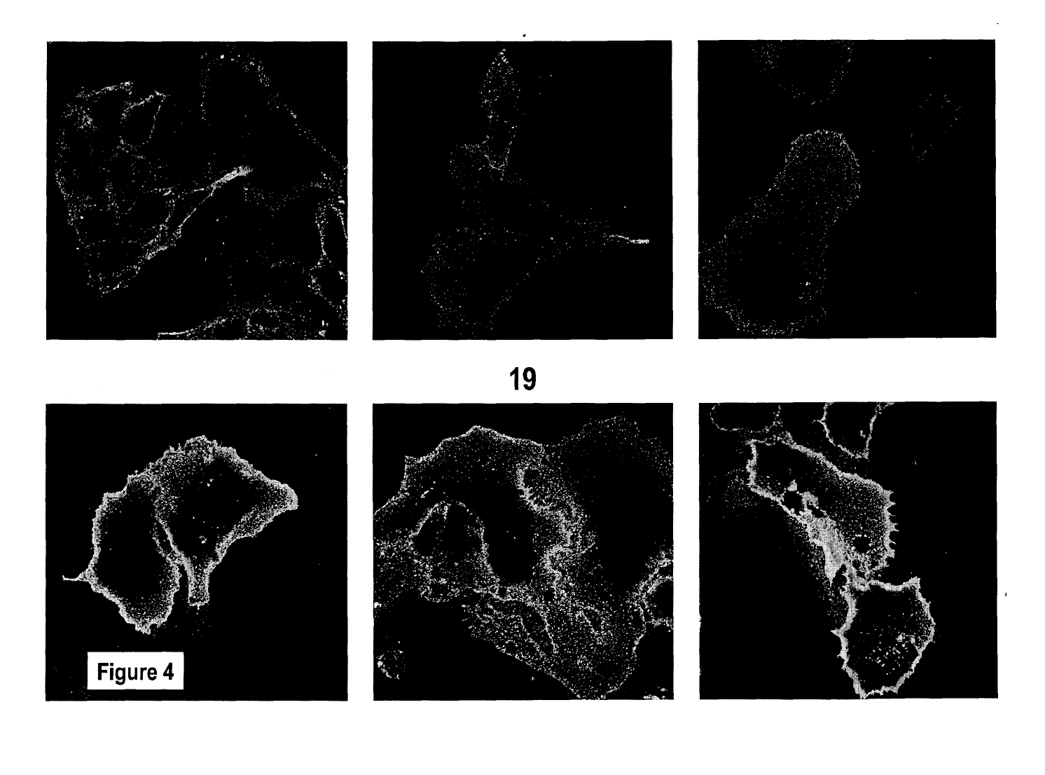

The PCSK9 inhibitory compounds were characterized by immunofluorescence for cell-surface LDLR and for human transferrin receptor, as control, in HepG2 naïve cells. Cells were plated on Poly-L-Lysine-coated (50 ug/ml) round microscope cover slips 1.12 mm thickness (Fisherbrand 12CIR #1) that were placed in a 24-well cell culture plate. At 24 h following seeding, cells were incubated in the absence (DMSO) or in the presence of 33.3 μM of PCSK9 inhibitor. After a 20 hour incubation period, the cells were fixed with 3.7% paraformaldehyde. Immunofluorescence of human LDLR (green labeling) was performed under non-permeabilizing conditions. Cells were blocked with 1% BSA, followed by overnight incubation at 4° C. with primary antibody (1:200 goat polyclonal anti-hLDLR in 1% BSA, R&D Systems). Antigen-antibody complexes were revealed by 1-hour incubation at room temperature with Alexa fluor-tagged secondary antibody and mounted in ProLong Gold Antifade Reagent (Molecular Probes, Invitrogen). Immunofluorescence analyses were performed with a confocal miscroscope (Zeiss LSM-710). Cell nuclei were stained with DAPI (blue labeling). LDLR was stained with anti-LDLR Abs (green labeling). Inhibition of PCSK9 activity on LDLR degradation was detected by an increase of LDLR at the cell surface, as exemplified in FIG. 4 for compound 19.

### Example 95

### Dil-LDL Assay

The PCSK9 inhibitory compounds were also characterized using a Dil-LDL fluorescent uptake assay as described in Poirier et al., J. Biol. Chem. 284: 28856-28864, 2009. The method entails the fluorescence measurement of the Dil-LDL cellular incorporation via LDLR internalization (a measurement of cell surface LDLR activity) in human hepatocyte derived HuH7 or HepG2 cell lines, or human embryonic HEK293 cells, in the presence or absence of compounds.

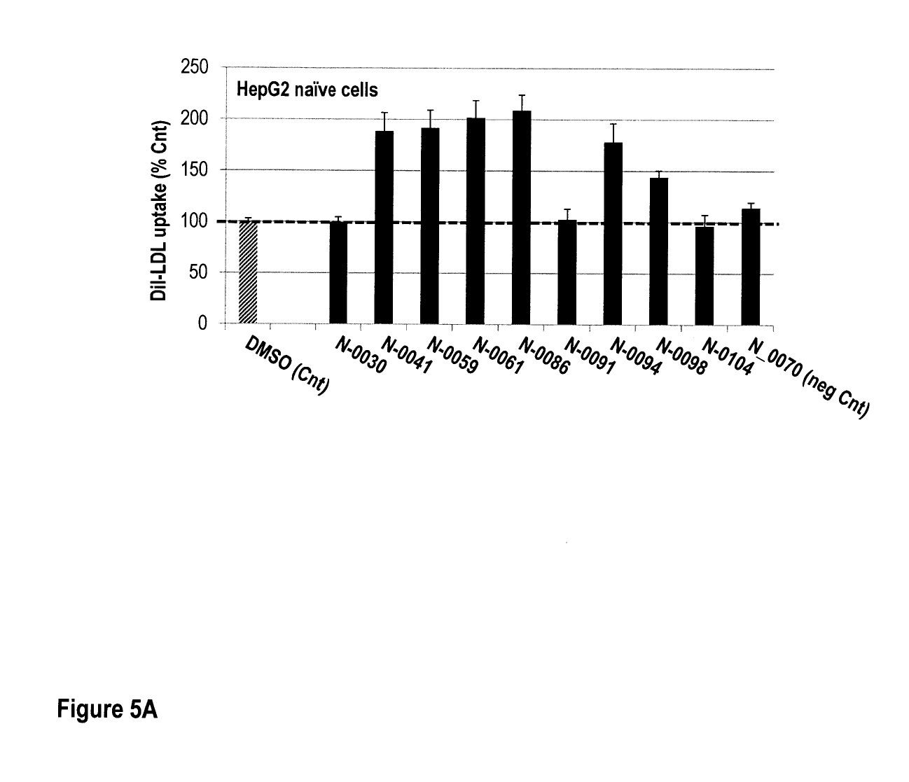

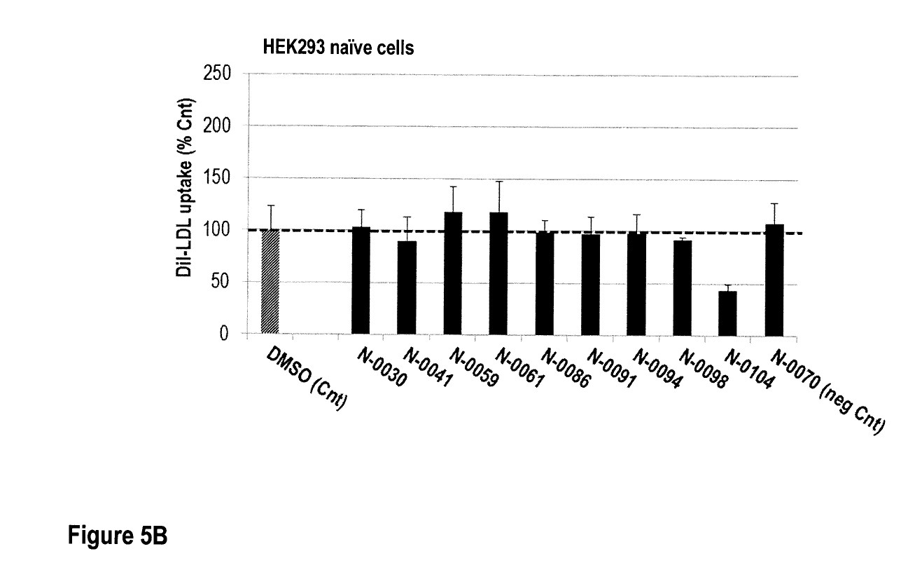

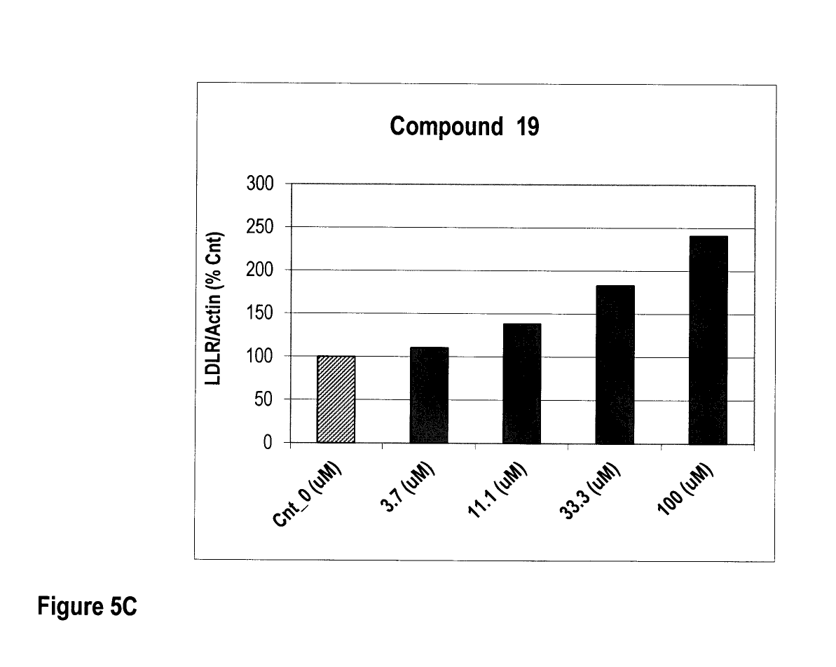

HepG2 naïve cells and HEK293 naïve cells were plated in 96-well plates (CellBind™ black plate with clear bottom (Corning; Cat #3340)). At 20 h post-seeding, cells were incubated in the absence (DMSO or negative control) or in the presence of different concentrations of compounds. Each condition was prepared in triplicates. After 6 hours incubation, Dil-LDL (Biomedical Technologies (Cat #BT-904)) was added to the cell media, and cells returned to tissue culture incubator for another 18 hours. Plates were scanned (bottom read) on a SpectraMax GeminiEM™ plate reader (Molecular Devices). For each well, raw Dil-LDL uptake was measured as the average fluorescence intensity (RFU) (ex: 520 nm/em: 575 nm, cutoff: 550 nm) of 9 readings in 3 different points in the well. Dil-LDL uptake in each well was corrected for total number of cells by performing a CyQuant™ cell assay (Invitrogen, Cat #C7026) according to the manufacturer's instructions. Corrected Dil-LDL uptake is reported as a % of DMSO control and was obtained from triplicate wells. The inhibition of the PCSK9 activity on LDLR degradation is detected by an increase in the Dil-LDL uptake (exemplified in FIG. 5A for compounds 19, 22a, 23, 24, 21a, 21b in HepG2 naïve cells). Compound i was selected as a negative control and compounds 1, 17 and 2 display no measurable activity in this assay in HepG2 cells. The specificity of the compounds for PCSK9 is exemplified in FIG. 5B, which shows that in HEK293 cells, known to lack PCSK9 expression, the same set of compounds have no effect on Dil-LDL uptake.

TABLE III
Dil-LDL uptake in HepG2 cells in the presence of selected
compounds at a concentration of 10 μM presented as a
percentage of that obtained in the absence thereof (DMSO)

| | Dil-LDL uptake |
|---|---|
| Example # | (% DMSO Cnt) |
| 24 | 224 ± 33 |
| 29 | 207 ± 21 |
| 52 | 254 ± 71 |
| 58 | 261 ± 48 |
| 59 | 253 ± 75 |
| 60 | 224 ± 43 |
| 66 | 237 ± 59 |
| 70 | 224 ± 47 |
| 75 | 172 ± 48 |

In order to further test whether these compounds can also inhibit the function of a gain of function mutant D374Y of PCSK9, the cells expressing PCSK9-D374Y are analyzed by using a Dil-LDL fluorescent uptake assay. The inhibitory effect of compounds is also characterized using a liver-derived mouse cell line FL-83B (ATCC, CRL-2390) in the Dil-LDL uptake assay.

### Example 96

### Western Blot

Compounds were tested for their ability to inhibit the LDLR enhanced degradation of PCSK9 in human hepatocyte cell lines HepG2. Cells were incubated for 24 h in the absence or presence of tested PCSK9 inhibitory compounds. Following incubation, cells were lysed in RIPA. Proteins were separated by SDS-polyacrylamide gel electrophoresis and blotted on polyvinylidene difluoride (PVDF, Perkin Elmer) membranes (GE Healthcare). Membranes were then incubated in the presence human LDLR antibody (1:1000, R&D Systems) and 1-actin antibody (1:2500, Sigma). Appropriate horseradish peroxidase-conjugated secondary antibody (1:10,000, Sigma) was used for detection with enhanced chemiluminescence using the ECL plus kit (GE Healthcare). The inhibition of the PCSK9 function in these assays was detected by an increased ratio of total LDLR/p-Actin, as measured by Western blot analyses. Quantification of protein bands was obtained using Image J™ software. FIG. 5C exemplifies the Western blot analysis of total LDLR in HepG2 naïve cells incubated for 24 h in the absence (Cnt_{—}0) or presence of increasing concentrations of compound 19. Total LDLR levels normalized to R-actin are plotted for each condition as % control (Cnt).

### Example 97

### FACS Assay

The ability of PCSK9 inhibitory compounds to prevent the activity of PCSK9 on LDLR at the cell surface was also measured by using flow cytometry analyses as described in Benjannet S. et al. J. Biol. Chem. 285: 40965-40978, 2010. The level of LDLR at the HepG2 cell surface was measured by using anti human LDLR antibody (1:100, mAb-C7, Santa Cruz Biotechnology) and a secondary Alexa Fluor 647 donkey anti-mouse (Molecular Probes) antibody. Viable cells (PI-negative) were then analyzed by FACS for both PI and Alexa Fluor 647 using the FACS BD LSR (BD Biosciences). Cell surface LDLR was reported relative to control (untreated cells). The inhibition of the PCSK9 function in these assays was detected by an increased number of positive cells which correlated with an increased expression of LDLR at the cell surface.

More particularly, Human hepatoblastoma HepG2 cells (4×10⁵ cells/well) were seeded in 12 well plates (Greiner BioOne) in complete media and cultured for 20 hours, at 37° C., followed by a 1 hour wash in serum free media at 37° C. Next, the wash media was removed and the cells were incubated overnight (24 hours) with 0.9 ml/well incubation media containing compounds at 10 uM or DMSO control (0.4% final). After one rinse with buffer A followed by another rinse with EDTA solution (2.5 mM EDTA-2Na), the cells were detached by incubation in the presence of fresh EDTA solution (1 ml) at 37° C. for 20 minutes. All the steps following cell detachment were conducted on ice or 4° C. To terminate the detachment, the cells were transferred in a 15 ml tube containing 3 ml of buffer A and centrifuged immediately for 5 minutes at 900 rpm (100 g) (4° C.). The cells were then resuspended in 500 ul buffer A together with 1:250 human LDLR antibody and incubated on ice for 10 minutes. Cells were washed once with 5 ml buffer A, centrifuged for 5 minutes, resuspended in 500 ul buffer A containing 1:500 Alexa Fluor 647 secondary antibody and incubated on ice for 5 min. After the staining, cells were washed once with 5 ml buffer A, centrifuged for 5 minutes and resuspended in 300 ul buffer A containing 1.67 ug/ml propidium iodide. A fixed number (2,000 cells) of viable cells (propidium iodide negative) were then analyzed by FACS for Alexa Fluor 647 (cell surface LDLR) using a FACS-CyAn ADP flow cytometer (Beckman Coulter (DAKO)) and Summit software (Summit Software Inc.).

### Materials:

Human HepG2 cell line: ATCC, HB-8065
Seeding media: complete media—EMEM (high glucose+sodium pyruvate) (Wisent)+10% FBS
Wash media: serum free media—EMEM (high glucose+sodium pyruvate) (Wisent)
Incubation media with compounds: EMEM (high glucose+sodium pyruvate) (Wisent)+0.07% BSA (Sigma-Aldrich)
BufferA: 1×D-PBS without calcium and magnesium (Wisent 311-425-CL)+0.5% BSA (Sigma A7409-50ml)+1 ug/L Glucose (Wisent 609-036-EL).
Primary antibody: human LDLR antibody, monoclonal Mouse IgG₁ clone #472413 (MAB2148, R&D Systems)
Secondary antibody: Alexa Fluor® 647 goat anti-mouse IgG (H+L)*2 mg/mL*(A21235, Molecular Probes)
Propidium iodide: 1 mg/ml solution (P4864, Sigma)

Results are presented in Table IV.

TABLE IV
FACS-LDLR expressed as a percentage of LDLR positive
cells in the presence and absence (DMSO) of selected
compounds at a concentration of 10 μM

| | FACS-LDLR |
|---|---|
| Example # | (% DMSO Cnt) |
| 24 | 116 ± 5 |
| 29 | 233 ± 50 |
| 52 | 138 ± 20 |
| 58 | 166 ± 28 |
| 59 | 265 ± 35 |
| 60 | 138 ± 8 |
| 66 | 122 ± 11 |
| 70 | 118 ± 18 |
| 75 | 174 ± 41 |

The PCSK9 inhibitory compounds are also tested on mouse and human primary hepatocytes in order to measure their effect on cell surface LDLR. The advantage of using the mouse primary hepatocytes is that it allows for measurement of the specificity of the compound in the context of a wild type or knockout mouse expressing or lacking PCSK9, respectively.

### Example 98

### Characterisation of the Inhibition of PCSK9-Induced LDLR Degradation in Animal Models

Candidate PCSK9 inhibitory compounds are then used in mice models expressing human PCSK9 to assess their ability in lowering plasma cholesterol levels in vivo.

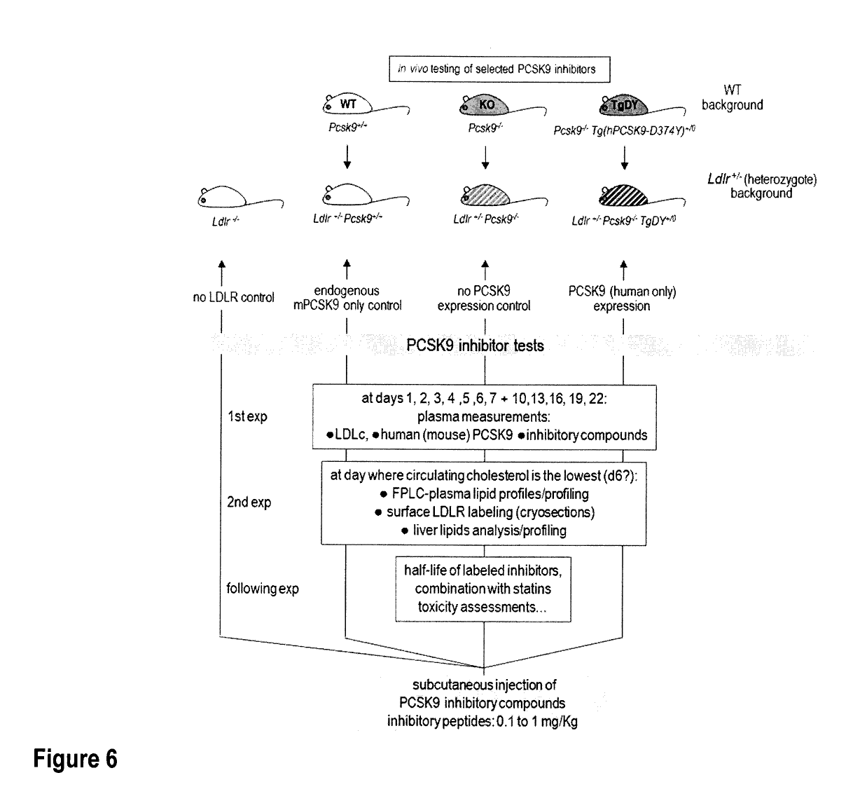

The compounds are tested in mice overexpressing human PCSK9. Transgenic lines (Herbert, B., et al. (2010). Arteriosclerosis, Thrombosis, and Vascular Biology, 30 (7):1333-1339) that carry a ˜190 kb of human genomic DNA expressing human PCSK9 (WT, D374Y low or D374Y high) from its own promoter are constructed. Further crosses generate a mouse strain expressing the human PCSK9 transgene in a Pcsk9^{-/-} Ldlr^{+/-} background (Ldlr heterozygote). This eliminates interference through the endogenous expression of the mouse PCSK9 genes and increases their LDLc levels (FIG. 6). By multiple backcrosses, these model mice are obtained in a pure C57BL/6 background for the homogeneity and reproducibility of the analyses. As control for specificity, the effect of the transgenes in an Ldlr^{-/-} background is tested.

Mouse Injections:

The compounds are injected intravenously to WT, Pcsk9^{-/-}, Ldlr^{-/-} and Pcsk9-Tg mice, in 6 mice/genotype (FIG. 6). Total cholesterol (TC), LDLc and PCSK9 levels are measured every day during the first week and every third day for the next 2 weeks. The level of remaining uncomplexed PCSK9 in plasma is also measured by immunoprecipitation using a previously described antibody (Zaid, A., et al. (2008). Hepatology, 48(2): 646-65). In another set of experiments, 6 mice at the time point of lowest LDLc (4-7 days post-injection) are sacrificed and their FPLC plasma lipid profiles, as well as liver LDLR protein, are analyzed. Any overt toxicity and/or morbidity effect is carefully monitored. The controls of Pcsk9^{-/-}, Ldlr^{-/-} mice permit to verify that the effect observed is PCSK9-dependent.

Effect of Statin+PCSK9 Inhibitory Compounds:

The combination of atorvastatin and inhibitory compound is evaluated, as statins increase PCSK9 expression while lowering that of the LDLR (Dubuc, G., et al. (2004). Arteriosclerosis, Thrombosis, and Vascular Biology, 24(8): 1454-1459; Lakoski, S. G., et al. (2009). The Journal of Clinical Endocrinology and Metabolism, 94(7): 2537-2543), and it was shown that statins decrease even further LDLc of Pcsk9^{-/-} mice (Rashid, S., et al. (2005). Proc Natl Acad Sci USA 102(15):5374-5379).

1. Compound of Formula (I): 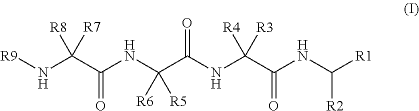
or a pharmaceutically acceptable salt, hydrate, solvate, or racemic mixture or stereoisomer thereof,
wherein:
R1 is —CH(OH)Rₐ or —B(OR_{b})(OR_{c});
R2 is —H, —CH₂R_{d}, —CHR_{d}(Rₑ), —CH₂(CH₂)ₘC(O)Rⱼ, —CH₂(CH₂)ₘC(O)N(R_{d})Rₑ or —CH₂(CH₂)ₘS(O)ₙN(R_{d})Rₑ;
R3, R4, R5, R6, R7 and R8 are identical or different, and are independently hydrogen or one of the following groups: a C1-6 alkyl, a C1-6 haloalkyl, a C1-6 thioalkyl, a C1-6 aminoalkyl, an alkenyl, an alkynyl, a cycloalkyl, an heterocyclyl, an aryl, and an heteroaryl group, wherein said group is optionally substituted with one or more C1-6 alkyl, C3-8 cycloalkyl, C1-6 haloalkyl, aryl, an heteroaryl, —CN, —C(O)N(R_{f})R_{g}, —C(O)OR_{f}, —C(R_{f})(R_{g})ORₕ, —OR_{f}, —OC(O)OR_{f}, —OC(O)NR(R_{g}), —SR_{f}, —S(O)ₙR_{f}, —S(O)ₙN(R_{f})R_{g}, —S(O)ₙN(R_{f})C(O)R_{g}, —N(R_{f})R_{g}, —N(R_{f})C(O)R_{g}, —N(R_{f})C(O)OR_{g}, —N(R_{f})C(O)N(R_{g})(Rₕ), —N(R_{f})S(O)ₙR_{g}, and —N(R_{f})S(O)N(R_{g})Rₕ substituents;
when (R3 and R4) or (R5 and R6) or (R7 and R8) are not hydrogen, the bracketed pairs can also be linked with —C(O)—, —CO₂—, —C(O)N(H)—, —C(O)N(Rₓ)—, —O—, —NH—, —N(Rₓ)—, —S—, —S(O)ₙ—, —S(O)ₙN(H)—, —S(O)ₙN(Rₓ)— radicals to form cyclic structures;
R9 is RᵢC(O)—, RᵢS(O)ₙ—, RᵢOC(O)—, RᵢNHC(O)—, RᵢNHS(O)ₙ—, Rₖ(Rₗ)NC(O)—, Rₗ(Rₗ)NS(O)ₙ—; RₘORᵢC(O)—, RₘC(O)RᵢC(O)— or one or more amino acids residues;
Rₐ is C1-3 alkyl, C1-2 fluoroalkyl or cyclopropyl;
R_{b} and R_{c} are identical or different, and are independently H or C1-6 alkyl, or can be connected together to form a cyclic 5- or 6-membered ring structure, or fused with additional aliphatic or aromatic ring systems, the cyclic 5- or 6-membered ring structure or aliphatic or aromatic ring systems being optionally substituted with one or more C1-6 alkyl and/or C1-6 haloalkyl substituents;
R_{d} and Rₑ are identical or different, and are independently H or one of the following groups: a C1-3 alkyl, a C1-3 haloalkyl or a C3-4 cycloalkyl group, or can be connected together directly or with —C(O)—, —C(O)O—, —C(O)N(Rₓ)— —O—, —N(Rₓ)—, —S—, —S(O)ₙ—, or —S(O)ₙN(Rₓ)— radicals to form cyclic 3-8 membered ring structures;
R_{f}, R_{g}, Rₕ, Rₖ and Rₗ are identical or different, and are independently H or one of the following groups: a C1-6 alkyl, a C1-6 haloalkyl or a C3-4 cycloalkyl group, or can be connected together directly or with —C(O)—, —C(O)O—, —C(O)N(Rₓ)— —O—, —N(Rₓ)—, —S—, —S(O)ₙ— or —S(O)ₙN(Rₓ)— radicals to form cyclic 3-8 membered ring structures;
Rᵢ is a C1-10 alkyl, a C1-10 heteroalkyl, a C3-8 cycloalkyl, a C1-10 haloalkyl, a heterocyclyl, an aryl, a heteroaryl, a C1-10 alkyl-C3-8 cycloalkyl, a C1-10 alkyl-heterocyclyl, a C1-10 alkyl-aryl, a C1-10 alkyl-heteroaryl, a C1-10 heteroalkyl-C3-8 cycloalkyl, a C1-10 heteroalkyl-heterocyclyl, a C1-10 heteroalkyl-aryl or a C1-10 heteroalkyl-heteroaryl group, wherein the group is optionally substituted with one or more of a halogen, C1-6 aminoalkyl, C1-6 heteroalkyl, C1-6 alkyl, C3-8 cycloalkyl, C1-6 haloalkyl, aryl, heteroaryl and heterocyclyl groups;
Rₘ is a C1-10 alkyl, a C1-10 heteroalkyl, a C3-8 cycloalkyl, a C1-10 haloalkyl, a heterocyclyl, an aryl or a heteroaryl group, wherein the group is optionally substituted with one or more of a halogen, C1-6 aminoalkyl, C1-6 heteroalkyl, C1-6 alkyl, C3-8 cycloalkyl, C1-6 haloalkyl, aryl, heteroaryl and heterocyclyl groups;
Rⱼ is OR_{d} or N(R_{d})(Rₑ);
Rₓ is H, C1-6 alkyl, C1-6 haloalkyl, C3-4 cycloalkyl, —C(O)R_{y}, —C(O)OR_{y}, —C(O)NH₂, —C(O)NH(R_{y}) or —C(O)NHS(O)ₙR_{y};
R_{y} is C1-6 alkyl, C1-6 haloalkyl or C3-4 cycloalkyl;
m is an integer of value 0 or 1; and
n is an integer of value 1 or 2,
provided that:
1) when R1 is —CH(OH)Rₐ, R2 is —CHR_{d}(Rₑ), —CH₂(CH₂)ₘC(O)Rⱼ, —CH₂(CH₂)ₘC(O)N(R_{d})Rₑ or —CH₂(CH₂)ₘS(O)ₙN(R_{d})Rₑ; and
2) when R1 is —B(OR_{b})(OR_{c}), R2 is —H, —CH₃, —CHR_{d}(Rₑ), —CH₂(CH₂)ₘC(O)Rⱼ, —CH₂(CH₂)ₘC(O)N(R_{d})Rₑ or —CH₂(CH₂)ₘS(O)ₙN(R_{d})Rₑ.

2. The compound of claim 1, wherein:
(A)
a. R1 is —B(OR_{b})(OR_{c}), or —CH(OH)Rₐ;
b. R2 is H or —CH₂(CH₂)ₘC(O)Rⱼ;
c. R3 is H or C1-6 alkyl substituted or not;
d. R4 is H, C1-6 alkyl substituted or not, C1-6 thioalkyl substituted or not, aryl substituted or not or cycloalkyl substituted or not;
e. R5 is H;
f. R6 is H, C1-6 alkyl substituted or not, C1-6 thioalkyl substituted or not, aryl substituted or not, or cycloalkyl substituted or not;
g. R7 is H;
h. R8 is H, C1-6 alkyl substituted or not, C1-6 thioalkyl substituted or not, aryl substituted or not, or cycloalkyl substituted or not; and/or
i. R9 is RiOC(O)—, RiC(O)—, RₘORᵢC(O)—, RₘC(O)RᵢC(O)— or RᵢS(O)ₙ—; or
(B)
a. R1 is —B(OR_{b})(OR_{c}) or —CH(OH)Rₐ;
b. R2 is H or —CH₂(CH₂)C(O)Rⱼ;
c. R3 is H or C1-6 alkyl substituted or not;
d. R4 is H, C1-6 alkyl substituted or not, C1-6 thioalkyl substituted or not, aryl substituted or not or cycloalkyl substituted or not;
e. R5 is H;
f. R6 is H, C1-6 alkyl substituted or not, C1-6 thioalkyl substituted or not, aryl substituted or not, or cycloalkyl substituted or not;
g. R7 is H;
h. R8 is H, C1-6 alkyl substituted or not, C1-6 thioalkyl substituted or not, aryl substituted or not, or cycloalkyl substituted or not; and
i. R9 is RiOC(O)—, RiC(O)—, RₘORᵢC(O)—, RₘC(O)RᵢC(O)—, RᵢS(O)ₙ—.

3. (canceled)

4. The compound of claim 1, wherein:
a. R1 is —B(OR_{b})(OR_{c}) or —CH(OH)Rₐ;
b. R2 is H or —CH₂(CH₂)ₘC(O)Rⱼ;
c. R3 is H or C1-6 alkyl substituted or not;
d. R4 is H, C1-6 alkyl substituted or not, C1-6 thioalkyl substituted or not, aryl substituted or not, or cycloalkyl substituted or not;
e. R6 is H, C1-6 alkyl substituted or not, C1-6 thioalkyl substituted or not, aryl substituted or not, or cycloalkyl substituted or not;
f. R8 is H, C1-6 alkyl substituted or not, C1-6 thioalkyl substituted or not, aryl substituted or not, or cycloalkyl substituted or not; and/or
g. R9 is RiOC(O)—, RiC(O)—, RₘORᵢC(O)—, RₘC(O)RᵢC(O)—, or RᵢS(O)ₙ—.

5. The compound of claim 4, wherein:
a. R1 is:
i. —B(OR_{b})(OR_{c}) and Rb and Rc are H, or Rb and Rc are connected to form a cyclic 5 membered ring structure or fused with an aliphatic ring system; or
ii. —CH(OH)Rₐ and Ra is C1-2 fluoroalkyl, or Ra is C1-3 alkyl;
b. R2 is —CH₂(CH₂)ₘC(O)Rⱼ and Rj is OR_{d} or NH₂;
c. R3 is unsubstituted C1-6 alkyl;
d. R4 is unsubstituted C1-6 alkyl, substituted C1-6 alkyl, CH₃S(CH₂)₂—, or phenyl;
e. R6 is substituted C1-6 alkyl, unsubstituted C1-6 alkyl, CH₃S(CH₂)₂—, phenyl, or benzocyclopentyl;
f. R8 is unsubstituted C1-6 alkyl, substituted C1-6 alkyl, CH₃S(CH₂)₂—, unsubstituted aryl, or unsubstituted cycloalkyl; and/or
g. R9 is:
i. RiOC(O)— and Ri is C1-10 alkyl, substituted or not;
ii. RiC(O)— and Ri is aryl substituted or not or heteroaryl substituted or not; or C1-10 alkyl, substituted or not;
iii. RₘORᵢC(O)— and Rᵢ is substituted or unsubstituted C1-10 alkyl;
iv. RₘC(O)RᵢC(O)— and Rᵢ is aryl; or
v. RᵢS(O)ₙ— and R; is substituted or unsubstituted aryl.

6. (canceled)

7. The compound of claim 5, wherein:
a. R1 is 2,9,9-trimethyl-3,5-dioxa-4-boratricyclo[6.1.1.0^{2,6}]decan-4-yl, or —CH(OH)Rₐ wherein Ra is —CF₃, —CH₂F, or —CH₃;
b. Rd is CH₃;
c. R3 is CH₃;
d. R4 is —CH₃, CH₃CH₂—, —CH₂CH(CH₃)₂, aryl substituted C1-6 alkyl, or —(CH₂)₂C(O)NH₂;
e. R6 is aryl substituted C1-6 alkyl, CH₃, or —CH(CH₃)₂;
f. R8 is —CH(CH₃)₂, —CH₃, aryl substituted C1-6 alkyl, phenyl, cyclopentyl, or cyclopropyl; and/or
g. R9 is:
i. RiOC(O)— and Ri is unsubstituted C1-10 alkyl-, or substituted C1-10 alkyl-;
ii. RiC(O)— and Ri is aryl substituted or not, heteroaryl substituted or not, substituted C1-10 alkyl-, or unsubstituted C1-10 alkyl-;
iii. RₘORᵢC(O)— and Rᵢ is —CH₂—;
iv. RₘC(O)RᵢC(O)— and Rᵢ is phenyl; or
v. RᵢS(O)ₙ— and Rᵢ is substituted aryl.

8.-30. (canceled)

31. The compound of claim 7, wherein:
a. R4 is phenyl-CH₂—;
b. R6 is -alkyl-phenyl, or —CH₂-indole,
c. R8 is —CH₂-phenyl; and/or
d. R9 is:
i. RiOC(O)— and Ri is CH₃—, or phenyl-CH₂—;
ii. RiC(O)— and Ri is substituted aryl, unsubstituted aryl, substituted heteroaryl, unsubstituted heteroaryl, aryl-C1-10 alkyl-, or CH₃(CH₂)₈—; or
iii. RᵢS(O)ₙ— and Rᵢ is substituted phenyl.

32.-42. (canceled)

43. The compound of claim 31, wherein:
a. R6 is —CH₂-phenyl, or —(CH₂)₂-phenyl; and/or
b. R9 is:
i. RiC(O)— and Ri is aryl-phenyl-, heterocyclyl-phenyl-, alkyl-phenyl-, fluorophenyl, phenyl, aryl-heteroaryl-, heteroaryl-heteroaryl-, pyridine, pyrazine, indole, or phenyl-C1-10 alkyl-; or
ii. RᵢS(O)ₙ— and Rᵢ is phenyl-phenyl.

44. The compound of claim 42, wherein:
a. R6 is —CH₂-hydroxyphenyl; and/or
b. R9 is RiC(O)— and Ri is phenyl-phenyl-, heteroaryl-phenyl-, morpholine-phenyl-, (CH₃)₂CH-phenyl-, OHCH₂-phenyl-, phenyl-heteroaryl-, pyridine-isothiazole-, phenyl-alkyne-, phenyl-CH₂—, or fluoroalkyl-diazirin-phenyl-(C1-10 alkyl)-.

45.-86. (canceled)

87. The compound of claim 44, wherein:
a. Ri is diazirine-phenyl-, pyridine-phenyl-, oxadiazole-phenyl-, phenyl-pyrazole-, phenyl-methylpyrazole-, phenyl-thiazole-, phenyl-pyridine-, phenyl-furazan-, fluoromethoxy-phenyl-CH₂—; or trifluoromethyl-diazirin-phenyl-CH₂—.

88. The compound of claim 87, wherein:
a. Ri is trifluoromethyl-diazirin-phenyl-, trifluoromethoxy-phenyl-CH₂—, 4-[3-trifluoromethyl)-3H-diazirin-3-yl]phenyl-CH₂ or 3-[3-trifluoromethyl)-3H-diazirin-3-yl]phenyl-CH₂—.

89.-121. (canceled)

122. The compound of claim 88, wherein:
a. Ri is 4-(trifluoromethoxy)phenyl-CH₂—.

123.-131. (canceled)

132. The compound of claim 129, wherein:
a. R9 is RₘORᵢC(O)— and Rₘ is substituted or unsubstituted C1-10 alkyl; or substituted or unsubstituted aryl; or
b. R9 is RₘC(O)RᵢC(O)— and Rₘ is heteroaryl, or morpholine.

133. The compound of claim 132, wherein:
a. R9 is RₘORᵢC(O)— and Rₘ is substituted —CH₂—, aryl-CH₂—, phenyl-CH₂—, or phenyl.

134.-147. (canceled)

148. The compound of claim 1, which is:

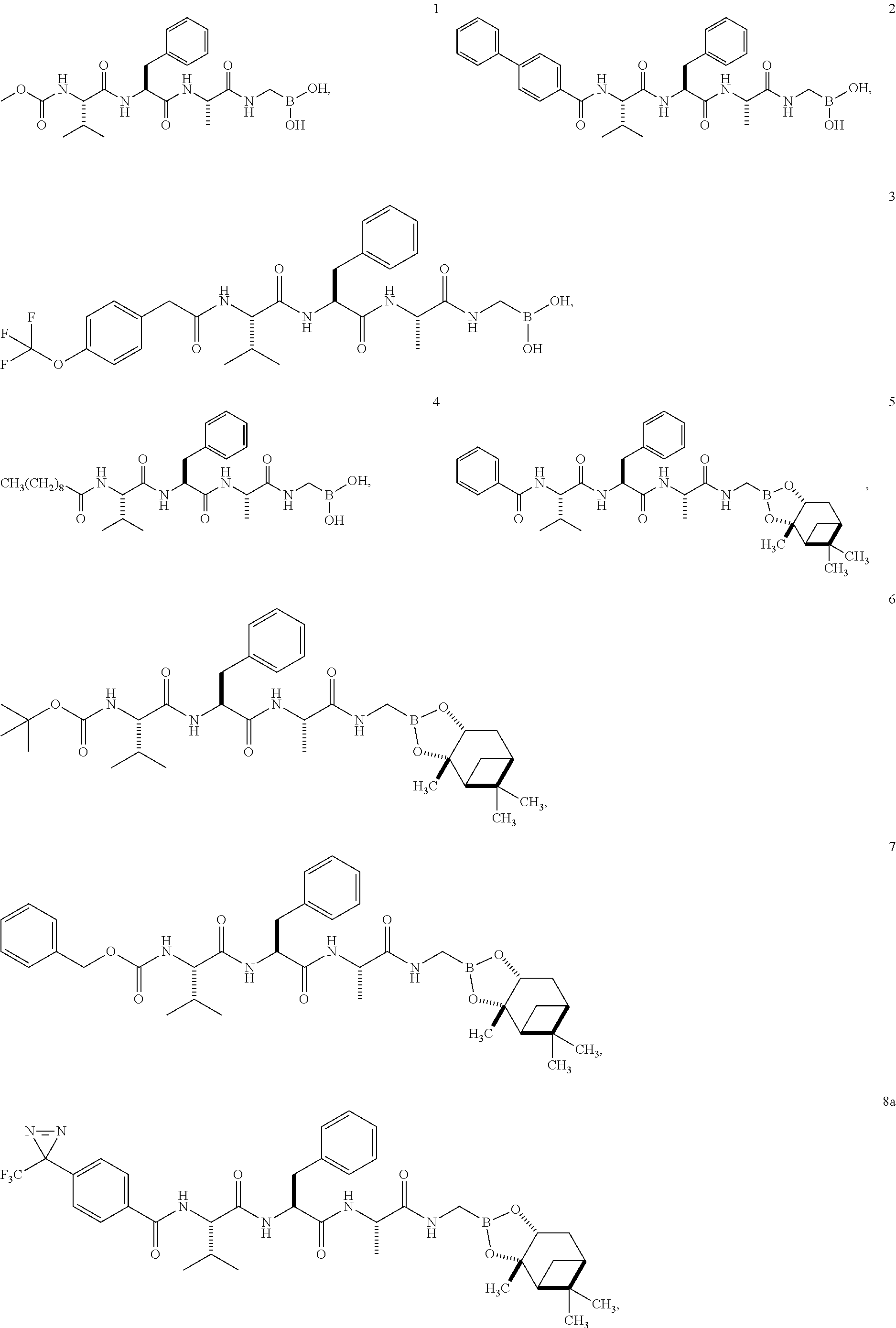

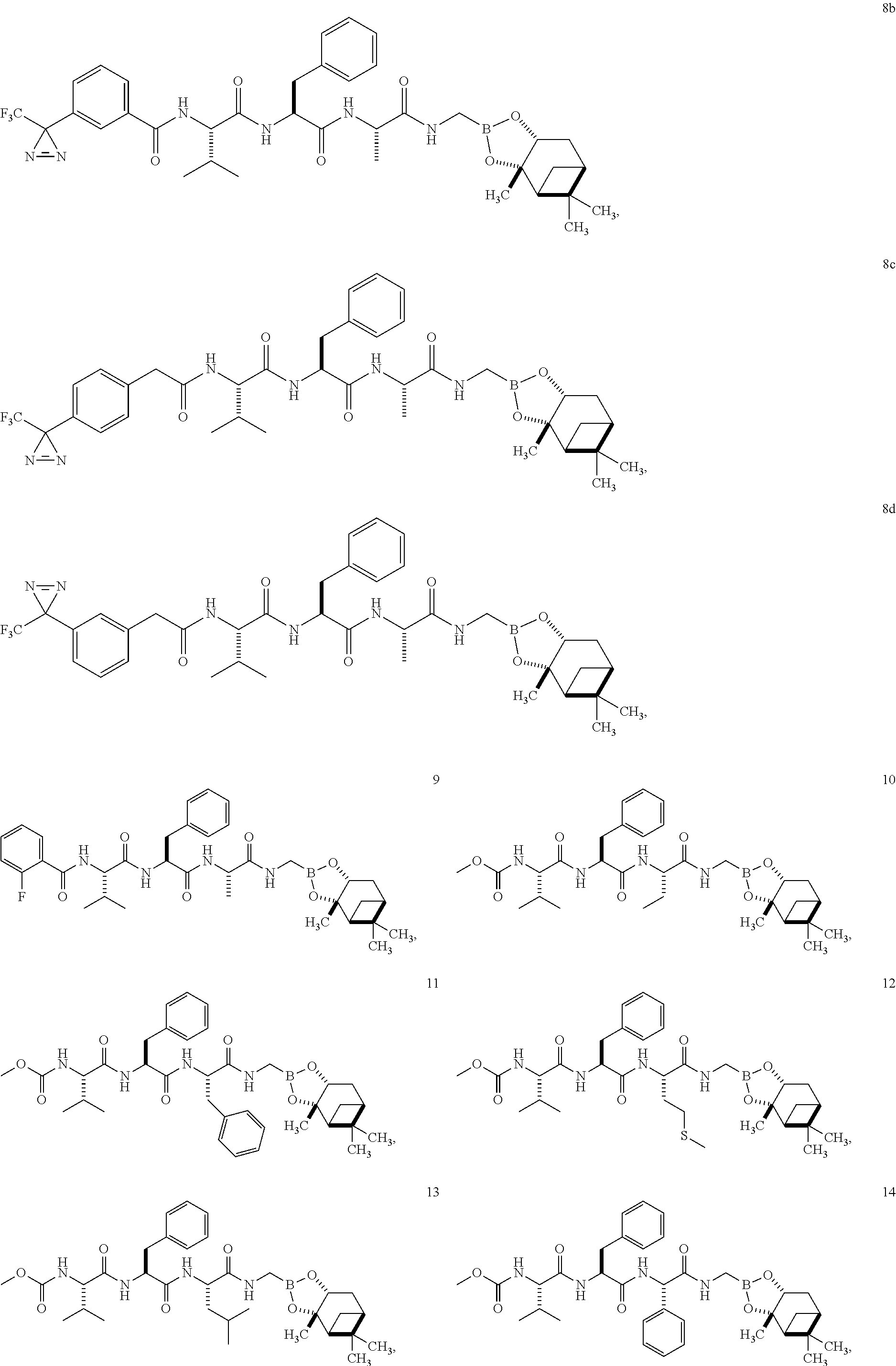

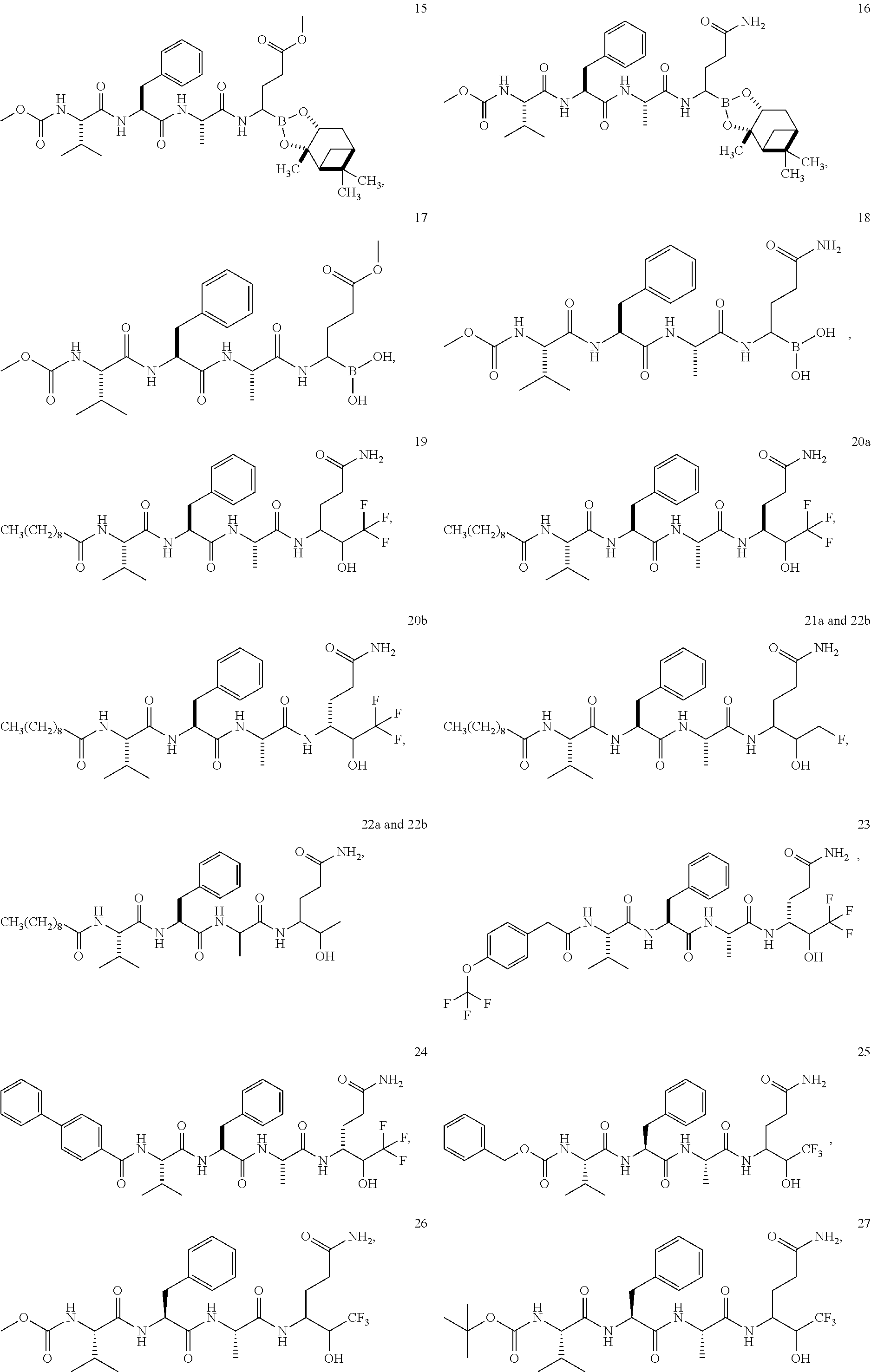

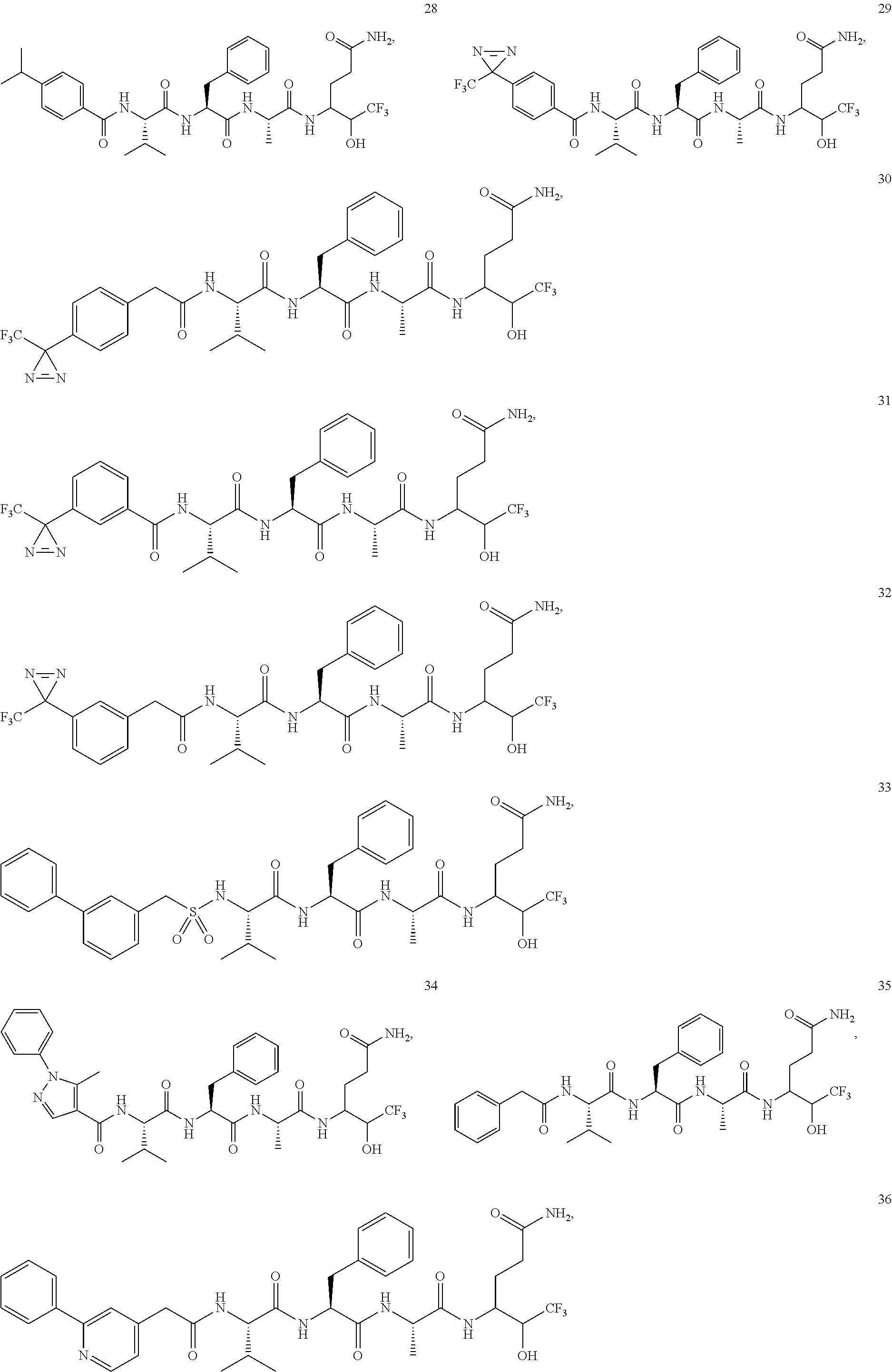

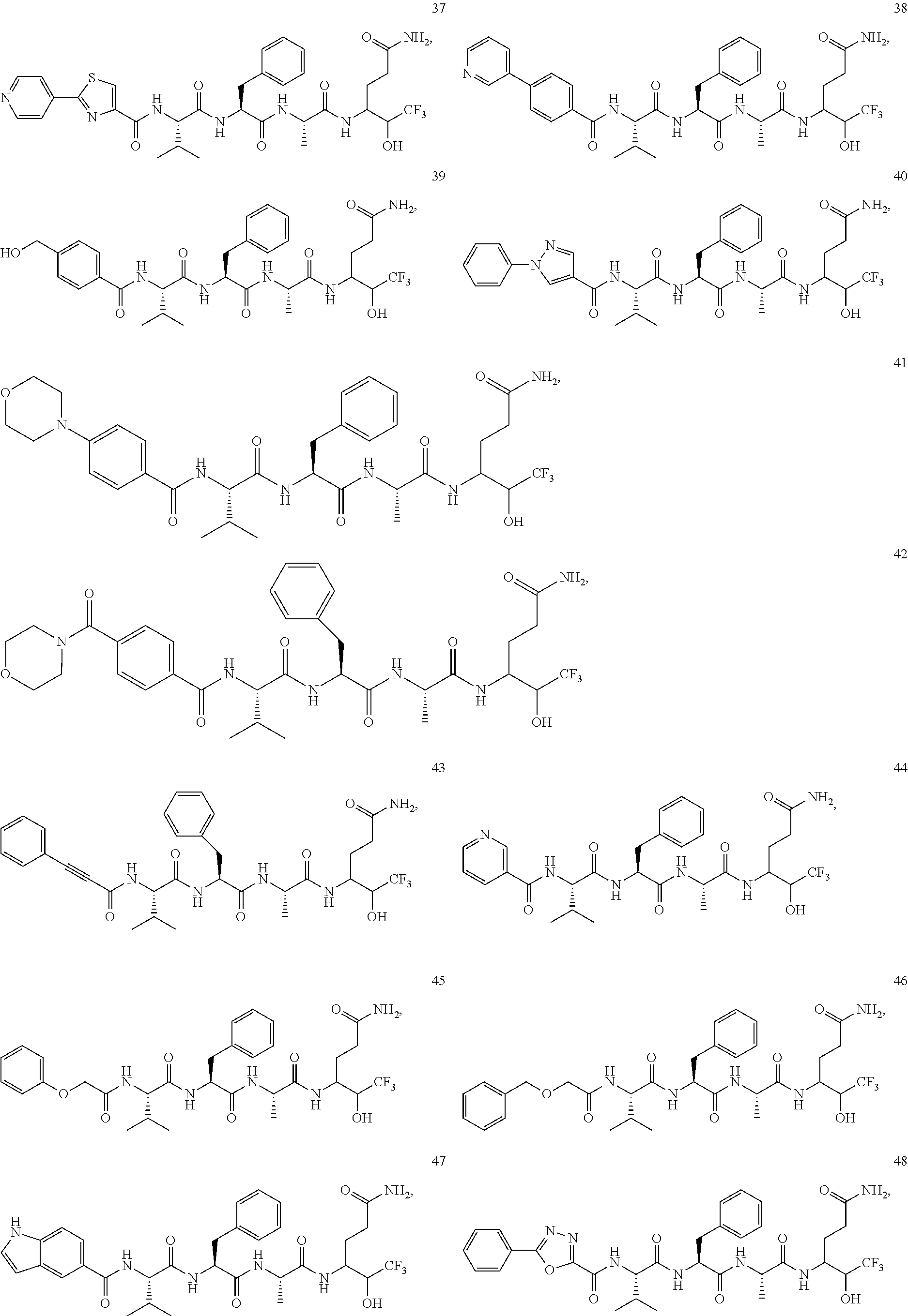

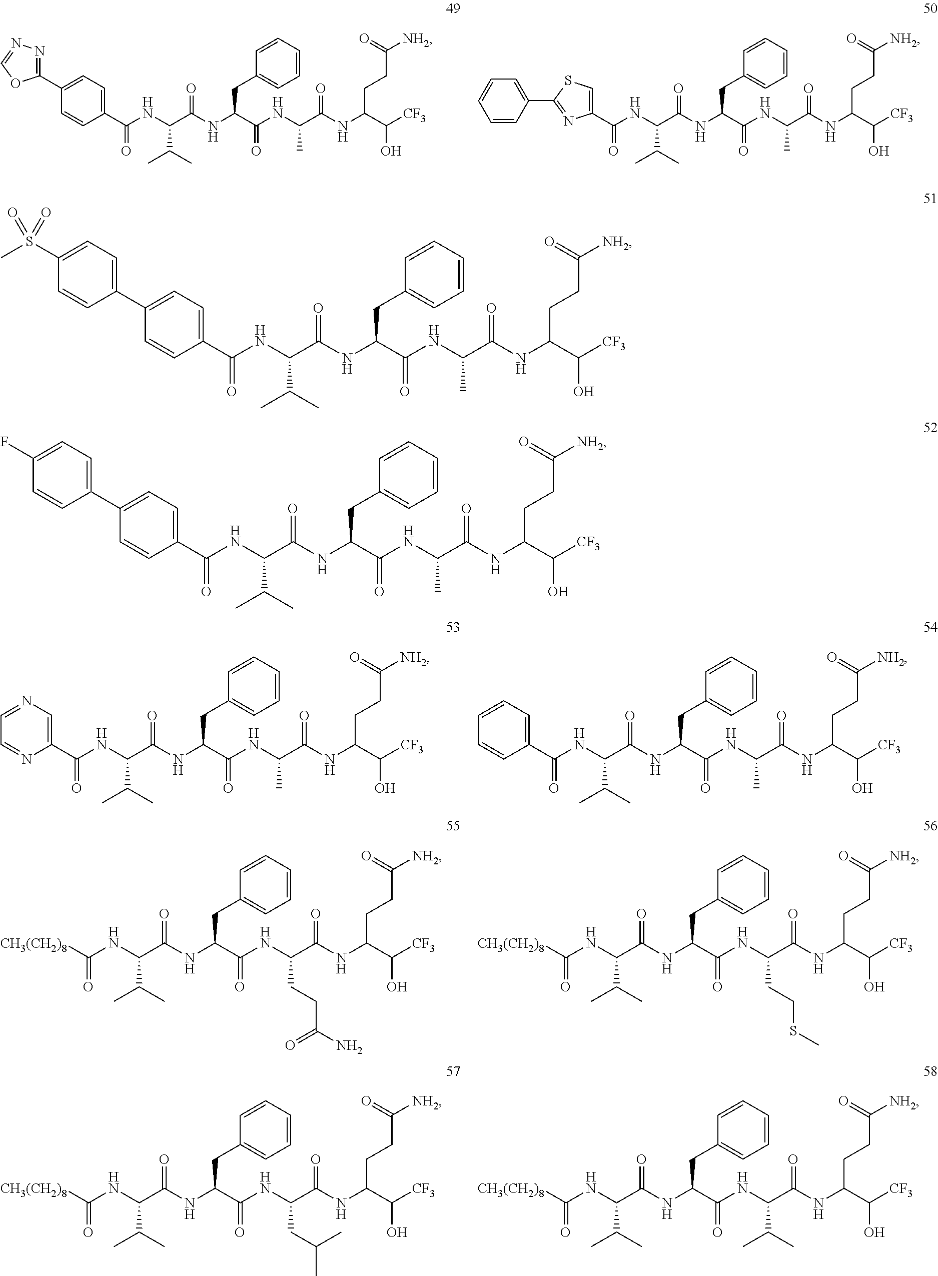

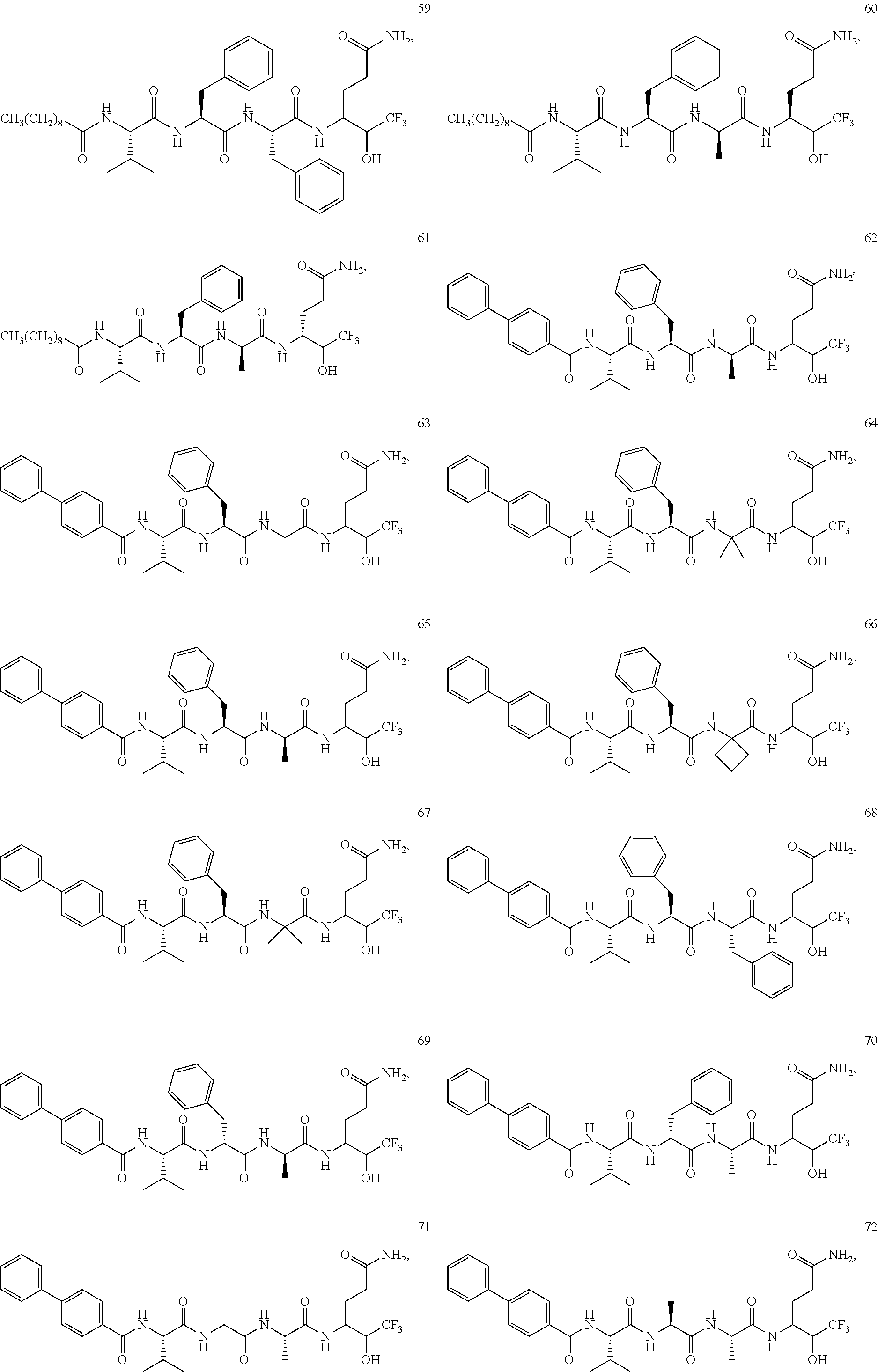

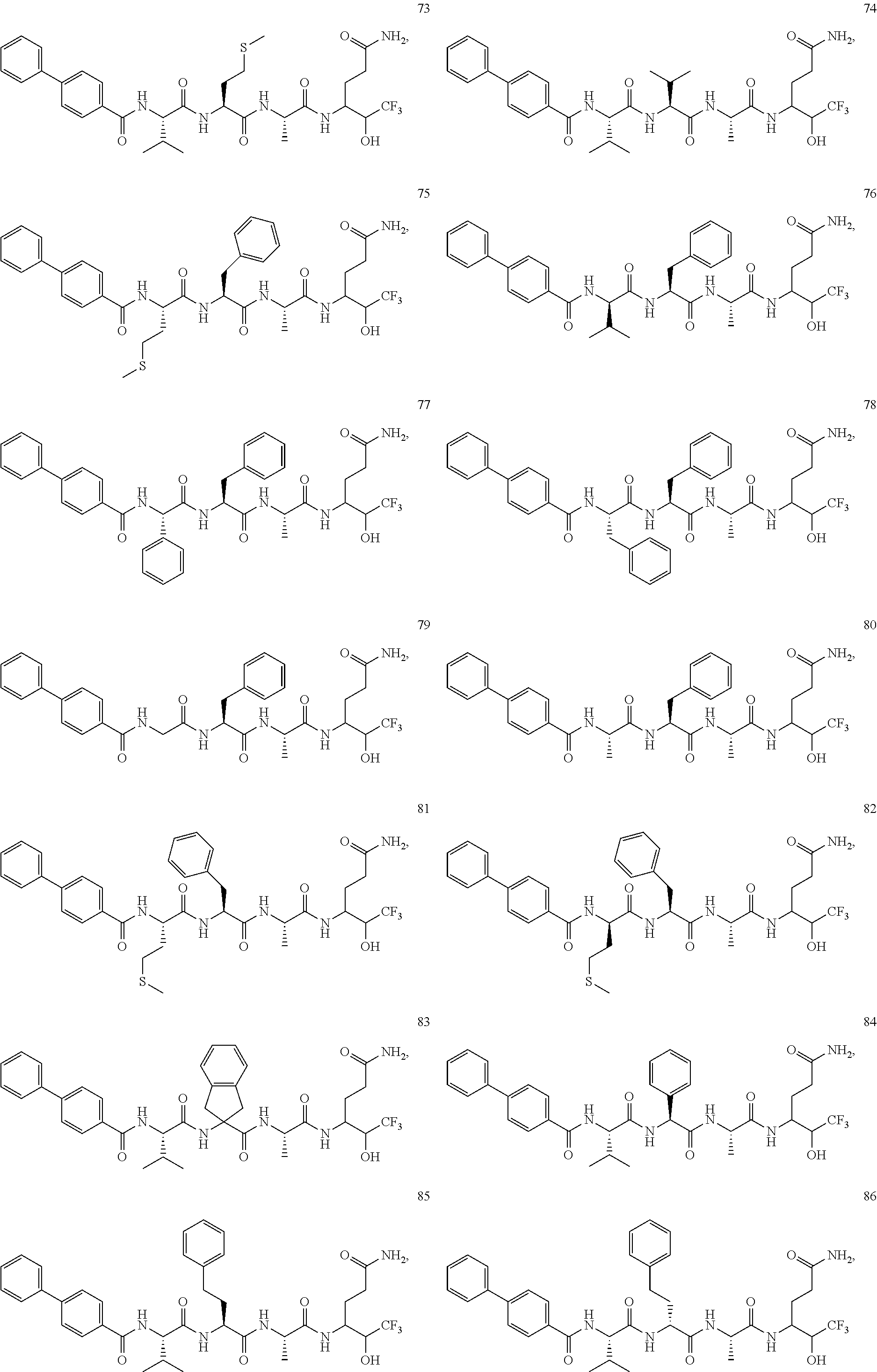

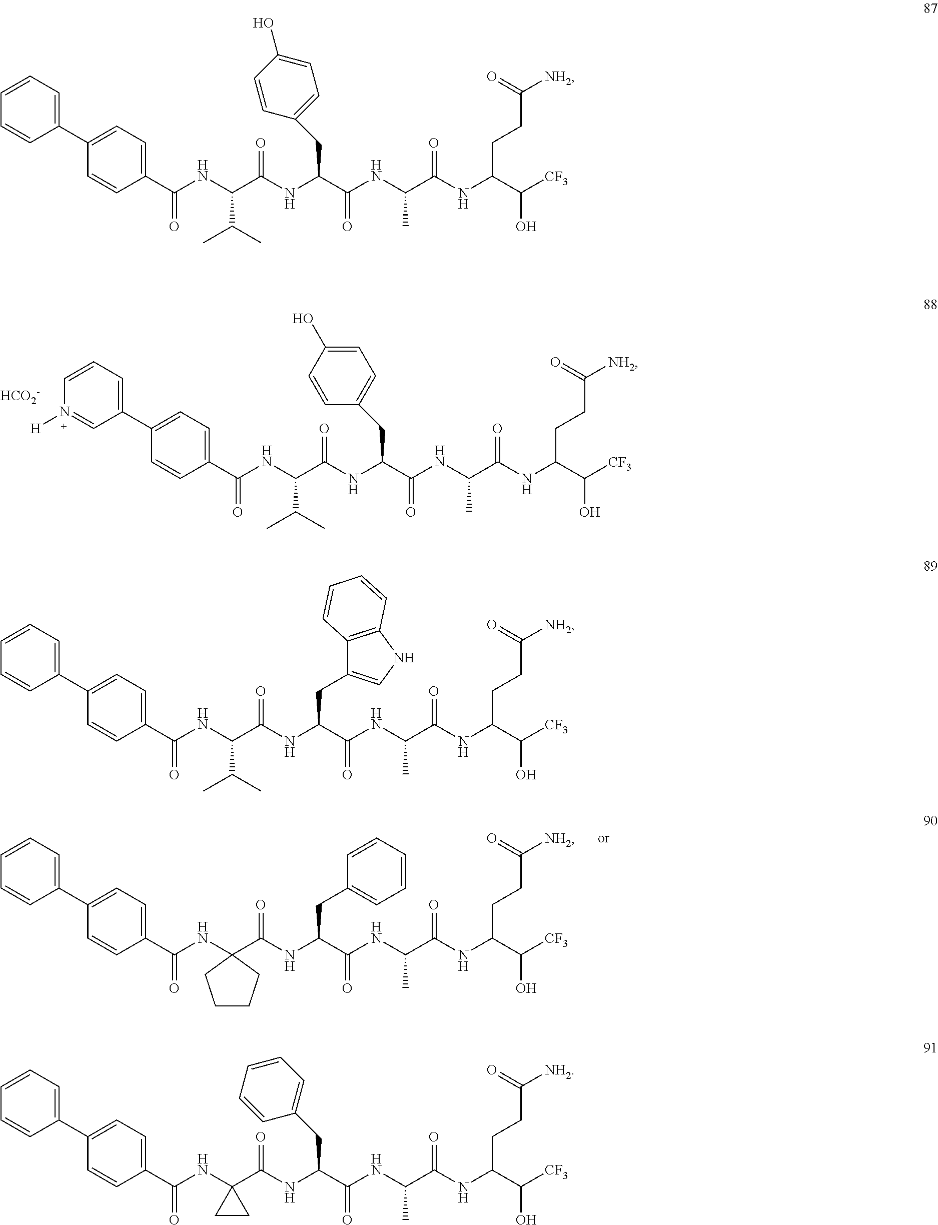

149. A pharmaceutical composition comprising at least one of the compounds as defined in claim 1.

150. The pharmaceutical composition of claim 149, further comprising (a) at least one other compound as defined in claim 1; and/or (b) at least one other active ingredient which improves a patient's lipid profile.

151. (canceled)

152. The pharmaceutical composition of claim 149, further comprising a pharmaceutically acceptable carrier or excipient.

153.-156. (canceled)

157. A method for preventing or treating an low density lipid (LDL)-cholesterol-related disease or disorder, comprising administering to a subject in need thereof a therapeutically effective amount of the compound as defined in claim 1, or of a pharmaceutical composition comprising said compound, with the proviso that the compound is not:

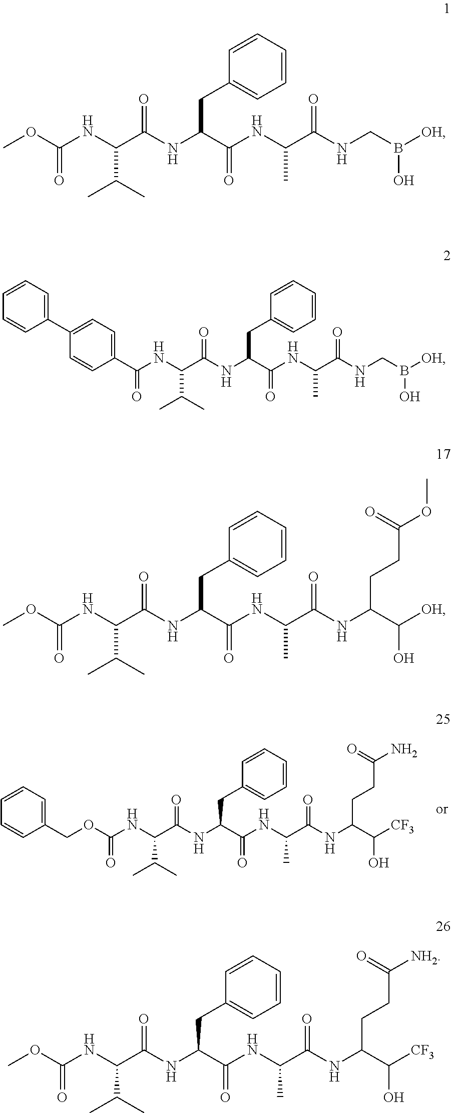

158. The method of claim 157, wherein the LDL-cholesterol-related disease or disorder is hypercholesterolemia.

159.-162. (canceled)

163. A kit for preventing or treating a low density lipid (LDL)-cholesterol-related disease or disorder in a subject comprising
(i) at least one of the compounds as defined in claim 1 or of a pharmaceutical composition comprising said compound, and
(i) (a) at least one other active ingredient which improve a patient's lipid profile;
(b) at least one other compound as defined in claim 1 or a pharmaceutical composition comprising said compound;
(c) a container for the compound and/or active ingredients; and/or
(d) instructions to use the compound for preventing or treating the LDL-cholesterol-related disease or disorder in the subject, with the proviso that the compound is not:

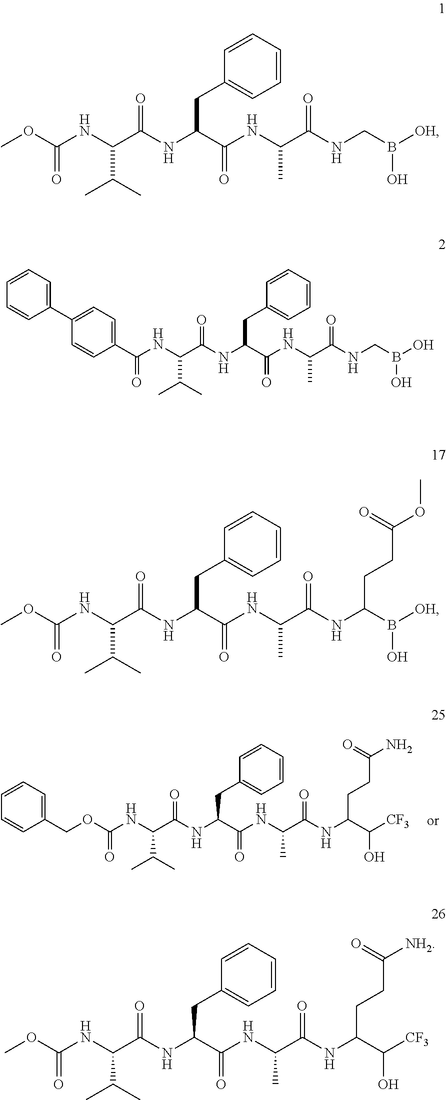

164. (canceled)
